(19) United States
(12) Patent Application Publication
(10) Pub. No.: US 2026/0265781 A1
(43) Pub. Date: Sep. 10, 2026

(54) GENERATION OF LANDING PAD CELL LINES

(71) Applicant: Bristol-Myers Squibb Company, Princeton, NJ (US)

(72) Inventors: Duncan Lochiel McVEY, Princeton, NJ (US); Charu GARG, Princeton, NJ (US); Chaojie WANG, Princeton, NJ (US); Gabriele TREMML, Princeton, NJ (US); Anurag KHETAN, Princeton, NJ (US)

(73) Assignee: Bristol-Myers Squibb Company, Princeton, NJ (US)

(21) Appl. No.: 18/724,562

(22) PCT Filed: Dec. 28, 2022
(86) PCT No.: PCT/US2022/082485

§ 371 Date: Jun. 26, 2024

Related U.S. Application Data
(60) Provisional application No. 63/294,605, filed on Dec. 29, 2021.

(51) Int. Cl. C12N 15/90; C07K 14/435; C07K 16/00; C12N 9/00; C12N 9/22; C12N 15/11; C12N 15/85; C12Q 1/6876

(52) U.S. Cl. CPC C12N 15/907; C07K 14/43595; C07K 16/00; C12N 9/226; C12N 9/93; C12N 15/11; C12N 15/85; C12Q 1/6876; C12Y 603/01002; C12N 2310/20; C12N 2840/20; C12Q 2600/154; C12Q 2600/158

(57) ABSTRACT

The disclosure provides methods to generate landing pad cells for targeted gene integration comprising integrating a landing pad plasmid into the genome of a parental cell at a targeted-integration site, for example, using homologous recombination. In one aspect, two site-specific recombination sites (SSRSs) flank a polynucleotide sequence; and, two homologous recombination sites are located 5′ and 3′ terminally with respect to the SSRSs. The two homologous recombination sites of the landing pad plasmid can recombine with corresponding homologous recombination sites on the parental plasmid, thereby integrating the landing pad plasmid at an internal location within the parental plasmid. The methods disclosed allow the generation of high expressing cell lines by identifying hot spots for targeted-integration in hot cell lines. The disclosure provides also cell and kits comprising cells and/or reagents for the generation of landing pad cells of the present disclosure. Also provided are novel hot spots for targeted integration.

The content of the electronically submitted sequence listing (Name: 3338_196PC02_Seglisting_ST26.txt; Size: 45,039,473 Bytes; and Date of Creation: Dec. 27, 2022) is herein incorporated by reference in its entirety.

### FIELD

The present disclosure provides methods for the generation of landing pad cells suitable for targeted gene integration.

### BACKGROUND

Historically cell lines have been made by transfecting cells with an expression plasmid DNA, usually linearized, that integrate in essentially a random fashion in the cellular genome. Since the plasmid provides a selective advantage such as drug resistance or auxotroph complementation only those cells with the expression plasmid survive. There is a desire to minimize the time it takes and increase predictability to make a cell line expressing a biologic of interest while maintaining an acceptable level of performance such as high titer, post-translational modifications, expression stability, cell density and viability in a bioreactor to name a few parameters.

One way to achieve this goal is the use of a technology termed Targeted Integration (TI) during cell line development in which the expression cassette(s) or an expression plasmid is inserted into the same locus of a cell line. The locus that is targeted is also referred to as the landing pad and the cell line with the landing pad as the landing pad cell line. The expression cassette(s) or expression plasmid(s) can be integrated into the landing pad by site directed recombination using Cre/Lox technology and the like sometimes referred to as recombination mediated cassette exchange (RMCE) or site specific recombination (SSR); or by using homologous recombination stimulated by Clustered Regularly Interspaced Short Palindromic Repeats (CRISPR), TALEN, or other such site-specific nucleases.

A major challenge to TI is identifying the cell line and locus to use. The cell line itself must be able to perform well and the landing pad needs to be in a locus where high transcription occurs and transcription of the biologic is not silenced such as by epigenetic modifications (a “hot spot”). While a TI landing pad host cell line should have “hot spot” in the chromosome for high expression, it is understood that this “hot spot” needs the context of a “hot cell” which supports all of the intermediate steps required for the high protein expression of the biologic. The ability to generate and identify a landing pad cell line is very difficult in part due to variability caused by the inherent plasticity of the host cell genome.

Accordingly, there is a need for efficient methods to generate landing pad cells capable of reliable and reproducible protein expression.

### BRIEF SUMMARY

The present disclosure provides a method to select a parental cell suitable for the development of a landing pad cell line comprising (i) screening and selecting a cell line with a high expression titer of a gene of interest (GOI); and, (ii) further screening a cell of (i) and selecting a cell with a low copy number of a parental plasmid comprising the nucleic acid encoding the GOI, wherein the copy number is one or two. In some aspects, the parental plasmid comprises two site-specific recombination sites (SSRS), one SSRS, or no SSRS.

The present disclosure also provides a method to select a landing pad cell comprising (i) screening for the loss of the parental plasmid or a portion thereon in a parental cell line, and selecting a cell with such a loss (deletion); and, (ii) further screening a cell of (i) for the presence of a landing pad, and selection a cell in which a landing pad in present.

Also provided is a method to select a landing pad cell comprising (i) screening for the loss of at least one parental plasmid or a portion thereon in a parental cell line, and selecting a cell with such a loss (deletion); and, (ii) further screening a cell of (i) for the presence of at least one landing pad, and selection a cell in which a landing pad in present. In some aspects, the method further comprises screening the landing pad sequence in the landing pad cell for characteristics selected from the group consisting of (i) presence or absence of regions of low complexity or high complexity; (ii) presence or absence of retrotransposon sequences; (iii) presence or absence of Alu repeats; (iv) presence or absence of long interspersed nuclear elements (LINE); (v) presence or absence of CpG islands; (vi) levels of cytosine methylation; (vii) levels of histone acetylation; (viii) presence or absence of active transcription; and, (ix) any combination thereof.

Also provided is method of generating a landing pad cell comprising (i) deleting at least one parental plasmid or a portion thereof comprising a first GOI in a parental cell line, and (ii) introducing into the cell, following the at least one deletion, a landing pad plasmid or portion thereof comprising a landing pad. In some aspects, the landing pad plasmid or portion thereof comprising a landing pad is inserted at the site of a deletion of (i). In some aspects, the landing pad plasmid or portion thereof comprising a landing pad is inserted at a site that is not the site of a deletion of (i).

The present disclosure also provides a method of generating a landing pad cell comprising integrating a landing pad plasmid into the genome of a parental cell at a targeted-integration site using homologous recombination, wherein the sequences targeted for homologous recombination are located in the parental plasmid, wherein each landing pad plasmid comprises (1) a polynucleotide sequence comprising a nucleic acid encoding at least one selection marker and/or at least one nucleic acid sequence encoding a detectable marker; (2) two site-specific recombination sites (SSRS) flanking the polynucleotide sequence of (1); and, (3) two homologous recombination sites located 5′ and 3′ terminally with respect to the SSRSs of (2), which are homologous to corresponding homologous recombination sites in a parental plasmid; and, wherein the homologous recombination sites of the landing pad plasmid recombine with the corresponding homologous recombination sites of the parental plasmid, thereby integrating the landing pad plasmid at an internal location within the parental plasmid inserted in the parent cell genomic DNA. In some aspects, the parental plasmid is located in more than one genomic locus.

The present disclosure also provides a method for identifying a landing pad cell line comprising (1) removing at least a portion of the First GOI from a parental plasmid integrated in the genomic sequence of a parental cell; (2) integrating a landing pad plasmid at alternative genomic loci; (3) screening a library of candidate cells comprising at least one copy of the landing pad plasmid integrated at at least one alternative genomic loci, wherein a candidate cell line is evaluated for one or more of the following properties: (a) cell titer is above a predetermined threshold level; (b) landing pad plasmid or landing pad copy number is at predetermined value; (c) RNA expression level above a predetermined threshold level, (d) multiple plasmid copies, if present, have a specific plasmid configuration; (e) deletion of at least a portion of the First GOI from a parental plasmid; and, (f) presence of at least one landing pad with functional SSRS. In some aspects, the parental cell is a historical cell line. In some aspects, the library of candidate cells is a library generated via random integration of the landing pad sequence at multiple locations in the genome of a parental cell. In some aspects, the method selects a hot cell with the landing pad sequence integrated in a hot spot. In some aspects, the parental cell line is a CHO cell line.

The present disclosure also provides a method of generating an expression cell comprising integrating a second GOI plasmid into the genome of a landing pad cell according to any of the methods disclosed above by using site-specific recombinase recombination, wherein the resulting expression plasmid comprises (1) a polynucleotide sequence comprising a nucleic acid encoding a second GOI; and, (2) two SSRS flanking the polynucleotide of (1); wherein the site-specific recombination sites of the landing pad plasmid recombine with the corresponding site-specific recombination sites of the second GOI plasmid, thereby integrating the GOI plasmid at an internal location within the landing pad plasmid of the landing pad cell.

Also disclosed is a method of generating an expression cell comprising:
(a) integrating a landing pad plasmid or portion thereof into the genome of a parental cell at a targeted-integration site using homologous recombination, wherein the sequences targeted for homologous recombination are located in the parental plasmid, wherein each landing pad plasmid or portion thereof comprises (1a) a polynucleotide sequence comprising a nucleic acid encoding at least one selection marker and/or at least one nucleic acid sequence encoding a detectable marker; (2a) two SSRS flanking the polynucleotide sequence of (1a); and, (3a) two homologous recombination sites located 5′ and 3′ terminally with respect to the SSRSs of (2a), which are homologous to corresponding homologous recombination sites in a parental plasmid; and, wherein the homologous recombination sites of the landing pad plasmid or portion thereof recombine with the corresponding homologous recombination sites of the parental plasmid in the landing pad at a different genomic locus, thereby integrating the landing pad plasmid or portion thereof at an internal location within the landing pad at the different genomic locus in the parent cell genomic DNA; and,
(b) integrating a second GOI plasmid into the genome of the landing pad cell using site-specific recombinase recombination, wherein the expression plasmid comprises (1b) a polynucleotide sequence comprising a nucleic acid encoding a GOI; and, (2b) two SSRS flanking the polynucleotide of (1b); wherein the site-specific recombination sites of the landing pad plasmid recombine with the corresponding site-specific recombination sites of GOI plasmid, thereby integrating the GOI plasmid at an internal location within the landing pad plasmid of the landing pad cell.

Also provided is a method of generating a landing pad cell comprising:
(a) removing at least a portion of a parental plasmid from a first hot spot location in a parental cell line; and,
(b) integrating a landing pad plasmid into a second hot spot location in the genome of the parental cell at a targeted-integration site using homologous recombination or random integration, wherein the sequences targeted for homologous recombination or random integration were present in the landing pad plasmid, wherein each landing pad plasmid comprises (1b) a polynucleotide sequence comprising a nucleic acid encoding at least one selection marker and/or at least one nucleic acid sequence encoding a detectable marker; (2b) two site-specific recombination sites (SSRS) flanking the polynucleotide sequence of (1b); and, (3b) two homologous recombination sites located 5′ and 3′ terminally with respect to the SSRSs of (2b), which are homologous to corresponding homologous recombination sites in parental cell line genome.

Also provided is a method of generating an expression cell comprising:
(a) removing a parental plasmid or a portion thereof from a first hot spot location in a parental cell line,
(b) integrating a landing pad plasmid into a second hot spot location in the genome of the parental cell at a targeted-integration site using homologous recombination, wherein the sequences targeted for homologous recombination were present in the parent cell line, wherein each landing pad plasmid comprises (1b) a polynucleotide sequence comprising a nucleic acid encoding at least one selection marker and/or at least one nucleic acid sequence encoding a detectable marker; (2b) two site-specific recombination sites (SSRS) flanking the polynucleotide sequence of (1b); and, (3b) two homologous recombination sites located 5′ and 3′ terminally with respect to the SSRSs of (2b), which are homologous to corresponding homologous recombination sites in a parental cell line, wherein the homologous recombination sites of the landing pad plasmid recombine with the corresponding homologous recombination sites of the parental cell line, thereby integrating the landing pad plasmid at an internal location within the parental cell genomic DNA; and,
(c) integrating a GOI plasmid into the genome of the landing pad cell using site-specific recombinase recombination, wherein the expression plasmid comprises (1c) a polynucleotide sequence comprising a nucleic acid encoding a first GOI; and, (2c) two SSRS flanking the polynucleotide of (1c); wherein the site-specific recombination sites of the landing pad plasmid recombine with the corresponding site-specific recombination sites of GOI plasmid, thereby integrating the GOI plasmid at an internal location within the landing pad plasmid of the landing pad cell.

In some aspects of the methods disclosed above, the landing pad cell comprises a plasmid having a topology corresponding to the description:

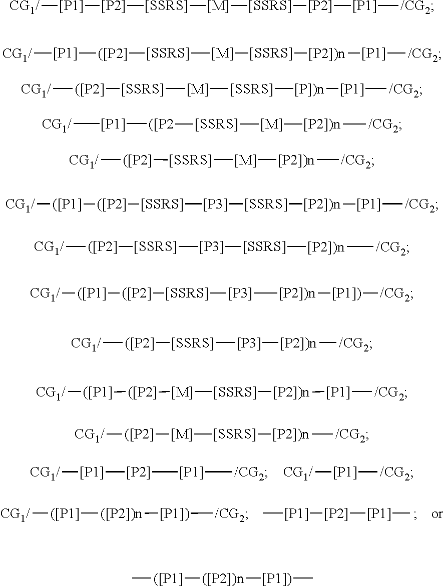

wherein:
CG₁ and CG₂ are parental cell genomic sequences flanking the inserted plasmid;
[P1] is a polynucleotide sequence derived from a parental plasmid;
[P2] are polynucleotide sequences derived from a landing pad plasmid;
[M] is a polynucleotide sequence comprising at least one selection marker and/or at least one nucleic acid sequence encoding a detectable marker; and,
[SSRS] are site-specific recombination sites (SSRS).
n is an integer between 1 and 10 between 1 and 10, such as 1, 2, 3, 4, 5, 6, 7, 8, 9 or 10.

In some aspects of the methods disclosed above, the topology of the plasmid integrated in the expression cells corresponds to the description:

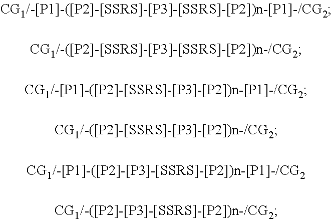

wherein
CG₁ and CG₂ are parental cell genomic sequences flanking the inserted plasmid;
[P1] is a polynucleotide sequence derived from a parental plasmid;
[P2] are polynucleotide sequences derived from a landing pad plasmid;
[P3] is a polynucleotide sequence derived from a plasmid comprising a gene of interest (GOI); and,
[SSRS] are site-specific recombination sites (SSRS).
n is an integer between 1 and 10, such as 1, 2, 3, 4, 5, 6, 7, 8, 9 or 10.

In some aspects of the methods disclosed above, the homologous recombination is mediated by a CRISPR/Cas system, a TALEN system, or a ZFN system. In some aspects, the CRISPR/Cas system further comprises a single guide RNA (sgRNA). In some aspects of the methods disclosed above, the site-specific recombinase recombination site (SSRS) is a Tyr-recombinase site, a Tyr-integrase site, a Serine-resolvase/invertase site, or a Serine-integrase site. In some aspects, the Tyr-recombinase site comprises a Cre, Dre, Flp, KD, B3, or B3 Tyr-recombinase site. In some aspects, the Tyr-integrase site comprises a λ (Lambda), HK022, or HP1 Tyr-integrase site. In some aspects, the Serine-resolvase/invertase site comprises a γδ (Gammadelta), ParA, Tn3, or Gin Serine-resolvase/integrase site. In some aspects, the Serine-integrase site comprises a PhiC31, Bxb1, pr R4 Serine-integrase site. In some aspects, the Tyr-recombinase site comprises a Cre Tyr-recombinase site. In some aspects, the SSRS is a LoxP site. In some aspects, the LoxP site comprises a nucleic acid sequence set forth in SEQ ID NO:1 (wild type LoxP). In some aspects, the LoxP site comprises a mutant LoxP site. In some aspects, the mutant LoxP site comprises a nucleic acid sequence set forth in SEQ ID NO:2 (mutant LoxP). In some aspects, the mutant LoxP site comprises a nucleic acid selected from the group consisting of SEQ ID NO: 3 (Lox 511); SEQ ID NO: 4 (Lox 5171); SEQ ID NO: 5 (Lox 2272); SEQ ID NO: 6 (Lox M2); SEQ ID NO: 7 (Lox M3); SEQ ID NO: 8 (Lox M7); SEQ ID NO: 9 (Lox M11); SEQ ID NO: 10 (Lox 71); and, SEQ ID NO: 11 (Lox 66). In some aspects, the mutant LoxP site comprises any LoxP site disclosed in the present specification. In some aspects, the Tyr-recombinase site comprises a Flp Tyr-recombinase site. In some aspects, the SSRS is a short flippase recognition target (FRT) site. In some aspects, the SSRS comprises any FRT site sequence disclosed in the present specification. In some aspects, the Serine-integrase site comprises an att site, e.g., an attP or attB site. In some aspects, the SSRS comprises any att site disclosed in the present application.

In some aspects of the methods disclosed above, the at least one selection marker and/or at least one nucleic acid sequence encoding a detectable marker is glutamine synthetase (GS) and/or dihydrofolate reductase (DHFR). In some aspects, the at least one selection marker and/or at least one nucleic acid sequence encoding a detectable marker is a drug resistance gene. In some aspects, the drug resistance gene is an antibiotic resistance gene. In some aspects, the antibiotic resistance gene is a puromycin resistance gene. In some aspects, the puromycin resistance gene is puromycin-N-acetyltransferase. In some aspects, the at least one selection marker and/or at least one nucleic acid sequence encoding a detectable marker comprises a protein. In some aspects, the protein is a fluorescent protein. In some aspects, the fluorescent protein is mCherry. In some aspects, the fluorescent protein comprises GFP, ZsGreen1, AcGFP1, EGFP, GFPuv, AcGFP, EBFP, EYFP, ECFP, tdTomato, mCherry, DsRed, AmCyan, ZsGreen, ZsYellow, DsRed2, DsRed-Express, HcRed, AsRed, mOrange, mOrange2, mPlum, mStrawberry, mBanana, YFP, mRaspberry, HcRed1, E2-Crimson, or any combination thereof.

In some aspect of the methods disclosed above, the cell is a Chinese Hamster Ovary (CHO) cell. In some aspects, the cell is HEK293 or NS0.

In some aspects of the methods disclosed above, the nucleic acid encoding the GOI encodes at least one polypeptide. In some aspects, the at least one polypeptide is an antibody or a fusion protein. In some aspects, the expression plasmid comprises one, two, or more than two copies of the GOI, a detectable marker, or a combination thereof.

In some aspects, the methods disclosed above further comprise determining the expression of the GOI, detectable marker, or combination thereof. In some aspects, the expression of the GOI is determined quantitatively and/or qualitatively. In some aspects, the expression of the GOI is determined by cell sorting, FACS, cell surface staining, Western blot, Northern blot, column chromatography, capillary electrophoresis, microfluidics, UV absorbance, immunohistochemistry, cell size, secreted protein levels, transcript levels, or any combination thereof.

In some aspects of the methods disclosed herein, the landing pad plasmid or expression plasmid is integrated with a copy number of 1 in the genome of the cell. In some aspects, the landing pad plasmid or expression plasmid is integrated with a copy number of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 in the genome of the cell.

In some aspects of the methods disclosed herein (i) the 5′ homologous recombination site comprises a polynucleotide sequence of SEQ ID NO: 18 or a subsequence thereof, and the 3′ homologous recombination site comprises a polynucleotide sequence of SEQ ID NO: 19 or a subsequence thereof; (ii) the 5′ homologous recombination site comprises a polynucleotide sequence of SEQ ID NO: 114 or a subsequence thereof, and the 3′ homologous recombination site comprises a polynucleotide sequence of SEQ ID NO: 115 or a subsequence thereof; or (iii) the 5′ homologous recombination site and the 3′ homologous recombination site comprise polynucleotide sequences flanking the parental plasmid.

In some aspects of the methods disclosed herein the parental plasmid comprises an open reading frame (ORF) encoding a first GOI such as an antibody.

The present disclosure provides a landing pad cell comprising a plasmid having a topology corresponding to the description

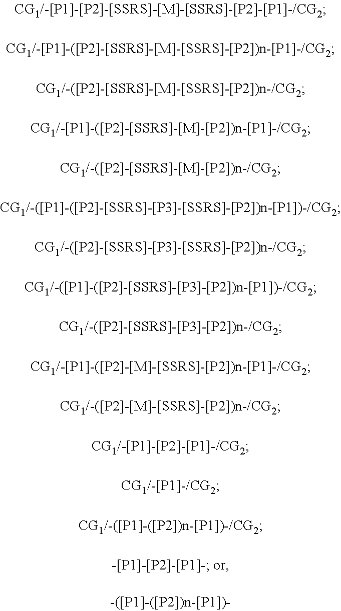

wherein
CG₁ and CG₂ are parental cell genomic sequences flanking the inserted plasmid;
[P1] is a polynucleotide sequence derived from a parental plasmid;
[P2] are polynucleotide sequences derived from a landing pad plasmid;
[M] is a polynucleotide sequence comprising at least one selection marker and/or at least one nucleic acid sequence encoding a detectable marker; and,
[SSRS] are site-specific recombination sites (SSRS).
n is an integer between 1 and 10 between 1 and 10, such as 1, 2, 3, 4, 5, 6, 7, 8, 9 or 10.

The present disclosure provides an expression cell comprising a plasmid with a topology corresponding to the description

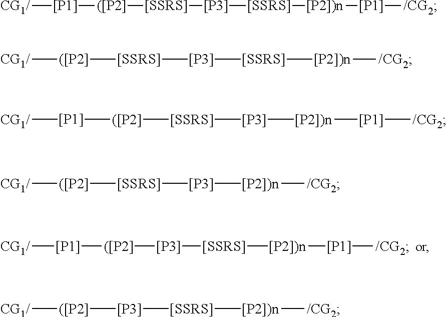

wherein
CG₁ and CG₂ are parental cell genomic sequences flanking the inserted plasmid;
[P1] is a polynucleotide sequence derived from a parental plasmid;
[P2] are polynucleotide sequences derived from a landing pad plasmid;
[P3] is a polynucleotide sequence derived from a plasmid comprising a gene of interest (GOI); and,
[SSRS] are site-specific recombination sites (SSRS).
n is an integer between 1 and 10, such as 1, 2, 3, 4, 5, 6, 7, 8, 9 or 10.

The present disclosure provides a cell line produced by any of the methods disclosed herein. Also provided is a kit comprising a cell disclosed herein or a cell generated according to any of the methods disclosed herein and instructions for their use.

The present disclosure also provides an isolated cell comprising a polynucleotide sequence which comprises a nucleic acid encoding a gene of interest (GOI) integrated at a specific locus of the genome of the cell, wherein the locus is a nucleotide position or polynucleotide subsequence within SEQ ID NO: 20 or SEQ ID NO: 116.

Also provided is a method comprising introducing into CHO cells a polynucleotide sequence which comprises a nucleic acid encoding a gene of interest (GOI) and obtaining a CHO cell wherein the nucleic acid is integrated at a specific locus of the genome of the CHO cell, wherein the locus is a nucleotide position or polynucleotide subsequence within SEQ ID NO: 20 or SEQ ID NO: 116. Also provided is a method comprising providing a cell comprising a polynucleotide sequence which comprises a nucleic acid encoding a gene of interest (GOI), wherein the polynucleotide sequence comprises the nucleic acid encoding a gene of interest (GOI) operably linked to a promoter, wherein the nucleic acid is integrated at a specific locus of the genome of the CHO cell, and wherein the locus is a nucleotide position or polynucleotide subsequence within SEQ ID NO: 20 or SEQ ID NO: 116. In some aspects of the methods or isolated cells disclosed herein (i) the nucleotide subsequence within SEQ ID NO: 20 comprises the sequence set forth in SEQ ID NO:21; or (ii) the nucleotide subsequence within SEQ ID NO: 116 comprises the sequence set forth in SEQ ID NO: 117. In some aspects of the methods or isolated cells disclosed herein (i) the nucleotide subsequence from within SEQ ID NO: 20 consists of the sequence set forth in SEQ ID NO:21; or (ii) the nucleotide subsequence from within SEQ ID NO: 116 consists of the sequence set forth in SEQ ID NO: 117. In some aspects of the methods or isolated cells disclosed herein (i) the nucleotide subsequence from within SEQ ID NO: 20 is a subsequence upstream (towards the 5′ end of SEQ ID NO: 20) with respect to the sequence set forth in SEQ ID NO:21; or (ii) the nucleotide subsequence from within SEQ ID NO: 116 is a subsequence upstream (towards the 5′ end of SEQ ID NO: 116) with respect to the sequence set forth in SEQ ID NO: 117. In some aspects of the methods or isolated cells disclosed herein (i) the nucleotide subsequence from within SEQ ID NO: 20 is a subsequence downstream (towards the 3′ end of SEQ ID NO: 20) with respect to the sequence set forth in SEQ ID NO:21, or (ii) the nucleotide subsequence from within SEQ ID NO: 116 is a subsequence downstream (towards the 3′ end of SEQ ID NO: 116) with respect to the sequence set forth in SEQ ID NO:117.

In some aspects, the methods, cells, cell lines, or kits disclosed herein comprise or comprise the use of at least two landing pad plasmids or at least two expression plasmids. In some aspects, the two landing pad plasmids or two expression plasmids are in a configuration selected from the group consisting of head-to-head, tail-to-tail, tail-to-head, and head-to-tail. In some aspects, each expression plasmid comprises at least a nucleic acid encoding a gene of interest (GOI). In some aspects, all GOI are the same. In some aspects, all GOI are different. In some aspects, at least one GOI is different from the rest. In some aspects, a first GOI comprises a heavy chain (HC) of an antibody, and a second GOI comprises a light (LC) of an antibody. In some aspects, at least one expression plasmid is bicistronic. In some aspects, the bicistronic expression plasmid encodes a first GOI comprising a HC of an antibody, and a second GOI comprising a LC of an antibody. In some aspects, at least one landing pad plasmid is addressable. In some aspects, each landing pad plasmid comprises two Lox sites. In some aspects, the Lox sites are Lox P and Lox 511. In some aspects, each landing pad plasmid comprises a Lox site and an Frt site. In some aspects, each landing pad plasmid comprises one or two aat sites. In some aspects, each landing pad plasmid is addressable. In some aspects, each addressable landing pad plasmid comprises a pair of addressable SSRS which are unique to the landing pad. In some aspects, at least one pair of addressable SSRS is a pair of Lox sites. In some aspects, at least one pair of Lox sites is Lox 511 and Lox P. In some aspects, at least one pair of Lox sites is Lox m3 and Lox m7. In some aspects, a first addressable landing pad plasmid comprises an Lox 511 and Lox P pair of Lox sites, and a second addressable landing pad plasmid comprises an Lox m3 and Lox m7 pair of Lox sites. In some aspects, each addressable landing pad plasmid comprises a non cross-compatible att site.

The present disclosure also provides a cell comprising a heterologous polynucleotide sequence which comprises a nucleic acid encoding a gene of interest (GOI), a selection marker, a detectable marker, or a combination thereof integrated at a specific locus of the genome of the cell, wherein the locus is a nucleotide position or polynucleotide subsequence within SEQ ID NO: 20 or SEQ ID NO: 116 or an orthologous sequence thereof. Also provided is a cell comprising a heterologous polynucleotide sequence which comprises a nucleic acid encoding a gene of interest (GOI), a selection marker, a detectable marker, or a combination thereof integrated at a specific locus of the genome of the cell, wherein the locus is a nucleotide position or polynucleotide subsequence within SEQ ID NO: 21 or SEQ ID NO: 117 or an orthologous sequence thereof. Also provided is a cell comprising a heterologous polynucleotide sequence which comprises a nucleic acid encoding a gene of interest (GOI), a selection marker, a detectable marker, or a combination thereof integrated at a specific locus of the genome of the cell, wherein the locus is a polynucleotide subsequence that overlaps or encompasses SEQ ID NO: 20 or SEQ ID NO: 116 or an orthologous sequence thereof. Also provided is a cell comprising a heterologous polynucleotide sequence which comprises a nucleic acid encoding a gene of interest (GOI), a selection marker, a detectable marker, or a combination thereof integrated at a specific locus of the genome of the cell, wherein the locus is a polynucleotide subsequence that overlaps or encompasses SEQ ID NO: 21 or SEQ ID NO: 117 or an orthologous sequence thereof. In some aspects, the cell is a CHO cell. In some aspects, the orthologous sequence has about 70%, about 75%, about 80%, about 85%, about 90%, about 95%, about 96%, about 96%, about 97%, about 98% or about 99% sequence identity to SEQ ID NO: 20, 21, 116, 117 or subsequence thereof. In some aspects, sequence identity is determined via pairwise alignment using an implementation of the Needleman-Wunsch algorithm. In some aspects, the cell comprises two landing pad plasmids or two expression plasmids. In some aspects, the cell comprises more than two landing pad plasmids or more than two expression plasmids. In some aspects, the two landing pad plasmids are addressable.

### BRIEF DESCRIPTION OF THE DRAWINGS/FIGURES

FIG. 1 is a schematic representation depicting a standard expressing cell line development strategy in which a cell is transfected with an expression plasmid resulting in its integrating at random locations in the cell's genome.

FIG. 2 summarizes a strategy used to identify two parental cell lines suitable for landing pad cell development. The parental cell lines 1 and 2 are cell lines that express a monoclonal antibody from the parental plasmid directed at protein 1 and to protein 2, respectively. The arrow and GS represent glutamine synthetase complementation, mAb=monoclonal Antibody; LC=light chain; HC=heavy chain; Copy number=number expression plasmids in the cell line (each expression plasmid contains a LC and HC expression cassette. Copy number determined by qPCR using GAPDH as internal control); spPCR=splinkeret PCR (technology that allows for the identification of plasmid junction sequences). Level of LC RNA and HC RNA was normalized to that found for antibody against protein 1 (i.e., Antibody against protein 1=1.00).

FIG. 3 is a simplified depiction of the parental plasmids showing the configuration found in both cell line 1 and cell line 2. The parental plasmids in both cell lines are in a head to tail configuration. The configuration in cell line 1 and cell line 2 was established by Southern blot analysis and determination of plasmid sequence junctions in which the plasmid-plasmid fusion was detected. The arrow and GS represent glutamine synthetase complementation.

FIGS. 4A and 4B show respectively two strategies to generate a landing pad cell line comprising site directed recombination sites such as LoxP. In both strategies the landing pad plasmid is introduced into the cell by homologous recombination stimulated by restricting the parental cell line's genome with a site-specific nuclease, e.g., a CRISPR-associated nuclease (Cas), represented by the scissors. In FIG. 4A the parental cell line is identified based on its performance and number of sites the expression plasmid/cassette are found in its genome. In FIG. 4B the parental cell line of FIG. 4A is used as a landing pad cell line. In both FIGS. 4A and 4B knowledge of the sequence of the cellular genome is needed, and sequences homologous to the cellular genome (Homology 1, Homology 2) cloned to flank the expression cassette. mCherry represents the open reading frame that encodes a fluorescent marker, LoxP sites are sequences used by the Cre recombinase, the arrow and GS represent glutamine synthetase complementation, arrow and Puro represent puromycin resistance.

FIGS. 5A and 5B schematically present the universal TI strategy of the present disclosure. The TI technology disclosed herein comprises the use of site-specific endonuclease(s) directed at parental plasmid sequences in the parental cell line (FIG. 5A) or in the landing pad cell line (FIG. 5B) to stimulate homologous recombination with a second DNA. The parental cell line in (FIG. 5A) can also serve as a landing pad cell line (FIG. 5B). These strategies lie in contrast with technology where knowledge and use of genomic sequences is required (see, e.g., FIGS. 4A, and 4B). The boxes with vertical and wavy lines next to the genome sequences represent regions of homology between different plasmids. The solid box next to each homology region a sequence present in the parental cell line of FIG. 5A or landing pad cell line of FIG. 5B targeted by CRISPR/Cas, but absent in the plasmids to be recombined into these cell lines. The scissors represent CRISPR/Cas, mCherry open reading frame encodes a fluorescent marker, LoxP sites are sequences used by the Cre recombinase, the arrow and GS represent glutamine synthetase complementation, arrow and Puro represent puromycin resistance.

FIG. 5C depicts the sequence organization of an expression plasmid (P4) in an expression cell generated according to the methods disclosed herein. The diagrams show the location of sequences originating from the parental plasmid (P1), from the landing pad plasmid (P2), and the second GOI plasmid (P3). “Cellular genome” indicates flanking genomic sequences.

FIG. 5D shows he universal TI strategy using a single SSRS site. Here, site specific endonuclease is directed at the parental plasmid sequences in the parental cell line to stimulate homologous recombination. The boxes with vertical and wavy lines next to the genome sequences represent regions of homology between different plasmids. The solid box next to each homology region a sequence present in the parental cell line targeted by a Sequence Specific endonuclease, e.g., CRISPR/Cas, but absent in the plasmids to be recombined into these cell lines. A single SSRS site is present at the landing pad cell line, shown here is using attB as an example. Through the single SSRS site, the GOI plasmid (P3) will be inserted into the targeted locus. The scissors represent a Sequence Specific endonuclease, e.g., CRISPR/Cas, mCherry open reading frame encodes an exemplary fluorescent marker, attB and attP sites are sequences used by integrases. The arrow represents a promoter, and GS represent glutamine synthetase complementation. An is a polyA signal sequence. mAb is a monoclonal antibody expression cassette, including its own promoter and polyA signal.

FIG. 5E shows a TI strategy using the cellular genomic sequence for homologous recombination to create the landing pad. Here, site specific endonuclease is directed at the cellular genomic sequence to stimulate homologous recombination. The genome sequence represent regions of homology between the landing pad plasmid and the parental cell. The solid box next to each homology region a sequence present in the parental cell line targeted by CRISPR/Cas, but absent in the plasmids to be recombined into these cell lines. A single SSRS site is present at the landing pad cell line, shown here is using attB as an example. Through the single SSRS recombination, the GOI plasmid (P3) will be inserted into the targeted locus. The scissors represent CRISPR/Cas, mCherry open reading frame encodes a fluorescent marker. attB and attP sites are sequences used by integrases. The arrow represent a promoter, and GS represent glutamine synthetase complementation. An is a polyA signal sequence. mAb represents a monoclonal antibody expression cassette, including its own promoter and polyA.

FIG. 5F depicts the sequence organization of an expression plasmid (P5) in an expression cell line generated according to methods described in FIG. 5E or using random integration into a new genomic locus. The diagrams show the location of sequences originating from the landing pad plasmid (P2), and the second GOI plasmid (P3). “Cellular genome” indicates flanking genomic sequences. Since plasmid P1 is either fully removed or does not present in the locus, there is no P1 portion in this expression plasmid configuration.

FIGS. 6A and 6B summarize the generation of a landing pad cell line according to the present disclosure. FIG. 6A shows replacement of a plasmid encoding a monoclonal antibody (mAb) in a parental cell line with a portion of the landing pad plasmid (e.g., linear plasmid comprising open reading frame encoding mCherry and puromycin resistance gene, flanked by LoxP sites) to generate the landing pad cell line expressing a marker (e.g., mCherry). The description of components in FIG. 6A can be found above, in the description of FIGS. 5A and 5B. FIG. 6B shows the increased frequency in generating the mCherry landing pad cell line stimulated by the presence of the single guide RNA (sgRNA) required by the CRISPR/Cas technology. The percent of clones (~25%) with desired phenotype of a single mCherry expression cassette and no mAb being present. The landing pad cell line used for TI was identified by its expression level (mean fluorescent intensity MFI), transcript levels, and stability of these two parameters when cells were passaged.

FIGS. 7A and 7B show the practical application of the methodologies for targeted integration presented in FIGS. 5A, 5B, 6A, and 6B cell. In FIG. 7A the mCherry expression cassette is exchanged with one expressing antibody against protein 3 (mAb 3) with the use of Cre recombinase. The cells that expressed only mAb 3 were single cell cloned by Berkley Lights (BL), and FACS technologies. The cells were expanded and assessed for protein expression in an AMBR® 15, AMBR® 250 bioreactor systems and by 24 deep well fed batch (24DW FB). FIG. 7B shows that the resulting cell population after selection for GS complementation was screened by FACS for cell surface expression of protein 3 (vertical axis), and expression of mCherry (horizontal axis). 5.24% of the cells expressed protein 3 only, 90.06% expressed both proteins, 4.12% expressed only mCherry, and 0.68% expressed neither protein. Cells that only have cell surface staining of mAb against protein 3 (mAb3) are the desired cells. The productivities obtained from the clones screened is summarized in text next to the FACS data.

FIGS. 8A and 8B summarizes a Universal Targeted Integration (UTI) technology that can be implemented using four different strategies (Strategy A, Strategy B, Strategy C, and Strategy D). The universal TI technology disclosed herein comprises the use of site-specific endonuclease(s) directed at parental plasmid sequences in the Parental Cell line not present in the landing pad plasmid to stimulate homologous recombination with the landing pad plasmid. An advantage of this strategy no knowledge of the flanking genomic DNA sequence is needed. In this UTI technology as depicted in FIG. 8A, the parental expression plasmid in the parental cell line is either replaced by a landing pad (Strategy A), or the parental expression plasmid is deleted and the landing pad inserted in an alternative locus (loci) in the cellular genome (Strategy B). In both cases a site-specific endonuclease is used to stimulate recombination. Once the Landing Pad Cell line is created it is used to make Expression Cell Lines in which the landing pad is replaced with the Second GOI using Cre recombinase. In this UTI technology as depicted in FIG. 8B, a single SSRS site is used for creating expression cell line in Strategy C and Strategy D. The boxes with vertical and wavy lines represent regions of homology between different plasmids. The solid box represents a sequence present in the parental expression plasmid targeted, e.g., by CRISPR/Cas, but absent in the plasmids to be recombined into these cell lines. The scissors represent CRISPR/Cas. mCherry open reading frame encodes a fluorescent marker. LoxP and Lox511 sites are sequences used by the Cre recombinase. attB and attP sites are sequences used by the integrase. The arrow and GS encode for GS complementation. Arrow and Puro encode for puromycin resistance. The depiction of these 4 alternative strategies is exemplary, and components shown in the drawings (e.g., CRISPR/Cas, mCherry, Lox sites, att sites) can be replaced with functional equivalents disclosed in the present specification.

FIG. 9 shows summary of data in making landing pad cell line using the strategy illustrated in FIGS. 8A and 8B. The pictures show the increased frequency in generating the mCherry landing pad cell line stimulated by the presence of the single guide RNA (sgRNA) required by the CRISPR/Cas technology. 25% percent of clones have the desired phenotype with the mCherry expression cassette and no mAb from the parental cell line being present. The landing pad cell lines used for TI were identified by their mCherry gene copy number, expression level (mean fluorescent intensity MFI), transcript levels, and stability of these two parameters when cells were passaged.

FIG. 10 summarizes results of experiments using twelve Landing Pad Cell lines to construct expression cell lines using a Second GOI plasmid that encodes for two copies of light chain and two copies of the heavy chain of a mAb and a plasmid that encodes Cre. The percent of expression cell lines is the percent of mCherry negative (Red(−)) cells in the bulk culture after selection.

FIGS. 11A and 11B summarize results of experiment using five landing pad cell lines that were taken through Cell Line Development. After single cell cloning, 32 expression cell lines from each landing pad cell line were chosen at random, expanded and tested in a 24 deep well plate (DWP) 14 day fed batch assay. This allows for a comprehensive characterization of the potential of the Landing Pad Cell Line. Data is summarized in FIG. 11A and represented in a box and whiskers graph in FIG. 11B.

FIG. 12A shows a head-to-head duo-landing pad configuration. Each landing pad contains two distinct SSRS sites for directional recombination. One GOI (mAb) was inserted into each landing pad locus through recombination. The resulting mAb expression plasmid is still in a head-to-head configuration.

FIG. 12B is a depiction of duo-landing pad configurations and effect of Cre recombinase on duo-landing pad. One landing pad is shown as an arrow with diagonal lines and the other as an arrow with a woven pattern. Both landing pads use LoxP and Lox511. The head to head and tail to tail configuration remain intact in the presence of Cre. In the other two configurations one of the landing pads can be permanently deleted. The purpose of having two or more landing pads is, e.g., to be able to make bi-specific mAbs and increase titers. When under the control the same regulatory sequences (e.g., same promoter) multiple landing pads have a high probability of having same activity. In some aspects, multiple landing pads can be present, e.g., 3, 4 or more, in 1:1 ratios, or in alternative rations, e.g., 1:2 or 2:1.

FIG. 13 illustrates the outcome of TI of Second GOI in head-to-head and tail-to-tail duo-landing pad configuration. One landing pad is shown as an arrow with diagonal lines and the other as an arrow with a woven pattern. Both landing pads use LoxP and Lox511. The second GOI is shown as a solid rectangle. In both cases the expression cell lines have two Second GOIs.

FIG. 14 illustrates the outcome of TI of Second GOI in tail-to-head and head-to-tail duo-landing pad configuration. One landing pad is shown as an arrow with diagonal lines and the other as an arrow with a woven pattern. Both landing pads use LoxP and Lox511. The second GOI is shown as a solid rectangle. In both cases two different expression cell lines are created, one with one Second GOI and the second with two Second GOIs.

FIG. 15 shows a depiction of duo-landing pad configurations with Frt and Lox sites and effect of Cre and Flp recombinase on duo-landing pad. One landing pad is shown as an arrow with diagonal lines and the other as an arrow with a woven pattern. Both landing pads use LoxP and Frt. The head to head and tail-to-tail configuration remain intact in the presence of Cre+Flp. In the other two configurations one of the landing pads can be permanently deleted. This is equivalent to what was observed in FIG. 12B.

FIG. 16 shows a depiction of duo-landing pad configurations using the same aatP site to flank all landing pads and outcome after Second GOI Plasmid and Int are transfected into the cell. One landing pad is shown as an arrow with diagonal lines and the other as an arrow with a woven pattern. The GOI is shown as a solid rectangle.

FIGS. 17A and 17B schematically present that the duo-landing pad can be used to increase diversity of expression of the different subunits that assemble to make a desired complex biologic. The solid and dashed arrows represent different components needed to make the biologic. For illustration purposes the complex biologic needs at least one of each arrow. Each GOI plasmids can contain different configurations of each subunit of the complex biologic, i.e. arrow, of the complex biologic. The Second GOIs can be comprised of multiple arrows in different orders or each arrow by itself. The second GOI plasmids are transfected into the duo-landing pad cell line along with the recombinase. To modify levels of subunit expression different combinations of Second GOI plasmids are transfected into the duo-landing pad cell line. Illustrated are different transfections with Second GOIs to get gene copy ratios of 1:2, 1:1, and 2:1 of the solid to dashed arrows after TI is complete. For example, in FIG. 17A in the 1:2 ratio, one Second GOI contains one copy of the dashed arrow and the other Second GOI plasmid contains a solid and dashed arrow in one of two configurations. As shown in FIG. 17A, this would require two independent transfection of the duo-landing pad cell line. It is clear this is not an exhaustive list of possible outcomes nor inputs. Also not illustrated are configurations where the complex biologic is not made as only a subset is integrated into the landing pad, i.e. only one solid or only dashed arrow. FIG. 17B shows a simplified illustration using addressable landing pads with unique SSRSs. One landing pad is comprised of Lox 511 and Lox P, and the second with Lox sites 2272 and M3. The second GOI plasmids would be specifically targeted to one landing pad or the other using corresponding Lox sites.

FIG. 18 illustrates utility of having the duo-landing pad with addressable landing pads. One landing pad is shown as an arrow with diagonal lines and the other as an arrow with a woven pattern. The Second GOI plasmids are shown as a solid rectangle or a rectangle with vertical lines. In this example each landing pad is flanked by a unique combination of Lox sites that only recombine with themselves. The example is illustrative and other recombinases and their target sites can be used. Having addressable landing pads ensures all four configurations of the duo-landing pad has the prescribed Second GOI without the loss of one of the landing pads in the tail to head and head to tail configurations as shown in FIG. 12B.

FIG. 19 shows an illustration demonstrating that having the duo-landing pad with a single aatB site in each landing pad eliminates landing pad deletion. One landing pad is shown as an arrow with diagonal lines and the other as an arrow with a woven pattern. The Second GOI plasmid is shown as a solid rectangle. The duo-landing pad becomes addressable if the attP sites used are not cross compatible

FIG. 20 shows proof of concept (POC) of targeted integration with duo-landing pad cell line. The mCherry expression cassette is exchanged with a Second GOI mAb expression cassette using the Cre recombinase as outlined in FIG. 8b. The resulting cell population after GS complementation selection was screened by FACS for expression of mCherry (horizontal axis) and cell surface expression of the mAb (vertical axis). Here 5.24% of the cells express mAb only, 90.06% express both proteins, 4.12% express only mCherry, and 0.68% express neither protein.

FIG. 21 shows targeted integration of GOI yields higher producing cells versus random integration. Second GOI plasmid form mAb A and B were integrated into a host cell either by random or targeted integration. The landing pad cell line is a direct descendant of the cell line used for random integration. The titers of the cell populations used for single cell cloning were determined. The targeted integration population have titers approximately three to four fold higher than those for random integration demonstrating the value of this technology to generate landing pad cell lines that can outperform industry standard of random integration

FIG. 22 shows summary of use of duo-Landing Pad Cell Line to make expression cell lines using Second GOI plasmid that contains 1 LC+1 HC, or 2 LC+2 HC. Second GOI plasmids comprising either 1 LC+1 HC, or 2 LC+2 HC for mAb A and B were used in TI cell line development. The productivity of the top 6 clones from each group is shown. In both cases increasing the LC and HC copy number improved the average titer by 25% to 37%, and median titer by 35% to 37%.

### DETAILED DESCRIPTION

The present disclosure provides methods to generate landing pad cells in which a linear plasmid, e.g., a linear plasmid, comprising a gene of interest (e.g., one or more open reading frames encoding an antibody) can be inserted into the genome of a host cell without requiring previous knowledge about host cell genomic sequences for its targeted insertion. Although a linear plasmid is often preferred, circular plasmid can be used to generate the landing pad cells.

The terms “targeted insertion” and “targeted integration” are interchangeably used to refer to gene targeting methods employed to direct insertion or integration of a gene or nucleic acid sequence to a specific location on the genome, i.e., to direct the gene or nucleic acid sequence to a specific site between two nucleotides in a contiguous polynucleotide chain. Targeted insertion may also be performed to introduce a small number of nucleotides or to introduce an entire gene cassette, which includes, e.g., multiple genes, regulatory elements, and/or nucleic acid sequences. “Insertion” and “integration,” and grammatical variants thereof, are used interchangeably throughout this specification. In some aspects, targeted integration can be conducted via recombination, e.g., site-specific recombination, homologous recombination, or a combination thereof.

According to these methods, a cell line, e.g., a cell line historically known to display advantageous properties regarding the expression of a protein of interest (e.g., high recombinant protein yield, low protein degradation or misfolding, specific glycosylating patterns or other properties related to post-translational modification) can be used as parental cell line to generate a landing pad cell line which can be used to express other genes of interest. The parental cell line is ideally a cell that is a hot cell (i.e., produces high titers of recombinant proteins), and has one or more hot spots (genomic areas in which the introduction of a foreign nucleic acid encoding a protein of interest will not be disruptive and will result in high levels of recombinant protein expression). As part of the parental cell selection process disclosed herein, two hots spots were identified.

A plasmid in a parental cell (parental plasmid) comprising, an expression cassette integrated in the genome of the parental cell line is partially removed by excising it (e.g., via homologous recombination) between two locations (e.g., recombination sites) which are internal to the parental plasmid (i.e., without cutting/disrupting the parent cell genomic DNA), and the excised region is replaced with another DNA sequence (landing pad plasmid) which comprises two new recombination sites flanking at least one marker (e.g., a selectable and/or a screenable marker). This method yields a landing pad cell which can be used to insert a nucleic acid sequence (e.g., expression plasmid or gene of interest plasmid) comprising a different gene of interest (i.e., a gene of interest different from the gene of interest present in the parental cell) via recombination at the two newly introduced recombination sites. Different strategies related to this general process are disclosed in the present application.

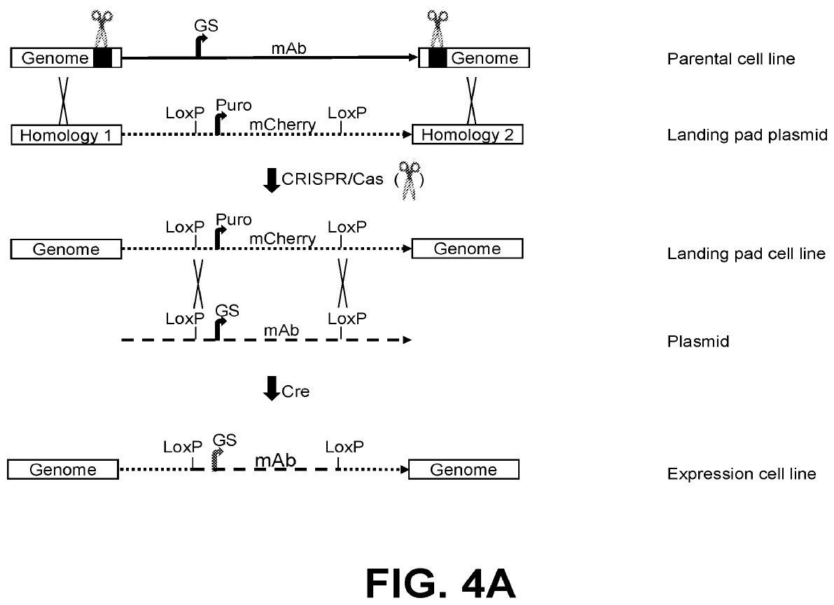

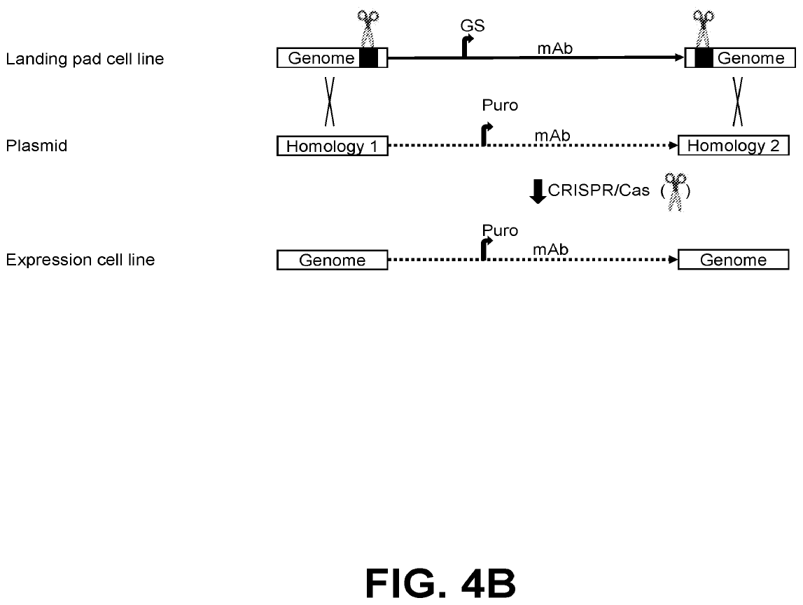

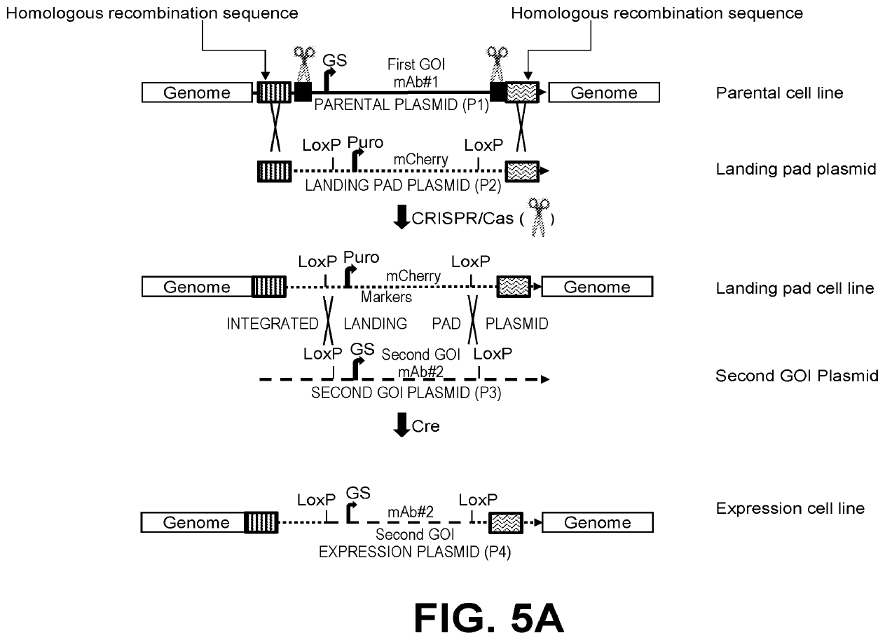

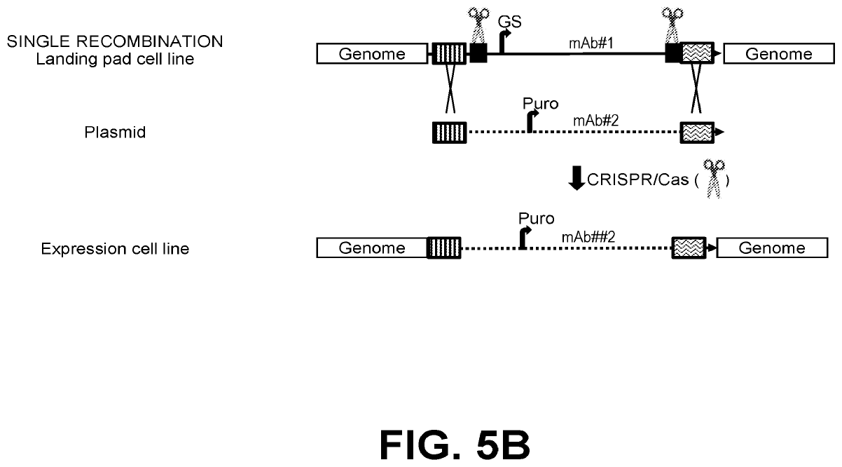

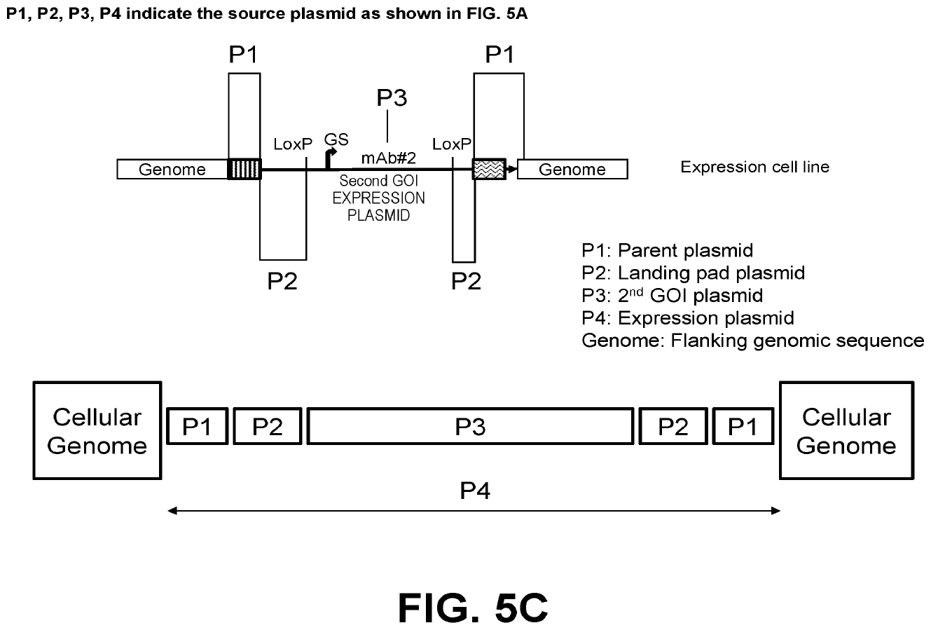

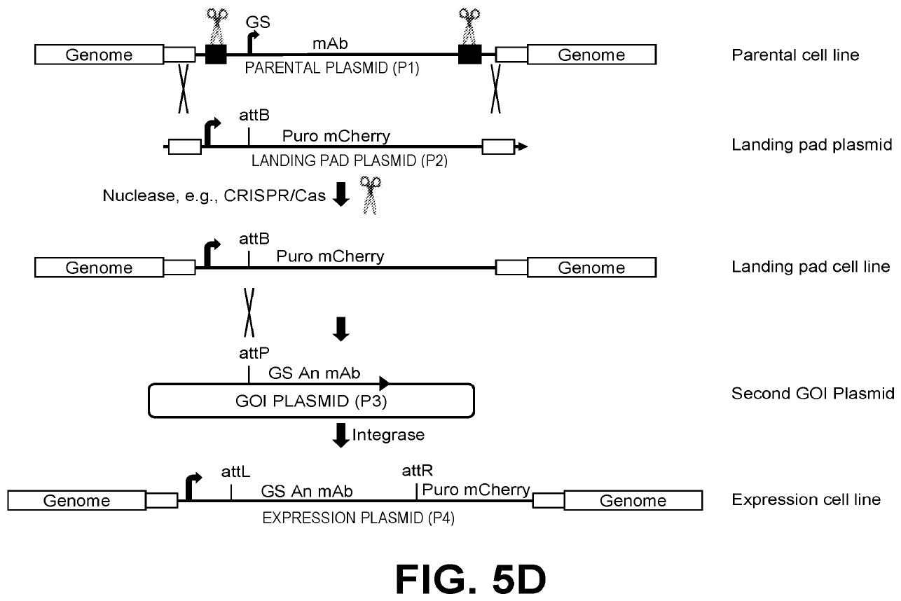

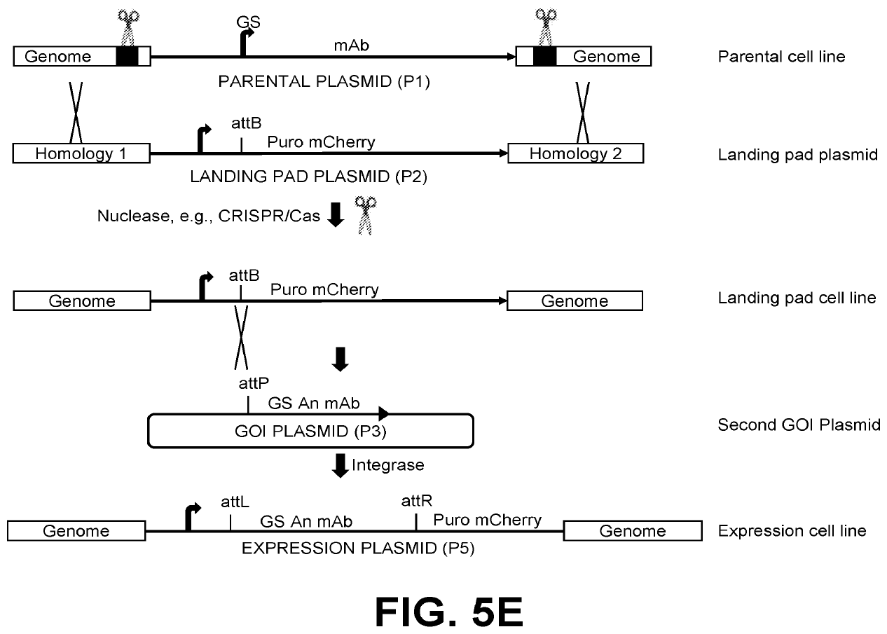

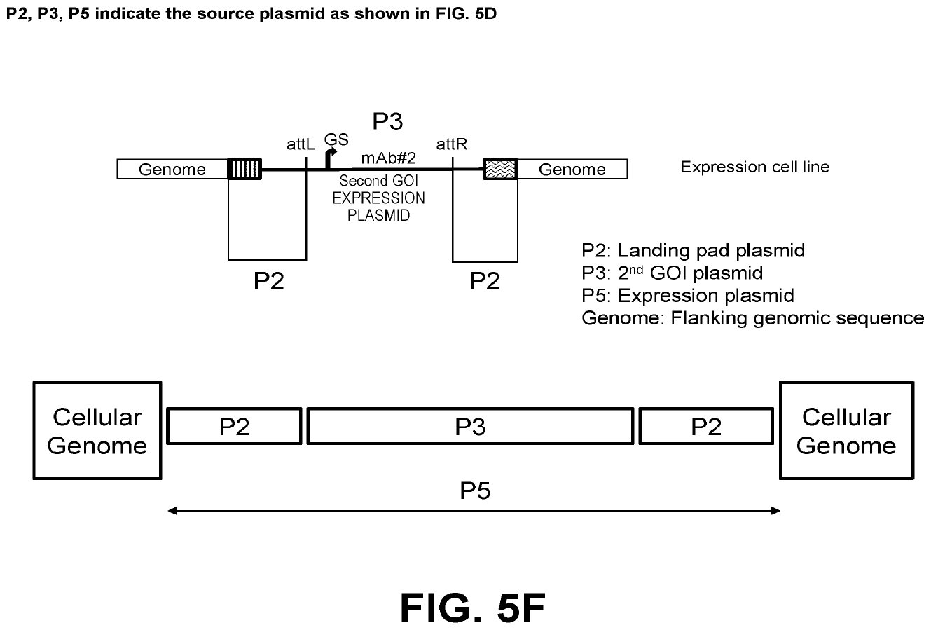

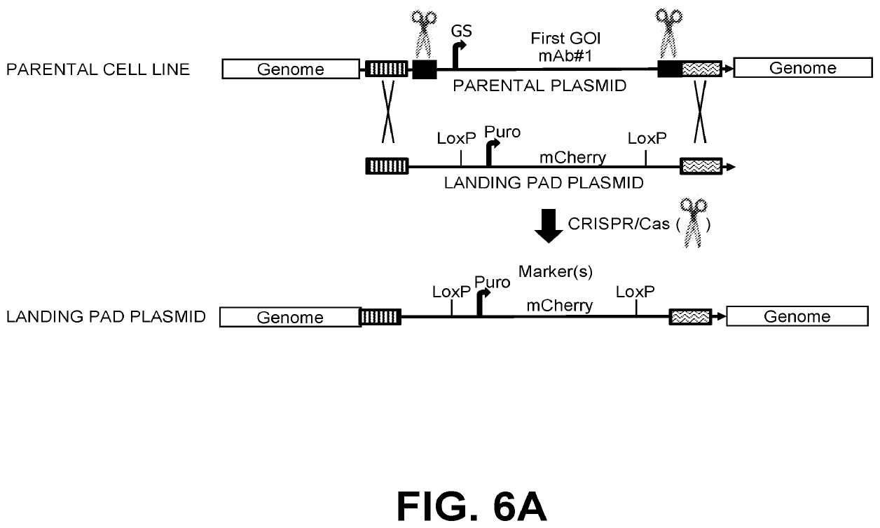

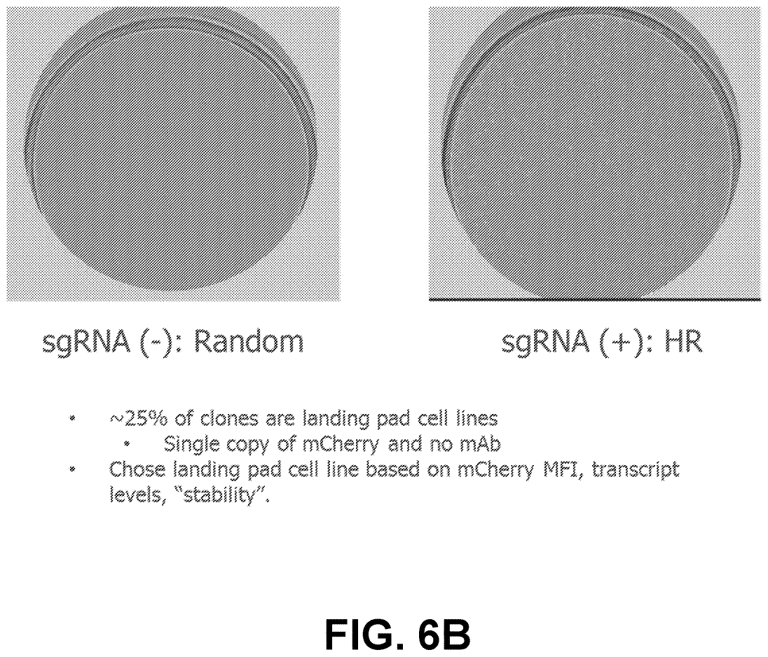

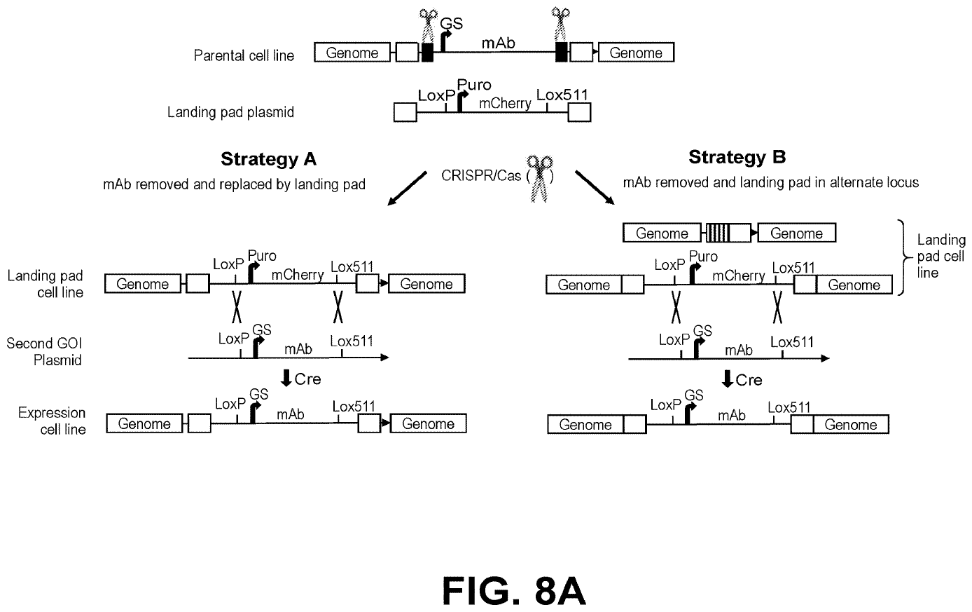

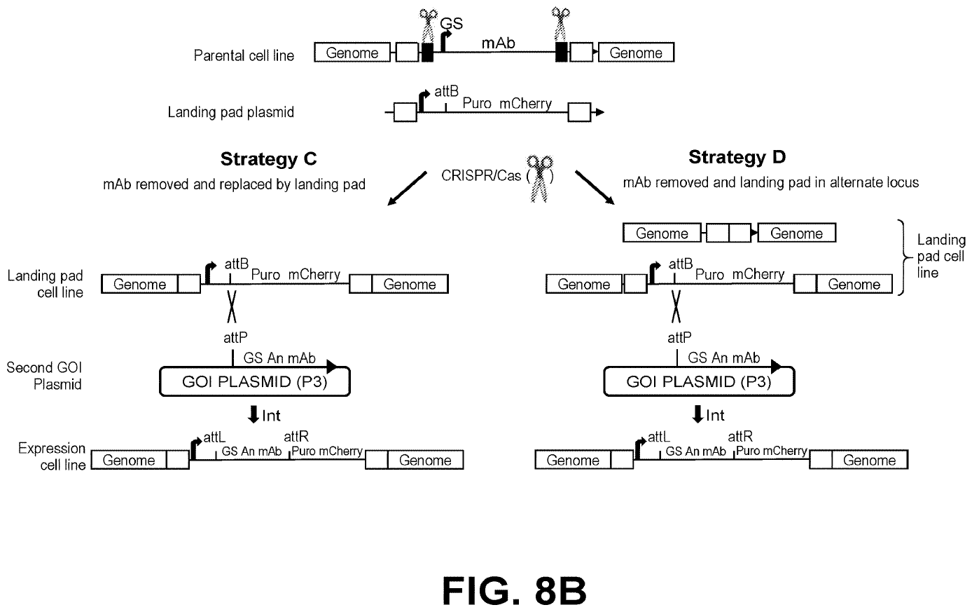

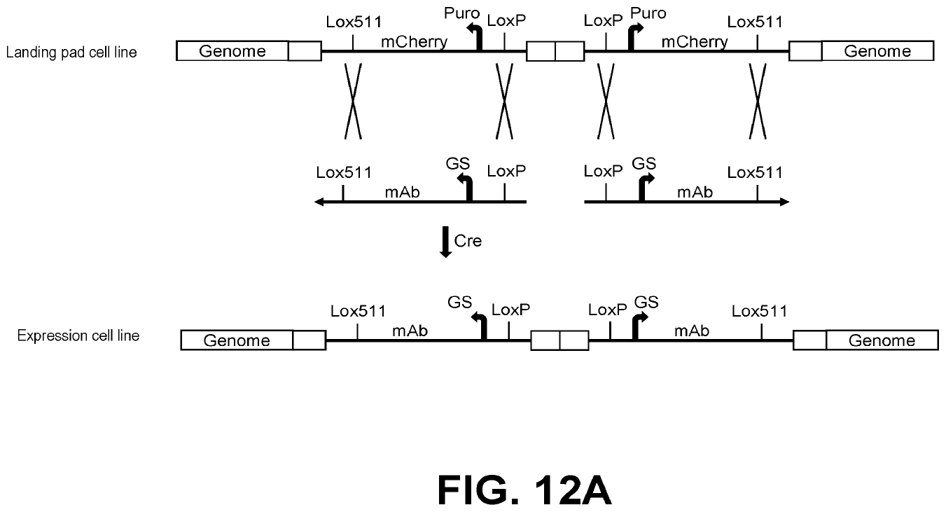

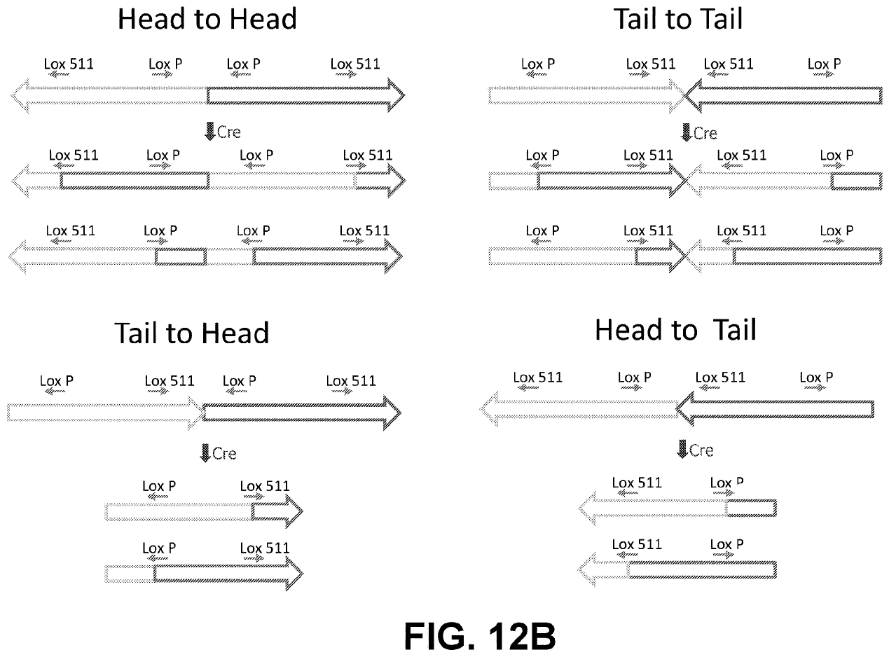

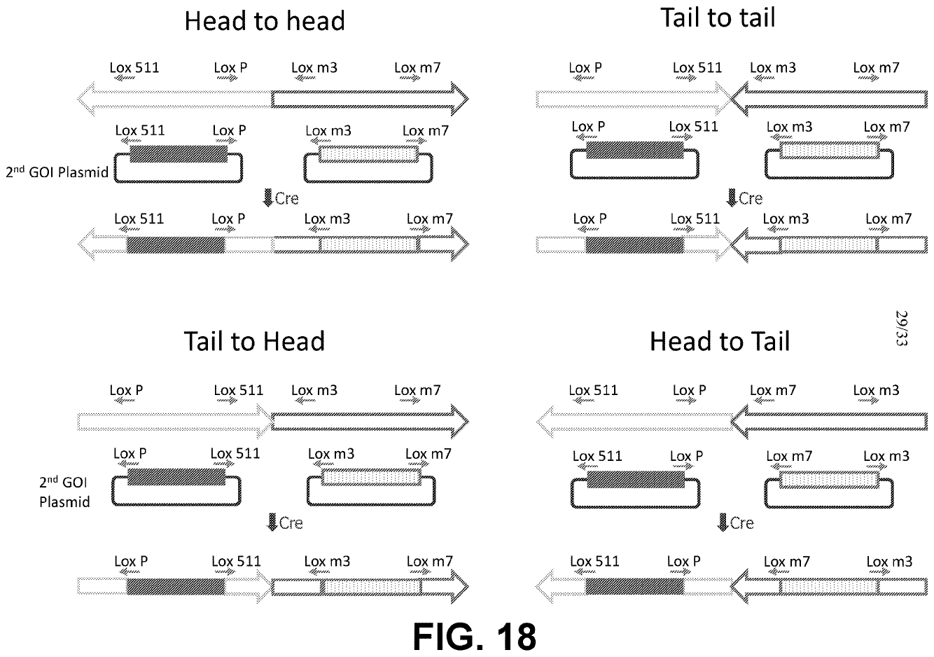

These methods are universal in nature allowing the use of any particularly advantageous parental cell line as a landing pad cell line based on the knowledge of the sequence of the parental plasmid. The knowledge of the plasmid present in the parental cell line (parental plasmid), generally a commercial plasmid known in the art, readily allows the selection of recombination sites suitable for the introduction of a landing pad plasmid or portion thereof in the genome of a parental cell. In turn, the newly introduced recombination sites in the landing pad plasmid can be used to integrate a plasmid or a portion thereof, e.g., a linear or circular plasmid, comprising a gene of interest into the genome of the parental cell, thus yielding an expression cell. See, e.g., FIG. 4A, FIG. 5A, FIG. 6A, and FIG. 8A. Universal Targeted Integration strategies are depicted, e.g., in FIG. 8A (Strategy A and Strategy B) and FIG. 8B (Strategy C and Strategy D). Also provided are constructs comprising multiple landing pads in different configuration (see, e.g., FIG. 12B), wherein each pad can be uniquely identified by using unique SSRS combination (see, e.g., FIG. 18).

The identification of parental cell lines that are “hot cell lines” (i.e., have a high yield of recombinant protein or another advantageous property) and the subsequently identification of the “hot spot” where a parental plasmid was inserted, supports methods for making and using landing pad cells and the improved expression of alternative relevant biologics such as monoclonal antibodies using these methods and/or landing pad cells where no knowledge of the sequence of the parental cell genome is required.

Accordingly, the present disclosure also provides landing pad cells, landing pad plasmids, and kits comprising reagents, e.g., to generate a landing pad cell line, and/or to generate an expression cell line. In addition to providing landing pad cells containing a single landing pad plasmid, the present disclosure provides landing pad cells comprising multiple landing pads. In some aspects, the multiple landing pads in a landing pad cell of the present disclosure can be addressable, e.g., by containing site-specific recombinant sites or combinations thereof that uniquely identify each landing pad.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

The singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

It is understood that wherever aspects are described herein with the language “comprising,” otherwise analogous aspects described in terms of “consisting of” and/or “consisting essentially of” are also provided.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As described herein, any concentration range, percentage range, ratio range or integer range is to be understood to include the value of any integer within the recited range and, when appropriate, fractions thereof (such as one tenth and one hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Systeme International de Unites (SI) accepted form. The headings provided herein are not limitations of the various aspects of the disclosure, which can be had by reference to the specification as a whole. Accordingly, the terms defined are more fully defined by reference to the specification in its entirety.

Abbreviations used herein are defined throughout the present disclosure. Various aspects of the disclosure are described in further detail in the following subsections.

Nucleotides are referred to by their commonly accepted single-letter codes. Unless otherwise indicated, nucleic acids are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

The terms “polynucleotide” or “nucleic acid” are used herein interchangeably and refer to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including mRNAs and gRNAs, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

The terms “nucleic acid sequence” and “nucleotide sequence” are used interchangeably and refer to a contiguous nucleic acid sequence. The sequence can be either single stranded or double stranded DNA or RNA, e.g., a gRNA.

As used herein, the term “subsequence” refers to a subset of contiguous nucleotides in a sequence (either the physical sequence or its symbolic representation).

The methods disclosed herein can be used, e.g., for the production of a biologic such as an antibody.

As use herein, the term “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. Therefore, e.g., the term “anti-PD-1 antibody” includes a full antibody having two heavy chains and two light chains that specifically binds to PD-1 and antigen-binding portions of the full antibody. Non limiting examples of the antigen-binding portions are shown elsewhere herein. In some aspects of the present disclosure, the anti-PD-1 antibody is nivolumab or an antigen-binding portion thereof.

In some aspects, the antibody is a bispecific antibody. A “bispecific antibody” is a particular type of “bispecific molecule” or “bispecific binding molecule.” The term “bispecific antibody” means an antibody that is able to bind to at least two antigenic determinants (e.g., epitopes) through two different antigen-binding sites. In certain aspects, the bispecific antibody is capable of concurrently binding two antigenic determinants (e.g., epitopes). In some aspects, a bispecific antibody binds one antigen (or epitope) on one of its binding arms (one pair of heavy chain/light chain), and binds a different antigen (or epitope) on its second binding arm (a different pair of heavy chain/light chain). In some aspects, a bispecific antibody can have two distinct antigen binding arms (in both specificity and CDR sequences), and is monovalent for each antigen to which it binds. Bispecific antibodies include, e.g., those generated by quadroma technology (Milstein & Cuello (1983) Nature 305(5934):537-40), by chemical conjugation of two different monoclonal antibodies (Staerz et al. (1985) Nature 314(6012):628-31), or by knob-into-hole or similar approaches which introduces mutations in the Fc region (Holliger et al. (1993) Proc. Natl. Acad. Sci. U.S.A. 90(14): 6444-6448).

A wide variety of recombinant antibody formats have been developed in the recent past, e.g. trivalent or tetravalent bispecific antibodies. Examples include the fusion of an IgG antibody format and single chain domains (for different formats see e.g. Coloma, M. J., et al, Nature Biotech 15 (1997), 159-163; WO 2001/077342; Morrison, S. L., Nature Biotech 25 (2007), 1233-1234; Holliger. P. et. al, Nature Biotech. 23 (2005), 1 126-1 136; Fischer, N., and Leger, O., Pathobiology 74 (2007), 3-14; Shen, J., et. al, J. Immunol. Methods 318 (2007), 65-74; Wu, C, et al., Nature Biotech. 25 (2007), 1290-1297). Bispecific antibodies include trivalent or tetravalent bispecific antibodies produced according to the methods disclosed in WO2009/080251; WO2009/080252; WO 2009/080253; WO2009/080254; WO2010/112193; WO2010/115589; WO2010/136172; WO2010/145792; WO2010/145793 and WO2011/117330, all of which are herein incorporated by reference in their entireties. A person of ordinary skill in the art would understand that higher order valencies can also be used.

A wide variety of recombinant bispecific antibody formats have been developed in the recent past, e.g. by fusion of, e.g. an IgG antibody format and single chain domains (see Kontermann R E, mAbs 4:2, (2012) 1-16). Bispecific antibodies wherein the variable domains VL and VH or the constant domains CL and CH1 are replaced by each other are described in WO2009080251 and WO2009080252.

An approach to circumvent the problem of mispaired byproducts, which is known as ‘knobs-into-holes’, aims at forcing the pairing of two different antibody heavy chains by introducing mutations into the CH3 domains to modify the contact interface. On one chain bulky amino acids were replaced by amino acids with short side chains to create a ‘hole’. Conversely, amino acids with large side chains were introduced into the other CH3 domain, to create a ‘knob’. By coexpressing these two heavy chains (and two identical light chains, which have to be appropriate for both heavy chains), high yields of heterodimer formation (‘knob-hole’) versus homodimer formation (‘hole-hole’ or ‘knob-knob’) was observed (Ridgway J B, Presta L G, Carter P; and WO1996027011). The percentage of heterodimer could be further increased by remodeling the interaction surfaces of the two CH3 domains using a phage display approach and the introduction of a disulfide bridge to stabilize the heterodimers (Merchant A. M, et al, Nature Biotech 16 (1998) 677-681; Arwell S, Ridgway J B, Wells J A, Carter P., J Mol Biol 270 (1997) 26-35). New approaches for the knobs-into-holes technology are described in e.g. in EP 1870459A1. Xie, Z., et al, J Immunol Methods 286 (2005) 95-101 refers to a format of bispecific antibody using scFvs in combination with knobs-into-holes technology for the Fc part. See, Godar et al. (2018) “Therapeutic bispecific antibody formats: a patent applications review (1994-2017)” Expert. Opin. Ther. Pat. 28(3):251-276, and Brinkmann & Kontermann (2017) “The making of bispecific antibodies” mAbs 9:182-212; both of which are herein incorporated by reference in their entireties. See also, Ridgway et al (1996) Protein Eng 9:617-21; Atwell et al (1997) J. Mol. Biol. 270:26-35; Merchant et al (1998) Nat. Biotechnol. 16:677-681; Moore et al (2011) MAbs 3:546-55; Von Kreudenstein et al (2013) MAbs 5:646-54; Gunasekaran et al (2010) J. Biol. Chem. 285:19637-47; Geuijen et al (2014) J. Clin. Oncology 32:suppl: 560; Strop et al (2012) J. Mol. Biol. 420:204-19; Choi et al (2013) Mol. Cancer Ther. 12:2748-59; Choi et al (2015) Mol. Immunol. 65:377-83; Labrijn et al (2013) Proc. Natl. Acad. Sci. USA 110:5145-50; Davis et al (2010) Protein Eng. 23:195-202; Moretti et al (2013) BMC Proceedings 7(Suppl 6):09; and Leaver-Fey et al (2016) Structure 24:641-51, all of which are herein incorporated by reference in their entireties. Light chair pairing strategies are disclosed, e.g., in Schaefer et al (2011) Proc Natl Acad Sci USA. 108(27):11187-92; Lewis et al. (2014) Nat Biotechnol. 32(2):191-8; Mazor et al. (2015) MAbs. 7(2):377-89; Liu et al. (2015) J Biol Chem. 290(12):7535-62; Dillon et al. (2017) MAbs. 9(2):213-230; and U.S. Pat. No. 9,914,785, all of which are herein incorporated by reference in their entireties.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to an antigen, e.g., PD-1, is substantially free of antibodies that bind specifically to antigens other than PD-1). An isolated antibody that binds specifically to PD-1 may, however, have cross-reactivity to other antigens, such as PD-1 molecules from different species. Moreover, an isolated antibody can be substantially free of other cellular material and/or chemicals.

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic or other techniques known to those skilled in the art.

A “human antibody” (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibody” and “fully human antibody” and are used synonymously.

A “humanized antibody” refers to an antibody in which some, most or all of the amino acids outside the CDRs of a non-human antibody are replaced with corresponding amino acids derived from human immunoglobulins. In one aspect of a humanized form of an antibody, some, most or all of the amino acids outside the CDRs have been replaced with amino acids from human immunoglobulins, whereas some, most or all amino acids within one or more CDRs are unchanged. Small additions, deletions, insertions, substitutions or modifications of amino acids are permissible as long as they do not abrogate the ability of the antibody to bind to a particular antigen. A “humanized antibody” retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen antibody” refers to an antibody that binds specifically to the antigen. For example, an anti-PD-1 antibody binds specifically to a PD-1 antigen, and an anti-PD-L1 antibody binds specifically to a PD-L1 antigen.

An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments of a full-length antibody. Examples of binding fragments encompassed within the term “antigen-binding portion” of an antibody, e.g., an anti-PD-1 antibody or an anti-PD-L1 antibody, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

In some aspects, the biologic can be a protein, a polypeptide or a polynucleotide. In some aspects, the biologic is an enzyme, a receptor, a receptor ligand, a protein antibiotic, a fusion protein, a structural protein, a regulatory protein, a vaccine, a growth factor, a hormone, or a cytokine. In some aspects, the biological can comprise one or more heterologous moieties, e.g., moieties to extend the plasma half-life of the biologic, moieties to facilitate transport across membranes or the brain blood barrier, moieties to increase or decrease the clearance rate, or moieties to direct the biologic to a particular cell or tissue type (i.e., a targeting moiety).

A polynucleotide, vector, polypeptide, cell, or any composition disclosed herein which is “isolated” is a polynucleotide, vector, polypeptide, cell, or composition which is in a form not found in nature. Isolated polynucleotides, vectors, polypeptides, or compositions include those which have been purified to a degree that they are no longer in a form in which they are found in nature. In some aspects, a polynucleotide, vector, polypeptide, or composition, which is isolated, is substantially pure.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term “percent sequence identity” between two polypeptide or polynucleotide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. When comparing DNA and RNA, thymine (T) and uracil (U) can be considered equivalent.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity “% ID” of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

The terms “gene,” “coding sequence,” “encoding nucleic acid,” “open reading frame,” “ORF,” and grammatical variants thereof are used interchangeably in the present disclosure and refer to nucleic acids (RNA or DNA molecule) that comprise a nucleotide sequence which encodes a gene of interest (GOI), which is generally a protein, e.g., a biologic such as an antibody. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

As use herein the term “gene of interest,” abbreviates as “GOI” refers to an exogenous protein to be expressed by a cell disclosed herein. In some aspects, the GOI is a biologic, for example an antibody or a portion thereof. In some aspects, the GOI comprises one or more open reading frames, e.g., encoding one or more recombinant proteins, operably linked to one or more promoter and/or other regulatory sequences. In some aspect, a cell disclosed herein can contain a first GOI, which can be replaced by a second GOI. In some aspects, the first GOI (e.g., a GOI located on the parental plasmid) and the second GOI (e.g., a GOI located on the second GOI plasmid) belong to the same molecule class. For example, if the first GOI was an antibody, the second GOI may also be antibody since the parent cell line efficiently expressed that type of recombinant protein. In some aspect, the GOI is a nucleic acid, e.g., a therapeutic nucleic acid. In some aspects, the terms GOI and ORF can be used interchangeable, in particular when a GOI in encoded by a single ORF. In some aspects, a GOI can be encoded by more than one ORF. In some aspects, the GOI cam be a detectable molecule, for example, a marker.

“Complement” or “complementary” as used herein refers to Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “vector,” “expression vector,” “plasmid,” and grammatical variants thereof are used interchangeably in the present disclosure and refer to polynucleotide exogenous to the genome of a host cell, which is inserted into a particular location in the genome of a host cell. In general, the plasmid comprises a plurality of elements such a recombination sites (e.g., homologous recombination sites and/or site-specific recombination sites), markers (e.g., detection markers and/or selection markers), one or more expression cassettes, or any combination thereof. In some aspects, the plasmid can be a linear plasmid. In other aspects, the plasmid can be a circular plasmid, e.g., an intact circular plasmid.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression.

A “host cell,” as used herein, denotes an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for a heterologous nucleic acid, therefore becoming a recombinant host cell). Accordingly, the term host cell also includes the progeny of the original host cell (i.e., the host cell prior to receiving a heterologous nucleic acid) which has been transformed by the heterologous nucleic acid, i.e., recombinant host cells. It is understood that the progeny of a single cell may not necessarily be completely identical in morphology or in genomic or total DNA complement as the original parent, due to natural, accidental, or deliberate mutation.

A “recombinant host cell” or “genetically modified host cell” is a host cell into which has been introduced a heterologous nucleic acid, e.g., an expression vector. For example, a eukaryotic host cell becomes a recombinant or genetically modified eukaryotic host cell (e.g., a mammalian host cell), by virtue of the introduction of an exogenous nucleic acid into the eukaryotic host cell.

As used herein, the terms “hot cell,” “hot clone,” and “hot cell line” respectively refers to cell, clone, or cell line with has an advantageous property, e.g., it has a high yield of recombinant protein compared to other cells, clones, or cell lines expressing the same recombinant protein. For example, a hot cell, hot clone, or hot cell line can express higher amounts of recombinant protein, can express higher levels of correctly folded recombinant protein, can express a recombinant protein with lower levels of high molecular weight aggregated, can express a recombinant protein with lower levels of fragmentation, or any combination thereof or some other property that is desirable.

As used herein, the term “hot spot” refers to a genomic location (locus) were an exogenous sequence, e.g., a plasmid comprising a polynucleotide sequence encoding a protein for recombinant expression, can be inserted and wherein (i) transcription of the exogenous sequence is not silenced (e.g., by epigenetic modifications) and (ii) transcription of the exogenous sequence occurs at high levels, compared to the transcription levels observed when the exogenous sequence is inserted at other locations (e.g., a reference location). In some aspects, the hot spot does not contain a functional ORF. Thus, in some aspects, the hot spot does not contain an actively transcribed gene or genes. Hot spots lacking actively transcribed genes are particularly advantageous since their partial or total deletion to insert a polynucleotide sequence encoding an exogenous gene (a gene of interest) does not disrupt endogenous protein production. In some aspects, a hot spot of the present disclosure is located adjacent to an actively transcribed gene or between two actively transcribed genes, i.e., the hot spot can be flanked by two actively transcribed gene. In some aspects, inserting a polynucleotide sequence encoding an exogenous gene (a gene of interest) in a hot spot of the present disclosure does not affect the expression of one or more actively transcribed genes adjacent or flanking the hot spot. In some aspects, inserting a polynucleotide sequence encoding an exogenous gene (a gene of interest) in a hot spot of the present disclosure reduces the expression of one or more actively transcribed genes adjacent or flanking the hot spot by less than about 50%, less than about 45%, less than about 40%, less than about 35%, less than about 30%, less than about 25%, less than about 20%, less than about 15%, or less than about 10%.

As used herein, the term “addressable” as applied to a polynucleotide sequence disclosed herein, e.g., a landing pad sequence disclosed herein, refers to a polynucleotide sequence which is uniquely identified by the presence of a unique site-specific recombination site (SSRS) or combination thereof. For example, a first landing pad having the Lox 511 and Lox P sites and a second landing pad having the Lox m3 and Lox m7 sites would be addressable with respect to each other. Thus, in some aspects, a landing pad can be addressable due to the presence of a specific combination of two SSRS. In other aspects, a landing pad can be addressed with respect to a second landing pad via a single SSRS; for example, a first landing pad may have a first single aatP site and a second landing pad may have a second single aatP site, wherein the aatP sites are not cross-compatible. In some aspects of the present disclosure multiple concatenated landing pads can be present, wherein each landing pad is uniquely addressable thanks to the present of a unique SSRS or combination thereof that specifically identifies (addresses) a given landing pad.

As used herein, the term “addressable SSRS” refers to a unique SSRS or a combination thereof that can specifically be targeted for recombination. As used herein, the term “addressable landing pad plasmid” refer to landing pad plasmid comprising an addressable SSRS or combination thereof that can specifically be targeted for recombination.

As used herein the term “non cross-compatible” when applied to a pair of site-specific recombination sites refer to sites that are deficient in recombination with alternative SSRS, i.e., cannot recombine or only some residual cross reactivity with alternative SSRS. For example, two Lox sites such as LoxP and Lox511 have reduced recombination potential with each other would be considered non cross-compatible. Similarly, two attP-aatB pair of sites that have reduced recombination potential with each other would be consider non cross-compatible.

As used herein, the terms “head-to-head,” “tail-to-tail,” “tail-to-head,” and “head-to-tail” refer to the relative orientations of two polynucleotide sequences, e.g., two landing pads, landing pad plasmids, expression plasmids, or genes of interests in a genetic construct disclosed herein. The term “head” refers to the 5′ end of a nucleic acid sequence and the term “tail” refers to the 3′ end of a nucleic acid sequence. Thus, a 3′-5′ 5′-3′ configuration is head-to-head since (considering a 5′ to 3′ end to the construct) both 5′ ends of the original sequences (heads) are next to each other. A 5′-3′ 3′-5′ configuration would consequently be tail-to-tail, 5′-3′ 5′-3′ would be tail-to-head, and 3′-5′ 3′-5′ would be head-to-tail.

### II. Landing Pad Cells

The present disclosure provides landing pad cells that can be used for the recombinant expression of at least one gene of interest (GOI). In some aspects, these cell lines comprise a “landing pad,” i.e., a specific polynucleotide sequence or sequences inserted in the genome of a parental cell which can be replaced, e.g., via recombination, with another specific polynucleotide sequence or sequences comprising a nucleotide sequence encoding at least one GOI. In some aspects, instead of replacing a polynucleotide sequence, e.g., via recombination, the specific polynucleotide sequence or sequences comprising a nucleotide sequence encoding at least one GOI can be inserted at a location within the landing pad, e.g., via an aat site.

As a general description of one of the processes disclosed herein, a parental cell line (e.g., a “historic” cell line known to efficiently express a particular biologic) is modified by replacing completely or partially an exogenous polynucleotide sequence comprising a parental or first GOI (i.e., the “parental plasmid”) with a second exogenous polynucleotide sequence (i.e., the “landing pad plasmid or portion thereof”). The resulting cell line, incorporating the landing pad plasmid or portion thereof instead of the entire parental plasmid, would be a “landing pad cell.” In some aspects, the landing pad plasmids of the present disclosure comprise flanking sequences from the parental plasmid.

In turn, the landing pad plasmid in the landing pad cell can be replaced (e.g., partially) via recombination with another polynucleotide comprising a different or second GOI (“GOI plasmid”), thus yielding an “expression cell.” See, e.g., the processes depicted in FIG. 5A and FIG. 5B, and TABLE 1.

TABLE 1
Elements and recombination events

| | | Gene of interest | Recombination |
|---|---|---|---|
| Cell | Original plasmid | (GOI) | with |
| Parental cell | Parental plasmid* | First GOI | Landing pad |
| | P1 | | plasmid P2 |
| Landing pad | Landing pad | None | Second GOI |
| cell | plasmid P2 | (optional Marker) | plasmid P3 |
| Expression | Expression | Second GOI | n.a. |
| cell | plasmid P4 | | |

*In some aspects, the parental plasmid is referred to as “first GOI plasmid.”

Accordingly, in some aspects, the present disclosure provides expression cells comprising at least one expression plasmid (P4), e.g., a linear plasmid, integrated in the genomic sequence, wherein each expression plasmid comprises
(i) a polynucleotide sequence derived from an expression plasmid (P4), which comprises a nucleic acid encoding a gene of interest (Second GOI);
(ii) two SSRS flanking the polynucleotide sequence of (i) (e.g., if a recombinase system such as Lox is used), or a single SSRS (e.g., if an integrase system such as att is used);
(iii) polynucleotide sequences positioned distally with respect to the polynucleotide of (i) and SSRS of (ii), wherein both flanking polynucleotide sequences of (iii) are derived from a landing pad plasmid (P2);
(iv) polynucleotide sequences distally flanking the polynucleotide sequences of (iii), wherein both flanking polynucleotide sequences of (iv) are derived from a parental plasmid (P1).

The term “site-specific recombinant site,” abbreviated “SSRS,” as used herein includes nucleotide sequences that can be recognized by site-specific recombinases and function as substrates for recombination events. In some aspects, a construct disclosed herein (e.g., a landing pad plasmid or an expression plasmid) can comprise two SSRS, one located upstream and one located downstream with respect to a nucleic acid encoding a GOI or a marker. In some aspects, a construct disclosed herein (e.g., a landing pad plasmid or an expression plasmid) can comprise a single SSRS located either upstream or downstream with respect to a nucleic acid encoding a GOI or a marker. In some aspects, a construct disclosed herein (e.g., a landing pad plasmid or an expression plasmid) can comprise more than two SSRS, wherein all of them are located upstream with respect to a nucleic acid encoding a GOI or a marker, all of them are located downstream with respect to a nucleic acid encoding a GOI or a marker, or some of them are located upstream and some of them are located downstream with respect to a nucleic acid encoding a GOI or a marker.

In the formulas disclosed in the present application including two SSRS, it is to be understood that if instead of a recombination system requiring two SSRS (such as lox or Frt), recombination takes place using a system requiring a single SSRS (e.g., att), then one of the two SSRS in the formula is optional and can be absent. The single SSRS site when one of the SSRS sites in the formula above is absent may be either the SSRS upstream or the SSRS downstream with respect to the [M] or [P3] component. In some aspects, the single SSRS is an att site.

The term “site-specific recombinase” as used herein includes a group of enzymes capable of effecting recombination between “recombination sites”, wherein the two recombination sites are located within a single nucleic acid molecule, or on separate nucleic acid molecules. Examples of “site-specific recombinases” include, but are not limited to Cre, Flp, and Dre recombinases. In some aspects, the site-specific recombinase is an integrase, e.g., λ (lambda) integrase. In some aspects, the site-specific recombinase is a Bxb integrase, e.g., Bxb1 integrase. Bxb1, an integrase encoded by mycobacteriophage Bxb1, is a member of the serine-recombinase family and catalyzes strand exchange between attP and attB, the attachment sites for the phage and bacterial host, respectively.

The present disclosure provides landing pad cells comprising at least one plasmid, e.g., a linear plasmid or a circular plasmid or a combination thereof, integrated in their genomic sequence, wherein each plasmid comprises
(i) a polynucleotide sequence derived from a landing pad plasmid (P2), which comprises at least one marker and two site-specific recombination sites (SSRS) flanking the at least one marker (if a recombinase system such as Lox is used or a single SSRS if an integrase system such as att is used); and,
(ii) polynucleotide sequences flanking the polynucleotide sequences of (i), wherein both flanking polynucleotide sequences of (ii) are derived from a parental plasmid (P1).

It is to be understood that in cases where a single SSRS is present, the description of its location as “flanking” another element in the formula, e.g., a [P2]∘[P3] element (i.e., elements encoding a marker or GOI), refers to the immediate location of the SSRS upstream or downstream with respect to the flanked element. As an example, the [SSRS] in the formula CG₁/-[P1]-[P2]-[SSRS]-[P3]-[P2]-[P1]-/CG₂, would be flanking [P3], which would encode a GOI.

The present disclosure provides an expression cell comprising a plasmid, e.g., a linear plasmid, inserted in its genomic sequence wherein the topology of the plasmid corresponds to the formula

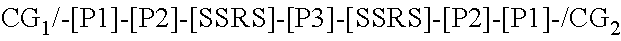

wherein
CG₁ and CG₂ are parental cell genomic sequences flanking the inserted linear plasmid;
[P1] is a polynucleotide sequence derived from a parental plasmid;
[P2] are polynucleotide sequences derived from a landing pad plasmid;
[P3] is a polynucleotide sequence derived from a second GOI plasmid comprising a gene of interest (GOI); and,
[SSRS] are site-specific recombination sites (SSRS).

The present disclosure also contemplates landing pad cells comprising multiple plasmids, e.g., landing plasmids or portions thereof. Thus, present disclosure also provides landing pad cells comprising at least one plasmid, e.g., one, two, three or more linear plasmids or a circular plasmids, integrated in their genomic sequence, wherein each plasmid comprises a polynucleotide sequence derived from a landing pad plasmid (P2), which comprises at least one marker and two site-specific recombination sites (SSRS) flanking the at least one marker; and, polynucleotide sequences flanking the polynucleotide sequences of (i), wherein both flanking polynucleotide sequences of (ii) are derived from a parental plasmid (P1).

Accordingly, the present disclosure provides an expression cell comprising a plasmid, e.g., a linear plasmid, inserted in its genomic sequence wherein the topology of the plasmid corresponds to the formula

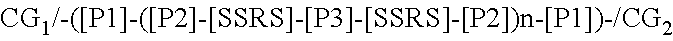

wherein
CG₁ and CG₂ are parental cell genomic sequences flanking the inserted linear plasmid;
[P1] is a polynucleotide sequence derived from a parental plasmid;
[P2] are polynucleotide sequences derived from a landing pad plasmid;
[P3] is a polynucleotide sequence derived from a plasmid comprising a gene of interest (GOI);
[SSRS] are site-specific recombination sites (SSRS); and,
n is an integer between 1 and 10.

In some [P3] can comprise a single GOI or multiple GOI. In some aspects, either the 5′ [SSRS] or the 3′ [SSRSA] is optional. In some aspects, the expression cell comprises a plasmid wherein the plasmid is an expression plasmid.

In some aspects, n is 1. In some aspects, n is 2. In some aspects, n is 3. In some aspects, n is 4. In some aspects, n is 5. In some aspects, n is 6. In some aspects, n is 7. In some aspects, n is 8. In some aspects, n is 9. In some aspects, n is 10. In some aspects, all the plasmids are identical. In some aspects, all the plasmids are different. In some aspects, at least one plasmid is different.

In some aspects, CG₁ comprises a polynucleotide sequence of SEQ ID NO:18 or a fragment thereof. In some aspects, CG₂ comprises a polynucleotide sequence of SEQ ID NO:19 or a fragment thereof.

In some aspects, CG₁ comprises a polynucleotide sequence of SEQ ID NO:114 or a fragment thereof. In some aspects, CG₂ comprises a polynucleotide sequence of SEQ ID NO:115 or a fragment thereof.

The present disclosure provides a landing pad cell comprising at least one plasmid, e.g., a linear plasmid, integrated in its genomic sequence, wherein the plasmid comprises
a. a polynucleotide sequence comprising a nucleic acid encoding at least one selection marker and/or at least one nucleic acid sequence encoding a detectable marker;
b. two SSRS flanking the polynucleotide sequence of (1); and,
c. two homologous recombination sites located 5′ and 3′ terminally with respect to the SSRSs of (2), which are homologous to corresponding homologous recombination sites in a parental plasmid.

In some aspects, the topology of the plasmid, e.g., a linear plasmid, in the landing pad cell corresponds to the formula

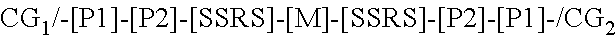

wherein
CG₁ and CG₂ are parental cell genomic sequences flanking the inserted linear plasmid;
[P1] is a polynucleotide sequence derived from a parental plasmid;
[P2] are polynucleotide sequences derived from a landing pad plasmid;
[M] is a polynucleotide sequence comprising at least one marker, e.g., a screenable, a selectable marker, or a combination thereof; and,
[SSRS] are site-specific recombination sites (SSRS).

In some aspects, the topology of the plasmid, e.g., a linear plasmid, in the landing pad cell corresponds to the formula

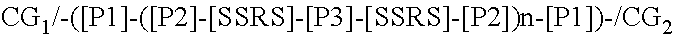

wherein
CG₁ and CG₂ are parental cell genomic sequences flanking the inserted linear plasmid;
[P1] is a polynucleotide sequence derived from a parental plasmid;
[P2] are polynucleotide sequences derived from a landing pad plasmid;
[M] is a polynucleotide sequence comprising at least one marker, e.g., a screenable, a selectable marker, or a combination thereof;
[SSRS] are site-specific recombination sites (SSRS); and,
n is an integer between 1 and 10.

In some aspects, n is 1. In some aspects, n is 2. In some aspects, n is 3. In some aspects, n is 4.

In some aspects, n is 5. In some aspects, n is 6. In some aspects, n is 7. In some aspects, n is 8.

In some aspects, n is 9. In some aspects, n is 10. In some aspects, all the plasmids are identical.

In some aspects, all the plasmids are different. In some aspects, at least one plasmid is different.

The present disclosure also provides a landing pad cell comprising a plasmid, e.g., a linear plasmid, inserted in its genomic sequence wherein the topology of the plasmid corresponds to the description

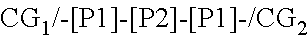

wherein
CG₁ and CG₂ are parental cell genomic sequences flanking the inserted plasmid;
[P1] is a polynucleotide sequence derived from a parental plasmid;
[P2] is a polynucleotide sequence derived from a landing pad plasmid comprising at least one marker and two site-specific recombination sites (SSRS) flanking the at least one marker.

The present disclosure also provides a landing pad cell comprising plasmids, e.g., a linear plasmid, inserted in its genomic sequence wherein the topology of the plasmid corresponding to the following descriptions

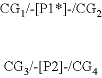

wherein
CG₁ and CG₂ are parental cell genomic sequences flanking the inserted plasmid in a first hot spot;
CG₃ and CG₄ are parental cell genomic sequences flanking the inserted plasmid in a second hot spot;
[P1*] is a polynucleotide sequence derived from a parental plasmid with at least a partial deletion;
[P2] is a polynucleotide sequence derived from a landing pad plasmid comprising at least one marker and two site-specific recombination sites (SSRS) flanking the at least one marker.

The present disclosure also provides a landing pad cell comprising a plasmid, e.g., a linear plasmid, inserted in its genomic sequence wherein the topology of the plasmid corresponds to the description

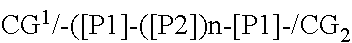

wherein
CG₁ and CG₂ are parental cell genomic sequences flanking the inserted plasmid;
[P1] is a polynucleotide sequence derived from a parental plasmid;
[P2] are polynucleotide sequences derived from a landing pad plasmid comprising at least one marker and two site-specific recombination sites (SSRS) flanking the at least one marker; and,
n is an integer between 1 and 10. In some aspects, n is 1. In some aspects, n is 2. In some aspects, n is 3. In some aspects, n is 4. In some aspects, n is 5. In some aspects, n is 6. In some aspects, n is 7. In some aspects, n is 8. In some aspects, n is 9. In some aspects, n is 10. In some aspects, all the plasmids are identical. In some aspects, all the plasmids are different. In some aspects, at least one plasmid is different.

The present disclosure also provides a landing pad cell comprising plasmids, e.g., a linear plasmid, inserted in its genomic sequence wherein the topology of the plasmids corresponds to the following description

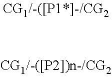

wherein
CG₁ and CG₂ are parental cell genomic sequences flanking the inserted plasmid in a first hot spot;
CG₃ and CG₄ are parental cell genomic sequences flanking the inserted plasmid in a second hot spot;
[P1*] is a polynucleotide sequence derived from a parental plasmid with at least a partial deletion;
[P2] are polynucleotide sequences derived from a landing pad plasmid comprising at least one marker and two site-specific recombination sites (SSRS) flanking the at least one marker; and,
n is an integer between 1 and 10. In some aspects, n is 1. In some aspects, n is 2. In some aspects, n is 3. In some aspects, n is 4. In some aspects, n is 5. In some aspects, n is 6. In some aspects, n is 7. In some aspects, n is 8. In some aspects, n is 9. In some aspects, n is 10. In some aspects, all the plasmids are identical. In some aspects, all the plasmids are different. In some aspects, at least one plasmid is different.

In some aspects, CG₁ comprises a polynucleotide sequence of SEQ ID NO:18; or a fragment thereof. In some aspects, CG₂ comprises a polynucleotide sequence of SEQ ID NO:19 or a fragment thereof.

In some aspects, CG₃ comprises a polynucleotide sequence of SEQ ID NO:114; or a fragment thereof. In some aspects, CG₄ comprises a polynucleotide sequence of SEQ ID NO:115 or a fragment thereof.

In some aspects, for example, when the linear plasmid is inserted into a hot spot which is different from the original hot spot in the parental cell line, the CG₁ and CG₂ genomic sequences (parental cell genomic sequences flanking the inserted linear plasmid) would be replaced by CG₃ and CG₄ genomic sequences, respectively, corresponding to genomic sequences flanking the inserted linear plasmid in the alternative hot spot.

The present disclosure also provides a landing pad plasmid for targeted integration into a host cell's genome comprising a plasmid, e.g., a linear plasmid, wherein the topology of the plasmid corresponds to the formula

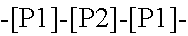

wherein
[P1] is are polynucleotide sequences derived from a parental plasmid integrated in the host comprising homologous recombination sites; and,
[P2] is a polynucleotide sequence comprising at least one marker and two site-specific recombination sites (SSRS) flanking the at least one marker.

The present disclosure also provides a landing pad plasmid for targeted integration into a host cell's genome comprising a plasmid, e.g., a linear plasmid, wherein the topology of the plasmid corresponds to the formula

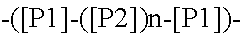

wherein
[P1] is are polynucleotide sequences derived from a parental plasmid integrated in the host comprising homologous recombination sites; [P2] is a polynucleotide sequence comprising at least one marker and two site-specific recombination sites (SSRS) flanking the at least one marker; and n is an integer between 1 and 10. In some aspects, n is 1. In some aspects, n is 2. In some aspects, n is 3. In some aspects, n is 4. In some aspects, n is 5. In some aspects, n is 6. In some aspects, n is 7. In some aspects, n is 8. In some aspects, n is 9. In some aspects, n is 10. In some aspects, all the plasmids are identical. In some aspects, all the plasmids are different. In some aspects, at least one plasmid is different.

It is to be understood that the abbreviated topology of the plasmids disclosed herein (e.g., -[P1]-[P2]-[P1]-) can be described using the terms “description” or “formula” interchangeably.

The present disclosure also provides a method of generating a landing pad cell comprising:
(a) integrating a landing pad plasmid or a portion thereof into the genome of a parental cell at a targeted-integration site using homologous recombination, wherein the landing pad plasmid comprises
   (1) a polynucleotide sequence comprising a nucleic acid encoding at least one selection marker and/or at least one nucleic acid sequence encoding a detectable marker;
   (2) two SSRS flanking the polynucleotide sequence of (1); and,
   (3) two homologous recombination sites located 5′ and 3′ terminally with respect to the SSRSs of (2), which are homologous to corresponding homologous recombination sites in a parental plasmid; and,
wherein the homologous recombination sites of the landing pad plasmid recombine with the corresponding homologous recombination sites of parental plasmid, thereby integrating the landing pad plasmid at an internal location within the parental plasmid inserted in the parent cell genomic DNA.

The targeted integration process disclosed herein comprises substituting a polynucleotide subsequence located between two recombination sites in a plasmid with another polynucleotide subsequence located between two corresponding recombination sites in another plasmid. Thus, the targeted integration of a landing pad plasmid in the parental plasmid, as exemplified, e.g., in FIGS. 4A, 5A, 8A, and 8B, replaces a subsequence of the parental plasmid with a corresponding subsequence from the landing pad plasmid, leaving remnants from the parental plasmid sequence between the recombination sites at the genomic sequence. This targeted integration does not require complete substitution of a plasmid with another plasmid.

Similarly, when a second GOI plasmid is recombined with the landing pad plasmid, the subsequences between the SSRS (e.g., LoxP sites) would be exchanged, but remnants of the landing pad plasmid would remain between the recombination sites and the genomic sequence. In this case, the sequence derived from the second GOI plasmid would be flanked by sequences originating from the landing pad plasmid, which in turn would be flanked by sequences originating from the parental plasmid.

From these explanations follows the fact that references through the present application to the insertion of a plasmid into another plasmid generally do not entail the complete replacement of one plasmid with the other. Instead, a plasmid is completely or in part replaced by another plasmid, or an excised plasmid is excised completely or in part.

In some aspects, the present disclosure provides a method of generating an expression cell comprising:
(a) integrating a second GOI plasmid, e.g., a linear plasmid, into the genome of a landing pad cell of the present disclosure at a targeted-integration site using site-specific recombinase recombination, wherein the expression plasmid (P4) comprises
   (1) a polynucleotide sequence comprising a nucleic acid encoding a GOI; and,
   (2) two SSRS flanking the polynucleotide of (1);
   wherein the site-specific recombinase recombination sites of the landing pad plasmid recombine with the corresponding site-specific recombinase recombination sites of GOI plasmid, thereby integrating the GOI plasmid at an internal location within the landing pad plasmid inserted in the landing pad cell genomic DNA.

The present disclosure also provides a method of generating an expression cell comprising:
(a) integrating a second GOI plasmid, e.g., a linear plasmid, into the genome of a parental cell comprising a parental plasmid at a targeted-integration site using homologous recombination, wherein the expression plasmid comprises
   (1) a polynucleotide sequence comprising a nucleic acid encoding a GOI; and,
   (2) two SSRS flanking the polynucleotide of (1);
wherein the site-specific recombinase recombination sites of the parental plasmid recombine with the corresponding site-specific recombinase recombination sites of GOI plasmid, thereby integrating the GOI plasmid at an internal location within the parental plasmid inserted in the parental cell genomic DNA.

The present disclosure also provides a method of generating an expression cell comprising:
integrating a second GOI plasmid, e.g., a linear plasmid, into the genome of a parental cell comprising a parental plasmid at a targeted-integration site using homologous recombination, wherein the resulting expression plasmid comprises a polynucleotide sequence comprising a nucleic acid encoding a GOI; and,
wherein the parental plasmid recombines with the GOI plasmid, thereby integrating the GOI plasmid at an internal location within the parental plasmid inserted in the parental cell genomic DNA.

The present disclosure also provides a method of generating an expression cell comprising:
integrating a second GOI plasmid, e.g., a linear plasmid, into the genome of a parental cell comprising a parental plasmid at a targeted-integration site using homologous recombination,
wherein the parental plasmid recombines with flanking genomic sequences, thereby integrating the GOI plasmid within the parental plasmid inserted in the parental cell genomic DNA.

It is to be understood, that although the methods disclosed herein relate to two nested nuclease-mediated recombination events, e.g., homologous recombination between the parental plasmid in the parental cell and the landing pad plasmid, and site-specific recombinase recombination between the landing pad plasmid in the landing pad cell and the second GOI plasmid, other combinations of recombination events would be equally applicable, e.g., a first homologous recombination event between P1 and P2, and the second homologous recombination between P2 and P3.

It is also to be understood that teachings related to the integration of a plasmid in the context of the present disclosure, e.g., a landing pad plasmid, or a GOI plasmid, are intend to encompass the insertion of multiple plasmids (e.g., 2, 3, 4, 5, 6, 7, 8, 9, or 10), which can be the same or different, an also differ with respect to their orientation in the final constructs (e.g., whether each one of the plasmids in the final constructs is in a 5′-3′ orientation or 3′-5′ orientation with respect to the other plasmids in the original construct and in the final construct).

In some aspects, the present disclosure also provides a method of generating an expression cell comprising:
integrating a GOI plasmid, e.g., a linear plasmid, into the genome of a parental cell using homologous recombination, wherein the expression plasmid comprises a polynucleotide sequence comprising a nucleic acid encoding a GOI; and,
wherein the GOI plasmid is integrated via homologous recombination at a location determined to correspond to a hot spot. In some aspects, at least a portion of the parental plasmid is removed. In some aspect, the entire parental plasmid is removed.

In some aspects, the present disclosure also provides methods to identify a starting cell (parental cell line) in an efficient manner to make a landing pad cell line capable of yielding high titers. Thus, the present disclosure provides methods to select a parental cell to generate expression cells, e.g., as disclosed in Example 1 and Example 2.

In some aspects, the methods disclosed herein comprise removing at least a portion of at least one parental plasmid and introducing one or more landing pad plasmids or portion thereof with landing pads into cellular genome. In some aspects, the methods disclosed herein comprise removing only a portion of at least one parental plasmid and introducing one or more landing pad plasmids or portion thereof with landing pads into the cellular genome.

In some aspect, the method to select a parental cell line suitable for the development of a landing cell line of the present disclosure comprises:
(i) selecting a cell line with a high expression titer of a gene of interest;
(ii) further selecting a cell with a low copy number of the ORF encoding the gene of interest.
In some aspects, the parental cell has one or two copies of the ORF encoding the gene of interest. In some aspects, the parental cell has more than two copies of the ORF encoding the gene of interest.

In some aspects, the method to select a landing pad cell line comprises screening for the loss of the parental plasmid or a portion thereon, and selection of a cell with such loss (deletion). In some aspects, the method to select a parental cell line further comprises screening for the presence of a landing pad, and selection of a cell in which a landing pad in present. In some aspects, the method further comprises screening the landing pad for characteristics such as the presence or absence of regions of low complexity or high complexity, presence or absence of retrotransposon sequences, presence or absence of Alu repeats, presence or absence of long interspersed nuclear elements (LINE), presence or absence of islands, levels of cytosine methylation, levels of histone acetylation, presence or absence of ORFs, and any combination thereof.

In some aspects, the cell is a CHO cell. In some aspects, the hot spot location comprises a sequence selected from SEQ ID NO:18, or a fragment thereof and SEQ ID NO:19, or a fragment thereof. In some aspects, the hot spot location comprises a sequence selected from SEQ ID NO:114 or a fragment thereof and SEQ ID NO:115, or a fragment thereof.

In some aspects, the GOI plasmid is inserted integrated via homologous recombination or random integration at a location within a genomic sequence of SEQ ID NO: 18. In some aspects, the GOI plasmid is inserted integrated via homologous recombination or random integration at a location within a genomic sequence within a genomic sequence of SEQ ID NO: 19. In some aspects, the GOI plasmid is inserted integrated via homologous recombination or random integration at a location within a genomic sequence of SEQ ID NO: 114. In some aspects, the GOI plasmid is inserted integrated via homologous recombination or random integration at a location within a genomic sequence of SEQ ID NO: 115.

In some aspects, the GOI plasmid the GOI plasmid is integrated via homologous recombination at a location within a genomic sequence wherein the 5′ homologous recombination site comprises a polynucleotide sequence of SEQ ID NO: 18 or a subsequence thereof and/or the 3′ homologous recombination site comprises a polynucleotide sequence of SEQ ID NO: 19 or a subsequence thereof.

In some aspects, the GOI plasmid the GOI plasmid is integrated via homologous recombination at a location within a genomic sequence wherein the 5′ homologous recombination site comprises a polynucleotide sequence of SEQ ID NO: 114 or a subsequence thereof and/or the 3′ homologous recombination site comprises a polynucleotide sequence of SEQ ID NO: 115 or a subsequence thereof.

In some aspects, the 5′ homologous recombination site comprises at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 510, at least about 520, at least about 530, at least about 540, at least about 550, at least about 560, at least about 570, at least about 570, at least about 580, at least about 590, or at least about 600 contiguous nucleotides from SEQ ID NO: 18 or SEQ ID NO:114.

In some aspects, the 3′ homologous recombination site comprises at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 510, at least about 520, at least about 530, at least about 540, at least about 550, at least about 560, at least about 570, at least about 570, at least about 580, at least about 590, or at least about 600 contiguous nucleotides from SEQ ID NO: 19 or SEQ ID NO: 115.

In some aspects, the 5′ homologous recombination site comprises at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 510, at least about 520, at least about 530, at least about 540, at least about 550, at least about 560, at least about 570, at least about 570, at least about 580, at least about 590, or at least about 600 contiguous nucleotides from SEQ ID NO: 18 or SEQ ID NO: 114 and the 3′ homologous recombination site comprises at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 510, at least about 520, at least about 530, at least about 540, at least about 550, at least about 560, at least about 570, at least about 570, at least about 580, at least about 590, or at least about 600 contiguous nucleotides from SEQ ID NO: 19 or SEQ ID NO:115.

In some aspects, the GOI is an antibody. In some aspects, the GOI comprises the heavy chain (HC) of an antibody. In some aspects, the GOI comprises the light chain (LC) of an antibody. In some aspects, the GOI comprises the HC and the LC of an antibody. In some aspects, the GOI comprises an antigen-binding portion of an antibody. In some aspects, the expression plasmid comprises one, two, or more copies of the GOI. In some aspects, the expression plasmid comprises one, two, or more expression cassettes. In some aspects, the expression plasmid is bicistronic. In some aspects, the expression plasmid is multicistronic.

In some aspects, the expression plasmid is integrated with a copy number of at least one (1) in the genome of the expression cell. In some aspects, the expression plasmid is integrated with a copy number of one (1) in the genome of the expression cell. In other aspects, the expression plasmid is integrated with a copy number of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 copies in the genome of the expression cell. In some aspects, there are more than 30 copies in the genome of the expression cell.

In other aspects, the expression plasmid is integrated with a copy number of about 1 to about 3, about 3 to about 6, about 6 to about 9, about 9 to about 12, about 12 to about 15, about 15 to about 18, about 18 to about 21, about 21 to about 24, about 24 to about 27, about 27 to about 30, about 5 to about 10, about 10 to about 15, about 15 to about 20, about 20 to about 25, about 25 to about 30, about 1 to about 10, about 5 to about 15, about 10 to about 20, about 15 to about 25, about 20 to about 30, about 1 to about 15, about 5 to about 20, about 10 to about 25, about 15 to about 30, about 1 to about 20, about 5 to about 25, about 10 to about 30 copies in the genome of the expression cell.

In some aspects, the method disclosed herein comprise determining the expression of a GOI produced by a host cell after the targeted integration of a second GOI plasmid (P3; see, e.g., FIG. 5A) in a landing pad cell line to generate an expression plasmid (P4; see, e.g., FIG. 5A). In some aspects, expression levels are determined quantitatively. In other aspects, expression is determined qualitatively. Expression of the GOI can be determined by using any method known in the art, e.g., cell sorting, FACS, cell surface staining, Western blot, Northern blot, column chromatography, capillary electrophoresis, microfluidics, UV absorbance, cell size, secreted protein levels, transcript levels, immunohistochemistry, or any combination thereof.

In the content of the present disclosure, the recombinant expression level of the second GOI can correspond to the expression from single expression cassette, or from the expression of multiple expression cassettes using an expression cell generated according to the methods of the present disclosure. In some aspects, the expression of the GOI can correspond to multiple cassettes comprising the GOI inserted in the same site

The present disclosure provides landing pad cells and expression cells produced according to the methods disclosed herein. In some aspects, the recombinant protein expression levels of a second GOI (e.g., a second biologic, such as second antibody) obtained when using an expression cell generated according to the methods of the present disclosure is at least about 50%, at least about 55%, at least about 60%, at least about 65%, at least about 70%, at least about 75%, at least about 80%, at least about 85%, at least about 90%, at least about 100%, at least about 110%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, at least about 200%, at least about 300%, at least about 400%, at least about 500%, at least about 600%, at least about 700%, at least about 800%, at least about 900%, or at least about 1000% of the recombinant protein expression level of a first GOI (e.g., a first biologic, such as first antibody) observed when the parental cell is cultured under the same conditions.

The present disclosure provides landing pad cells and expression cells produced according to the methods disclosed herein. In some aspects, the recombinant protein expression levels of a second GOI (e.g., a second biologic, such as second antibody) obtained when using an expression cell generated according to the methods of the present disclosure is about 50%, about 55%, about 60%, about 65%, about 70%, about 75%, about 80%, about 85%, about 90%, about 100%, about 110%, about 120%, about 125%, about 130%, about 135%, about 140%, about 145%, about 150%, about 155%, about 160%, about 165%, about 170%, about 175%, about 180%, about 185%, about 190%, about 195%, about 200%, about 300%, about 400%, about 500%, about 600%, about 700%, about 800%, about 900%, about 1000% or over 1000% of the recombinant protein expression level of a first GOI (e.g., a first biologic, such as first antibody) observed when the parental cell is cultured under the same conditions.

The present disclosure provides landing pad cells and expression cells produced according to the methods disclosed herein. In some aspects, the recombinant protein expression levels of a second GOI (e.g., a second biologic, such as second antibody) obtained when using an expression cell generated according to the methods of the present disclosure is about 50% to about 55%, about 55% to about 60%, about 65% to about 70%, about 70% to about 75%, about 75% to about 80%, about 80% to about 85%, about 85% to about 90%, about 90% to about 100%, about 100% to about 110%, about 110% to about 120%, about 120% to about 125%, about 125% to about 130%, about 130% to about 135%, about 135% to about 140%, about 140% to about 145%, about 145% to about 150%, about 150% to about 155%, about 155% to about 160%, about 160% to about 165%, about 165% to about 170%, about 170% to about 1175%, about 175% to about 180%, about 180% to about 185%, about 185% to about 190%, about 190% to about 195%, about 195% to about 200%, about 200% to about 300%, about 300% to about 400%, about 400% to about 500%, about 500% to about 600%, about 600% to about 700%, about 700% to about 800%, about 800% to about 900%, about 900% to about 1000%, or above 1000% of the recombinant protein expression level of a first GOI (e.g., a first biologic, such as first antibody) observed when the parental cell is cultured under the same conditions.

In some aspects of the present disclosure, the cells disclosed herein can be established as cell lines, i.e., a cell culture developed from a single cell and therefore consisting of cells with a uniform genetic makeup in which under certain conditions the cells proliferate indefinitely in the laboratory, and in the case of an expressing cell line, the gene or genes of interest are stably integrated in the genome of the cells.

In some aspects, targeted-integration site is located within the “Chr3 TI contig” or chromosome 3 targeted integration locus, defined as a polynucleotide from Chromosome 3 of Cricetulus griseus (Chinese hamster) comprising (i) a sequence at least about 90%, at least about 91%, at least about 92%, at least about 93%, at least about 94%, at least about 95%, at least about 96%, or at least about 96.6% identical to SEQ ID NO:23 (5′ end 5 kb sequence from the gi|1497155598|ref|NW_020822499.1 26 Mbase contig) at the 5′ end of the polynucleotide and (ii) a sequence at least 96.6% identical to SEQ ID NO:24 (3′ end 5 kb sequence from the gi|1497155598|ref|NW_020822499.1 26 Mbase contig) at the 3′ end of the polynucleotide, wherein the polynucleotide is between 25 Mbases (megabases) and 26.5 Mbases (megabases) in length.

In some aspects, targeted-integration site is located within the “Chr5 TI contig” or chromosome 5 targeted integration locus, defined as a polynucleotide from Chromosome 5 of Cricetulus griseus (Chinese hamster) comprising (i) a sequence at least about 90%, at least about 91%, at least about 92%, at least about 93%, at least about 94%, at least about 95%, at least about 96%, or at least about 96.6% identical to SEQ ID NO: 119 (5′ end 5 kb sequence from the NW_020822577.1 18 Mbase contig) at the 5′ end of the polynucleotide and (ii) a sequence at least 96.6% identical to SEQ ID NO: 120 (3′ end 5 kb sequence from the NW_020822577.1 18 Mbase contig) at the 3′ end of the polynucleotide, wherein the polynucleotide is between 17 Mbases (megabases) and 19 Mbases (megabases) in length.

Within the hot spot of SEQ ID NO: 22 (Refseq NW_020822499.1; available at ncbi.nlm.nih.gov/nuccore/NW_020822499.1?report=genbank and ncbi.nlm.nih.gov/nuccore/NW_020822499.1?report=fasta) and the hot spot of SEQ ID NO: 118 (Refseq NW_020822577.1; available at ncbi.nlm.nih.gov/nuccore/NW_020822577.1?report=genbank and ncbi.nlm.nih.gov/nuccore/NW_020822577.1?report=fasta) there are actively transcribed genes that are described in the CHO RNA-seq datasets (See Singh et al. Biotechnol J. 2018 October; 13(10):e1800070, and Lin et al. PLoS Comput Biol 16(12): e1008498, which are herein incorporated by reference in their entireties) and confirmed experimentally by the applicant. For the first hotspot of NW_020822499.1, the closest gene on the 5′ side of the of the deletion in which the landing pad resides is Prkg1 which is 269 kb upstream, and the closest gene on the 3′ of the deletion in which the landing pad resides is Mbl2 which is 43 kb downstream. No other active transcripts were identified between Prkg1 and Mbl2 by the applicant nor in the CHO RNA-seq data sets. For the second hotspot of NW_020822577.1, the closest gene on the 5′ of the deletion in which the landing pad resides is Ackr1 which is 209 kb upstream, and the closest gene on the 3′ of the deletion in which the landing pad resides is Crp which is 170 kb downstream. No other active transcripts were identified between Ackr1 and Crp by the applicant nor in the CHO RNA-seq data sets. In some aspects, the targeted-integration site is located within SEQ ID NO: 22 or SEQ ID NO: 118 at a position that does not affect the expression of an actively transcribed gene or genes. In some aspects, the actively transcribed gene or genes are located within the hot spot of SEQ ID NO: 22 or the hot spot of SEQ ID NO: 118. In some aspects, the actively transcribed gene or genes are located close to the 5′ end of the hot spot of SEQ ID NO: 22 or the hot spot of SEQ ID NO: 118. In some aspects, the actively transcribed gene or genes are located close to the hot spot of SEQ ID NO: 22 or the hot spot of SEQ ID NO: 118. In some aspects of the present disclosure, an actively transcribed gene is considered close to the 5′ end or 3′ end of a hot spot disclosed herein when the actively transcribed gene is locate at a distances of about 25 kb, 30 kb, 35 kb, 40 kb, 45 kb, 50 kb, 75 kb, 100 kb, 125 kb, 150 kb, 175 kb, 200 kb, 225 kb, 250 kb, 275 kb, 300 kb, 325 kb, 350 kb, 375 kb, 400 kb, 425 kb, 450 kb, 475 kb, or 500 kb from the 5′ end or 3′ end of a hot spot disclosed herein.

In some aspects, the targeted-integration site is located at a specific location in the sequence set forth in SEQ ID NO: 22 or in the Chr3 TI contig. In some aspects, the targeted-integration site is located at a specific location in the sequence set forth in SEQ ID NO: 22 or in the Chr3 TI contig, within nucleotide positions 1 (5′ start position) and 26,290,500 (3′ end position) of SEQ ID NO: 22.

In some aspects, the targeted-integration site is located at a specific location in the sequence set forth in SEQ ID NO: 118 or in the Chr5 TI contig. In some aspects, the targeted-integration site is located at a specific location in the sequence set forth in SEQ ID NO: 118 or in the Chr5 TI contig, within nucleotide positions 1 (5′ start position) and 18,231,092 (3′ end position) of SEQ ID NO: 118.

As used herein, the term “specific location” refers, e.g., to a specific position (e.g., single base) in the sequence set forth in SEQ ID NO: 22 or in the Chr3 TI contig, or in SEQ ID NO:118 or in the Chr5 TI contig in which integration would take place; e.g., a specific location at position 100 would mean that integration would take place by insertion between nucleotides 100 and 101. In some aspects, the term “specific location” refers to a specific range of nucleotides between two positions that would be excised when integration takes place; e.g., a specific location between positions 100 and 200 would mean that the original sequence comprising nucleotides 101 to 199 would be deleted and replaced by the integrated sequence.

In some aspects, the targeted-integration site is located between the positions in the sequence set forth in SEQ ID NO: 22 (corresponding to an exemplary targeted-integration site of SEQ ID NO:21) or in the Chr3 TI contig, or in SEQ ID NO:118 (corresponding to an exemplary targeted-integration site of SEQ ID NO: 117) or in the Chr5 TI contig. In some aspects, the boxed sequence, i.e., the sequence corresponding to the targeted-integration site, is replaced by an expression plasmid (e.g., a parental plasmid or landing pad plasmid as described herein). In some aspects, the expression plasmid (e.g., a parental plasmid or landing pad plasmid as described herein) is integrated on the negative strand corresponding to the sequence set forth in SEQ ID NO: 22 or in the Chr3 TI contig, or in SEQ ID NO:118 or in the Chr5 TI contig. Thus, in some aspects, the underlined sequences upstream (5′) and downstream (3′) from the boxed sequence correspond respectively to the 3′ and 5′ junction of an integrated expression plasmid integrated on the negative strand corresponding to the sequence set forth in SEQ ID NO: 22 or in the Chr3 TI contig, or in SEQ ID NO:118 or in the Chr5 TI contig.

In some aspects, the targeted-integration site is between position 1 and 1,000,000; between position 1,000,000 and 2,000,000; between position 2,000,000 and 3,000,000; between position 3,000,000 and 4,000,000; between position 4,000,000 and 5,000,000; between position 5,000,000 and 6,000,000; between position 6,000,000 and 7,000,000; between position 7,000,000 and 8,000,000; between position 8,000,000 and 9,000,000; between position 9,000,000 and 10,000,000; between position 10,000,000 and 11,000,000; between position 11,000,000 and 12,000,000; between position 12,000,000 and 13,000,000; between position 13,000,000 and 14,000,000; between position 14,000,000 and 15,000,000; between position 15,000,000 and 16,000,000; between position 16,000,000 and 17,000,000; between position 17,000,000 and 18,000,000; between position 18,000,000 and 19,000,000; between position 19,000,000 and 20,000,000; between position 20,000,000 and 21,000,000; between position 21,000,000 and 22,000,000; between position 22,000,000 and 23,000,000; between position 23,000,000 and 24,000,000; between position 24,000,000 and 25,000,000; between position 25,000,000 and 26,000,000; or between position 26,000,000 and 26,294,056 of the sequence set forth in SEQ ID NO: 22 or in the Chr3 TI contig, or in SEQ ID NO:118 or in the Chr5 TI contig.

In some aspects, the targeted-integration site is between position 1-100,000; 100,000-200,000; 200,000-300,000; 300,000-400,000; 400,000-500,000; 500,000-600,000; 600,000-700,000; 700,000-800,000; 800,000-900,000; 900,000-1,000,000; 1,000,000-1,100,000; 1,100,000-1,200,000; 1,200,000-1,300,000; 1,300,000-1,400,000; 1,400,000-1,500,000, 1,500,000-1,600,000; 1,600,000-1,700,000; 1,700,000-1,800,000; 1,800,000-1,900,000; 1,900,000-2,000,000; 2,000,000-2,100,000; 2,100,000-2,200,000; 2,200,000-2,300,000; 2,300,000-2,400,000; 2,400,000-2,500,000, 2,500,000-2,600,000; 2,600,000-2,700,000; 2,700,000-2,800,000; 2,800,000-2,900,000; 2,900,000-3,000,000; 3,000,000-3,100,000; 3,100,000-3,200,000; 3,200,000-3,300,000; 3,300,000-3,400,000; 3,400,000-3,500,000, 3,500,000-3,600,000; 3,600,000-3,700,000; 3,700,000-3,800,000; 3,800,000-3,900,000; 3,900,000-4,000,000; 4,000,000-4,100,000; 4,100,000-4,200,000; 4,200,000-4,300,000; 4,300,000-4,400,000; 4,400,000-4,500,000, 4,500,000-4,600,000; 4,600,000-4,700,000; 4,700,000-4,800,000; 4,800,000-4,900,000; 4,900,000-5,000,000; 5,000,000-5,100,000; 5,100,000-5,200,000; 5,200,000-5,300,000; 5,300,000-5,400,000; 5,400,000-5,500,000, 5,500,000-5,600,000; 5,600,000-5,700,000; 5,700,000-5,800,000; 5,800,000-5,900,000; 5,900,000-6,000,000; 6,000,000-6,100,000; 6,100,000-6,200,000; 6,200,000-6,300,000; 6,300,000-6,400,000; 6,400,000-6,500,000, 6,500,000-6,600,000; 6,600,000-6,700,000; 6,700,000-6,800,000; 6,800,000-6,900,000; 6,900,000-7,000,000; 7,000,000-7,100,000; 7,100,000-7,200,000; 7,200,000-7,300,000; 7,300,000-7,400,000; 7,400,000-7,500,000, 7,500,000-7,600,000; 7,600,000-7,700,000; 7,700,000-7,800,000; 7,800,000-7,900,000; 7,900,000-8,000,000; 8,000,000-8,100,000; 8,100,000-8,200,000; 8,200,000-8,300,000; 8,300,000-8,400,000; 8,400,000-8,500,000, 8,500,000-8,600,000; 8,600,000-8,700,000; 8,700,000-8,800,000; 8,800,000-8,900,000; 8,900,000-9,000,000; 9,000,000-9,100,000; 9,100,000-9,200,000; 9,200,000-9,300,000; 9,300,000-9,400,000; 9,400,000-9,500,000, 9,500,000-9,600,000; 9,600,000-9,700,000; 9,700,000-9,800,000; 9,800,000-9,900,000; 9,900,000-10,000,000; 10,000,000-10,100,000; 10,100,000-10,200,000; 10,200,000-10,300,000; 10,300,000-10,400,000; 10,400,000-10,500,000, 10,500,000-10,600,000; 10,600,000-10,700,000; 10,700,000-10,800,000; 10,800,000-10,900,000; 10,900,000-11,000,000; 11,000,000-11,100,000; 11,100,000-11,200,000; 11,200,000-11,300,000; 11,300,000-11,400,000; 11,400,000-11,500,000, 11,500,000-11,600,000; 11,600,000-11,700,000; 11,700,000-11,800,000; 11,800,000-11,900,000; 11,900,000-12,000,000; 12,000,000-12,100,000; 12,100,000-12,200,000; 12,200,000-12,300,000; 12,300,000-12,400,000; 12,400,000-12,500,000, 12,500,000-12,600,000; 12,600,000-12,700,000; 12,700,000-12,800,000; 12,800,000-12,900,000; 12,900,000-13,000,000; 13,000,000-13,100,000; 13,100,000-13,200,000; 13,200,000-13,300,000; 13,300,000-13,400,000; 13,400,000-13,500,000, 13,500,000-13,600,000; 13,600,000-13,700,000; 13,700,000-13,800,000; 13,800,000-13,900,000; 13,900,000-14,000,000; 14,000,000-14,100,000; 14,100,000-14,200,000; 14,200,000-14,300,000; 14,300,000-14,400,000; 14,400,000-14,500,000, 14,500,000-14,600,000; 14,600,000-14,700,000; 14,700,000-14,800,000; 14,800,000-14,900,000; 14,900,000-15,000,000; 15,000,000-15,100,000; 15,100,000-15,200,000; 15,200,000-15,300,000; 15,300,000-15,400,000; 15,400,000-15,500,000, 15,500,000-15,600,000; 15,600,000-15,700,000; 15,700,000-15,800,000; 15,800,000-15,900,000; 15,900,000-16,000,000; 16,000,000-16,100,000; 16,100,000-16,200,000; 16,200,000-16,300,000; 16,300,000-16,400,000; 16,400,000-16,500,000, 16,500,000-16,600,000; 16,600,000-16,700,000; 16,700,000-16,800,000; 16,800,000-16,900,000; 16,900,000-17,000,000; 17,000,000-17,100,000; 17,100,000-17,200,000; 17,200,000-17,300,000; 17,300,000-17,400,000; 17,400,000-17,500,000, 17,500,000-17,600,000; 17,600,000-17,700,000; 17,700,000-17,800,000; 17,800,000-17,900,000; 17,900,000-18,000,000; 18,000,000-18,100,000; 18,100,000-18,200,000; 18,200,000-18,300,000; 18,300,000-18,400,000; 18,400,000-18,500,000, 18,500,000-18,600,000; 18,600,000-18,700,000; 18,700,000-18,800,000; 18,800,000-18,900,000; 18,900,000-19,000,000; 19,000,000-19,100,000; 19,100,000-19,200,000; 19,200,000-19,300,000; 19,300,000-19,400,000; 19,400,000-19,500,000, 19,500,000-19,600,000; 19,600,000-19,700,000; 19,700,000-19,800,000; 19,800,000-19,900,000; 19,900,000-20,000,000; 20,000,000-20,100,000; 20,100,000-20,200,000; 20,200,000-20,300,000; 20,300,000-20,400,000; 20,400,000-20,500,000, 20,500,000-20,600,000; 20,600,000-20,700,000; 20,700,000-20,800,000; 20,800,000-20,900,000; 20,900,000-21,000,000; 21,000,000-21,100,000; 21,100,000-21,200,000; 21,200,000-21,300,000; 21,300,000-21,400,000; 21,400,000-21,500,000, 21,500,000-21,600,000; 21,600,000-21,700,000; 21,700,000-21,800,000; 21,800,000-21,900,000; 21,900,000-22,000,000; 22,000,000-22,100,000; 22,100,000-22,200,000; 22,200,000-22,300,000; 22,300,000-22,400,000; 22,400,000-22,500,000, 22,500,000-22,600,000; 22,600,000-22,700,000; 22,700,000-22,800,000; 22,800,000-22,900,000; 22,900,000-23,000,000; 23,000,000-23,100,000; 23,100,000-23,200,000; 23,200,000-23,300,000; 23,300,000-23,400,000; 23,400,000-23,500,000, 23,500,000-23,600,000; 23,600,000-23,700,000; 23,700,000-23,800,000; 23,800,000-23,900,000; 23,900,000-24,000,000; 24,000,000-24,100,000; 24,100,000-24,200,000; 24,200,000-24,300,000; 24,300,000-24,400,000; 24,400,000-24,500,000, 24,500,000-24,600,000; 24,600,000-24,700,000; 24,700,000-24,800,000; 24,800,000-24,900,000; 24,900,000-25,000,000; 25,000,000-25,100,000; 25,100,000-25,200,000; 25,200,000-25,300,000; 25,300,000-25,400,000; 25,400,000-25,500,000, 25,500,000-25,600,000; 25,600,000-25,700,000; 25,700,000-25,800,000; 25,800,000-25,900,000; 25,900,000-26,000,000; 26,000,000-26,100,000; 26,100,000-26,200,000; 26,200,000-26,294,056 of the sequence set forth in SEQ ID NO: 22 or in the Chr3 TI contig, or in SEQ ID NO:118 or in the Chr5 TI contig.

In some aspects, the targeted-integration site is between positions 1-1,000; 1,000-2,000; 2,000-3,000; 3,000-4,000; 4,000-5,000; 5,000-6,000; 6,000-7,000; 7,000-8,000; 8,000-9,000; 9,000-10,000; 10,000-11,000; 11,000-12,000; 12,000-13,000; 13,000-14,000; 14,000-15,000; 15,000-16,000; 16,000-17,000; 17,000-18,000; 18,000-19,000; 19,000-20,000; 20,000-21,000; 21,000-22,000; 22,000-23,000; 23,000-24,000; 24,000-25,000; 25,000-26,000; 26,000-27,000; 27,000-28,000; 28,000-29,000; 29,000-30,000; 30,000-31,000; 31,000-32,000; 32,000-33,000; 33,000-34,000; 34,000-35,000; 35,000-36,000; 36,000-37,000; 37,000-38,000; 38,000-39,000; 39,000-40,000; 40,000-41,000; 41,000-42,000; 42,000-43,000; 43,000-44,000; 44,000-45,000; 45,000-46,000; 46,000-47,000; 47,000-48,000; 48,000-49,000; 49,000-50,000; 50,000-51,000; 51,000-52,000; 52,000-53,000; 53,000-54,000; 54,000-55,000; 55,000-56,000; 56,000-57,000; 57,000-58,000; 58,000-59,000; 59,000-60,000; 60,000-61,000; 61,000-62,000; 62,000-63,000; 63,000-64,000; 64,000-65,000; 65,000-66,000; 66,000-67,000; 67,000-68,000; 68,000-69,000; 69,000-70,000; 70,000-71,000; 71,000-72,000; 72,000-73,000; 73,000-74,000; 74,000-75,000; 75,000-76,000; 76,000-77,000; 77,000-78,000; 78,000-79,000; 79,000-80,000; 80,000- 81,000; 81,000-82,000; 82,000-83,000; 83,000-84,000; 84,000-85,000; 85,000-86,000; 86,000-87,000; 87,000-88,000; 88,000-89,000; 89,000-90,000; 90,000-91,000; 91,000-92,000; 92,000-93,000; 93,000-94,000; 94,000-95,000; 95,000-96,000; 96,000-97,000; 97,000-98,000; 98,000-99,000; or 99,000-100,000 of any of the ranges 1-100,000 to 26,200,000-26,294,056 of the sequence set forth in SEQ ID NO: 22 or in the Chr3 TI contig, or in SEQ ID NO:118 or in the Chr5 TI contig shown above.

In some aspects, the targeted-integration site is between positions 1-10; 10-20; 20-30; 30-40; 40-50; 50-60; 60-70; 70-80; 80-90; 90-100; 100-110; 110-120; 120-130; 130-140; 140-150; 150-160; 160-170; 170-180; 180-190; 190-200; 200-210; 210-220; 220-230; 230-240; 240-250; 250-260; 260-270; 270-280; 280-290; 290-300; 300-310; 310-320; 320-330; 330-340; 340-350; 350-360; 360-370; 370-380; 380-390; 390-400; 400-410; 410-420; 420-430; 430-440; 440-450; 450-460; 460-470; 470-480; 480-490; 490-500; 500-510; 510-520; 520-530; 530-540; 540-550; 550-560; 560-570; 570-580; 580-590; 590-600; 600-610; 610-620; 620-630; 630-640; 640-650; 650-660; 660-670; 670-680; 680-690; 690-700; 700-710; 710-720; 720-730; 730-740; 740-750; 750-760; 760-770; 770-780; 780-790; 790-800; 800-810; 810-820; 820-830; 830-840; 840-850; 850-860; 860-870; 870-880; 880-890; 890-900; 900-910; 910-920; 920-930; 930-940; 940-950; 950-960; 960-970; 970-980; 980-990; 990-1000; 1-100; 100-200; 200-300; 300-400; 400-500; 500-600; 600-700; 700-800; 800-900; or 900-1000 of any of the subranges 1-1,000 to 99,000-100,000 of the sequence set forth in SEQ ID NO: 22 or in the Chr3 TI contig, or in SEQ ID NO:118 or in the Chr5 TI contig, shown above.

In some aspect, the targeted-integration site is located between position about 1 to about 10; about 10 to about 20; about 20 to about 30; about 30 to about 40; about 40 to about 50; about 50 to about 60; about 60 to about 70; about 70 to about 80; about 80 to about 90; about 90 to about 100; about 100 to about 110; about 110 to about 120; about 120 to about 130; about 130 to about 140; about 140 to about 150; about 150 to about 160; about 160 to about 170; about 170 to about 180; about 180 to about 190; about 190 to about 200; about 200 to about 210; about 210 to about 220; about 220 to about 230; about 230 to about 240; about 240 to about 250; about 250 to about 260; about 260 to about 270; about 270 to about 280; about 280 to about 290; about 290 to about 300; about 300 to about 310; about 310 to about 320; about 320 to about 330; about 330 to about 340; about 340 to about 350; about 350 to about 360; about 360 to about 370; about 370 to about 380; about 380 to about 390; about 390 to about 400; about 400 to about 410; about 410 to about 420; about 420 to about 430; about 430 to about 440; about 440 to about 450; about 450 to about 460; about 460 to about 470; about 470 to about 480; about 480 to about 490; about 490 to about 500; about 500 to about 510; about 510 to about 520; about 520 to about 530; about 530 to about 540; about 540 to about 550; about 550 to about 560; about 560 to about 570; about 570 to about 580; about 580 to about 590; about 590 to about 600; about 600 to about 610; about 610 to about 620; about 620 to about 630; about 630 to about 640; about 640 to about 650; about 650 to about 660; about 660 to about 670; about 670 to about 680; about 680 to about 690; about 690 to about 700; about 700 to about 710; about 710 to about 720; about 720 to about 730; about 730 to about 740; about 740 to about 750; about 750 to about 760; about 760 to about 770; about 770 to about 780; about 780 to about 790; about 790 to about 800; about 800 to about 810; about 810 to about 820; about 820 to about 830; about 830 to about 840; about 840 to about 850; about 850 to about 860; about 860 to about 870; about 870 to about 880; about 880 to about 890; about 890 to about 900; about 900 to about 910; about 910 to about 920; about 920 to about 930; about 930 to about 940; about 940 to about 950; about 950 to about 960; about 960 to about 970; about 970 to about 980; about 980 to about 990; about 990 to about 1000; about 1,000 to about 2,000; about 2,000 to about 3,000; about 3,000 to about 4,000; about 4,000 to about 5,000; about 5,000 to about 6,000; about 6,000 to about 7,000; about 7,000 to about 8,000; about 8,000 to about 9,000; about 9,000 to about 10,000; about 10,000 to about 11,000; about 11,000 to about 12,000; about 12,000 to about 13,000; about 13,000 to about 14,000; about 14,000 to about 15,000; about 15,000 to about 16,000; about 16,000 to about 17,000; about 17,000 to about 18,000; about 18,000 to about 19,000; about 19,000 to about 20,000; about 20,000 to about 21,000; about 21,000 to about 22,000; about 22,000 to about 23,000; about 23,000 to about 24,000; about 24,000 to about 25,000; about 25,000 to about 26,000; about 26,000 to about 27,000; about 27,000 to about 28,000; about 28,000 to about 29,000; about 29,000 to about 30,000; about 30,000 to about 31,000; about 31,000 to about 32,000; about 32,000 to about 33,000; about 33,000 to about 34,000; about 34,000 to about 35,000; about 35,000 to about 36,000; about 36,000 to about 37,000; about 37,000 to about 38,000; about 38,000 to about 39,000; about 39,000 to about 40,000; about 40,000 to about 41,000; about 41,000 to about 42,000; about 42,000 to about 43,000; about 43,000 to about 44,000; about 44,000 to about 45,000; about 45,000 to about 46,000; about 46,000 to about 47,000; about 47,000 to about 48,000; about 48,000 to about 49,000; about 49,000 to about 50,000; about 50,000 to about 51,000; about 51,000 to about 52,000; about 52,000 to about 53,000; about 53,000 to about 54,000; about 54,000 to about 55,000; about 55,000 to about 56,000; about 56,000 to about 57,000; about 57,000 to about 58,000; about 58,000 to about 59,000; about 59,000 to about 60,000; about 60,000 to about 61,000; about 61,000 to about 62,000; about 62,000 to about 63,000; about 63,000 to about 64,000; about 64,000 to about 65,000; about 65,000 to about 66,000; about 66,000 to about 67,000; about 67,000 to about 68,000; about 68,000 to about 69,000; about 69,000 to about 70,000; about 70,000 to about 71,000; about 71,000 to about 72,000; about 72,000 to about 73,000; about 73,000 to about 74,000; about 74,000 to about 75,000; about 75,000 to about 76,000; about 76,000 to about 77,000; about 77,000 to about 78,000; about 78,000 to about 79,000; about 79,000 to about 80,000; about 80,000 to about 81,000; about 81,000 to about 82,000; about 82,000 to about 83,000; about 83,000 to about 84,000; about 84,000 to about 85,000; about 85,000 to about 86,000; about 86,000 to about 87,000; about 87,000 to about 88,000; about 88,000 to about 89,000; about 89,000 to about 90,000; about 90,000 to about 91,000; about 91,000 to about 92,000; about 92,000 to about 93,000; about 93,000 to about 94,000; about 94,000 to about 95,000; about 95,000 to about 96,000; about 96,000 to about 97,000; about 97,000 to about 98,000; about 98,000 to about 99,000; about 99,000 to about 100,000; about 100,000 to about 200,000; about 200,000 to about 300,000; about 300,000 to about 400,000; about 400,000 to about 500,000; about 500,000 to about 600,000; about 600,000 to about 700,000; about 700,000 to about 800,000; about 800,000 to about 900,000; about 900,000 to about 1,000,000; about 1,000,000 to about 1,100,000; about 1,100,000 to about 1,200,000; about 1,200,000 to about 1,300,000; about 1,300,000 to about 1,400,000; about 1,400,000 to about 1,500,000; about 1,500,000 to about 1,600,000; about 1,600,000 to about 1,700,000; about 1,700,000 to about 1,800,000; about 1,800,000 to about 1,900,000; about 1,900,000 to about 2,000,000; about 2,000,000 to about 2,100,000; about 2,100,000 to about 2,200,000; about 2,200,000 to about 2,300,000; about 2,300,000 to about 2,400,000; about 2,400,000 to about 2,500,000; about 2,500,000 to about 2,600,000; about 2,600,000 to about 2,700,000; about 2,700,000 to about 2,800,000; about 2,800,000 to about 2,900,000; about 2,900,000 to about 3,000,000; about 3,000,000 to about 3,100,000; about 3,100,000 to about 3,200,000; about 3,200,000 to about 3,300,000; about 3,300,000 to about 3,400,000; about 3,400,000 to about 3,500,000; about 3,500,000 to about 3,600,000; about 3,600,000 to about 3,700,000; about 3,700,000 to about 3,800,000; about 3,800,000 to about 3,900,000; about 3,900,000 to about 4,000,000; about 4,000,000 to about 4,100,000; about 4,100,000 to about 4,200,000; about 4,200,000 to about 4,300,000; about 4,300,000 to about 4,400,000; about 4,400,000 to about 4,500,000; about 4,500,000 to about 4,600,000; about 4,600,000 to about 4,700,000; about 4,700,000 to about 4,800,000; about 4,800,000 to about 4,900,000; about 4,900,000 to about 5,000,000; about 5,000,000 to about 5,100,000; about 5,100,000 to about 5,200,000; about 5,200,000 to about 5,300,000; about 5,300,000 to about 5,400,000; about 5,400,000 to about 5,500,000; about 5,500,000 to about 5,600,000; about 5,600,000 to about 5,700,000; about 5,700,000 to about 5,800,000; about 5,800,000 to about 5,900,000; about 5,900,000 to about 6,000,000; about 6,000,000 to about 6,100,000; about 6,100,000 to about 6,200,000; about 6,200,000 to about 6,300,000; about 6,300,000 to about 6,400,000; about 6,400,000 to about 6,500,000; about 6,500,000 to about 6,600,000; about 6,600,000 to about 6,700,000; about 6,700,000 to about 6,800,000; about 6,800,000 to about 6,900,000; about 6,900,000 to about 7,000,000; about 7,000,000 to about 7,100,000; about 7,100,000 to about 7,200,000; about 7,200,000 to about 7,300,000; about 7,300,000 to about 7,400,000; about 7,400,000 to about 7,500,000; about 7,500,000 to about 7,600,000; about 7,600,000 to about 7,700,000; about 7,700,000 to about 7,800,000; about 7,800,000 to about 7,900,000; about 7,900,000 to about 8,000,000; about 8,000,000 to about 8,100,000; about 8,100,000 to about 8,200,000; about 8,200,000 to about 8,300,000; about 8,300,000 to about 8,400,000; about 8,400,000 to about 8,500,000; about 8,500,000 to about 8,600,000; about 8,600,000 to about 8,700,000; about 8,700,000 to about 8,800,000; about 8,800,000 to about 8,900,000; about 8,900,000 to about 9,000,000; about 9,000,000 to about 9,100,000; about 9,100,000 to about 9,200,000; about 9,200,000 to about 9,300,000; about 9,300,000 to about 9,400,000; about 9,400,000 to about 9,500,000; about 9,500,000 to about 9,600,000; about 9,600,000 to about 9,700,000; about 9,700,000 to about 9,800,000; about 9,800,000 to about 9,900,000; about 9,900,000 to about 10,000,000; about 10,000,000 to about 10,100,000; about 10,100,000 to about 10,200,000; about 10,200,000 to about 10,300,000; about 10,300,000 to about 10,400,000; about 10,400,000 to about 10,500,000; about 10,500,000 to about 10,600,000; about 10,600,000 to about 10,700,000; about 10,700,000 to about 10,800,000; about 10,800,000 to about 10,900,000; about 10,900,000 to about 11,000,000; about 11,000,000 to about 11,100,000; about 11,100,000 to about 11,200,000; about 11,200,000 to about 11,300,000; about 11,300,000 to about 11,400,000; about 11,400,000 to about 11,500,000; about 11,500,000 to about 11,600,000; about 11,600,000 to about 11,700,000; about 11,700,000 to about 11,800,000; about 11,800,000 to about 11,900,000; about 11,900,000 to about 12,000,000; about 12,000,000 to about 12,100,000; about 12,100,000 to about 12,200,000; about 12,200,000 to about 12,300,000; about 12,300,000 to about 12,400,000; about 12,400,000 to about 12,500,000; about 12,500,000 to about 12,600,000; about 12,600,000 to about 12,700,000; about 12,700,000 to about 12,800,000; about 12,800,000 to about 12,900,000; about 12,900,000 to about 13,000,000; about 13,000,000 to about 13,100,000; about 13,100,000 to about 13,200,000; about 13,200,000 to about 13,300,000; about 13,300,000 to about 13,400,000; about 13,400,000 to about 13,500,000; about 13,500,000 to about 13,600,000; about 13,600,000 to about 13,700,000; about 13,700,000 to about 13,800,000; about 13,800,000 to about 13,900,000; about 13,900,000 to about 14,000,000; about 14,000,000 to about 14,100,000; about 14,100,000 to about 14,200,000; about 14,200,000 to about 14,300,000; about 14,300,000 to about 14,400,000; about 14,400,000 to about 14,500,000; about 14,500,000 to about 14,600,000; about 14,600,000 to about 14,700,000; about 14,700,000 to about 14,800,000; about 14,800,000 to about 14,900,000; about 14,900,000 to about 15,000,000; about 15,000,000 to about 15,100,000; about 15,100,000 to about 15,200,000; about 15,200,000 to about 15,300,000; about 15,300,000 to about 15,400,000; about 15,400,000 to about 15,500,000; about 15,500,000 to about 15,600,000; about 15,600,000 to about 15,700,000; about 15,700,000 to about 15,800,000; about 15,800,000 to about 15,900,000; about 15,900,000 to about 16,000,000; about 16,000,000 to about 16,100,000; about 16,100,000 to about 16,200,000; about 16,200,000 to about 16,300,000; about 16,300,000 to about 16,400,000; about 16,400,000 to about 16,500,000; about 16,500,000 to about 16,600,000; about 16,600,000 to about 16,700,000; about 16,700,000 to about 16,800,000; about 16,800,000 to about 16,900,000; about 16,900,000 to about 17,000,000; about 17,000,000 to about 17,100,000; about 17,100,000 to about 17,200,000; about 17,200,000 to about 17,300,000; about 17,300,000 to about 17,400,000; about 17,400,000 to about 17,500,000; about 17,500,000 to about 17,600,000; about 17,600,000 to about 17,700,000; about 17,700,000 to about 17,800,000; about 17,800,000 to about 17,900,000; about 17,900,000 to about 18,000,000; about 18,000,000 to about 18,100,000; about 18,100,000 to about 18,200,000; about 18,200,000 to about 18,300,000; about 18,300,000 to about 18,400,000; about 18,400,000 to about 18,500,000; about 18,500,000 to about 18,600,000; about 18,600,000 to about 18,700,000; about 18,700,000 to about 18,800,000; about 18,800,000 to about 18,900,000; about 18,900,000 to about 19,000,000; about 19,000,000 to about 19,100,000; about 19,100,000 to about 19,200,000; about 19,200,000 to about 19,300,000; about 19,300,000 to about 19,400,000; about 19,400,000 to about 19,500,000; about 19,500,000 to about 19,600,000; about 19,600,000 to about 19,700,000; about 19,700,000 to about 19,800,000; about 19,800,000 to about 19,900,000; about 19,900,000 to about 20,000,000; about 20,000,000 to about 20,100,000; about 20,100,000 to about 20,200,000; about 20,200,000 to about 20,300,000; about 20,300,000 to about 20,400,000; about 20,400,000 to about 20,500,000; about 20,500,000 to about 20,600,000; about 20,600,000 to about 20,700,000; about 20,700,000 to about 20,800,000; about 20,800,000 to about 20,900,000; about 20,900,000 to about 21,000,000; about 21,000,000 to about 21,100,000; about 21,100,000 to about 21,200,000; about 21,200,000 to about 21,300,000; about 21,300,000 to about 21,400,000; about 21,400,000 to about 21,500,000; about 21,500,000 to about 21,600,000; about 21,600,000 to about 21,700,000; about 21,700,000 to about 21,800,000; about 21,800,000 to about 21,900,000; about 21,900,000 to about 22,000,000; about 22,000,000 to about 22,100,000; about 22,100,000 to about 22,200,000; about 22,200,000 to about 22,300,000; about 22,300,000 to about 22,400,000; about 22,400,000 to about 22,500,000; about 22,500,000 to about 22,600,000; about 22,600,000 to about 22,700,000; about 22,700,000 to about 22,800,000; about 22,800,000 to about 22,900,000; about 22,900,000 to about 23,000,000; about 23,000,000 to about 23,100,000; about 23,100,000 to about 23,200,000; about 23,200,000 to about 23,300,000; about 23,300,000 to about 23,400,000; about 23,400,000 to about 23,500,000; about 23,500,000 to about 23,600,000; about 23,600,000 to about 23,700,000; about 23,700,000 to about 23,800,000; about 23,800,000 to about 23,900,000; about 23,900,000 to about 24,000,000; about 24,000,000 to about 24,100,000; about 24,100,000 to about 24,200,000; about 24,200,000 to about 24,300,000; about 24,300,000 to about 24,400,000; about 24,400,000 to about 24,500,000; about 24,500,000 to about 24,600,000; about 24,600,000 to about 24,700,000; about 24,700,000 to about 24,800,000; about 24,800,000 to about 24,900,000; about 24,900,000 to about 25,000,000; about 25,000,000 to about 25,100,000; about 25,100,000 to about 25,200,000; about 25,200,000 to about 25,300,000; about 25,300,000 to about 25,400,000; about 25,400,000 to about 25,500,000; about 25,500,000 to about 25,600,000; about 25,600,000 to about 25,700,000; about 25,700,000 to about 25,800,000; about 25,800,000 to about 25,900,000; about 25,900,000 to about 26,000,000 upstream or downstream from the sequence set forth in SEQ ID NO:21 or SEQ ID NO:117.

In some aspect, the targeted to about integration site is located at least about 10; at least about 20; at least about 30; at least about 40; at least about 50; at least about 60; at least about 70; at least about 80; at least about 90; at least about 100; at least about 110; at least about 120; at least about 130; at least about 140; at least about 150; at least about 160; at least about 170; at least about 180; at least about 190; at least about 200; at least about 210; at least about 220; at least about 230; at least about 240; at least about 250; at least about 260; at least about 270; at least about 280; at least about 290; at least about 300; at least about 310; at least about 320; at least about 330; at least about 340; at least about 350; at least about 360; at least about 370; about 380; at least about 390; at least about 400; at least about 410; at least about 420; at least about 430; at least about 440; at least about 450; at least about 460; at least about 470; at least about 480; at least about 490; at least about 500; at least about 510; at least about 520; at least about 530; at least about 540; at least about 550; at least about 560; at least about 570; at least about 580; at least about 590; at least about 600; at least about 610; at least about 620; at least about 630; at least about 640; at least about 650; at least about 660; at least about 670; at least about 680; at least about 690; at least about 700; at least about 710; at least about 720; at least about 730; at least about 740; at least about 750; at least about 760; at least about 770; at least about 780; at least about 790; at least about 800; at least about 810; at least about 820; at least about 830; at least about 840; at least about 850; at least about 860; at least about 870; at least about 880; at least about 890; at least about 900; at least about 910; at least about 920; at least about 930; at least about 940; at least about 950; at least about 960; at least about 970; at least about 980; at least about 990; at least about 1,000; at least about 2,000; at least about 3,000; at least about 4,000; at least about 5,000; at least about 6,000; at least about 7,000; at least about 8,000; at least about 9,000; at least about 10,000; at least about 11,000; at least about 12,000; at least about 13,000; at least about 14,000; at least about 15,000; at least about 16,000; at least about 17,000; at least about 18,000; at least about 19,000; at least about 20,000; at least about 21,000; at least about 22,000; at least about 23,000; at least about 24,000; at least about 25,000; at least about 26,000; at least about 27,000; at least about 28,000; at least about 29,000; at least about 30,000; at least about 31,000; at least about 32,000; at least about 33,000; at least about 34,000; at least about 35,000; at least about 36,000; at least about 37,000; at least about 38,000; at least about 39,000; at least about 40,000; at least about 41,000; at least about 42,000; at least about 43,000; at least about 44,000; at least about 45,000; at least about 46,000; at least about 47,000; at least about 48,000; at least about 49,000; at least about 50,000; at least about 51,000; at least about 52,000; at least about 53,000; at least about 54,000; at least about 55,000; at least about 56,000; at least about 57,000; at least about 58,000; at least about 59,000; at least about 60,000; at least about 61,000; at least about 62,000; at least about 63,000; at least about 64,000; at least about 65,000; at least about 66,000; at least about 67,000; at least about 68,000; at least about 69,000; at least about 70,000; at least about 71,000; at least about 72,000; at least about 73,000; at least about 74,000; at least about 75,000; at least about 76,000; at least about 77,000; at least about 78,000; at least about 79,000; at least about 80,000; at least about 81,000; at least about 82,000; at least about 83,000; at least about 84,000; at least about 85,000; at least about 86,000; at least about 87,000; at least about 88,000; at least about 89,000; at least about 90,000; at least about 91,000; at least about 92,000; at least about 93,000; at least about 94,000; at least about 95,000; at least about 96,000; at least about 97,000; at least about 98,000; at least about 99,000; at least about 100,000; at least about 200,000; at least about 300,000; at least about 400,000; at least about 500,000; at least about 600,000; at least about 700,000; at least about 800,000; at least about 900,000; at least about 1,000,000; at least about 1,100,000; at least about 1,200,000; at least about 1,300,000; at least about 1,400,000; at least about 1,500,000; at least about 1,600,000; at least about 1,700,000; at least about 1,800,000; at least about 1,900,000; at least about 2,000,000; at least about 2,100,000; at least about 2,200,000; at least about 2,300,000; at least about 2,400,000; at least about 2,500,000; at least about 2,600,000; at least about 2,700,000; at least about 2,800,000; at least about 2,900,000; at least about 3,000,000; at least about 3,100,000; at least about 3,200,000; at least about 3,300,000; at least about 3,400,000; at least about 3,500,000; at least about 3,600,000; at least about 3,700,000; at least about 3,800,000; at least about 3,900,000; at least about 4,000,000; at least about 4,100,000; at least about 4,200,000; at least about 4,300,000; at least about 4,400,000; at least about 4,500,000; at least about 4,600,000; at least about 4,700,000; at least about 4,800,000; at least about 4,900,000; at least about 5,000,000; at least about 5,100,000; at least about 5,200,000; at least about 5,300,000; at least about 5,400,000; at least about 5,500,000; at least about 5,600,000; at least about 5,700,000; at least about 5,800,000; at least about 5,900,000; at least about 6,000,000; at least about 6,100,000; at least about 6,200,000; at least about 6,300,000; at least about 6,400,000; at least about 6,500,000; at least about 6,600,000; at least about 6,700,000; at least about 6,800,000; at least about 6,900,000; at least about 7,000,000; at least about 7,100,000; at least about 7,200,000; at least about 7,300,000; at least about 7,400,000; at least about 7,500,000; at least about 7,600,000; at least about 7,700,000; at least about 7,800,000; at least about 7,900,000; at least about 8,000,000; at least about 8,100,000; at least about 8,200,000; at least about 8,300,000; at least about 8,400,000; at least about 8,500,000; at least about 8,600,000; at least about 8,700,000; at least about 8,800,000; at least about 8,900,000; at least about 9,000,000; at least about 9,100,000; at least about 9,200,000; at least about 9,300,000; at least about 9,400,000; at least about 9,500,000; at least about 9,600,000; at least about 9,700,000; at least about 9,800,000; at least about 9,900,000; at least about 10,000,000; at least about 10,100,000; at least about 10,200,000; at least about 10,300,000; at least about 10,400,000; at least about 10,500,000; at least about 10,600,000; at least about 10,700,000; at least about 10,800,000; at least about 10,900,000; at least about 11,000,000; at least about 11,100,000; at least about 11,200,000; at least about 11,300,000; at least about 11,400,000; at least about 11,500,000; at least about 11,600,000; at least about 11,700,000; at least about 11,800,000; at least about 11,900,000; at least about 12,000,000; at least about 12,100,000; at least about 12,200,000; at least about 12,300,000; at least about 12,400,000; at least about 12,500,000; at least about 12,600,000; at least about 12,700,000; at least about 12,800,000; at least about 12,900,000; at least about 13,000,000; at least about 13,100,000; at least about 13,200,000; at least about 13,300,000; at least about 13,400,000; at least about 13,500,000; at least about 13,600,000; at least about 13,700,000; at least about 13,800,000; at least about 13,900,000; at least about 14,000,000; at least about 14,100,000; at least about 14,200,000; at least about 14,300,000; at least about 14,400,000; at least about 14,500,000; at least about 14,600,000; at least about 14,700,000; at least about 14,800,000; at least about 14,900,000; at least about 15,000,000; at least about 15,100,000; at least about 15,200,000; at least about 15,300,000; at least about 15,400,000; at least about 15,500,000; at least about 15,600,000; at least about 15,700,000; at least about 15,800,000; at least about 15,900,000; at least about 16,000,000; at least about 16,100,000; at least about 16,200,000; at least about 16,300,000; at least about 16,400,000; at least about 16,500,000; at least about 16,600,000; at least about 16,700,000; at least about 16,800,000; at least about 16,900,000; at least about 17,000,000; at least about 17,100,000; at least about 17,200,000; at least about 17,300,000; at least about 17,400,000; at least about 17,500,000; at least about 17,600,000; at least about 17,700,000; at least about 17,800,000; at least about 17,900,000; at least about 18,000,000; at least about 18,100,000; at least about 18,200,000; at least about 18,300,000; at least about 18,400,000; at least about 18,500,000; at least about 18,600,000; at least about 18,700,000; at least about 18,800,000; at least about 18,900,000; at least about 19,000,000; at least about 19,100,000; at least about 19,200,000; at least about 19,300,000; at least about 19,400,000; at least about 19,500,000; at least about 19,600,000; at least about 19,700,000; at least about 19,800,000; at least about 19,900,000; at least about 20,000,000; at least about 20,100,000; at least about 20,200,000; at least about 20,300,000; at least about 20,400,000; at least about 20,500,000; at least about 20,600,000; at least about 20,700,000; at least about 20,800,000; at least about 20,900,000; at least about 21,000,000; at least about 21,100,000; at least about 21,200,000; at least about 21,300,000; at least about 21,400,000; at least about 21,500,000; at least about 21,600,000; at least about 21,700,000; at least about 21,800,000; at least about 21,900,000; at least about 22,000,000; at least about 22,100,000; at least about 22,200,000; at least about 22,300,000; at least about 22,400,000; at least about 22,500,000; at least about 22,600,000; at least about 22,700,000; at least about 22,800,000; at least about 22,900,000; at least about 23,000,000; at least about 23,100,000; at least about 23,200,000; at least about 23,300,000; at least about 23,400,000; at least about 23,500,000; at least about 23,600,000; at least about 23,700,000; at least about 23,800,000; at least about 23,900,000; at least about 24,000,000; at least about 24,100,000; at least about 24,200,000; at least about 24,300,000; at least about 24,400,000; at least about 24,500,000; at least about 24,600,000; at least about 24,700,000; at least about 24,800,000; at least about 24,900,000; at least about 25,000,000; at least about 25,100,000; at least about 25,200,000; at least about 25,300,000; at least about 25,400,000; at least about 25,500,000; at least about 25,600,000; at least about 25,700,000; at least about 25,800,000; at least about 25,900,000, at least 26,000,000 nucleobases downstream or upstream from the sequence set forth in SEQ ID NO:21 or SEQ ID NO:117.

In some aspect, the targeted to about integration site is located about 10; about 20; about 30; about 40; about 50; about 60; about 70; about 80; about 90; about 100; about 110; about 120; about 130; about 140; about 150; about 160; about 170; about 180; about 190; about 200; about 210; about 220; about 230; about 240; about 250; about 260; about 270; about 280; about 290; about 300; about 310; about 320; about 330; about 340; about 350; about 360; about 370; about 380; about 390; about 400; about 410; about 420; about 430; about 440; about 450; about 460; about 470; about 480; about 490; about 500; about 510; about 520; about 530; about 540; about 550; about 560; about 570; about 580; about 590; about 600; about 610; about 620; about 630; about 640; about 650; about 660; about 670; about 680; about 690; about 700; about 710; about 720; about 730; about 740; about 750; about 760; about 770; about 780; about 790; about 800; about 810; about 820; about 830; about 840; about 850; about 860; about 870; about 880; about 890; about 900; about 910; about 920; about 930; about 940; about 950; about 960; about 970; about 980; about 990; about 1,000; about 2,000; about 3,000; about 4,000; about 5,000; about 6,000; about 7,000; about 8,000; about 9,000; about 10,000; about 11,000; about 12,000; about 13,000; about 14,000; about 15,000; about 16,000; about 17,000; about 18,000; about 19,000; about 20,000; about 21,000; about 22,000; about 23,000; about 24,000; about 25,000; about 26,000; about 27,000; about 28,000; about 29,000; about 30,000; about 31,000; about 32,000; about 33,000; about 34,000; about 35,000; about 36,000; about 37,000; about 38,000; about 39,000; about 40,000; about 41,000; about 42,000; about 43,000; about 44,000; about 45,000; about 46,000; about 47,000; about 48,000; about 49,000; about 50,000; about 51,000; about 52,000; about 53,000; about 54,000; about 55,000; about 56,000; about 57,000; about 58,000; about 59,000; about 60,000; about 61,000; about 62,000; about 63,000; about 64,000; about 65,000; about 66,000; about 67,000; about 68,000; about 69,000; about 70,000; about 71,000; about 72,000; about 73,000; about 74,000; about 75,000; about 76,000; about 77,000; about 78,000; about 79,000; about 80,000; about 81,000; about 82,000; about 83,000; about 84,000; about 85,000; about 86,000; about 87,000; about 88,000; about 89,000; about 90,000; about 91,000; about 92,000; about 93,000; about 94,000; about 95,000; about 96,000; about 97,000; about 98,000; about 99,000; about 100,000; about 200,000; about 300,000; about 400,000; about 500,000; about 600,000; about 700,000; about 800,000; about 900,000; about 1,000,000; about 1,100,000; about 1,200,000; about 1,300,000; about 1,400,000; about 1,500,000; about 1,600,000; about 1,700,000; about 1,800,000; about 1,900,000; about 2,000,000; about 2,100,000; about 2,200,000; about 2,300,000; about 2,400,000; about 2,500,000; about 2,600,000; about 2,700,000; about 2,800,000; about 2,900,000; about 3,000,000; about 3,100,000; about 3,200,000; about 3,300,000; about 3,400,000; about 3,500,000; about 3,600,000; about 3,700,000; about 3,800,000; about 3,900,000; about 4,000,000; about 4,100,000; about 4,200,000; about 4,300,000; about 4,400,000; about 4,500,000; about 4,600,000; about 4,700,000; about 4,800,000; about 4,900,000; about 5,000,000; about 5,100,000; about 5,200,000; about 5,300,000; about 5,400,000; about 5,500,000; about 5,600,000; about 5,700,000; about 5,800,000; about 5,900,000; about 6,000,000; about 6,100,000; about 6,200,000; about 6,300,000; about 6,400,000; about 6,500,000; about 6,600,000; about 6,700,000; about 6,800,000; about 6,900,000; about 7,000,000; about 7,100,000; about 7,200,000; about 7,300,000; about 7,400,000; about 7,500,000; about 7,600,000; about 7,700,000; about 7,800,000; about 7,900,000; about 8,000,000; about 8,100,000; about 8,200,000; about 8,300,000; about 8,400,000; about 8,500,000; about 8,600,000; about 8,700,000; about 8,800,000; about 8,900,000; about 9,000,000; about 9,100,000; about 9,200,000; about 9,300,000; about 9,400,000; about 9,500,000; about 9,600,000; about 9,700,000; about 9,800,000; about 9,900,000; about 10,000,000; about 10,100,000; about 10,200,000; about 10,300,000; about 10,400,000; about 10,500,000; about 10,600,000; about 10,700,000; about 10,800,000; about 10,900,000; about 11,000,000; about 11,100,000; about 11,200,000; about 11,300,000; about 11,400,000; about 11,500,000; about 11,600,000; about 11,700,000; about 11,800,000; about 11,900,000; about 12,000,000; about 12,100,000; about 12,200,000; about 12,300,000; about 12,400,000; about 12,500,000; about 12,600,000; about 12,700,000; about 12,800,000; about 12,900,000; about 13,000,000; about 13,100,000; about 13,200,000; about 13,300,000; about 13,400,000; about 13,500,000; about 13,600,000; about 13,700,000; about 13,800,000; about 13,900,000; about 14,000,000; about 14,100,000; about 14,200,000; about 14,300,000; about 14,400,000; about 14,500,000; about 14,600,000; about 14,700,000; about 14,800,000; about 14,900,000; about 15,000,000; about 15,100,000; about 15,200,000; about 15,300,000; about 15,400,000; about 15,500,000; about 15,600,000; about 15,700,000; about 15,800,000; about 15,900,000; about 16,000,000; about 16,100,000; about 16,200,000; about 16,300,000; about 16,400,000; about 16,500,000; about 16,600,000; about 16,700,000; about 16,800,000; about 16,900,000; about 17,000,000; about 17,100,000; about 17,200,000; about 17,300,000; about 17,400,000; about 17,500,000; about 17,600,000; about 17,700,000; about 17,800,000; about 17,900,000; about 18,000,000; about 18,100,000; about 18,200,000; about 18,300,000; about 18,400,000; about 18,500,000; about 18,600,000; about 18,700,000; about 18,800,000; about 18,900,000; about 19,000,000; about 19,100,000; about 19,200,000; about 19,300,000; about 19,400,000; about 19,500,000; about 19,600,000; about 19,700,000; about 19,800,000; about 19,900,000; about 20,000,000; about 20,100,000; about 20,200,000; about 20,300,000; about 20,400,000; about 20,500,000; about 20,600,000; about 20,700,000; about 20,800,000; about 20,900,000; about 21,000,000; about 21,100,000; about 21,200,000; about 21,300,000; about 21,400,000; about 21,500,000; about 21,600,000; about 21,700,000; about 21,800,000; about 21,900,000; about 22,000,000; about 22,100,000; about 22,200,000; about 22,300,000; about 22,400,000; about 22,500,000; about 22,600,000; about 22,700,000; about 22,800,000; about 22,900,000; about 23,000,000; about 23,100,000; about 23,200,000; about 23,300,000; about 23,400,000; about 23,500,000; about 23,600,000; about 23,700,000; about 23,800,000; about 23,900,000; about 24,000,000; about 24,100,000; about 24,200,000; about 24,300,000; about 24,400,000; about 24,500,000; about 24,600,000; about 24,700,000; about 24,800,000; about 24,900,000; about 25,000,000; about 25,100,000; about 25,200,000; about 25,300,000; about 25,400,000; about 25,500,000; about 25,600,000; about 25,700,000; about 25,800,000; about 25,900,000, 26,000,000 nucleobases downstream or upstream from the sequence set forth in SEQ ID NO:21 or SEQ ID NO: 117.

In some aspects, the targeted-integration site is located in a high complexity sequence in the sequence set forth in SEQ ID NO: 22 or in the Chr3 TI contig. In some aspects, the targeted-integration site comprises a high complexity sequence in the sequence set forth in SEQ ID NO: 22 or in the Chr3 TI contig.

In some aspects, the targeted-integration site is located in a high complexity sequence in the sequence set forth in SEQ ID NO: 118 or in the Chr5 TI contig. In some aspects, the targeted-integration site comprises a high complexity sequence in the sequence set forth in SEQ ID NO: 118 or in the Chr5 TI contig.

In some aspects, the targeted-integration site is not located in a low complexity sequence in the sequence set forth in SEQ ID NO: 22 or in the Chr3 TI contig. In some aspects, the targeted-integration site is not located within a retrotransposon sequence in the sequence set forth in SEQ ID NO: 22 or in the Chr3 TI contig. In some aspects, the targeted-integration site is not located within an Alu repeat (CHO Alu-equivalent) or the like in the sequence set forth in SEQ ID NO: 22 or in the Chr3 TI contig. In some aspects, the targeted-integration site is not located within a long interspersed nuclear element (LINE) in the sequence set forth in SEQ ID NO: 22 or in the Chr3 TI contig. In some aspects, the targeted integration site/locus does not contain CHO Alu-equivalent sequences.

In some aspects, the targeted-integration site is not located in a low complexity sequence in the sequence set forth in SEQ ID NO: 118 or in the Chr5 TI contig. In some aspects, the targeted-integration site is not located within a retrotransposon sequence in the sequence set forth in SEQ ID NO: 118 or in the Chr5 TI contig. In some aspects, the targeted-integration site is not located within an Alu repeat (CHO Alu-equivalent) or the like in the sequence set forth in SEQ ID NO: 118 or in the Chr5 TI contig. In some aspects, the targeted-integration site is not located within a long interspersed nuclear element (LINE) in the sequence set forth in SEQ ID NO: 118 or in the Chr5 TI contig. In some aspects, the targeted integration site/locus does not contain CHO Alu-equivalent sequences.

In some aspects, the targeted-integration site does not comprise of multiple low complexity sequence in the sequence set forth in SEQ ID NO: 22 or in the Chr3 TI contig. In some aspects, the targeted-integration site does not comprise a retrotransposon sequence in the sequence set forth in SEQ ID NO: 22 or in the Chr3 TI contig. In some aspects, the targeted-integration site does not comprise an Alu repeat in the sequence set forth in SEQ ID NO: 22 or in the Chr3 TI contig. In some aspects, the targeted-integration site does not comprise a long interspersed nuclear element (LINE) in the sequence set forth in SEQ ID NO: 22 or in the Chr3 TI contig.

In some aspects, the targeted-integration site does not comprise of multiple low complexity sequence in the sequence set forth in SEQ ID NO: 118 or in the Chr5 TI contig. In some aspects, the targeted-integration site does not comprise a retrotransposon sequence in the sequence set forth in SEQ ID NO: 118 or in the Chr5 TI contig. In some aspects, the targeted-integration site does not comprise an Alu repeat in the sequence set forth in SEQ ID NO: 118 or in the Chr5 TI contig. In some aspects, the targeted-integration site does not comprise a long interspersed nuclear element (LINE) in the sequence set forth in SEQ ID NO: 118 or in the Chr5 TI contig.

In some aspects, the targeted-integration site is not flanked by a low complexity sequence in the sequence set forth in SEQ ID NO: 22 or in the Chr3 TI contig. In some aspects, the targeted-integration site is not flanked by a retrotransposon sequence in the sequence set forth in SEQ ID NO: 22 or in the Chr3 TI contig. In some aspects, the targeted-integration site is not flanked by an Alu repeat in the sequence set forth in SEQ ID NO: 22 or in the Chr3 TI contig. In some aspects, the targeted-integration site is not flanked by a long interspersed nuclear element (LINE) in the sequence set forth in SEQ ID NO: 22 or in the Chr3 TI contig.

In some aspects, the targeted-integration site is not flanked by a low complexity sequence in the sequence set forth in SEQ ID NO: 118 or in the Chr5 TI contig. In some aspects, the targeted-integration site is not flanked by a retrotransposon sequence in the sequence set forth in SEQ ID NO: 118 or in the Chr5 TI contig. In some aspects, the targeted-integration site is not flanked by an Alu repeat in the sequence set forth in SEQ ID NO: 118 or in the Chr5 TI contig. In some aspects, the targeted-integration site is not flanked by a long interspersed nuclear element (LINE) in the sequence set forth in SEQ ID NO: 118 or in the Chr5 TI contig.

As used herein, the term “low complexity sequence” refers to a nucleic acid sequence characterized by the presence of repeated sequences, also known as repetitive elements, repeated units, or repeats. Conversely, the term “high complexity sequence” refers to a nucleic acid sequence characterized by the absence of multiple repeated sequences. The main types of repeated sequences are tandem repeat, and interspersed repeats, which include transposable elements such as retrotransposons.

Retrotransposons (also called Class I transposable elements or transposons via RNA intermediates) are a type of genetic component that copy and paste themselves into different genomic locations (transposon) by converting RNA back into DNA through the process reverse transcription using an RNA transposition intermediate. There are two main types of retrotransposon, long terminal repeats (LTRs) and non-long terminal repeats (non-LTRs). Retrotransposons are classified based on sequence and method of transposition. Non-LTRs mostly fall into two types—LINEs (Long interspersed nuclear elements) and SINEs (Short interspersed nuclear elements. Alus are the most common SINE in primates.

The Alu family is a family of repetitive elements in primate genomes, including the human genome. An Alu element is a short stretch of DNA originally characterized by the action of the Arthrobacter luteus (Alu) restriction endonuclease. Alu elements are the most abundant transposable elements, containing over one million copies dispersed throughout the human genome. Modern Alu elements are about 300 base pairs long and are therefore classified as short interspersed nuclear elements (SINEs) among the class of repetitive DNA elements. The typical structure is 5′-Part A-A₅TACA₆-Part B-PolyA Tail-3′ (SEQ ID NO:25), where Part A and Part B (also known as “left arm” and “right arm”) are similar nucleotide sequences. Two main promoter “boxes” are found in Alu: a 5′ A box with the consensus TGGCTCACGCC (SEQ ID NO:26), and a 3′ B box with the consensus GWTCGAGAC (IUPAC nucleic acid notation).

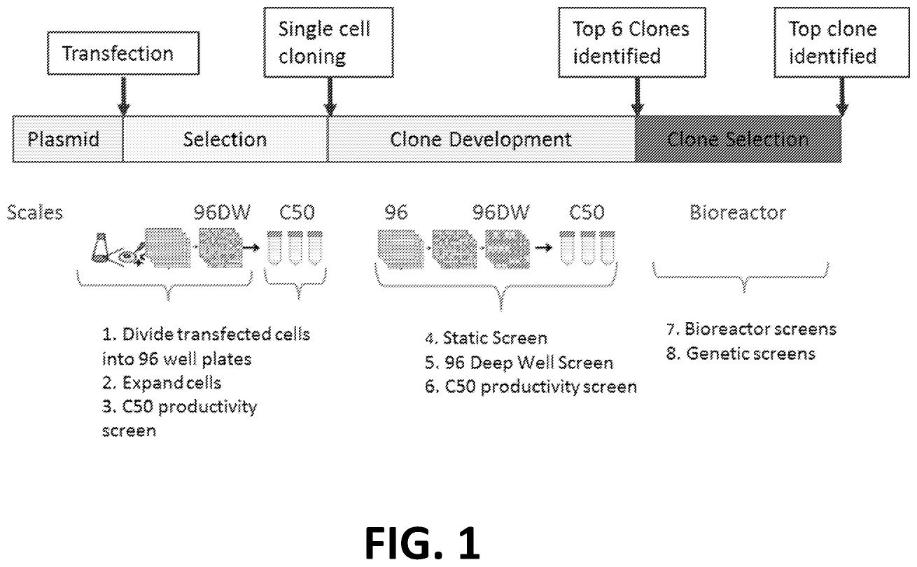

In the context of the present disclosure, references to Alu elements as applied to the Cricetulus griseus sequences disclosed herein refer to CHO Alu-equivalents, i.e., Alu-like elements present in the genome of Cricetulus griseus as described in Haynes et al. (1981) Molecular and Cellular Biology 1(7):573-583. Haynes et al. described a consensus sequence for a major interspersed deoxyribonucleic acid repeat in the genome of Chinese hamster ovary cells (CHO cells) which is extensively homologous to the human Alu sequence and the mouse B1 interspersed repetitious sequence. Because the CHO consensus sequence shows significant homology to the human Alu sequence it is termed the CHO Alu-equivalent sequence. A conserved structure surrounding CHO Alu-equivalent family members can be recognized. It is similar to that surrounding the human Alu and the mouse Bi sequences, and is represented as follows: direct repeat CHO-Alu-A-rich sequence-direct repeat. The consensus sequence of the CHO Alu-equivalent sequence is disclosed in FIG. 1 of the Haynes et al., which is herein incorporated by reference in its entirety.

Long interspersed nuclear elements (LINEs) (also known as long interspersed nucleotide elements or long interspersed elements) are a group of non-LTR (long terminal repeat) retrotransposons that are widespread in the genome of many eukaryotes. They make up around 21.1% of the human genome. LINEs make up a family of transposons, where each LINE is about 7,000 base pairs long. The only abundant LINE in humans is LINE1. The human genome contains an estimated 100,000 truncated and 4,000 full-length LINE-1 elements. Due to the accumulation of random mutations, the sequence of many LINEs has degenerated to the extent that they are no longer transcribed or translated.

In some aspects, the targeted-integration site does not comprise a CpG island in the sequence set forth in SEQ ID NO: 22 or in the Chr3 TI contig. In some aspects, the targeted-integration site is not within a CpG island in the sequence set forth in SEQ ID NO: 22 or in the Chr3 TI contig. In some aspects, the targeted-integration site is not flanked by CpG island in the sequence set forth in SEQ ID NO: 22 or in the Chr3 TI contig.

In some aspects, the targeted-integration site does not comprise a CpG island in the sequence set forth in SEQ ID NO: 118 or in the Chr5 TI contig. In some aspects, the targeted-integration site is not within a CpG island in the sequence set forth in SEQ ID NO: 118 or in the Chr5 TI contig. In some aspects, the targeted-integration site is not flanked by CpG island in the sequence set forth in SEQ ID NO: 118 or in the Chr5 TI contig.

CpG islands (or CG islands) are regions with a high frequency of CpG sites. Though objective definitions for CpG islands are limited, the usual formal definition is a region with at least 200 bp, a GC percentage greater than 50%, and an observed-to-expected CpG ratio greater than 60%. The “observed-to-expected CpG ratio” can be derived where the observed is calculated as: (number of CpGs) and the expected as (number of C*number of G)/length of sequence or ((number of C+number of G)/2)²/length of sequence.

In mammalian genomes, CpG islands are typically 300-3,000 base pairs in length, and have been found in or near approximately 40% of promoters of mammalian genes. Over 60% of human genes and almost all house-keeping genes have their promoters embedded in CpG islands.

Based on an extensive search on the complete sequences of human chromosomes 21 and 22, DNA regions greater than 500 bp are more likely to be the “true” CpG islands associated with the 5′ regions of genes if they had a GC content greater than 55%, and an observed-to-expected CpG ratio of 65%.

CpG islands are characterized by CpG dinucleotide content of at least 60% of that which would be statistically expected (~4-6%), whereas the rest of the genome has much lower CpG frequency (~1%), a phenomenon called CG suppression. Unlike CpG sites in the coding region of a gene, in most instances the CpG sites in the CpG islands of promoters are unmethylated if the genes are expressed. Most of the methylation differences between tissues, or between normal and cancer samples, occur a short distance from the CpG islands (at “CpG island shores”) rather than in the islands themselves.

Since histone acetylation and cytosine demethylation enhance transcription, targeted-integration sites are, e.g., located within loci with above average levels of acetylated histones and/or above average levels of unmethylated cytosines.

Accordingly, in some aspects, the targeted-integration site is located within, comprises, or is flanked by a subsequence in the sequence set forth in SEQ ID NO: 22 or in the Chr3 TI contig, or in SEQ ID NO:118 or in the Chr5 TI contig characterized by above average levels of unmethylated cytosines. As used herein, above average levels of unmethylated cytosines are considered with respect to the number of unmethylated cytosines over a certain polynucleotide length, e.g., per Kilobase. Thus, the percentage of unmethylated cytosines can be calculated, for example, for the sequence set forth in SEQ ID NO: 22 or SEQ ID NO:118, to obtain an average level of unmethylated cytosines. Then, subsequences in SEQ ID NO: 22 or SEQ ID NO:118 can be scored according to whether the percentage of unmethylated cytosines in the subsequences (e.g., 10 nt, 100 nt, 1000 nt, 10000 nt, more) are above or below the average number of unmethylated cytosines calculated for the entire sequence set forth in SEQ ID NO: 22 or SEQ ID NO:118.

Similarly, in some aspects, the targeted-integration site is located within, comprises, or is flanked by a subsequence in the sequence set forth in SEQ ID NO: 22 or in the Chr3 TI contig, or in SEQ ID NO:118 or in the Chr5 TI contig, characterized by being associated with histones having above average levels of acetylation. As used herein, above average levels of histone acetylation are considered with respect to the number of acetylated histones over a certain polynucleotide length, e.g., per Kilobase. Thus, the percentage of acetylated histones can be calculated, for example, for the sequence set forth in SEQ ID NO: 22 or SEQ ID NO:118, to obtain an average level of acetylated histones. Then, subsequences in SEQ ID NO: 22 or SEQ ID NO:118 can be scored according to whether the percentage of acetylated histones in the subsequences (e.g., 10 nt, 100 nt, 1000 nt, 10000 nt, or more) are above or below the average number of acetylated histones calculated for the entire sequence set forth in SEQ ID NO: 22 or SEQ ID NO:118.

Methods to identify markers of methylation and boundaries of transcription or open chromatin are provided, for example, in Sharmin et al. (2016) BMC Cancer 16:88; Wang et al. (2012) Nucleic Acids Res. 40:511-29; Papin et al. (2020) J. Mol. Biol. doi: 10.1016/j.jmb.2020.09.018; Li et al. (2013) BMC Genomics 14:553; Butcher & Beck (2015) Methods 72:21-8; Chen et al. (2020) Epigenetics 22:1-22; Keller et al. (2016) Mol. Biol. Evol. 33:1019-28; Symmons et al. (2014) Genome Res. 24:390-400; or Mifsud et al. (2015) Nat. Genet. 47:598-606; Collings & Anderson (2017) Epigenetics and Chromatin 10 doi.org/10.1186/s13072-017-0125-5 all of which are herein incorporated by reference in their entireties.

In some aspects, the targeted-integration site is located within, comprises, or is flanked by a subsequence in the sequence set forth in SEQ ID NO: 22 or in the Chr3 TI contig characterized by being a region with early initiation of replication.

In some aspects, the targeted-integration site is located within, comprises, or is flanked by a subsequence in the sequence set forth in SEQ ID NO: 118 or in the Chr5 TI contig characterized by being a region with early initiation of replication.

Early initiation of replication is associated with open chromatin and areas of transcription. Methods to identify origins of replication and their association with chromatin state and transcription are provided, for example, in Smith & Aladjem (2014) J. Mol. Biol. 426:3330-41; Dellino et al. (2013) Genome Res. 23: 1-11; Boos & Ferreira (2019) Genes 10:199; Boulos et al. (2015) FEBS Lett. 489:2944-57; or Gomez & Brockdorff (2004) Proc. Natl. Acad. Sci. USA 101:6923-6928; all of which are herein incorporated by reference in their entireties. Based on these methods, origins of replication within the sequence set forth in SEQ ID NO: 22 or in the Chr3 TI contig, or in SEQ ID NO: 118 or in the Ch5 TI contig can be classified and ranked as early, middle, and late initiation of replication regions. In some aspects, the targeted-integration site is located within, comprises, or is flanked by a subsequence in the sequence set forth in SEQ ID NO: 22 or in the Chr3 TI contig, or in SEQ ID NO: 118 or in the Ch5 TI contig characterized by being within the top 1%, 2%, 3%, 4%, 5%, 6%, 7%, 8%, 9%, 10%, 15%, 20%, 25%, 30%, 35%, 40%, 45%, or 50% of initiation of replication regions.

In some aspects, the targeted-integration site comprises the sequence set forth in SEQ ID NO:21, located at positions 20,002-20,019 of SEQ ID NO:20, or a portion thereof. In some aspects, the targeted-integration site is located within or comprises a subsequence of the sequence of SEQ ID NO:21 within SEQ ID NO:20.

In some aspects, the targeted-integration site comprises the sequence set forth in SEQ ID NO:117, or a portion thereof. In some aspects, the targeted-integration site is located within or comprises a subsequence of the sequence of SEQ ID NO:117 within SEQ ID NO:116.

In some aspects, the targeted-integration site is located upstream from SEQ ID NO:21 or SEQ ID NO:117.

In some aspects, the targeted-integration site is located downstream from SEQ ID NO:21 or SEQ ID NO:117.

In some aspects, the targeted-integration site is located about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900 or about 3000 nt upstream from SEQ ID NO: 21 or SEQ ID NO: 117.

In some aspects, the targeted-integration site is located about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900 or about 3000 nt downstream from SEQ ID NO: 21 or SEQ ID NO: 117.

In some aspects, the targeted-integration site is located within a sequence orthologous to the sequence set forth in SEQ ID NO: 20, or a fragment thereof, e.g., SEQ ID NO: 21. In some aspects, the targeted-integration site is located within a sequence paralogous to the sequence set forth in SEQ ID NO: 20, or a fragment thereof, e.g., SEQ ID NO: 21.

In some aspects, the targeted-integration site is located within a sequence orthologous to the sequence set forth in SEQ ID NO: 116, or a fragment thereof, e.g., SEQ ID NO: 117. In some aspects, the targeted-integration site is located within a sequence paralogous to the sequence set forth in SEQ ID NO: 116, or a fragment thereof, e.g., SEQ ID NO: 117.

In some aspects of the present disclosure, the targeted-integration site is located within SEQ ID NO: 20. SEQ ID NO: 20 is a subsequence of SEQ ID NO: 22 (26 Mbase sequence from chromosome 3 of Cricetulus griseus, Chinese hamster) comprising 20 Kb on each site of the integration site of SEQ ID NO: 21.

In some aspects of the present disclosure, the targeted-integration site is located within SEQ ID NO: 116. SEQ ID NO: 116 is a subsequence of SEQ ID NO: 118 (18 Mbase sequence from chromosome Chr5 of Cricetulus griseus, Chinese hamster) comprising 20 Kb on each site of the integration site of SEQ ID NO: 117.

In some aspects, the present disclosure provides an isolated cell comprising a polynucleotide sequence (an exogenous nucleic acid) which comprises a nucleic acid encoding a gene of interest (GOI) integrated within a specific locus of the genome of the cell, wherein the locus has at least about 70%, about 75%, about 80%, about 85%, about 90%, about 95%, about 96%, about 97%, about 98%, about 99%, or 100% sequence identity to SEQ ID NO: 20, to SEQ ID NO: 116 or a subsequence thereof.

It is to be understood that whereas the sequences disclosed herein are derived from Cricetulus griseus, the present disclosure also encompasses orthologous sequences from other species, e.g., human, mouse, rabbit, rat, pig, or dog. Thus, references to any of the sequences set forth in SEQ ID NOS: 14-24 and 110-120 also encompass variant sequences having at least about 70%, about 75%, about 80%, about 85%, about 90%, about 95%, about 96%, about 97%, about 98%, about 99%, or 100% sequence identity to those parent or reference sequences (i.e., any sequence set forth in SEQ ID NOS: 14-24 and 110-120 or a fragment or subsequence thereof), as determined, for example, via pairwise alignment using an implementation of the Needleman-Wunsch algorithm. As used herein the term “orthologous” refers to polynucleotides that have a similar nucleic acid sequence because they were separated by a speciation event, i.e., they represent homologous sequences in different organisms due to an ancestral relationship and therefore serve a similar function in different organisms. Thus, a sequence (or subsequence) that is orthologous to a sequences (or subsequence) disclosed herein is considered functionally equivalent, i.e., equally capable of being used a specific locus for targeted integration as a known sequence from Cricetulus griseus disclosed herein.

In some aspects, the present disclosure provides an isolated cell comprising a polynucleotide sequence (an exogenous nucleic acid) which comprises a nucleic acid encoding a gene of interest (GOI) integrated within a specific locus of the genome of the cell, wherein the locus has at least about 70%, about 75%, about 80%, about 85%, about 90%, about 95%, about 96%, about 97%, about 98%, about 99%, or 100% sequence identity to SEQ ID NO: 20, to SEQ ID NO: 116 or a subsequence thereof.

In some aspects, the subsequence is about 18, about 20, about 25, about 30, about 40, about 50, about 60, about 70, about 80, about 90, about 100, about 110, about 120, about 130, about 140, about 150, about 175, about 200, about 225, about 250, about 275, about 300, about 350, about 400, about 450, about 500, about 550, about 600, about 700, about 800, about 900, about 1000, about 1100, about 1200, about 1300, about 1400, about 1500, about 1600, about 1700, about 1800, about 1900, or about 2000 nucleotides in length, wherein the subsequence comprises the sequence set forth in SEQ ID NO: 21 or SEQ ID NO: 117.

In some aspects, the subsequence comprises about 10, about 20, about 30, about 40, about 50, about 60, about 70, about 80, about 90, about 100, about 110, about 120, about 130, about 140, about 150, about 175, about 200, about 225, about 250, about 275, about 300, about 350, about 400, about 450, about 500, about 550, about 600, about 700, about 800, about 900, about 1000, about 1100, about 1200, about 1300, about 1400, about 1500, about 1600, about 1700, about 1800, about 1900, or about 2000 nucleotides upstream with respect to the sequence set forth in SEQ ID NO: 21 or SEQ ID NO: 117.

In some aspects, the subsequence comprises about 10, about 20, about 30, about 40, about 50, about 60, about 70, about 80, about 90, about 100, about 110, about 120, about 130, about 140, about 150, about 175, about 200, about 225, about 250, about 275, about 300, about 350, about 400, about 450, about 500, about 550, about 600, about 700, about 800, about 900, about 1000, about 1100, about 1200, about 1300, about 1400, about 1500, about 1600, about 1700, about 1800, about 1900, or about 2000 nucleotides downstream with respect to the sequence set forth in SEQ ID NO: 21 or SEQ ID NO: 117.

In some aspects, the present disclosure provides an isolated cell comprising a polynucleotide sequence (an exogenous nucleic acid) which comprises a nucleic acid encoding a gene of interest (GOI) integrated within a specific locus of the genome of the cell, wherein the locus (e.g., a targeted-integration site of the present disclosure) is a nucleotide position or nucleotide sequence within SEQ ID NO: 20 or SEQ ID NO: 116. The present disclosure also provides a method comprising introducing into a mammalian cell, e.g., a CHO cell, a polynucleotide sequence (an exogenous nucleic acid) which comprises a nucleic acid encoding a gene of interest (GOI) and obtaining a mammalian cell, e.g., a CHO cell, wherein the exogenous nucleic acid is integrated into a specific locus of the genome of the CHO cell, the locus is a nucleotide position or nucleotide sequence within SEQ ID NO: 20 or SEQ ID NO: 116. Also provided is a method comprising (a) providing a cell comprising a polynucleotide sequence (an exogenous nucleic acid) which comprises a nucleic acid encoding a gene of interest (GOI), wherein the polynucleotide sequence comprises the nucleic acid encoding a gene of interest (GOI) operably linked to a promoter, and wherein the locus is a nucleotide position or nucleotide sequence within SEQ ID NO: 20 and SEQ ID NO: 116. In some aspects, the locus partially overlaps SEQ ID NO:20 or SEQ ID NO:16.

In some aspects, the polynucleotide sequence comprises the nucleic acid encoding a gene of interest (GOI) is integrated into a specific site at any position of SEQ ID NO: 20 or SEQ ID NO: 116.

In some aspects, the specific site is at a position within SEQ ID NO: 20 selected from the group consisting of nucleotides spanning positions numbers 1-1000, 1001-2000, 2001-3000, 3001-4000, 4001-5000, 5001-6000, 6001-7000, 7001-8000, 8001-9000, 9001-10000, 10001-11000, 11001-12000, 12001-13000, 13001-14000, 14001-15000, 15001-16000, 16001-17000, 17001-18000, 18001-19000, 190001-20000, 20001-21000, 21001-22000, 22001-23000, 23001-24000, 24001-25000, 25001-26000, 26001-27000, 27001-28000, 28001-29000, 29001-30000, 30001-31000, 31001-32000, 32001-33000, 33001-34000, 34001-35000, 35001-36000, 36001-37000, 37001-38000, 38001-39000, 39001-40000, or 40001-40020.

In some aspects, the specific site is at a position within SEQ ID NO: 116 selected from the group consisting of nucleotides spanning positions numbers 1-1000, 1001-2000, 2001-3000, 3001-4000, 4001-5000, 5001-6000, 6001-7000, 7001-8000, 8001-9000, 9001-10000, 10001-11000, 11001-12000, 12001-13000, 13001-14000, 14001-15000, 15001-16000, 16001-17000, 17001-18000, 18001-19000, 190001-20000, 20001-21000, 21001-22000, 22001-23000, 23001-24000, 24001-25000, 25001-26000, 26001-27000, 27001-28000, 28001-29000, 29001-30000, 30001-31000, 31001-32000, 32001-33000, 33001-34000, 34001-35000, 35001-36000, 36001-37000, 37001-38000, 38001-39000, 39001-40000, 40001-41000, 410001-42000, 42001-43000, 43001-44000, 44001-45000, 45001-46000, 46001-47000, 47001-48000, 49001-50000, 50001-51000, 51001-52000, 52001-53000, 53001-54000, 54001-55000, 55001-56000, 56001-5700. 57001-58000, 58001-59000, 59001-60000, 60001-61000, 61001-62000, 62001-63000, 63001-64000, 64001-65000, 65001-66000, 66001-67000, 67001-68000, 68001-69000, 69001-70000, 70001-71000, 71001-72000, 72001-7300, 73001-74000, 74001-75000, 75001-76000, 76001-77000, 77001-78000, 78001-79000, 79001-80000, 80001-81000, 81001-82000, 82001-83000, 83001-84000, 84001-85000, 85001-86000, 86001-87000, 87001-88000, 88001-89000, 89001-90000, 90001-91000, 91001-92000, 92001-93000, 93001-94000, 94001-95000, 95001- 96000, 96001-97000, 97001-98000, 98001-99000, 99001-100000, 100001-101000, 101001-102000, 102001-103000, 103001-104000, 104001-105000, 105001-106000, 106001-107000, 107001-108000, 108001-109000, 109001-110000, 110001-111000, 111001-112000, 112001-113000, 113001-114000, 114001-115000, 115001-116000, 116001-117000, 117001-118000, 118001-119000, 119001-120000, 120001-121000, 121001-122000, 122001-123000, 123001-124000, 124001-125000, 125001-126000, 126001-127000, 127001-128000, 128001-129000, 129001-130000, 130001-131000, 131001-132000, 132001-133000, 133001-134000, 134001-135000, 135001-136000, 136001-137000, 137001-138000, 138001-139000, 139001-140000, 140001-141000, 141001-142000, 142001-143000, 143001-144000, 144001-145000, 145001-146000, 146001-147000, 147001-148000, 148001-149000, 149001-150000, 150001-151000, 151001-152000, 152001-153000, 153001-154000, 154001-155000, 155001-156000, 156001-157000, 157001-158000, 158001-159000, 159001-160000, 160001-161000, 161001-162000, 162001-163000, 163001-164000, 164001-165000, 165001-166000, 166001-167000, 167001-168000, 168001-169000, 169001-170000, 170001-171000, 171001-172000, 172001-173000, 173001-174000, 174001-175000, 175001-176000, 176001-177000, 177001-178000, 178001-179000, 179001-180000, 180001-181000, 181001-182000, 182001-183000, 183001-184000, OR 184001-185000.

In some aspects, the specific site is at a position within SEQ ID NO: 20 selected from the group consisting of nucleotides spanning positions numbers 19000-21000, 18000-22000, 17000-23000, 16000-24000, 15000-25000, 14000-26000, 13000-27000, 12000-28000, 11000-29000, 10000-30000, 9000-31000, 8000-32000, 7000-33000, 6000-34000, 5000-35000, 4000-36000, 3000-37000, 2000-38000, 1000-39000, or 1-40020.

In some aspects, the specific site is at a position within SEQ ID NO: 20 selected from the group consisting of nucleotides spanning positions numbers 19000-19100, 19100-19200, 19200-19300, 19300-19400, 19400-19500, 19500-19600, 19600-19700, 19700-19800, 19800-19900, 19900-20000, 20000-20100, 20100-20200, 20200-20300, 20300-20400, 20400-20500, 20500-20600, 20600-20700, 20700-20800, 20800-20900, or 20900-21000.

In some aspects, the polynucleotide sequence comprises the nucleic acid encoding a gene of interest (GOI) is integrated into a specific site at any position of SEQ ID NO: 20, wherein the specific site is at a position within SEQ ID NO: 20 consisting of nucleotides spanning positions numbers 20000-20020, 19990-20030, 19980-20040, 19970-20050, 19960-20060, 19950-20070, 19940-20080, 19930-20090, 19920-20100, 19910-20110, 19900-20120, 19890-20120, 19880-20130, 19870-20140, 19860-20150, 19850-20160, 19840-20170, 19830-20180, 19820-20190, 19810-20200, 19800-20210, 19790-20220, 19780-20230, 19770-20230, 19760-20240, 19750-20250, 19740-20260, 19730-20270, 19720-20280, 19710-20290, 19700-20300, 19690-20310, 19680-20320, 19670-20330, 19660-20340, 19650-20350, 19640-20360, 19630-20370, 19620-20380, 19610-20390, 19600-20400, 19590-20410, 19580-20420, 19570-20430, 19560-20440, 19550-20450, 19540-20460, 19530-20470, 19520-20480, 19510-20490, or 19500-20500.

In some aspects, the polynucleotide sequence comprises the nucleic acid encoding a gene of interest (GOI) is integrated into a specific site (hot spot) at any position within SEQ ID NO: 20 (genomic sequence comprising Hot Spot 1) or within SEQ ID NO: 116 (genomic sequence comprising Hot Spot 2), or partially overlapping SEQ ID NO: 20 or SEQ ID NO: 116.

In some aspects, the specific site at a position within SEQ ID NO: 20 is selected from the group consisting of nucleotide positions or subsequences spanning positions number 20,002-20,019 (corresponding to the 18-mer sequence set forth in SEQ ID NO: 21).

In some aspects, the specific site is at a position within SEQ ID NO: 20 selected from the group consisting of nucleotide positions 19900, 19901, 19902, 19903, 19904, 19905, 19906, 19907, 19908, 19909, 19910, 19911, 19912, 19913, 19914, 19915, 19916, 19917, 19918, 19919, 19920, 19921, 19922, 19923, 19924, 19925, 19926, 19927, 19928, 19929, 19920, 19921, 19922, 19923, 19924, 19925, 19926, 19927, 19928, 19929, 19930, 19931, 19932, 19933, 19934, 19935, 19936, 19937, 19938, 19939, 19949, 19941, 19942, 19943, 19944, 19945, 19946, 19947, 19948, 19949, 19950, 19951, 19952, 19953, 19954, 19955, 19956, 19956, 19958, 19959, 19960, 19961, 19962, 19963, 19964, 19965, 19966, 19967, 19968, 19969, 19970, 19971, 19971, 19972, 19973, 19974, 19975, 19976, 19977, 19978, 19979, 19980, 19981, 19982, 19983, 19984, 19985, 19986, 19987, 19988, 19989, 19990, 19991, 19992, 19993, 19994, 19995, 19996, 19997, 19998, 19999, 20000, 20001, 20002, 20003, 20004, 20005, 20006, 20007, 20008, 20009, 20010, 20011, 20012, 20013, 20014, 20015, 20016, 20017, 20018, 20019, 20020, 20021, 20022, 20023, 20024, 20025, 20026, 20027, 20028, 20029, 20030, 20031, 20032, 20033, 20034, 20035, 20036, 20037, 20038, 20039, 20040, 20041, 20042, 20043, 20044, 20045, 20046, 20047, 20048, 20049, 20050, 20051, 20052, 20053, 20054, 20055, 20056, 20057, 20058, 20059, 20060, 20061, 20062, 20063, 20064, 20065, 20066, 20067, 20068, 20069, 20070, 20071, 20072, 20073, 20074, 20075, 20076, 20077, 20078, 20079, 20080, 20081, 20082, 20083, 20084, 20085, 20086, 20087, 20088, 20089, 20090, 20091, 20092, 20093, 20094, 20095, 20096, 20097, 20098, 20099, or 20100.

The present disclosure also provides methods that allowed the generation of landing pad cell lines and expression cell lines as well as the identification of additional hot spots in the genome of a parental cell line without any prior knowledge of the genomic sequences surrounding the parental plasmids. This universal TI technology makes use of site-specific endonuclease(s) directed at parental plasmid sequences in the Parental Cell line not present in the landing pad plasmid. An advantage of this strategy is that no knowledge of the flanking genomic DNA sequence is needed. For example, FIG. 4A shows the requirement of knowing genomic sequences targeted by CRISPR/Cas, indicated by solid boxes next to scissors which represent CRISPR/Cas. In contrast, FIGS. 8A and 8B shows that the sequences targeted by CRISPR/Cas are internal to the parental plasmid. The boxes with vertical and wavy lines represent regions of homology between different plasmids.

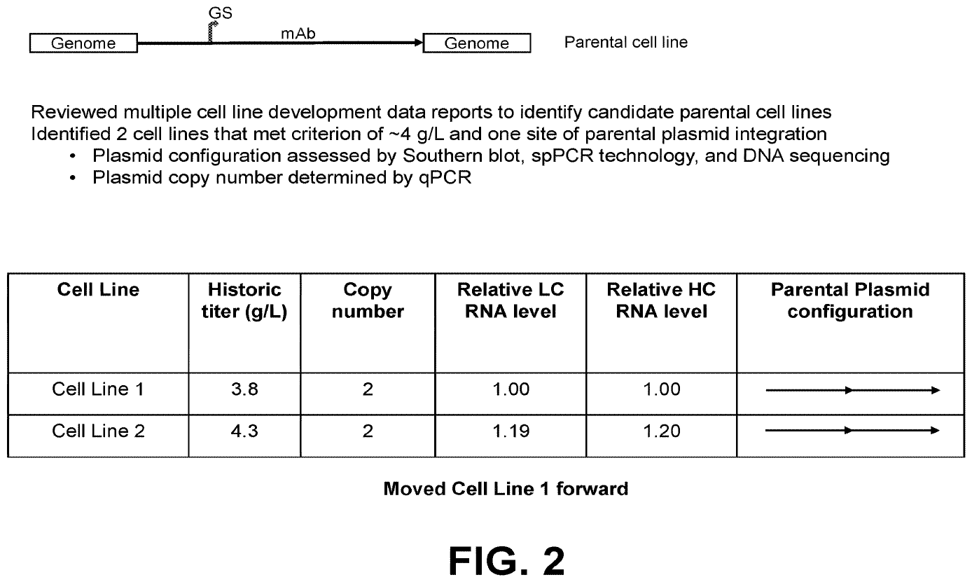

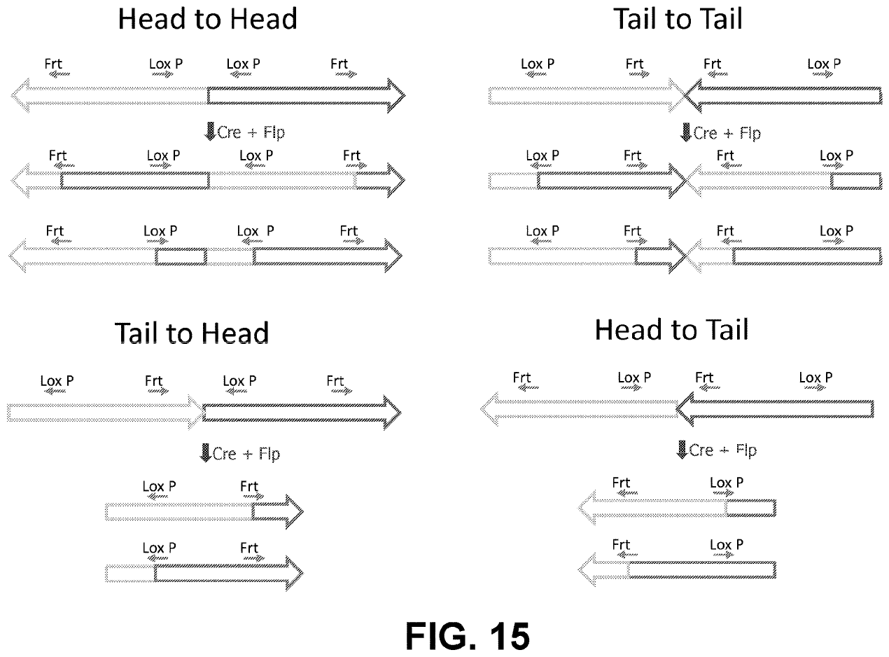

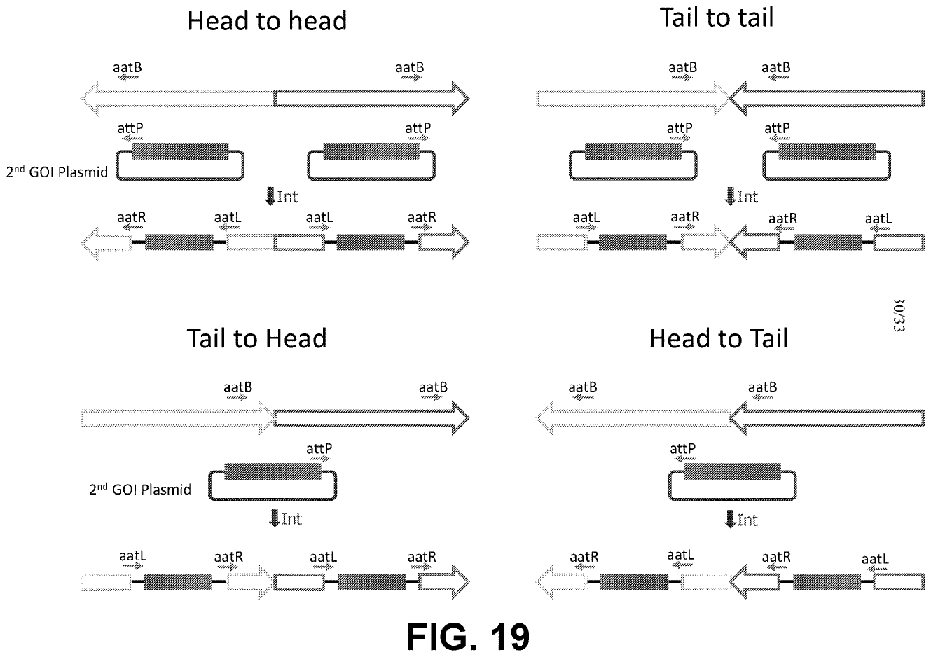

According to these new strategies, a parental cell line with a high expression titer (e.g., 3-4 g/L for an antibody) and low copy number (e.g., 2) would be selected first, e.g., as shown in FIG. 2 and related disclosures. Once such cell line, i.e., a “hot cell line” has been identified, the hot cell line can be used according to two different strategies. In both strategies, a landing pad plasmid encodes for a marker, e.g., a fluorescent marker such as b1mCherry, and expresses a selection marker, e.g., puromycin resistance, that is different from the parental plasmid present in the parental cell line, and the polynucleotide sequence encoding the marker is flanked by heterologous site-specific recombination sites (SSRS). The exemplary SSRS shown in FIGS. 12A, 12B, 13 and 14 are two Lox sites (LoxP and Lox511), which are targets of the Cre recombinase. However, alternative SSRS, e.g., Lox, Frt, att, or combinations thereof, may be used to practice these strategies as disclosed below. For example, Lox and Frt combinations are depicted in FIG. 15 and the use of att sites (attachment sites) is shown, e.g., in FIG. 19.

In the presence of the site specific endonuclease (e.g., CRISPR/Cas) and the landing pad plasmid, the first GOI (e.g., a mAb expression cassette) in the parental cell line is either replaced with the landing pad shown as mCherry flanked by Lox site (Strategy A), or is deleted and the landing pad plasmid is integrated into an alternative locus in the genome of the hot cell line (Strategy B). Thus, in Strategy A, the landing pad plasmid would be inserted in a hot spot which supports high expression, which would be the same hot spot used in the parental cell line. In Strategy B, the first GOI (e.g., a mAb expression cassette) in the parental cell line would be removed, and the landing pad plasmid inserted at alternative locations in the genome of the parental cell line. Since the parental cell line is a hot cell, identification of additional hot spots will result in landing pad cell lines able to generate expression cell lines with a preferred attributes such as high titer. See FIG. 8A.

The present disclosure provides a method for identifying a landing pad cell line comprising:
(1) removing the first GOI from a plasmid integrated in the genomic sequence of a parental cell (e.g., a hot cell), thus generating a population of parent cells without the first GOI;
(2) integrating a landing pad plasmid comprising at least one marker (e.g., Cherry) at alternative genomic loci in the population of parental cells of (1), thus generating a library or candidate cells; and,
(3) screening a library of candidate cells comprising at least one copy of the landing pad plasmid integrated at at least one alternative genomic loci, wherein a candidate cell line is selected if it meets a desired attribute such as (a) cell titer is above a predetermined threshold level; (b) plasmid copy number is at predetermined value; (c) RNA expression level as above a predetermined threshold level; or, (d) multiple plasmid copies, if present, have a specific plasmid configuration.

In some aspects, only cells containing a landing pad plasmid in a newly identified hot spot are selected. In some aspects, cell containing more than one landing pad plasmid in a newly identified hot spot are selected. In some aspects, the parental cell is a historical cell line, e.g., a cell line characterized by high titer in the expression of a GOI, for example, an antibody or an antigen-binding portion thereof. In some aspects, the library of candidate cells is a library generated via random integration of the landing pad sequence at multiple locations in the genome of a parental cell modified. e.g., by deleting/excising/removing an expression cassette encoding a protein of interests such as an antibody or antigen-binding portion thereof. In some aspects, the method selects a hot cell with at least one landing pad plasmid integrated in a new hot spot. In some aspects, the parental cell line is a CHO cell line.

The present disclosure provides a method of generating a landing pad cell comprising integrating a landing pad plasmid into the genome of a parental cell (e.g., a CHO hot cell) at a targeted-integration site using homologous recombination (e.g., using CRISPR/Cas), wherein the sequences targeted for homologous recombination are located in the parental plasmid, i.e., the sequences targeted for homologous recombination are not genomic sequences, wherein homologous recombination sites of the landing pad plasmid recombine with corresponding homologous recombination sites of the parental plasmid, thereby integrating the landing pad plasmid at an internal location within the parental plasmid inserted in the parent cell genomic DNA.

In some aspects, each landing pad plasmid comprises (i) a polynucleotide sequence comprising a nucleic acid encoding at least one selection marker and/or at least one nucleic acid sequence encoding a detectable marker; (ii) two site-specific recombination sites (SSRS) flanking the polynucleotide sequence of (i); and, (iii) two homologous recombination sites located 5′ and 3′ terminally with respect to the SSRSs of (ii), which are homologous to corresponding homologous recombination sites in the parental plasmid.

The present disclosure also provides a method of generating an expression cell comprising integrating a GOI plasmid (e.g., a plasmid encoding an antibody or antigen-binding portion thereof) into the genome of the landing pad cell disclosed above (e.g., a CHO hot cell) using site-specific recombinase recombination (e.g., using a Cre/Lox system), wherein site-specific recombination sites of the landing pad plasmid recombine with corresponding site-specific recombination sites of GOI plasmid, thereby integrating the GOI. In some aspects, the resulting expression plasmid comprises (i) a polynucleotide sequence comprising a nucleic acid encoding at least one GOI; and, (ii) two SSRS flanking the polynucleotide of (i).

Also provided is method of generating an expression cell comprising: (a) integrating a landing pad plasmid into the genome of a parental cell (e.g., a parent hot cell) at a targeted-integration site using homologous recombination, wherein the sequences targeted for homologous recombination are located in the parental plasmid, and, wherein homologous recombination sites in the landing pad plasmid recombine with corresponding homologous recombination sites of the parental plasmid in the landing pad at a different genomic locus, thereby integrating the landing pad plasmid at an internal location within the landing pad at the different genomic locus in the parent cell genomic DNA; and, (b) integrating a GOI plasmid into the genome of the landing pad cell using site-specific recombinase recombination, wherein the site-specific recombination sites of the landing pad plasmid recombine with the corresponding site-specific recombination sites of GOI plasmid, thereby integrating the GOI plasmid at an internal location within the landing pad plasmid of the landing pad cell. In some aspects of this method, each landing pad plasmid comprises (i) a at least one polynucleotide sequence comprising a nucleic acid encoding at least one selection marker and/or at least one nucleic acid sequence encoding a detectable marker; (ii) two SSRS flanking the polynucleotide sequence of (i); and, (iii) two homologous recombination sites located 5′ and 3′ terminally with respect to the SSRSs of (ii), which are homologous to corresponding homologous recombination sites in a parental plasmid. In some aspects of this method, the resulting expression plasmid comprises (i) a polynucleotide sequence comprising a nucleic acid encoding at least one GOI; and, (ii) two SSRS flanking the polynucleotide of (i).

Also provided is a method of generating a landing pad cell comprising: (a) removing a parental plasmid or a portion thereof from a first hot spot location in a parental cell line; and, (b) integrating a landing pad plasmid into a second hot spot location in the genome of the parental cell at a targeted-integration site using homologous recombination, wherein the sequences targeted for homologous recombination are present in the parental plasmid, and wherein the homologous recombination sites of the landing pad plasmid recombine with the corresponding homologous recombination sites of the parental plasmid, thereby integrating the landing pad plasmid at an internal location within the parental plasmid inserted in the parent cell genomic DNA. In some aspects of this method, the landing pad plasmid comprises (i) a polynucleotide sequence comprising a nucleic acid encoding at least one selection marker and/or at least one nucleic acid sequence encoding a detectable marker; (ii) two site-specific recombination sites (SSRS) flanking the polynucleotide sequence of (i); and, (iii) two homologous recombination sites located 5′ and 3′ terminally with respect to the SSRSs of (ii), which are homologous to corresponding homologous recombination sites in a parental plasmid. Step (a) would generate a population of cells derived from the parental cell line (e.g., a hot cell line) without the first GOI (e.g., an antibody that had a high expression level in the parental cell line). In Step (b), the insertion of the landing pad plasmid in the genomes of the population of cells of Step (a) would generate a population of cells which would contain that land cell pad integrated at multiple locations, which could in turn be screened to identify new hot cells and their corresponding hot spots.

Also provided is a method of generating a expression cell comprising: (a) removing a parental plasmid from a first hot spot location in a parental cell line, (b) integrating a landing pad plasmid into a second hot spot location in the genome of the parental cell at a targeted-integration site using homologous recombination, wherein the sequences targeted for homologous recombination were present in the parental plasmid, wherein each landing pad plasmid comprises, e.g., (i) a polynucleotide sequence comprising a nucleic acid encoding at least one selection marker and/or at least one nucleic acid sequence encoding a detectable marker; (ii) two site-specific recombination sites (SSRS) flanking the polynucleotide sequence of (i); and, (iii) two homologous recombination sites located 5′ and 3′ terminally with respect to the SSRSs of (ii), which are homologous to corresponding homologous recombination sites in a parental plasmid; and, wherein the homologous recombination sites of the landing pad plasmid recombine with the corresponding homologous recombination sites of the parental plasmid, thereby integrating the landing pad plasmid at an internal location within the parental plasmid inserted in the parent cell genomic DNA, and, (c) integrating a GOI plasmid into the genome of the landing pad cell using site-specific recombinase recombination, wherein the expression plasmid comprises, e.g., (i) a polynucleotide sequence comprising a nucleic acid encoding a GOI; and, (ii) two SSRS flanking the polynucleotide of (1); wherein the site-specific recombination sites of the landing pad plasmid recombine with the corresponding site-specific recombination sites of GOI plasmid, thereby integrating the GOI plasmid at an internal location within the landing pad plasmid of the landing pad cell.

In some aspects, the landing pad cell comprises a plasmid having a topology corresponding to the description

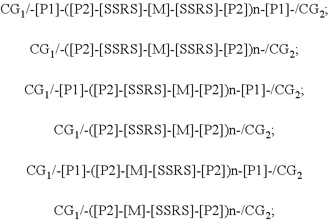

wherein CG are parental cell genomic sequences flanking the inserted plasmid; [P1] is polynucleotide sequence derived from a parental plasmid; [P2] are polynucleotide sequences derived from a landing pad plasmid; [M] is a polynucleotide sequence comprising at least one marker; [SSRS] are site-specific recombination sites (SSRS), and n is an integer between 1 and 10. In some aspects, n is 1. In some aspects, n is 2. In some aspects, n is 3. In some aspects, n is 4. In some aspects, n is 5. In some aspects, n is 6. In some aspects, n is 7. In some aspects, n is 8. In some aspects, n is 9. In some aspects, n is 10.

Notice that the labels [P1], [P2], and [SSRS] in any of the formulas in the present disclosure are just descriptors of the origin or type of component to represent the topology of the construct. The nucleic acid sequences of each [P1] and [P2] component are different, i.e., the nucleic acid sequence of the first [P1] is different from the nucleic acid sequence of the second [P1], but they share a common origin, i.e., the parental plasmid. Similarly, the nucleic acid sequence of the first [P2] is different from the nucleic acid sequence of the second [P2], but they share a common origin, i.e., the landing pad plasmid. In some aspects, the CG sequences in the landing pad cell are different from the CG sequences in the parental cell line, i.e., the plasmid in located in a hot spot that is different from the original hot spot in the parental cell line.

[SSRS] components are, e.g., Cre/Lox sites, and each one of them can have a different sequence. However, in some aspects, in any formulas presented throughout the present disclosure comprising a [SSRS] pair, one of the [SSRS] shown is optional. When integration is conducted using, e.g., a Serine-integrase, a single [SSRS] is required. Thus, in those specific aspects, a single att site, e.g., an attP site, may be present instead of a [SSRS] pair.

In some aspects, the topology of the plasmid integrated in the expression cells corresponds to the description

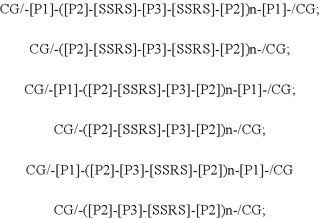

wherein CG are parental cell genomic sequences flanking the inserted plasmid; [P1] is a polynucleotide sequence derived from a parental plasmid; [P2] are polynucleotide sequences derived from a landing pad plasmid; [P3] is a polynucleotide sequence derived from a plasmid comprising a gene of interest (GOI); [SSRS] are site-specific recombination sites (SSRS); and n is an integer between 1 and 10. In some aspects, n is 1. In some aspects, n is 2. In some aspects, n is 3. In some aspects, n is 4. In some aspects, n is 5. In some aspects, n is 6. In some aspects, n is 7. In some aspects, n is 8. In some aspects, n is 9. In some aspects, n is 10. In some aspects, the CG sequences in the landing pad cell are different from the CG sequences in the parental cell line, i.e., the plasmid in located in a hot spot which is different from the original hot spot in the parental cell line.

In some aspects, the homologous recombination is mediated by a CRISPR/Cas system, a TALEN system, or a ZFN system, described in detail below. In some aspects, the homologous recombination system, e.g., CRISPR/Cas system, further comprises a single guide RNA (sgRNA). Depending on the homologous recombination used, additional components may be required as disclosed in detail below.

In some aspects, the site-specific recombinase recombination site (SSRS) is a Tyr-recombinase site, a Tyr-integrase site, a Serine-resolvase/invertase site, a Serine-integrase site, or a combination thereof. In some aspects, the Tyr-recombinase site comprises a Cre, Dre, Flp, KD, B3, or B3 Tyr-recombinase site. In some aspects, the Tyr-integrase site comprises a λ (Lambda), HK022, or HP1 Tyr-integrase site. In some aspects, the Serine-resolvase/invertase site comprises a γδ (Gammadelta), ParA, Tn3, or Gin Serine-resolvase/integrase site. In some aspects, the Serine-integrase site comprises a PhiC31, Bxb1, pr R4 Serine-integrase site. In some aspects, Tyr-recombinase site comprises a Cre Tyr-recombinase site. In some aspects, the SSRS is a LoxP site. In some aspects, the LoxP site comprises a nucleic acid sequence set forth in SEQ ID NO:1 (wild type LoxP). In some aspects, the LoxP site comprises a mutant LoxP site. In some aspects, the mutant LoxP site comprises a nucleic acid sequence set forth in SEQ ID NO:2 (mutant LoxP). In some aspects, the mutant LoxP site comprises a nucleic acid selected, e.g., from the group consisting of: SEQ ID NO: 3 (Lox 511); SEQ ID NO: 4 (Lox 5171); SEQ ID NO: 5 (Lox 2272); SEQ ID NO: 6 (Lox M2); SEQ ID NO: 7 (Lox M3); SEQ ID NO: 8 (Lox M7); SEQ ID NO: 9 (Lox M11); SEQ ID NO: 10 (Lox 71); and, SEQ ID NO: 11 (Lox 66). In some aspects, the Tyr-recombinase site comprises a Flp Tyr-recombinase site. In some aspects, the SSRS is a short flippase recognition target (FRT) site. In some aspects, the Serine-integrase site comprises an att site, e.g., an attP or attB site.

In some aspects, each SSRS of a pair of SSRS in a plasmid disclosed herein can belong to different classes. For example, the first SSRS can be, e.g., a Tyr-recombinase site, and the second SSRS can be, e.g., a Ser-integrase site. In some aspects, the SSRS pair comprises two sites selected from wild type LoxP, a mutant LoxP site, a Lox 511 site, a Lox 5171 site, a Lox 2272 site, a Lox M2 site, a Lox M3 site, a Lox M7 site, a Lox M11 site, a Lox 71 site, a Lox 66 site, or any combination thereof. In some aspects, the SSRS pair comprises a Lox P site and a Lox 511 site. In some aspects, the SSRS pair comprises a Lox P site and a Frt site. In some aspects, the SSRS pair comprises two aat sites, e.g., two attP sites. In some aspects, the SSRS pair comprises two aat sites, e.g., two attR sites. In some aspects, the SSRS pair comprises a Lox 2272 site and a Lox M3 site. In some aspects, the SSRS pair comprises a Lox m3 site and a Lox m7 site.

In some aspects, the plasmids disclosed herein comprise at least one single selection marker. In some aspects, the plasmids disclosed herein comprise a single selection marker. In some aspects, the plasmids disclosed herein comprise more than one single selection marker, e.g., two selection markers. In some aspects, the at least one selection marker is glutamine synthetase (GS). In some aspects, the at least one selection marker is dihydrofolate reductase (DHFR). In some aspects, the at least one selection marker comprise a glutamine synthetase (GS) marker and a dihydrofolate reductase (DHFR) marker. There are several selection markers which are suitable for generating stably transfected Chinese hamster ovary (CHO) cell lines. Due to their different modes of action, each selection marker has its own optimal selection stringency in different host cells for obtaining high productivity. See Yeo et al. (2017) Biotechnol J 12(12), which is herein incorporated by reference in its entirety.

In some aspects, the at least one selection marker is a drug resistance gene, e.g., an antibiotic resistance gene. In some aspects, the antibiotic resistance gene is selected from the group consisting of an actinomycin D resistance gene, a bleomycin resistance gene, a chloramphenicol resistance gene, a G418 resistance gene, a hydromycin resistance gene, a mitomycin C resistance gene, a mycophenolic acid resistance gene, a puromycin resistance gene, and any combination thereof, In some aspects, the antibiotic resistance gene is a puromycin resistance gene. In some aspects, the puromycin resistance gene is puromycin-N-acetyltransferase.

In some aspects, the at least one detectable marker comprises a protein, e.g., a fluorescent protein. In some aspects, the fluorescent protein is mCherry. In some aspects, the fluorescent protein is selected from the group consisting of GFP, ZsGreen1, AcGFP1, EGFP, GFPuv, AcGFP, EBFP, EYFP, ECFP, tdTomato, mCherry, DsRed, AmCyan, ZsGreen, ZsYellow, DsRed2, DsRed-Express, HcRed, AsRed, mOrange, mOrange2, mPlum, mStrawberry, mBanana, YFP, mRaspberry, HcRed1, E2-Crimson, and any combination thereof.

In some aspects, the parental cell is selected from the group consisting of a Chinese Hamster Ovary (CHO) cell, a HEK293 cells, and an NS0 cell, or their derivatives or equivalents. In some aspects, the CHO cell is a CHO DG44 cell or a CHO K1 cell.

In some aspects, the GOI encodes at least one polypeptide, e.g., an antibody or a fusion protein. In some aspects, the antibody specifically binds to T cell immunoglobulin and mucin domain-containing protein 3 (TIM3), a Tau protein such as an N-terminal fragment of tau (eTau), or an immune checkpoint protein such as PD-1 of PD-L1. In some aspects, the antibody is nivolumab. In some aspects, the GOI is the heavy chain (HC) of an antibody. In some aspects, the GOI is the light chain of an antibody (LC). In some aspects, the GOI comprises a HC and a LC of an antibody (e.g., in a bicistronic construct). In some aspects, the GOI is a bispecific antibody or a portion thereof, e.g., a HC or LC of a bispecific antibody or any combination thereof. In some aspects, the expression plasmid comprises one, two, or more than two copies of the GOI.

In some aspects, the methods disclosed herein comprise determining the expression of a GOI or marker disclosed herein. In some aspects, the expression of the GOI or marker is determined quantitatively and/or qualitatively. In some aspects, the expression of the GOI or marker is determined, for example, by cell sorting, FACS, cell surface staining, Western blot, Northern blot, column chromatography, capillary electrophoresis, microfluidics, UV absorbance, immunohistochemistry, cell size, secreted protein levels, transcript levels, or any combination thereof.

In some aspects, the landing pad plasmid (Second GOI plasmid) or expression plasmid (P4) is integrated with a copy number of 1 in the genome of the cell. In some aspects, the landing pad plasmid (Second GOI plasmid) or expression plasmid (P4) is integrated with a copy number of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 in the genome of the cell.

In some aspects, the 5′ homologous recombination site of a plasmid disclosed herein comprises a polynucleotide sequence of SEQ ID NO: 18 or a subsequence thereof and the 3′ homologous recombination site comprises a polynucleotide sequence of SEQ ID NO: 19 or a subsequence thereof.

In some aspects, the 5′ homologous recombination site of a plasmid disclosed herein comprises a polynucleotide sequence of SEQ ID NO: 114 or a subsequence thereof and the 3′ homologous recombination site comprises a polynucleotide sequence of SEQ ID NO: 115 or a subsequence thereof.

In some aspects, the isolated cell or population of isolated cells of the present disclosure comprise a polynucleotide sequence which comprises a nucleic acid encoding a gene of interest (GOI) integrated within a specific locus of the genome of the cell, wherein the locus comprises a nucleotide subsequence selected from SEQ ID NO: 20 and SEQ ID NO:116. In some aspects, the methods disclosed herein comprise introducing into cells, e.g., CHO cells or another suitable cell line, a polynucleotide sequence which comprises a nucleic acid encoding at least one gene of interest (GOI) and obtaining a cell, e.g., a CHO cell, wherein the exogenous nucleic acid is integrated into a specific locus of the genome of the cell, e.g., a CHO cell, the locus comprising a nucleotide subsequence selected from SEQ ID NO: 20 and SEQ ID NO: 116. In some aspects, the methods disclosed herein comprise (a) providing a cell comprising a polynucleotide sequence which comprises a nucleic acid encoding a gene of interest (GOI), wherein the polynucleotide sequence comprises the nucleic acid encoding a gene of interest (GOI) operably linked to a promoter, and wherein the locus comprises a nucleotide subsequence selected from SEQ ID NO: 20 and SEQ ID NO: 116. In some aspects, the nucleotide subsequence selected from SEQ ID NO: 20 comprises the sequence set forth in SEQ ID NO:21. In some aspects, the nucleotide subsequence selected from SEQ ID NO: 116 comprises the sequence set forth in SEQ ID NO:117. In some aspects, the nucleotide subsequence selected from SEQ ID NO: 20 consists of the sequence set forth in SEQ ID NO:21. In some aspects, the nucleotide subsequence selected from SEQ ID NO: 116 consists of the sequence set forth in SEQ ID NO:117. In some aspects, the nucleotide subsequence selected from SEQ ID NO: 20 is a subsequence upstream (towards the 5′ end of SEQ ID NO: 20) with respect to the sequence set forth in SEQ ID NO:21. In some aspects, the nucleotide subsequence selected from SEQ ID NO: 20 is a subsequence downstream (towards the 3′ end of SEQ ID NO: 20) with respect to the sequence set forth in SEQ ID NO:21. In some aspects, the nucleotide subsequence selected from SEQ ID NO: 116 is a subsequence upstream (towards the 5′ end of SEQ ID NO: 116) with respect to the sequence set forth in SEQ ID NO:117. In some aspects, the nucleotide subsequence selected from SEQ ID NO: 116 is a subsequence downstream (towards the 3′ end of SEQ ID NO: 116) with respect to the sequence set forth in SEQ ID NO:117.

The present disclosure provides landing pad cell lines that contain a single landing pad plasmid. However, landing pad cell lines with more than one landing pad plasmid provide an opportunity to further refine expression of multisubunit biologics such as bispecific monoclonal antibodies (mAbs). Accordingly, the cell screening methods disclosed herein can be used to identify landing pad cell lines with two landing pad plasmids in the same locus, i.e., duo-landing pad cells. This ensures equal expression from both landing pad plasmids as they reside in the same genomic locus.

The duo-landing pads of the present disclosure can integrate in four different orientations head-to-head, tail-to-tail, tail-to-head and head-to-tail. When a single site directed recombinase such as Cre/Lox or Flp/Frt is used, the head-to-head and tail-to-tail configurations are generally used since they are functionally undistinguishable from each other. Unlike in the tail-to-head and head-to-tail configurations that in the presence of Cre/Lox can result in deletion of one of the landing pads, the head-to-head and tail-to-tail configurations simply go through inversion resulting in the same starting configuration.

When a Second GOI plasmid is used with each of the four duo-landing pad configurations (head-to-head, tail-to-tail, tail-to-head and head-to-tail), the head-to-head and tail-to-tail configurations can each generate two cell lines where the sequences between the two recombination sites flanking the plasmid junction can be inverted, otherwise the two cell lines are the same. When the head to tail or tail to head configurations are used with the Second GOI plasmid cell lines with two Second GOI plasmids are produced. However, if there is sufficient amounts of Cre activity present one of the Second GOI plasmids can be removed resulting in a Second GOI plasmid cell line with a single Second GOI plasmid.

If the landing pad uses a Frt recognition site for Flp in place of of Lox site, e.g., Lox 511, and both Cre/Lox and Flp are used, the same outcome will result, with deletion in tail-to-head and head-to-tail orientations, while the head-to-head and tail-to-tail orientations go through inversions. However, recombining the Second GOI plasmid into the duo-landing pad using attP/attB with integrase in the tail-to-tail and head-to-head configurations results in no inversions, but in the tail-to-head and head-to-tail configurations the deletion of one of the landing pads can still occur. If each of the landing pads has a single attP site then a single integration of a Second GOI plasmid with a single attB site would occur resulting in no deletions occurring in any of the four duo-landing pad configurations.

As used herein, the term “single landing pad” refers to a landing pad that comprises a single Landing Pad Plasmid or Second GOI plasmid. As used herein, the term “duo-landing pad” refers to a landing pad that comprises two Landing Pad plasmids or Second GOI plasmids.

The use of duo-landing pads offers an alternative method to produce biologic comprising different GOI, e.g., an antibody comprising a heavy chain and a light chain. In one aspect, the present disclosure provides methods and compositions wherein a Second GOI plasmid comprises multiple expression cassettes encoding, e.g., the heavy chain and the light chain of an antibody. In another aspect, each expression cassette can be in a different Second GOI plasmid, and both Second GOI plasmids would be located in a duo-landing pad.

The use of a duo-Landing Pad Cell Line has advantages over a landing pad cell line with a single landing pad (i.e., a landing pad comprising a single Second GOI plasmid). In the case of the single landing pad cell line, all expression cassettes needed to make a multicomponent biologic must be placed in a single Second GOI Plasmid as the cell line only accommodates a single Second GOI. That is not the case with the duo-Landing Pad Cell Line. The duo-Landing Pad Cell line affords the opportunity to design in greater expression diversity levels providing the opportunity to create an Expression Cell Line with superior characteristics. The diversity can be generated in multiple ways using different configurations of the Second GOI Plasmids. In one instance the Second GOI Plasmids contain all expression cassettes needed to make the complex biologic in unique configurations. In a second instance the Second GOI Plasmids may contain a subset of the expression cassettes that need to reside in the same cell to make an expression cell line. In a third instance a combination of the two previous instances where one or more Second GOI Plasmids having all the expression cassettes in unique configurations needed to make the complex biologic along with a set of Second GOI Plasmids that contains a subset of all the expression cassettes in unique configuration(s).

It is understood that the same methods disclosed here to generate a duo-landing pad may be used to generate cell lines with higher order combinations of landing pad plasmids. For example, the methods disclosed herein to identify a landing pad cell line with two landing pad plasmids in a hot spot may be used to select landing pad cell lines having three, four, or more landing pad plasmid. The landing pad cells lines and expression cells having hot spots containing more than two landing pad plasmids can be used, for example, to produce biologics comprising more than two different subunits.

Although in some aspects duo-landing pad configurations can comprises both landing pads plasmids have the same recombinase or Int recognition sequence it is possible to make each landing pad plasmid have a unique recombination “address,” i.e., each landing pad plasmid becomes addressable. In the case of recombinases such as Cre and Flp four unique recognition sequences can be used. Accordingly, each landing pad plasmid would have a unique pairing of recognition sites. In some aspects, four incompatible Lox sites can be used. See Langer, S. J., Ghafoori, A. P., Byrd, M. and Leinwand, L. (2002) A genetic screen identifies novel non-compatible loxP sites. Nucleic Acids Res., 30, 3067-3077; Missirlis, P. I., Smailus, D. E. and Holt, R. A. (2006) A high-throughput screen identifying sequence and promiscuity characteristics of the loxP spacer region in Cre-mediated recombination. BMC Genomics, 7, 73.; and Siegel, R. W., Jain, R. and Bradbury, A. (2001) Using an in vivo phagemid system to identify non-compatible loxP sequences. FEBS Lett., 505, 467-473.

Examples of additional strategies include replacing two Lox sites with two incompatible Frt sites and using Cre with Frt (see Lauth, M., Spreafico, F., Dethleffsen, K. and Meyer, M. (2002) Stable and efficient cassette exchange under non-selectable conditions by combined use of two site-specific recombinases. Nucleic Acids Res., 30, e115), using an integrase with two to four incompatible aat sites (see Jusiak, B., Jagtap, K., Gaidukov, L., Duportet, X., Bandara, K., Chu, J., Zhang, L., Weiss, R. and Lu, T. K. (2019) Comparison of Integrases Identifies Bxb1-GA Mutant as the Most Efficient Site-Specific Integrase System in Mammalian Cells. ACS Synth Biol, 8, 16-24), using more than one integrase for example that of BxB1, and phiC3 (see Smith, M. C., Brown, W. R., McEwan, A. R. and Rowley, P. A. (2010) Site-specific recombination by phiC31 integrase and other large serine recombinases. Biochem. Soc. Trans., 38, 388-394), and combinations thereof. The use of a single att site in each landing pad is sufficient for insertion of the Second GOI Plasmids into each landing pad. In this case the Second GOI Plasmid is required to be circular as a linear plasmid would effectively restrict the chromosome. It is also clear the landing pad can contain multiple att sites so that each contains a unique address.

The duo-landing pad configuration with the landing pads with unique addresses can also be used to generate a more defined diversity of Expression Cell Lines compared to when they are not addressable, and higher diversity to a Landing Pad cell line with a single landing pad.

An additional application of the addressable landing pads is the option to have two independent biologics expressed each with its own independent function. One of the biologics could help the Expression Cell Line express the second biologic, or the first biologic could cause a particular post translational modification of the second biologic or modify some other component of the Expression Cell Line.

In some aspects, the methods, cells, cell lines, or kits disclosed herein comprise at least two landing pad plasmids or at least two expression plasmids in tandem. In other words, in some aspects the n value in the formula

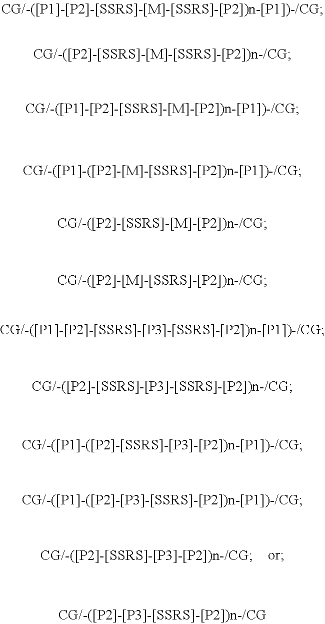

or any other formula disclosed herein containing an n value can be 2 or higher. In some specific aspects, n is 2. Thus, in some aspects, at least two landing pad plasmids or at least two expression plasmids arranged in tandem are present in the constructs disclosed herein. In some aspects, the n is an integer such as 2, 3, 4, 5, 6, 7, 8, 9 or 10. In some aspects, n is higher than 10, e.g., 11, 12, 13, 14, 15, 16, 17, 18, 19, 20, 21, 22, 23, 24, 25, 26, 27, 28, 29, or 30.

In some aspects, two landing pad plasmids or two expression plasmids are in a configuration selected from the group consisting of head-to-head, tail-to-tail, tail-to-head, and head-to-tail. In some aspects, each expression plasmid comprises at least a nucleic acid encoding a gene of interest (GOI). In some aspects, all GOI are the same. In some aspects, all GOI are different. In some aspects, at least one GOI is different from the rest. In some aspects, a first GOI is a HC of an antibody, and a second GOI is a LC of an antibody. In some aspects, at least one expression plasmid is bicistronic or polycistronic. In some aspects, the bicistronic expression plasmid encodes a first GOI comprising a HC of an antibody, and a second GOI comprising a LC of an antibody.

In some aspects, each landing pad plasmid in a duo-landing pad is addressable. In some aspects, each addressable landing pad plasmid comprises a pair of SSRS, which can be unique or incompatible. In some aspects, a landing pad plasmid comprises two Lox sites. In some aspects, the Lox sites are Lox P and Lox 511. In some aspects, each landing pad plasmid comprises a Lox site and an Frt site. In some aspects, each landing pad plasmid comprises one or two aat sites, e.g., two aatP sites.

In some aspects, each landing pad plasmid is addressable. In some aspects, each addressable landing pad plasmid comprises a pair of addressable SSRS which are unique to the landing pad. In some aspects, at least one pair of addressable SSRS is a pair of Lox sites. In some aspects, at least one pair of Lox sites is Lox 511 and Lox P. In some aspects, at least one pair of Lox sites is Lox m3 and Lox m7.

In some aspects, the methods, cell lines, cells or kits of the present disclosure comprise a first addressable landing pad plasmid comprises a Lox 511 and Lox P pair of Lox sites, and a second addressable landing pad plasmid comprises an Lox m3 and Lox m7 pair of Lox sites. In some aspects, each addressable landing pad plasmid comprises a non cross-compatible attP site.

In some aspects, the LoxP sites are selected from the group consisting of SEQ ID NOS:1-1 land 28-82 and any combinations thereof. In some aspects, the Frt sites are selected from the group consisting of SEQ ID NOS:12 and 83-91 and any combinations thereof. In some aspects, an addressable pad disclosed herein can comprise a SSRS or combination thereof selected from the group consisting of SEQ ID NOS: 1-13 and 28-109, and any combination thereof.

In some aspects, the att sites are selected from the group consisting of SEQ ID NOS: 92 to 109 and any combinations thereof. In some aspects, a pair of att sites comprises an attB site of SEQ ID NO: 92 and an attP site of SEQ ID NO: 93. In some aspects, a pair of att sites comprises an attB site of SEQ ID NO: 94 and an attP site of SEQ ID NO: 95. In some aspects, a pair of att sites comprises an attB site of SEQ ID NO: 96 and an attP site of SEQ ID NO: 97. In some aspects, a pair of att sites comprises an attB site of SEQ ID NO: 98 and an attP site of SEQ ID NO: 99. In some aspects, a pair of att sites comprises an attB site of SEQ ID NO: 100 and an attP site of SEQ ID NO: 101. In some aspects, a pair of att sites comprises an attB site of SEQ ID NO: 102 and an attP site of SEQ ID NO: 103. In some aspects, a pair of att sites comprises an attB site of SEQ ID NO: 104 and an attP site of SEQ ID NO: 105. In some aspects, a pair of att sites comprises an attB site of SEQ ID NO: 106 and an attP site of SEQ ID NO: 107. In some aspects, a pair of att sites comprises an attB site of SEQ ID NO: 108 and an attP site of SEQ ID NO: 109.

### Nucleases

As used herein, the term “nuclease” refers to an enzyme that possesses catalytic activity for DNA cleavage.

In some aspects, a nuclease agent can promote homologous recombination between two plasmids, e.g., linear plasmids, disclosed herein, e.g., a parental plasmid and a landing pad plasmid. In some aspects, the plasmid integrated in the genome of the parental cell line (parental plasmid, P1) and the landing pad plasmid (P2) contain regions of homology, and next to each homology region a sequence targeted by a nuclease, e.g., a CRISPR/Cas nuclease, is present in the parental plasmid integrated in the parental cell line, but absent in the landing pad plasmids to be recombined into the parent cell line.

The size of the recognition site for the nuclease mediating homologous recombination can vary, and includes, for example, recognition sites that are at least about 4, at least about 6, at least about 8, at least about 10, at least about 12, at least about 14, at least about 16, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 510, at least about 520, at least about 530, at least about 540, at least about 550, at least about 560, at least about 570, at least about 580, at least about 590, at least about 600, at least about 610, at least about 620, at least about 630, at least about 640, at least about 650, at least about 660, at least about 670, at least about 680, at least about 690, at least about 700, at least about 710, at least about 720, at least about 730, at least about 740, at least about 750, at least about 760, at least about 770, at least about 780, at least about 790, at least about 800, at least about 810, at least about 820, at least about 830, at least about 840, at least about 850, at least about 860, at least about 870, at least about 880, at least about 890, at least about 900, at least about 910, at least about 920, at least about 930, at least about 940, at least about 950, at least about 960, at least about 970, at least about 980, at least about 990, at least about 1000, at least about 1010, at least about 1020, at least about 1030, at least about 1040, at least about 1050, at least about 1060, at least about 1070, at least about 1080, at least about 1090, at least about 1100, at least about 1110, at least about 1120, at least about 1130, at least about 1140, at least about 1150, at least about 1160, at least about 1170, at least about 1180, at least about 1190, at least about 1200, at least about 2010, at least about 2020, or more nucleotides in length.

The size of the recognition site for the nuclease mediating homologous recombination can vary, and includes, for example, recognition sites that are about 4, about 6, about 8, about 10, about 12, about 14, about 16, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800, about 810, about 820, about 830, about 840, about 850, about 860, about 870, about 880, about 890, about 900, about 910, about 920, about 930, about 940, about 950, about 960, about 970, about 980, about 990, about 1000, about 1010, about 1020, about 1030, about 1040, about 1050, about 1060, about 1070, about 1080, about 1090, about 1100, about 1110, about 1120, about 1130, about 1140, about 1150, about 1160, about 1170, about 1180, about 1190, about 1200, about 2010, about 2020, or more nucleotides in length.

The size of the recognition site for the nuclease mediating homologous recombination can vary, and includes, for example, recognition sites that are between about 4 and about 10, about 10 and about 20, about 20 and about 30, about 30 and about 40, about 40 and about 50, about 50 and about 60, about 60 and about 70, about 70 and about 80, about 80 and about 90, about 90 and about 100, about 100 and about 125, about 125 and about 150, about 150 and about 175, about 175 and about 200, about 200 and about 225, about 225 and about 250, about 250 and about 275, about 275 and about 300, about 300 and about 325, about 325 and about 350, about 350 and about 375, about 375 and about 400, about 400 and about 425, about 425 and about 450, about 450 and about 475, about 475 and about 500, about 500 and about 525, about 525 and about 550, about 550 and about 575, about 575 and about 600, about 600 and about 625, about 625 and about 650, about 650 and about 675, about 675 and about 700, about 700 and about 725, about 725 and about 750, about 750 and about 775, about 775 and about 800, about 800 and about 825, about 825 and about 850, about 850 and about 875, about 875 and about 900, about 900 and about 925, about 925 and about 950, about 950 and about 975, about 975 and about 1000, about 1000 and about 1100, about 1100 and about 1200, about 1200 and about 1300, about 1300 and about 1400, about 1400 and about 1500, about 1500 and about 1600, about 1600 and about 1700, about 1700 and about 1800, about 1800 and about 1900, about 1900 and about 2000, or about 2000 and about 2100, or more nucleotides in length.

In one aspect, each monomer of the nuclease agent recognizes a recognition site of at least 9 nucleotides. In other aspects, the recognition site is from about 9 to about 12 nucleotides in length, from about 12 to about 15 nucleotides in length, from about 15 to about 18 nucleotides in length, or from about 18 to about 21 nucleotides in length, and any combination of such subranges (e.g., 9-18 nucleotides). The recognition site could be palindromic, that is, the sequence on one strand reads the same in the opposite direction on the complementary strand. It is recognized that a given nuclease agent can bind the recognition site and cleave that binding site or alternatively, the nuclease agent can bind to a sequence that is the different from the recognition site. Moreover, the term recognition site comprises both the nuclease agent binding site and the nick/cleavage site irrespective whether the nick/cleavage site is within or outside the nuclease agent binding site. In another variation, the cleavage by the nuclease agent can occur at nucleotide positions immediately opposite each other to produce a blunt end cut or, in other cases, the incisions can be staggered to produce single-stranded overhangs, also called “sticky ends,” which can be either 5′ overhangs, or 3′ overhangs.

In some aspects, one of the sequences in the parental plasmid missing in the landing pad plasmid is SEQ ID NO: 14, and the other is SEQ ID NO: 15.

In some aspects, one of the sequences in the parental plasmid missing in the landing pad plasmid is SEQ ID NO: 14.

In some aspects, one of the sequences in the parental plasmid missing in the landing pad plasmid is SEQ ID NO: 15.

Any nuclease agent that induces a nick or double-strand break into a desired recognition site can be used in the methods and compositions disclosed herein. A naturally-occurring or native nuclease agent can be employed so long as the nuclease agent induces a nick or double-strand break in a desired recognition site. Alternatively, a modified or engineered nuclease agent can be employed. An “engineered nuclease agent” comprises a nuclease that is engineered (modified or derived) from its native form to specifically recognize and induce a nick or double-strand break in the desired recognition site. Thus, an engineered nuclease agent can be derived from a native, naturally-occurring nuclease agent or it can be artificially created or synthesized. The modification of the nuclease agent can be as little as one amino acid in a protein cleavage agent or one nucleotide in a nucleic acid cleavage agent. In some aspects, the engineered nuclease induces a nick or double-strand break in a recognition site, wherein the recognition site was not a sequence that would have been recognized by a native (non-engineered or non-modified) nuclease agent. Producing a nick or double-strand break in a recognition site or other DNA can be referred to herein as “cutting” or “cleaving” the recognition site or other DNA.

### Homologous Recombination Systems

In some aspects of the present disclosure, the homologous recombination is mediated by a CRISPR/Cas system, a TALEN system, a ZFN system, a mega nuclease, or a restriction endonuclease.

### CRISPR/Cas

In some aspects, the nuclease agent employed for homologous recombination in the various methods and compositions disclosed herein can comprise a CRISPR/Cas system. Note that the depiction of CRISPR/Cas in the figures as the “default” homologous recombination system is merely exemplary, and the processes schematized in the figures can be performed using an alternative homologous recombination system, e.g., a TALEN system, a ZFN system, a mega nuclease, or a restriction endonuclease. Such CRISPR/Cas systems can employ, for example, a Cas9 nuclease, which in some instances, is codon-optimized for the desired cell type in which it is to be expressed. Such systems can also employ a guide RNA (gRNA) that comprises two separate molecules. An exemplary two-molecule gRNA comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA” or “scaffold”) molecule.

A crRNA comprises both the DNA-targeting segment (single stranded) of the gRNA and a stretch of nucleotides that forms one half of a double stranded RNA (dsRNA) duplex of the protein-binding segment of the gRNA. A corresponding tracrRNA (activator-RNA) comprises a stretch of nucleotides that forms the other half of the dsRNA duplex of the protein-binding segment of the gRNA. Thus, a stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. The crRNA additionally provides the single stranded DNA-targeting segment. Accordingly, a gRNA comprises a sequence that hybridizes to a target sequence, and a tracrRNA. Thus, a crRNA and a tracrRNA (as a corresponding pair) hybridize to form a gRNA. If used for modification within a cell, the exact sequence and/or length of a given crRNA or tracrRNA molecule can be designed to be specific to the species in which the RNA molecules will be used.

Naturally occurring genes encoding the three elements (Cas9, tracrRNA and crRNA) are typically organized in operon(s). Naturally occurring CRISPR RNAs differ depending on the Cas9 system and organism but often contain a targeting segment of between 21 to 72 nucleotides length, flanked by two direct repeats (DR) of a length of between 21 to 46 nucleotides (see, e.g., WO2014/131833). In the case of S. pyogenes, the DRs are 36 nucleotides long and the targeting segment is 30 nucleotides long. The 3′ located DR is complementary to and hybridizes with the corresponding tracrRNA, which in turn binds to the Cas9 protein.

Alternatively, the system further employs a fused crRNA-tracrRNA construct (i.e., a single transcript) that functions with the codon-optimized Cas9. This single RNA is often referred to as a guide RNA or gRNA. Within a gRNA, the crRNA portion is identified as the “target sequence” for the given recognition site and the tracrRNA is often referred to as the “scaffold.” Briefly, a short DNA fragment containing the target sequence is inserted into a guide RNA expression plasmid. The gRNA expression plasmid comprises the target sequence (in some aspects around 20 nucleotides), a form of the tracrRNA sequence (the scaffold) as well as a suitable promoter that is active in the cell and necessary elements for proper processing in eukaryotic cells. Many of the systems rely on custom, complementary oligonucleotides that are annealed to form a double stranded DNA and then cloned into the gRNA expression plasmid.

The gRNA expression cassette and the Cas9 expression cassette are then introduced into the cell. See, for example, Mali P et al. (2013) Science 2013 Feb. 15; 339(6121):823-6; Jinek M et al. Science 2012 Aug. 17; 337(6096):816-21; Hwang W Y et al. Nat Biotechnol 2013 March; 31(3):227-9; Jiang W et al. Nat Biotechnol 2013 March; 31(3):233-9; and Cong L et al. Science 2013 Feb. 15; 339(6121):819-23, each of which is herein incorporated by reference. See also, for example, WO/2013/176772A1, WO/2014/065596A1, WO/2014/089290A1, WO/2014/093622A2, WO/2014/099750A2, and WO/2013142578A1, each of which is herein incorporated by reference.

In some aspects, the Cas9 nuclease can be provided in the form of a protein. In some aspects, the Cas9 protein can be provided in the form of a complex with the gRNA. In other aspects, the Cas9 nuclease can be provided in the form of a nucleic acid encoding the protein. The nucleic acid encoding the Cas9 nuclease can be RNA (e.g., messenger RNA (mRNA)) or DNA. In some aspects, the gRNA can be provided in the form of RNA. In other aspects, the gRNA can be provided in the form of DNA encoding the RNA. In some aspects, the gRNA can be provided in the form of separate crRNA and tracrRNA molecules, or separate DNA molecules encoding the crRNA and tracrRNA, respectively.

In one aspect, the methods for generating a landing pad cell disclosed herein further comprise introducing into the cell: (a) a first expression construct comprising a first promoter operably linked to a first nucleic acid sequence encoding a CRISPR-associated (Cas) protein; (b) a second expression construct comprising a second promoter operably linked to a genomic target sequence linked to a guide RNA (gRNA), wherein the genomic target sequence is flanked by a Protospacer Adjacent Motif. Optionally, the genomic target sequence is flanked on the 3′ end by a Protospacer Adjacent Motif (PAM) sequence.

In some aspects, the gRNA comprises a third nucleic acid sequence encoding a Clustered Regularly Interspaced Short Palindromic Repeats (CRISPR) RNA (crRNA) and a trans-activating CRISPR RNA (tracrRNA). In one aspect, the Cas protein is a type I Cas protein. In one aspect, the Cas protein is a type II Cas protein. In one aspect, the type II Cas protein is Cas9. In one aspect, the type II Cas, e.g., Cas9, is a human codon-optimized Cas.

In certain aspects, the Cas protein is a “nickase” that can create single strand breaks (i.e., “nicks”) at the target site without cutting both strands of double stranded DNA (dsDNA). Cas9, for example, comprises two nuclease domains—a RuvC-like nuclease domain and an HNH-like nuclease domain—which are responsible for cleavage of opposite DNA strands. Mutation in either of these domains can create a nickase. Examples of mutations creating nickases can be found, for example, WO/2013/176772A1 and WO/2013/142578A1, each of which is herein incorporated by reference.

In certain aspects, two separate Cas proteins (e.g., nickases) specific for a target site on each strand of dsDNA can create overhanging sequences complementary to overhanging sequences on another nucleic acid, or a separate region on the same nucleic acid. The overhanging ends created by contacting a nucleic acid with two nickases specific for target sites on both strands of dsDNA can be either 5′ or 3′ overhanging ends. For example, a first nickase can create a single strand break on the first strand of dsDNA, while a second nickase can create a single strand break on the second strand of dsDNA such that overhanging sequences are created. The target sites of each nickase creating the single strand break can be selected such that the overhanging end sequences created are complementary to overhanging end sequences on a different nucleic acid molecule. The complementary overhanging ends of the two different nucleic acid molecules can be annealed by the methods disclosed herein. In some aspects, the target site of the nickase on the first strand is different from the target site of the nickase on the second strand.

In some aspects, the first nucleic acid comprises a mutation that disrupts at least one amino acid residue of nuclease active sites in the Cas protein, wherein the mutant Cas protein generates a break in only one strand of the target DNA region, and wherein the mutation diminishes non-homologous recombination in the target DNA region. In one aspect, the first nucleic acid that encodes the Cas protein further comprises a nuclear localization signal (NLS). In one aspect, the nuclear localization signal is a SV40 nuclear localization signal.

### TALEN

In some aspects, the nuclease agent employed for homologous recombination in the various methods and compositions disclosed herein can comprise a TALEN system. Thus, in one aspect, the nuclease agent is a Transcription Activator-Like Effector Nuclease (TALEN). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease, such as, for example, FokI.

The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. (2010) doi:10.1093/nar/gkg704; and Miller et al. (2011) Nature Biotechnology 29:143-148; all of which are herein incorporated by reference.

Examples of suitable TAL nucleases, and methods for preparing suitable TAL nucleases, are disclosed, e.g., in US Patent Application No. 2011/0239315 A1, 2011/0269234 A1, 2011/0145940 A1, 2003/0232410 A1, 2005/0208489 A1, 2005/0026157 A1, 2005/0064474 A1, 2006/0188987 A1, and 2006/0063231 A1 (each hereby incorporated by reference).

In various aspects, TAL effector nucleases are engineered that cut in or near a target nucleic acid sequence in, e.g., a genomic locus of interest, wherein the target nucleic acid sequence is at or near a sequence to be modified by a targeting vector. The TAL nucleases suitable for use with the various methods and compositions provided herein include those that are specifically designed to bind at or near target nucleic acid sequences to be modified by targeting vectors as described herein.

In one aspect, each monomer of the TALEN comprises 12-25 TAL repeats, wherein each TAL repeat binds a 1 bp subsite. In one aspect, the nuclease agent is a chimeric protein comprising a TAL repeat-based DNA binding domain operably linked to an independent nuclease. In one aspect, the independent nuclease is a FokI endonuclease. In one aspect, the nuclease agent comprises a first TAL-repeat-based DNA binding domain and a second TAL-repeat-based DNA binding domain, wherein each of the first and the second TAL-repeat-based DNA binding domain is operably linked to a FokI nuclease, wherein the first and the second TAL-repeat-based DNA binding domain recognize two contiguous target DNA sequences in each strand of the target DNA sequence separated by about 6 bp to about 40 bp cleavage site, and wherein the FokI nucleases dimerize and make a double strand break at a target sequence.

In one aspect, the nuclease agent comprises a first TAL-repeat-based DNA binding domain and a second TAL-repeat-based DNA binding domain, wherein each of the first and the second TAL-repeat-based DNA binding domain is operably linked to a FokI nuclease, wherein the first and the second TAL-repeat-based DNA binding domain recognize two contiguous target DNA sequences in each strand of the target DNA sequence separated by a 5 bp or 6 bp cleavage site, and wherein the FokI nucleases dimerize and make a double strand break.

### Zinc-Finger Nuclease (ZFN)

In some aspects, the nuclease agent employed for homologous recombination in the various methods and compositions disclosed herein can comprise a zinc-finger nuclease (ZFN) system. In one aspect, each monomer of the ZFN comprises 3 or more zinc finger-based DNA binding domains, wherein each zinc finger-based DNA binding domain binds to a 3 bp subsite. In other aspects, the ZFN is a chimeric protein comprising a zinc finger-based DNA binding domain operably linked to an independent nuclease. In one aspect, the independent endonuclease is a FokI endonuclease. In one aspect, the nuclease agent comprises a first ZFN and a second ZFN, wherein each of the first ZFN and the second ZFN is operably linked to a FokI nuclease, wherein the first and the second ZFN recognize two contiguous target DNA sequences in each strand of the target DNA sequence separated by about 6 bp to about 40 bp cleavage site or about a 5 bp to about 6 bp cleavage site, and wherein the FokI nucleases dimerize and make a double strand break. See, for example, US20060246567; US20080182332; US20020081614; US20030021776; WO/2002/057308A2; US20130123484; US20100291048; and, WO/2011/017293A2, each of which is herein incorporated by reference.

### Meganucleases

In some aspects, the nuclease agent employed for homologous recombination in the various methods and compositions disclosed herein can comprise a meganuclease system. Meganucleases have been classified into four families based on conserved sequence motifs, the families are the “LAGLIDADG,” “GIY-YIG,” “H-N-H,” and “His-Cys box” families. These motifs participate in the coordination of metal ions and hydrolysis of phosphodiester bonds.

HEases are notable for their long recognition sites, and for tolerating some sequence polymorphisms in their DNA substrates. Meganuclease domains, structure and function are known, see for example, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764.

In some examples a naturally occurring variant, and/or engineered derivative meganuclease is used. Methods for modifying the kinetics, cofactor interactions, expression, optimal conditions, and/or recognition site specificity, and screening for activity are known, see for example, Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames et al., (2005) Nucleic Acids Res 33:e178; Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346.

Any meganuclease can be used herein, including, but not limited to, I-SceI, I-SceII, I-SceIII, I-SceIV, I-SceV, I-SecVI, I-SceVII, I-CeuI, I-CeuAIIP, I-CreI, I-CrepsbIP, I-CrepsbIIP, I-CrepsbIIIP, I-CrepsbIVP, I-TliI, I-PpoI, PI-PspI, F-SceI, F-SceII, F-SuvI, F-TevI, F-TevII, I-AmaI, I-Anil, I-ChuI, I-CmoeI, I-CpaI, I-CpaII, I-CsmI, I-CvuI, I-CvuAIP, I-DdiI, I-DdiII, I-DirI, I-DmoI, I-HmuI, I-HmuII, I-HsNIP, I-LlaI, I-MsoI, I-NaaI, I-NanI, I-NcIIP, I-NgrIP, I-NitI, I-NjaI, I-Nsp236IP, I-PakI, I-PboIP, I-PcuIP, I-PcuAI, I-PcuVI, I-PgrIP, I-PobIP, I-PorIIP, I-PbpIP, I-SpBetaIP, I-ScaI, I-SexIP, I-SneIP, I-SpomI, I-SpomCP, I-SpomIP, I-SpomIIP, I-SquIP, I-Ssp6803I, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-MtuI, PI-MtuHIP, PI-MtuHIIP, PI-PfuI, PI-PfuII, PI-PkoI, PI-PkoII, PI-Rma43812IP, PI-SpBetaIP, PI-SceI, PI-TfuI, PI-TfuII, PI-ThyI, PI-TliI, PI-TliII, or any active variants or fragments thereof.

In one aspect, the meganuclease recognizes double-stranded DNA sequences of 12 to 40 base pairs. In one aspect, the meganuclease recognizes one perfectly matched target sequence in one of the heterologous plasmids described herein. In one aspect, the meganuclease is a homing nuclease. In one aspect, the homing nuclease is a “LAGLIDADG” family of homing nuclease. In one aspect, the “LAGLIDADG” family of homing nuclease is selected from I-SceI, I-CreI, and I-Dmol.

### Restriction Endonucleases

In some aspects, the nuclease agent employed for homologous recombination in the various methods and compositions disclosed herein can comprise a restriction endonuclease, which includes Type I, Type II, Type III, and Type IV endonucleases. Type I and Type III restriction endonucleases recognize specific recognition sites, but typically cleave at a variable position from the nuclease binding site, which can be hundreds of base pairs away from the cleavage site (recognition site). In Type II systems the restriction activity is independent of any methylase activity, and cleavage typically occurs at specific sites within or near to the binding site. Most Type II enzymes cut palindromic sequences, however Type IIa enzymes recognize non-palindromic recognition sites and cleave outside of the recognition site, Type IIb enzymes cut sequences twice with both sites outside of the recognition site, and Type IIs enzymes recognize an asymmetric recognition site and cleave on one side and at a defined distance of about 1-20 nucleotides from the recognition site. Type IV restriction enzymes target methylated DNA. Restriction enzymes are further described and classified, for example in the REBASE database (webpage at rebase.neb.com; Roberts et al., (2003) Nucleic Acids Res 31:418-20), Roberts et al., (2003) Nucleic Acids Res 31:1805-12, and Belfort et al., (2002) in Mobile DNA II, pp. 761-783, Eds. Craigie et al., (ASM Press, Washington, D.C.).

The nuclease agent may be introduced into the cell by any means known in the art. The polypeptide encoding the nuclease agent may be directly introduced into the cell. Alternatively, a polynucleotide encoding the nuclease agent can be introduced into the cell. When a polynucleotide encoding the nuclease agent is introduced into the cell, the nuclease agent can be transiently, conditionally or constitutively expressed within the cell. Thus, the polynucleotide encoding the nuclease agent can be contained in an expression cassette and be operably linked to a conditional promoter, an inducible promoter, a constitutive promoter, or a tissue-specific promoter. Such promoters of interest are discussed in further detail elsewhere herein. Alternatively, the nuclease agent is introduced into the cell as an mRNA encoding or comprising a nuclease agent.

Active variants and fragments of nuclease agents (i.e., an engineered nuclease agent) are also provided. Such active variants can comprise at least 65%, 70%, 75%, 80%, 85%, 90%, 91%, 92%, 93%, 94%, 95%, 96%, 97%, 98%, 99% or more sequence identity to the native nuclease agent, wherein the active variants retain the ability to cut at a desired recognition site and hence retain nick or double-strand-break-inducing activity. For example, any of the nuclease agents described herein can be modified from a native endonuclease sequence and designed to recognize and induce a nick or double-strand break at a recognition site that was not recognized by the native nuclease agent. Thus in some aspects, the engineered nuclease has a specificity to induce a nick or double-strand break at a recognition site that is different from the corresponding native nuclease agent recognition site. Assays for nick or double-strand-break-inducing activity are known and generally measure the overall activity and specificity of the endonuclease on DNA substrates containing the recognition site.

When the nuclease agent is provided to the cell through the introduction of a polynucleotide encoding the nuclease agent, such a polynucleotide encoding a nuclease agent can be modified to substitute codons having a higher frequency of usage in the cell of interest, as compared to the naturally occurring polynucleotide sequence encoding the nuclease agent. For example, the polynucleotide encoding the nuclease agent can be modified to substitute codons having a higher frequency of usage in a given prokaryotic or eukaryotic cell of interest, including a bacterial cell, a yeast cell, a human cell, a non-human cell, a non-rat eukaryotic cell, a mammalian cell, a rodent cell, a non-rat rodent cell, a mouse cell, a rat cell, a hamster cell or any other host cell of interest, as compared to the naturally occurring polynucleotide sequence.

### Homologous Recombination Sequences

A crucial advantage of the methods and compositions of the present disclosure is the possibility of generating a landing pad cell without the need of information regarding the genomic context in which the landing pad plasmid or a portion thereof is going to be inserted. This is possible because the methods disclosed herein rely on the targeted incorporation of the landing pad plasmid or a portion thereof in a location occupied by a parental plasmid. Sequence information regarding the parental plasmid is generally available or known in the art (e.g., commercial plasmids). Thus, it is possible to rely on such information to generate homologous recombination sequences that would guide the exchange of an internal subsequence of the parental plasmid with a landing pad plasmid sequence or a portion thereof via homologous recombination.

In some aspects of the present disclosure, the 5′ homologous recombination site comprises a polynucleotide sequence of SEQ ID NO: 16.

In some aspects of the present disclosure, the 3′ homologous recombination site comprises a polynucleotide sequence of SEQ ID NO: 17.

In some aspects of the present disclosure, the 5′ homologous recombination site comprises a polynucleotide sequence of SEQ ID NO: 16, and the 3′ homologous recombination site comprises a polynucleotide sequence of SEQ ID NO: 17.

In some aspects, the 5′ homologous recombination site comprises at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 510, at least about 520, at least about 530, at least about 540, at least 550, or at least about 553 contiguous nucleotides from SEQ ID NO: 16.

In some aspects, the 3′ homologous recombination site comprises at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 510, at least about 520, at least about 530, at least about 540, at least about 550, at least about 560, at least about 570, at least about 580, at least about 590, at least about 600, at least about 610, at least about 620, at least about 630, at least about 640, at least about 650, at least about 660, at least about 670, at least about 680, at least about 690, at least about 700, at least about 710, at least about 720, at least about 730, at least about 740, at least about 750, at least about 760, at least about 770, at least about 780, at least about 790, at least about 800, at least about 810, at least about 820, at least about 830, at least about 840, at least about 850, at least about 860, at least about 870, at least about 880, at least about 890, at least about 900, at least about 1000, at least about 1010, at least about 1020, at least about 1030, at least about 1040, at least about 1050, at least about 1060, at least about 1070, at least about 1080, at least about 1090, at least about 1100, at least about 1110, at least about 1120, at least about 1130, at least about 1140, at least about 1150, at least about 1160, at least about 1170, at least about 1180, at least about 1190, at least about 1200, at least about 1210, at least about 1220, or at least about 1221 contiguous nucleotides from SEQ ID NO: 17.

In some aspects, the 5′ homologous recombination site comprises at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 510, at least about 520, at least about 530, at least about 540, at least 550, or at least about 553 contiguous nucleotides from SEQ ID NO: 16; and the 3′ homologous recombination site comprises at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 510, at least about 520, at least about 530, at least about 540, at least about 550, at least about 560, at least about 570, at least about 580, at least about 590, at least about 600, at least about 610, at least about 620, at least about 630, at least about 640, at least about 650, at least about 660, at least about 670, at least about 680, at least about 690, at least about 700, at least about 710, at least about 720, at least about 730, at least about 740, at least about 750, at least about 760, at least about 770, at least about 780, at least about 790, at least about 800, at least about 810, at least about 820, at least about 830, at least about 840, at least about 850, at least about 860, at least about 870, at least about 880, at least about 890, at least about 900, at least about 1000, at least about 1010, at least about 1020, at least about 1030, at least about 1040, at least about 1050, at least about 1060, at least about 1070, at least about 1080, at least about 1090, at least about 1100, at least about 1110, at least about 1120, at least about 1130, at least about 1140, at least about 1150, at least about 1160, at least about 1170, at least about 1180, at least about 1190, at least about 1200, at least about 1210, at least about 1220, or at least about 1221 contiguous nucleotides from SEQ ID NO: 17.

In some aspects of the present disclosure, the 5′ homologous recombination site comprises a polynucleotide sequence of SEQ ID NO: 18 or SEQ ID NO: 114. In some aspects of the present disclosure, the 3′ homologous recombination site comprises a polynucleotide sequence of SEQ ID NO: 19 or SEQ ID NO: 115. In some aspects of the present disclosure, the 5′ homologous recombination site comprises a polynucleotide sequence of SEQ ID NO: 18 or SEQ ID NO:114, and the 3′ homologous recombination site comprises a polynucleotide sequence of SEQ ID NO: 19 or SEQ ID NO: 115.

In some aspects, the 5′ homologous recombination site comprises at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, or at least about 508 contiguous nucleotides from SEQ ID NO: 18 or SEQ ID NO: 114.

In some aspects, the 3′ homologous recombination site comprises at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, or at least about 298 contiguous nucleotides from SEQ ID NO: 19 or SEQ ID NO: 115.

In some aspects, the 5′ homologous recombination site comprises at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, or at least about 508 contiguous nucleotides from SEQ ID NO: 18 or SEQ ID NO: 114; and the 3′ homologous recombination site comprises at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, or at least about 298 contiguous nucleotides from SEQ ID NO: 19 or SEQ ID NO: 115.

In some aspects, a homologous recombination sequence (i.e., a DNA-targeting segment that targets a free plasmid, e.g., a landing pad plasmid or second GOI plasmid of FIG. 5A, to an integrated plasmid such as the parent plasmid or integrated landing pad plasmid of FIG. A) can have a length of from about 12 nucleotides to about 100 nucleotides. For example, the homologous recombination sequence can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the homologous recombination sequence can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt, from about 100 nt to about 125 nt, from about 125 nt to about 150 nt, from about 150 nt to about 175 nt, from about 175 nt to about 200 nt, from about 200 nt to about 225 nt, from about 225 nt to about 250 nt, from about 250 nt to about 300 nt, from about 300 nt to about 350 nt, from about 350 nt to about 400 nt, from about 400 nt to about 450 nt, from about 450 nt to about 500 nt, from about 500 nt to about 600 nt, from about 600 nt to about 700 nt, from about 700 nt to about 800 nt, from about 800 nt to about 900 nt, from about 900 nt to about 1000 nt, from about 1000 nt to about 1100 nt, from about 1100 nt to about 1200 nt, from about 1200 nt to about 1300 nt, from about 1300 nt to about 1400 nt, or from about 1400 nt to about 1500 nt.

In some aspects, the nucleotide sequence of the homologous recombination sequence in a free plasmid that is complementary to a nucleotide sequence of a homologous recombination sequence in an integrated plasmid can have a length at least about 12 nt, at least about 15 nt, at least about 18 nt, at least about 19 nt, at least about 20 nt, at least about 21 nt, at least about 25 nt, at least about 30 nt, at least about 35 nt, at least about 40 nt, at least about 45 nt, at least about 50 nt, at least about 55 nt, at least about 60 nt, at least about 65 nt, at least about 70 nt, at least about 75 nt, at least about 80 nt, at least about 85 nt, at least about 90 nt, at least about 95 nt, at least about 100 nt, at least about 200 nt, at least about 300 nt, at least about 400 nt, at least about 500 nt, at least about 600 nt, at least about 700 nt, at least about 800 nt, at least about 900 nt, at least about 1000 nt, at least about 1100 nt, at least about 1200 nt, at least about 1300 nt, at least about 1400 nt, or at least about 1500 nt.

In some aspects, the nucleotide sequence of the homologous recombination sequence in a free plasmid that is complementary to a nucleotide sequence of a homologous recombination sequence in an integrated plasmid can have a length of from about 12 nucleotides (nt) to about 100 nt, from about 12 nt to about 90 nt, from about 12 nt to about 80 nt, from about 12 nt to about 70 nt, from about 12 nt to about 60 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, or from about 12 nt to about 20 nt.

In some aspects, the nucleotide sequence of the homologous recombination sequence in a free plasmid that is complementary to a nucleotide sequence of a homologous recombination sequence in an integrated plasmid can have a length of from about 12 nucleotides (nt) to about 100 nt, from about 12 nt to about 90 nt, from about 12 nt to about 80 nt, from about 12 nt to about 70 nt, from about 12 nt to about 60 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, or from about 12 nt to about 20 nt.

In some aspects, the nucleotide sequence of the homologous recombination sequence in a free plasmid that is complementary to a nucleotide sequence of a homologous recombination sequence in an integrated plasmid can have a length of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400,410,420,430,440,450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 2000, 2500, 3000, 3500, 4000, 4500, 5000, or 5500 nucleotides.

The percent complementarity between the nucleotide sequence of the homologous recombination sequence in a free plasmid and the nucleotide sequence of the corresponding homologous recombination sequence in an integrated plasmid can be at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% complementary (i.e., fully complementary).

In some aspects, the distance between an homologous recombination sequence in an integrated plasmid and an adjacent genomic sequence is at least 1 nt, at least 2 nt, at least 3 nt, at least 4 nt, at least 5 nt, at least 6 nt, at least 7 nt, at least 8 nt, at least 9 nt, at least 10 nt, at least 11 nt, at least 12 nt, at least 13 nt, at least 14 nt, at least 15 nt, at least 16 nt, at least 17 nt, at least 18 nt, at least 19 nt, at least 20 nt, at least 21 nt, at least 22 nt, at least 23 nt, at least 24 nt, at least 25 nt, at least 26 nt, at least 27 nt, at least 28 nt, at least 29 nt, at least 30 nt, at least 31 nt, at least 32 nt, at least 33 nt, at least 34 nt, at least 35 nt, at least 36 nt, at least 37 nt, at least 38 nt, at least 39 nt, at least 40 nt, at least 41 nt, at least 42 nt, at least 43 nt, at least 44 nt, at least 45 nt, at least 46 nt, at least 47 nt, at least 48 nt, at least 49 nt, at least 50 nt, at least 51 nt, at least 52 nt, at least 53 nt, at least 54 nt, at least 55 nt, at least 56 nt, at least 57 nt, at least 58 nt, at least 59 nt, at least 60 nt, at least 61 nt, at least 62 nt, at least 63 nt, at least 64 nt, at least 65 nt, at least 66 nt, at least 67 nt, at least 68 nt, at least 69 nt, at least 70 nt, at least 71 nt, at least 72 nt, at least 73 nt, at least 74 nt, at least 75 nt, at least 76, at least 77 nt, at least 78 nt, at least 79 nt, at least 80 nt, at least 81 nt, at least 82 nt, at least 83 nt, at least 84 nt, at least 85 nt, at least 86 nt, at least 87 nt, at least 88 nt, at least 89 nt, at least 90 nt, at least 91, at least 92 nt, at least 93 nt, at least 94 nt, at least 95 nt, at least 96, at least 97 nt, at least 98 nt, at least 99 nt, at least 100 nt, at least 150 nt, at least 200 nt, at least 250 nt, at least 300 nt, at least 350 nt, at least 400 nt, at least 450 nt, at least 500 nt, about least 550 nt, at least 600 nt, at least 650 nt, at least 700 nt, at least 750 nt, at least 800 nt, at least 850 nt, at least 900 nt, at least 950 nt, at least 1000 nt, at least 1100 nt, at least 1200 nt, at least 1300 nt, at least 1400 nt, at least 1500 nt, at least 1600 nt, at least 1700 nt, at least 1800 nt, at least 1900 nt, at least 2000 nt, at least 2100 nt, at least 2200 nt, at least 2300 nt, at least 2400 nt, at least 2500 nt, at least 3000 nt, at least 3500 nt, at least 4000 nt, at least 4500 nt, at least 5000 nt, or at least 5000 nt.

In some aspects, the distance between an homologous recombination sequence in an integrated plasmid and an adjacent genomic sequence is 1 nt, 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, 50 nt, 51 nt, 52 nt, 53 nt, 54 nt, 55 nt, 56 nt, 57 nt, 58 nt, 59 nt, 60 nt, 61 nt, 62 nt, 63 nt, 64 nt, 65 nt, 66 nt, 67 nt, 68 nt, 69 nt, 70 nt, 71 nt, 72 nt, 73 nt, 74 nt, 75 nt, 76, 77 nt, 78 nt, 79 nt, 80 nt, 81 nt, 82 nt, 83 nt, 84 nt, 85 nt, 86 nt, 87 nt, 88 nt, 89 nt, 90 nt, 91, 92 nt, 93 nt, 94 nt, 95 nt, 96, 97 nt, 98 nt, 99 nt, 100 nt, 150 nt, 200 nt, 250 nt, 300 nt, 350 nt, 400 nt, 450 nt, 500 nt, 55 nt, 600 nt, 650 nt, 700 nt, 750 nt, 800 nt, 850 nt, 900 nt, 960 nt, 1000 nt, 1100 nt, 1200 nt, 1300 nt, 1400 nt, 1500 nt, 1600 nt, 1700 nt, 1800 nt, 1900 nt, 2000 nt, 2100 nt, 2200 nt, about 2300 nt, about 2400 nt, 2500 nt, 3000 nt, 3500 nt, 4000 nt, 4500 nt, 5000 nt, or 5000 nt.

In some aspects, the distance between an homologous recombination sequence in an integrated plasmid and an adjacent genomic sequence is between about 10 nt and about 20 nt, about 10 nt and about 20 nt, about 20 nt and about 30 nt, about 30 nt and about 40 nt, about 40 nt and about 50 nt, about 50 nt and about 60 nt, about 60 nt and about 70 nt, about 70 nt and about 80 nt, about 80 nt and about 90 nt, about 90 nt and about 100 nt, about 100 nt and about 200 nt, about 200 nt and about 300 nt, about 300 nt and about 400 nt, about 400 nt and about 500 nt, about 500 nt and about 600 nt, about 600 nt and about 700 nt, about 700 nt and about 800 nt, about 800 nt and about 900 nt, about 900 nt and about 1000 nt, about 1000 nt and about 1100 nt, about 1100 nt and about 1200 nt, about 1200 nt and about 1300 nt, about 1300 nt and about 1400 nt, about 1400 nt and about 1500 nt, about 1500 nt and about 1600 nt, about 1600 nt and about 1700 nt, about 1700 nt and about 1800 nt, about 1800 nt and about 1900 nt, about 1900 nt and about 2000 nt, about 2000 nt and about 2100 nt, about 2100 nt and about 2200 nt, about 2200 nt and about 2300 nt, about 2300 nt and about 2400 nt, about 2400 nt and 2500 nt, about 2500 nt and about 3000 nt, about 3000 nt and about 3500 nt, about 3500 nt and about 4000 net, about 4000 nt and about 4500 nt, about 4500 nt and about 5000 nt, or about 5000 nt and about 5000 nt.

### Site-Specific Recombination Systems

In some aspects of the present disclosure, e.g., in the recombination event between a landing pad plasmid and a second GOI plasmid, the recombination process takes place through the use of a site-specific recombination system.

The site-specific recombinase can be introduced into the cell by any means, including by introducing the recombinase polypeptide into the cell or by introducing a polynucleotide encoding the site-specific recombinase into the host cell. The polynucleotide encoding the site-specific recombinase can be located within the insert nucleic acid or within a separate polynucleotide. The site-specific recombinase can be operably linked to a promoter active in the cell including, for example, an inducible promoter, a promoter that is endogenous to the cell, a promoter that is heterologous to the cell, a cell-specific promoter, a tissue-specific promoter, or a developmental stage-specific promoter.

In some aspects, the site-specific recombination sites flank a polynucleotide encoding a selection marker and/or a reporter gene contained within the insert nucleic acid. In such instances following integration of a landing pad plasmid nucleic acid at a targeted locus in the parental cell, e.g., via CRISP/Cas mediated homologous recombination, the sequences between the site-specific recombination sites (e.g., LoxP sites) can be removed or exchanged via site-specific recombination with a corresponding sequence a GOI located between site-specific recombination sites in a second GOI plasmid.

Site-specific recombination, also known as conservative site-specific recombination, is a type of genetic recombination in which DNA strand exchange takes place between segments possessing at least a certain degree of sequence homology. Site-specific recombinases (SSRs) perform rearrangements of DNA segments by recognizing and binding to short DNA sequences (sites), at which they cleave the DNA backbone, exchange the two DNA helices involved and rejoin the DNA strands. While in some site-specific recombination systems of just a recombinase enzyme and the recombination sites is enough to perform all these reactions, in other systems a number of accessory proteins and/or accessory sites are also needed. Multiple genome modification strategies, among these recombinase-mediated cassette exchange (RMCE), an advanced approach for the targeted introduction of transcription units into predetermined genomic loci, rely on the capacities of SSRs.

Site-specific recombination systems are highly specific, fast and efficient, even when faced with complex eukaryotic genomes.

Recombination sites are typically between 30 and 200 nucleotides in length and consist of two motifs with a partial inverted-repeat symmetry, to which the recombinase binds, and which flank a central crossover sequence at which the recombination takes place. The pairs of sites between which the recombination occurs are usually identical, but there are exceptions (e.g., attP and attB of X integrase).

In some aspects, the site-specific recombinase recombination is mediated by a Tyr-recombinase mediated system, a Tyr-integrase mediated system, a Serine-resolvase/invertase mediated system, or a Serine-integrase mediated system. In some aspects, the Tyr-recombinase mediated system comprises a Cre, Dre, Flp, KD, B3, or B3 Tyr-recombinase. In some aspects, the Tyr-integrase mediated system comprises a λ (Lambda), HK022, or HP1 Tyr-integrase. In some aspects, the Serine-resolvase/invertase mediated system comprises a γδ (Gammadelta), ParA, Tn3, or Gin Serine-resolvase/integrase. In some aspects, the Serine-integrase mediated system comprises a PhiC31, Bxb1, pr R4 Serine-integrase.

In some specific aspects, the Tyr-recombinase mediated system comprises a Cre Tyr-recombinase. Cre-Lox recombination is a site-specific recombinase technology, used to carry out deletions, insertions, translocations and inversions at specific sites in the DNA of cells. It allows the DNA modification to be targeted to a specific cell type or be triggered by a specific external stimulus. The system consists of a single enzyme, Cre recombinase, that recombines a pair of short target sequences called the Lox sequences. This system can be implemented without inserting any extra supporting proteins or sequences. The Cre enzyme and the original Lox site called the LoxP sequence are derived from bacteriophage P1.

LoxP (locus of X-over P1) is a site on the bacteriophage P1 consisting of 34 bp. The site includes an asymmetric 8 bp sequence, variable except for the middle two bases, in between two sets of symmetric, 13 bp sequences. The exact sequence is given below; ‘N’ indicates bases which may vary, and lowercase letters indicate bases that have been mutated from the wild-type. The 13 bp sequences are palindromic but the 8 bp spacer is not, thus giving the loxP sequence a certain direction. Usually loxP sites come in pairs for genetic manipulation. If the two loxP sites are in the same orientation, the floxed sequence (sequence flanked by two loxP sites) is excised; however, if the two loxP sites are in the opposite orientation, the floxed sequence is inverted. If there exists a floxed donor sequence, the donor sequence can be swapped with the original sequence. This technique, called recombinase-mediated cassette exchange, can used in the methods of the present disclosure to swap the polynucleotide sequence located between two LoxP site in the landing pad plasmid with the polynucleotide sequence located between two LoxP sites in the second GOI plasmid. Accordingly, in some aspects, the SSRS is a LoxP site.

In some aspects, the LoxP comprises a nucleic acid sequence of SEQ ID NO: 1, i.e., a wild-type LoxP site. In other aspects, the LoxP site is a mutant LoxP site corresponding to SEQ ID NO: 2, wherein N can be any nucleotide (e.g., A, T, C or G).

In some aspects, the mutant LoxP site comprises a nucleic acid selected from the group consisting of SEQ ID NO: 3 (lox 511); SEQ ID NO: 4 (lox 5171); SEQ ID NO: 5 (lox 2272); SEQ ID NO: 6 (M2); SEQ ID NO: 7 (M3); SEQ ID NO: 8 (M7); SEQ ID NO: 9 (M11); SEQ ID NO: 10 (lox 71); SEQ ID NO: 11 (lox 66); and SEQ ID NOS: 28 to 82.

In some aspects, the two LoxP sites used according to the present disclosure can be two LoxP sites selected from the group consisting of SEQ ID NO:1, 2, 3, 4, 5, 6, 7, 8, 9, 10, 11, 28-82 or any combination thereof. In some aspects, the LoxP sites in a pair of LoxP sites are identical. In some aspects, the LoxP sites in a pair of LoxP sites are different.

In some aspects, both LoxP sites are wild-type LoxP sites. See Araki, K (1997). “Targeted integration of DNA using mutant lox sites in embryonic stem cells”. Nucleic Acids Research. 25 (4): 868-872, which is herein incorporated by reference in its entirety.

In other aspects, the Tyr-recombinase mediated system comprises a Flp Tyr-recombinase. Flp-FRT recombination is a site-directed recombination technology, increasingly used to manipulate an organism's DNA under controlled conditions in vivo. It is analogous to Cre-lox recombination but involves the recombination of sequences between flippase recognition target (FRT) sites by the recombinase flippase (Flp) derived from the 2μ plasmid of baker's yeast Saccharomyces cerevisiae.

Although the basic chemical reaction is the same for both Tyrosine and Serine recombinases, there are some differences between them. Tyrosine recombinases, such as Cre or FLP, cleave one DNA strand at a time at points that are staggered by 6-8 bp, linking the 3′ end of the strand to the hydroxyl group of the tyrosine nucleophile. Strand exchange then proceeds via a crossed strand intermediate analogous to the Holliday junction in which only one pair of strands has been exchanged. The mechanism and control of Serine recombinases is much less well understood. This group of enzymes was only discovered in the mid-1990s and is still relatively small. The now classical members gamma-delta and Tn3 resolvase, but also new additions like φC31-, Bxb1-, and R4 integrases, cut all four DNA strands simultaneously at points that are staggered by 2 bp. During cleavage, a protein-DNA bond is formed via a transesterification reaction, in which a phosphodiester bond is replaced by a phosphoserine bond between a 5′ phosphate at the cleavage site and the hydroxyl group of the conserved serine residue (S10 in resolvase). Contrary to members of the Tyr-class the recombination pathway converts two different substrate sites (attP and attB) to site-hybrids (attL and attR). This explains the irreversible nature of this particular recombination pathway, which can only be overcome by auxiliary “recombination directionality factors” (RDFs).

In some aspects, the SSRS is a flippase recognition target (FRT) site. The 34 bp minimal FRT site sequence has the sequence set forth in SEQ ID NO: 12 for which flippase (Flp) binds to both 13-bp arms of SEQ ID NO: 13 flanking the 8 bp spacer, i.e. the site-specific recombination (region of crossover) in reverse orientation. FRT-mediated cleavage occurs just ahead from the asymmetric 8 bp core region (5′-tctagaaa-3′) on the top strand and behind this sequence on the bottom strand. Several variant FRT sites exist, but recombination can usually occur only between two identical FRTs but generally not among non-identical (“heterospecific”) FRTs. In some aspects, a FRT site disclosed herein is selected from SEQ ID NOS: 12, 13, and 83 to 91. In some aspects, a pair of FRT sites disclosed herein is selected from SEQ ID NOS: 12, 13, and 83 to 91. In some aspects, the FRT sites in a pair of FRT sites are identical. In some aspects, the FTR sites in a pair of FRT sites are different.

In some aspects, an att site disclosed herein is selected from SEQ ID NOS: 92 to 109. In some aspects, the att site is an attB site. In some aspects, the att site in an attP site.

In some aspects, a plasmid of the present disclosure, e.g., a GOI plasmid or a landing pad plasmid, can comprise a single SSRS. In some aspects, a plasmid of the present disclosure, e.g., a GOI plasmid or a landing pad plasmid, can comprises more than one SSRS. In some aspects, a plasmid of the present disclosure, e.g., a GOI plasmid or a landing pad plasmid, can comprise two SSRS. In some aspects, a plasmid of the present disclosure, e.g., a GOI plasmid or a landing pad plasmid, can comprise two SSRS corresponding to the same site-specific recombinase system. In some aspects, a plasmid of the present disclosure, e.g., a GOI plasmid or a landing pad plasmid, can comprise two Tyr-recombinase sites. In some aspects, a plasmid of the present disclosure, e.g., a GOI plasmid or a landing pad plasmid, can comprise two a Tyr-integrase sites. In some aspects, a plasmid of the present disclosure, e.g., a GOI plasmid or a landing pad plasmid, can comprise two a Serine-resolvase/invertase sites. In some aspects, a plasmid of the present disclosure, e.g., a GOI plasmid or a landing pad plasmid, can comprise two or a Serine-integrase site.

In some aspects, a plasmid of the present disclosure, e.g., a GOI plasmid or a landing pad plasmid, can comprise two SSRS corresponding to different site-specific recombinase systems. In some aspects, a plasmid of the present disclosure, e.g., a GOI plasmid or a landing pad plasmid, can comprise two Tyr-recombinase sites. In some aspects, a plasmid of the present disclosure, e.g., a GOI plasmid or a landing pad plasmid, can comprise two Tyr-integrase sites. In some aspects, a plasmid of the present disclosure, e.g., a GOI plasmid or a landing pad plasmid, can comprise two a Serine-resolvase/invertase sites. In some aspects, a plasmid of the present disclosure, e.g., a GOI plasmid or a landing pad plasmid, can comprise two or a Serine-integrase site.

In some aspects, a plasmid of the present disclosure, e.g., a GOI plasmid or a landing pad plasmid, can comprise a Tyr-recombinase site and a Tyr-integrase site. In some aspects, a plasmid of the present disclosure, e.g., a GOI plasmid or a landing pad plasmid, can comprise Tyr-recombinase site and a Serine-resolvase/invertase site. In some aspects, a plasmid of the present disclosure, e.g., a GOI plasmid or a landing pad plasmid, can comprise a Tyr-recombinase site and a Serine-integrase site. In some aspects, a plasmid of the present disclosure, e.g., a GOI plasmid or a landing pad plasmid, can comprise a Tyr-integrase site and a Serine-resolvase/invertase site. In some aspects, a plasmid of the present disclosure, e.g., a GOI plasmid or a landing pad plasmid, can comprise a Tyr-integrase site and a Serine-integrase site. In some aspects, a plasmid of the present disclosure, e.g., a GOI plasmid or a landing pad plasmid, can comprise a Serine-resolvase/invertase site and Serine-integrase site.

TABLE 2
Exemplary SSRS. Each LoxP sequence comprises a left inverted repeat sequence
(positions 1-13), a spacer (positions 14-21) and a right inverted repeat sequence
(positions 22-34).

| LoxP sites | | SEQ ID |
|---|---|---|
| Site | Sequence | NO |
| lox WT | ATAACTTCGTATAATGTATGCTATACGAAGTTAT | 1 |
| (wild type, canonical loxP) | | |
| mutant LoxP | ATAACTTCGTATANNNTANNNTATACGAAGTTAT | 2 |
| (generic mutant LoxP; | | |
| N = A, T, C ,or G) | | |
| lox 66 | taccgTTCGTATAATGTATGCTATACGAAGTTAT | 11 |
| lox 71 | ATAACTTCGTATAATGTATGCTATACGAAcggta | 10 |
| lox 511 | ATAACTTCGTATAATGTATaCTATACGAAGTTAT | 3 |
| lox 2272 | ATAACTTCGTATAAaGTATCCTATACGAAGTTAT | 5 |
| lox 5171 | ATAACTTCGTATAATGTgTaCTATACGAAGT | 4 |
| | TAT | |
| m2 | ATAACTTCGTATAAgaaAccaTATACGAAGT | 6 |
| | TAT | |
| m3 | ATAACTTCGTATAtaaTAccaTATACGAAGT | 7 |
| | TAT | |
| m7 | ATAACTTCGTATAAgaTAgaaTATACGAAGT | 8 |
| | TAT | |
| m11 | ATAACTTCGTATAcgaTAccaTATACGAAGT | 9 |
| | TAT | |
| L3 | ATAACTTCGTATAAAGTCTCCTATACGAAGT | 28 |
| | TAT | |
| LoxFAS | ATAACTTCGTATAGAAAGGTATATACGAAGT | 29 |
| | TAT | |
| 2L | ATAACTTCGTATAGCATACATTATACGAAGT | 30 |
| | TAT | |
| a0 | TACCGTTCGTATAATGTATGCTATACGAAGT | 31 |
| | TAT | |
| a1 | TACCGTTCGTATAGTATAGTATATACGAAGT | 32 |
| | TAT | |
| a2 | TACCGTTCGTATAGGCTATAGTATACGAAGT | 33 |
| | TAT | |
| a3 | TACCGTTCGTATATTGTATGGTATACGAAGT | 34 |
| | TAT | |
| a4 | TACCGTTCGTATAGGATAGTATATACGAAGT | 35 |
| | TAT | |
| a5 | TACCGTTCGTATAACATACGCTATACGAAGT | 36 |
| | TAT | |
| a6 | TACCGTTCGTATAGGGTAGGCTATACGAAGT | 37 |
| | TAT | |
| a7 | TACCGTTCGTATAATGTATACTATACGAAGT | 38 |
| | TAT | |
| a8 | TACCGTTCGTATAGGATACTTTATACGAAGT | 39 |
| | TAT | |
| a9 | TACCGTTCGTATAAGAAACCATATACGAAGT | 40 |
| | TAT | |
| a10 | TACCGTTCGTATATAATACCATATACGAAGT | 41 |
| | TAT | |
| a11 | TACCGTTCGTATAACCAAAGATATACGAAGT | 42 |
| | TAT | |
| a12 | TACCGTTCGTATAACCATAATTATACGAAGT | 43 |
| | TAT | |
| b0 | ATAACTTCGTATAATGTATGCTATACGAACG | 44 |
| | GTA | |
| b1 | ATAACTTCGTATAGTATAGTATATACGAACG | 45 |
| | GTA | |
| b2 | ATAACTTCGTATAGGCTATAGTATACGAACG | 46 |
| | GTA | |
| b3 | ATAACTTCGTATATTGTATGGTATACGAACG | 47 |
| | GTA | |
| b4 | ATAACTTCGTATAGGATAGTATATACGAACG | 48 |
| | GTA | |
| b5 | ATAACTTCGTATAACATACGCTATACGAACG | 49 |
| | GTA | |
| b6 | ATAACTTCGTATAGGGTAGGCTATACGAACG | 50 |
| | GTA | |
| b7 | ATAACTTCGTATAATGTATACTATACGAACG | 51 |
| | GTA | |
| b8 | ATAACTTCGTATAGGATACTTTATACGAACG | 52 |
| | GTA | |
| b9 | ATAACTTCGTATAAGAAACCATATACGAACG | 53 |
| | GTA | |
| b10 | ATAACTTCGTATATAATACCATATACGAACG | 54 |
| | GTA | |
| b11 | ATAACTTCGTATAACCAAAGATATACGAACG | 55 |
| | GTA | |
| b12 | ATAACTTCGTATAACCATAATTATACGAACG | 56 |
| | GTA | |
| c0 | ATAAATTGCTATAATGTATGCTATACGAAGT | 57 |
| | TAT | |
| c1 | ATAAATTGCTATAGTATAGTATATACGAAGT | 58 |
| | TAT | |
| c2 | ATAAATTGCTATAGGCTATAGTATACGAAGT | 59 |
| | TAT | |
| c3 | ATAAATTGCTATATTGTATGGTATACGAAGT | 60 |
| | TAT | |
| c4 | ATAAATTGCTATAGGATAGTATATACGAAGT | 61 |
| | TAT | |
| c5 | ATAAATTGCTATAACATACGCTATACGAAGT | 62 |
| | TAT | |
| c6 | ATAAATTGCTATAGGGTAGGCTATACGAAGT | 63 |
| | TAT | |
| c7 | ATAAATTGCTATAATGTATACTATACGAAGT | 64 |
| | TAT | |
| c8 | ATAAATTGCTATAGGATACTTTATACGAAGT | 65 |
| | TAT | |
| c9 | ATAAATTGCTATAAGAAACCATATACGAAGT | 66 |
| | TAT | |
| c10 | ATAAATTGCTATATAATACCATATACGAAGT | 67 |
| | TAT | |
| c11 | ATAAATTGCTATAACCAAAGATATACGAAGT | 68 |
| | TAT | |
| c12 | ATAAATTGCTATAACCATAATTATACGAAGT | 69 |
| | TAT | |
| do | ATAACTTCGTATAATGTATGCTATACGAATA | 70 |
| | ATT | |
| d1 | ATAACTTCGTATAGTATAGTATATACGAATA | 71 |
| | ATT | |
| d2 | ATAACTTCGTATAGGCTATAGTATACGAATA | 72 |
| | ATT | |
| d3 | ATAACTTCGTATATTGTATGGTATACGAATA | 73 |
| | ATT | |
| d4 | ATAACTTCGTATAGGATAGTATATACGAATA | 74 |
| | ATT | |
| d5 | ATAACTTCGTATAACATACGCTATACGAATA | 75 |
| | ATT | |
| d6 | ATAACTTCGTATAGGGTAGGCTATACGAATA | 76 |
| | ATT | |
| d7 | ATAACTTCGTATAATGTATACTATACGAATA | 77 |
| | ATT | |
| d8 | ATAACTTCGTATAGGATACTTTATACGAATA | 78 |
| | ATT | |
| d9 | ATAACTTCGTATAAGAAACCATATACGAATA | 79 |
| | ATT | |
| d10 | ATAACTTCGTATATAATACCATATACGAATA | 80 |
| | ATT | |
| d11 | ATAACTTCGTATAACCAAAGATATACGAATA | 81 |
| | ATT | |
| d12 | ATAACTTCGTATAACCATAATTATACGAATA | 82 |
| | ATT | |

FRT sites

| Site | Sequence | |
|---|---|---|
| Frt WT | GAAGTTCCTATTCTCTAGAAAGTATAGGAAC | 12 |
| | TTC | |
| F3 | GAAGTTCCTATTCTTCAAATAGTATAGGAAC | 83 |
| | TTC | |
| F5 | GAAGTTCCTATTCTTCAAAAGGTATAGGAAC | 84 |
| | TTC | |
| F10 | GAAGTTCCTATTCACTAGAATGTATAGGAAC | 85 |
| | TTC | |
| F11 | GAAGTTCCTATTCTGAACTAAGTATAGGAAC | 86 |
| | TTC | |
| F12 | GAAGTTCCTATTCTTTCTGAAGTATAGGAAC | 87 |
| | TTC | |
| F13 | GAAGTTCCTATTCTCATATAAGTATAGGAAC | 88 |
| | TTC | |
| F14 | GAAGTTCCTATTCTATCAGAAGTATAGGAAC | 89 |
| | TTC | |
| F15 | GAAGTTCCTATTCTTATAGGAGTATAGGAAC | 90 |
| | TTC | |
| F16 | GAAGTTCCTATTCTCCGGGCAGTATAGGAAC | 91 |
| | TTC | |

attB/attP sites

| | |
|---|---|
| Site | Sequence |

fC31 (Streptomyces phage phiC31)

| | | |
|---|---|---|
| attB34 | GGTGCCAGGGCGTGCCCTTGGGCTCCCCGGG | 92 |
| | CGCG | |
| attP39 | CCCCAACTGGGGTAACCTTTGAGTTCTCTCA | 93 |
| | GTTGGGGG | |

fC31 (Streptomyces phage phiC31)

| | | |
|---|---|---|
| attB51 | CCGCGGTGCGGGTGCCAGGGCGTGCCCTTGG | 94 |
| | GCTCCCCGGGCGCGTACTCC | |
| attP50 | AGTAGTGCCCCAACTGGGGTAACCTTTGAGT | 95 |
| | TCTCTCAGTTGGGGGCGTA | |

BxB1 (Mycobacteriophage Bxb1)

| | | |
|---|---|---|
| attB | GGCCGGCTTGTCGACGACGGCGGTCTCCGTC | 96 |
| | GTCAGGATCATCCGG | |
| attP | GTGGTTTGTCTGGTCAACCACCGCGGTCTCA | 97 |
| | GTGGTGTACGGTACAAACCCA | |

Bxb1-GA

| | | |
|---|---|---|
| attB | CGGCCGGCTTGTCGACGACGGCGGACTCCGT | 98 |
| | CGTCAGGATCATCCGGCCGCCAC | |
| attP | CGTGGTTTGTCTGGTCAACCACCGCGGACTC | 99 |
| | AGTGGTGTACGGTACAAACCCAT | |

R4

| | | |
|---|---|---|
| attB | GCGCCCAAGTTGCCCATGACCATGCCGAAGC | 100 |
| | AGTGGTAGAAGGGCACCGGCAGACAC | |
| attP | AGGCATGTTCCCCAAAGCGATACCACTTGAA | 101 |
| | GCAGTGGTACTGCTTGTGGGTACACTCTGC | |

fBT1

| | | |
|---|---|---|
| attB | GTCCTTGACCAGGTTTTTGACGAAAGTGATC | 102 |
| | CAGATGATCCAGCTCCACACCCCGAACGC | |
| attP | GGTGCTGGGTTGTTGTCTCTGGACAGTGATC | 103 |
| | CATGGGAAACTACTCAGCACCACCAATG | |

WB

| | | |
|---|---|---|
| attB | AAGGTAGCGTCAACGATAGGTGTAACTGTCG | 104 |
| | TGTTTGTAACGGTACTTCCAACAGCTGGCG | |
| attP | TAGTTTTAAAGTTGGTTATTAGTTACTGTGA | 105 |
| | TATTTATCACGGTACCCAATAACCAATGAA | |

SPBc

| | | |
|---|---|---|
| attB | AGTGCAGCATGTCATTAATATCAGTACAGAT | 106 |
| | AAAGCTGTATCTCCTGTGAACACAATGGGTG | |
| attP | AAAGTAGTAAGTATCTTAAAAAACAGATAAA | 107 |
| | GCTGTATATTAAGATACTTACTAC | |

TP901

| | | |
|---|---|---|
| attB | TGATAATTGCCAACACAATTAACATCTCAAT | 108 |
| | CAAGGTAAATGCTTTTTCGTTTT | |
| attP | AATTGCGAGTTTTTATTTCGTTTATTTCAAT | 109 |
| | TAAGGTAACTAAAAAACTCCTTT | |

### Markers

In some aspects, the site-specific recombination sites in a landing pad plasmid flank a polynucleotide encoding a marker (e.g., a selection or selectable marker and/or a detectable or screenable marker such as a reporter gene). In such instances, following integration of the insert nucleic acid (area located between both SSRS) from the second GOI plasmid the sequences between the site-specific recombination sites on the landing pad plasmid are removed.

Marker systems exist in two broad categories: selectable markers and screenable markers. Selectable markers are typically genes for antibiotic resistance, which give the transformed organism (usually a single cell) the ability to live in the presence of an antibiotic. Screenable markers, also called reporter genes, typically cause a color change or other visible change in the cells of the transformed organism. This allows the investigator to quickly screen a large group of cells for the ones that have been transformed.

In some aspects, the selection marker is contained in a selection cassette. In one aspects, the at least one selection marker is glutamine synthetase (GS) and/or dihydrofolate reductase (DHFR).

In some aspects, the at least one selection marker is a drug resistance gene. In some aspects, the drug resistance gene is an antibiotic resistance gene, e.g., a puromycin resistance gene such as puromycin-N-acetyltransferase. Any selection markers known in the art can be used in the methods and compositions of the present disclosure. Such selection markers include, but are not limited, to neomycin phosphotransferase (neo), hygromycin B phosphotransferase (hyg), puromycin-N-acetyltransferase (puro), blasticidin S deaminase (bsr), xanthine/guanine phosphoribosyl transferase (gpt), herpes simplex virus thymidine kinase (HSV-k), or any combination thereof. In some aspects, the selection marker can be, e.g., a resistance gene to puromycin, neomycin, hygromycin B, blasticidin S, phleomycin, ZEOCIN™ (phleomycin D1), or G418 (geneticin).

In some aspects, the landing pad plasmid can comprise a detectable marker (e.g., a reporter gene) which encodes a protein. In some aspects, the nucleic acid sequence encoding the detectable marker is contained in a selection cassette. In some aspects, the nucleic acid sequence encoding the detectable marker is operably linker to a promoter.

In some aspects, the protein is a reporter protein, e.g., a fluorescent protein. In a particular aspect, the fluorescent protein is mCherry. In some aspects, the fluorescent protein is selected from the group consisting of green fluorescent protein (GFP), ZsGreen1, AcGFP1, enhanced green fluorescent protein (EGFP), GFPuv, AcGFP, enhanced blue fluorescent protein (EBFP), enhanced yellow fluorescent protein (EYFP), enhanced cyan fluorescent protein (ECFP), tdTomato, mCherry, DsRed, AmCyan, ZsGreen, ZsYellow, DsRed2, DsRed-Express, HcRed, AsRed, mOrange, mOrange2, mPlum, mStrawberry, mBanana, yellow fluorescent protein (YFP), mRaspberry, HcRed1, E2-Crimson, J-Red, mKO, mCitrine, Venus, YPet, Emerald, CyPet, Cerulean, cyan fluorescent protein (CFP), T-Sapphire, or any combination thereof. In some aspects, the reporter protein is luciferase or alkaline phosphatase.

Such reporter genes can be operably linked to a promoter active in the cell. Such promoters can be an inducible promoter, a promoter that is endogenous to the reporter gene or the cell, a promoter that is heterologous to the reporter gene or to the cell, a cell-specific promoter, a tissue-specific promoter, or a developmental stage-specific promoter.

### Cells

In principle the methods disclosed herein can be used of targeted gene integration into the genome of any eukaryotic cells. “Eukaryotic cell” includes, for example, mammalian cells, insect cells, avian cells, amphibian cells, e.g., frog oocytes, fish cells, fungal and yeast cells.

As used herein, the term “mammalian cell” is meant to include any cell obtained from a human or non-human mammal, including but not limited to porcine, ovine, bovine, rodents, ungulates, pigs, sheep, lambs, goats, cattle, deer, mules, horses, monkeys, dogs, cats, rats, and mice.

In some aspects, the cells are hybridoma cells, monoclonal antibody producing cells, virus-producing cells, transfected cells, cancer cells, and/or recombinant peptide producing cells.

Specific mammalian cells include, e.g., Cos, CHO (e.g., CHO-K1), MDCK, HEK293, HEK293T (human embryonic kidney cells expressing the large T-cell antigen), NIH3T3, Swiss3T3, BHK (e.g., BHK-21), L929 mouse fibroblast cells, AHT-107 hybridoma cells, mouse myeloma cells, monkey-fibroblast cells, X63 myeloma cells, HeLa cells, NSO hybridoma cells, U-937 cells, MK2.7 cells, PER-C6 cells, 5L8 hybridoma cells, Daudi cells, E14 cells, HL-60 cells, K562 cells, Jurkat cells, THP-1 cells, Sp2/0 cells, or any other cell type disclosed herein or known to one skilled in the art.

Additional mammalian cell types can include, but are not limited to, including primary epithelial cells (e.g., keratinocytes, cervical epithelial cells, bronchial epithelial cells, tracheal epithelial cells, kidney epithelial cells and retinal epithelial cells) and established cell lines and their strains (e.g., 293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LS 180 cells, LS 174T cells, NCI-H-548 cells, RPMI 2650 cells, SW-13 cells, T24 cells, WI-28 VA13, 2RA cells, WISH cells, BS-C-I cells, LLC-MK.sub.2 cells, Clone M-3 cells, 1-10 cells, RAG cells, TCMK-1 cells, Y-1 cells, LLC-PK.sub.1 cells, PK(15) cells, GH.1 cells, GH3 cells, L2 cells, LLC-RC 256 cells, MH.sub.1C1 cells, XC cells, MDOK cells, VSW cells, and TH-I, B1 cells, or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, MiCl.sub.1 cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDM.sub.1C3 cells, KLN205 cells, McCoy cells, Mouse L cells, Strain 2071 (Mouse L) cells, L-M strain (Mouse L) cells, L-MTK (Mouse L) cells, NCTC clones 2472 and 2555, SCC-PSA1 cells, Swiss/3T3 cells, Indian muntac cells, SIRC cells, CII cells, and Jensen cells, or derivatives thereof).

Any number of cancer cell lines are familiar to those skilled in the art. Representative examples of cancer cell lines that can be cultivated by the method of the present invention include but are not limited to the following cancer cell lines: human myeloma (e.g., KMM-1, KMS-11, KMS-12-PE, KMS-12-BM, KMS-18, KMS-20, KMS-21-PE, U266, RPMI8226); human breast cancer (e.g., KPL-1, KPL4, MDA-MB-231, MCF-7, KPL-3C, T47D, SkBr3, HS578T, MDA4355, Hs 606 (CRL-7368), Hs 605.T (CRL-7365) Hs 742.T (CRL-7482), BT474, HBL-100, HCC202, HCC1419, HCC1954, MCF7, MDA-361, MDA436, MDA453, SK-BR-3, ZR-75-30, UACC-732, UACC-812, UACC-893, UACC-3133, MX-1 and EFM-192A); ductal (breast) carcinoma (e.g., HS 57HT (HTB-126), HCC1008 (CRL-2320), HCC1954 (CRL-2338; HCC38 (CRL-2314), HCC1143 (CRL-2321), HCC1187 (CRL-2322), HCC1295 (CRL-2324), HCC1599 (CRL-2331), HCC1937 (CRL-2336), HCC2157 (CRL-2340), HCC2218 (CRL-2343), Hs574.T (CRL-7345), Hs 742.T (CRL-7482); skin cancer (e.g., COLO 829 (CRL-1974), TE 354.T (CRL-7762), Hs 925.T (CRL-7677)); human prostate cancer (e.g., MDA PCa 2a and MDA PCa 2b); bone cancer (e.g., Hs 919.T (CRL-7672), Hs 821.T (CRL-7554), Hs 820.T (CRL- 7552), Hs 704.T (CRL-7444), Hs 707(A).T (CRL-7448), Hs 735.T (CRL-7471), Hs 860.T (CRL-7595), Hs 888.T.(CRL-7622); Hs 889.T (CRL-7626); Hs 890.T (CRL-7628), Hs 709.T (CRL-7453)); human lymphoma (e.g., K562); human cervical carcinoma (e.g., HeLA); lung carcinoma cell lines (e.g., H125, H522, H1299, NCI-H2126 (ATCC CCL-256), NCI-H1672 (ATCC CRL-5886), NCI-2171 (CRL-5929); NCI-H2195 (CRL05931); lung adenocarcinoma (e.g., NCI-H1395 (CRL-5856), NCI-H1437 (CRL-5872), NCI-H2009 (CRL-5911), NCI-H2122 (CRL-5985), NCI-H2087 (CRL-5922); metastatic lung cancer (e.g., bone) (e.g., NCI-H209 (HTB-172); colon carcinoma cell lines (e.g., LN235, DLD2, Colon A, LIM2537, LIM1215, LIM1863, LIM1899, LIM2405 LIM2412, SK-CO1 (ATCC HTB-77), HT29 (ATCC HTB38), LoVo (ATCC CCL-229), SW1222 (ATCC HB-11028), and SW480 (ATCC CCL-228); ovarian cancer (e.g., OVCAR-3 (ATCC HTB-161) and SKOV-3 (ATCC HTB-77); mesothelioma (e.g., NCI-h2052 (CRL-5915); neuroendocrine carcinoma (e.g., HCI-H1770 (e.g., CRL-5893); gastric cancer (e.g., LIM1839); glioma (e.g., T98, U251, LN235); head and neck squamous cell carcinoma cell lines (e.g., SCC4, SCC9 and SCC25); medulloblastoma (e.g., Daoy, D283 Med and D341 Med); testicular non-seminoma (e.g., TERA1); prostate cancer (e.g., 178-2BMA, Du145, LNCaP, and PC-3). Other cancer cell lines are well known in the art.

In some aspects, the cell is a hybridoma disclosed in TABLE 2 of U.S. Publ. No. 2006/0073591, which is herein incorporated by reference in its entirety.

In some aspects, the eukaryotic cell is selected from the group consisting of mammalian cells, fibroblasts, pluripotent cells, non-human pluripotent cells, rodent multipotential cells, mouse or rat embryonic stem (ES) cells, human pluripotent cell, human adult stem cells, embryologically restricted human progenitor cells, or human induced pluripotent stem (iPS) cells.

Yeast useful for expression include by way of example Saccharomyces, Schizosaccharomyces, Hansenula (e.g., Hansenula polymorpha), Candida, Torulopsis, Yarrowia, Pichia (e.g., Pichia pastoris, Pichia guillermordii, Pichia methanolica, Pichia inositovera).

The cells can be transfected using standard methods known in the art, such as but not limited to Ca²⁺ phosphate or lipid-based systems.

### Genes of Interest

In some aspects, the gene of interest (GOI) in comprises one or more open reading frames, e.g., encoding one or more recombinant proteins, operably linked to one or more promoter and/or other regulatory sequences. In some aspects, the first GOI (gene of interest located on the parental plasmid) and the second GOI (gene of interest located on the second GOI plasmid) belong to the same molecule class. For example, if the first GOI was an antibody, the second GOI may also be antibody since the parent cell line efficiently expressed that type of recombinant protein.

In some aspects, the GOI comprises one or more polynucleotide sequences encoding a biologic, for example, and antibody or an antigen-binding portion thereof.

In some aspects, the GOI comprises a polynucleotide sequence encoding a protein comprising amino acid sequences identical to or substantially similar to all or part of one of the following proteins: tumor necrosis factor (TNF), flt3 ligand (WO 94/28391), erythropoeitin, thrombopoeitin, calcitonin, IL-2, angiopoietin-2 (Maisonpierre et al. (1997), Science 277(5322): 55-60), ligand for receptor activator of NF-kappa B (RANKL, WO 01/36637), tumor necrosis factor (TNF)-related apoptosis-inducing ligand (TRAIL, WO 97/01633), thymic stroma-derived lymphopoietin, granulocyte colony stimulating factor, granulocyte-macrophage colony stimulating factor (GM-CSF, Australian Patent No. 588819), mast cell growth factor, stem cell growth factor (U.S. Pat. No. 6,204,363), epidermal growth factor, keratinocyte growth factor, megakaryote growth and development factor, RANTES, human fibrinogen-like 2 protein (FGL2; NCBI accession no. NM-00682; Ruegg and Pytela (1995), Gene 160:257-62) growth hormone, insulin, insulinotropin, insulin-like growth factors, parathyroid hormone, interferons including α-interferons, γ-interferon, and consensus interferons (U.S. Pat. Nos. 4,695,623 and 4,897,471), nerve growth factor, brain-derived neurotrophic factor, synaptotagmin-like proteins (SLP 1-5), neurotrophin-3, glucagon, interleukins, colony stimulating factors, lymphotoxin-β, leukemia inhibitory factor, and oncostatin-M. See, e.g., Human Cytokines: Handbook for Basic and Clinical Research, all volumes (Aggarwal and Gutterman, eds. Blackwell Sciences, Cambridge, Mass., 1998); Growth Factors: A Practical Approach (McKay and Leigh, eds., Oxford University Press Inc., New York, 1993); and The Cytokine Handbook, Vols. 1 and 2 (Thompson and Lotze eds., Academic Press, San Diego, Calif, 2003), which are herein incorporated by reference in their entireties.

In some aspects, the GOI comprises a polynucleotide sequence encoding a protein comprising all or part of the amino acid sequence of a receptor for any of the above-mentioned proteins, an antagonist to such a receptor or any of the above-mentioned proteins, and/or proteins substantially similar to such receptors or antagonists. These receptors and antagonists include: both forms of tumor necrosis factor receptor (TNFR, referred to as p55 and p75, U.S. Pat. Nos. 5,395,760 and 5,610,279), Interleukin-1 (IL-1) receptors (types I and II; EP Patent No. 0460846, U.S. Pat. Nos. 4,968,607, and 5,767,064), IL-1 receptor antagonists (U.S. Pat. No. 6,337,072), IL-1 antagonists or inhibitors (U.S. Pat. Nos. 5,981,713, 6,096,728, and 5,075,222) IL- 2 receptors, IL-4 receptors (EP Patent No. 0 367 566 and U.S. Pat. No. 5,856,296), IL-15 receptors, IL-17 receptors, IL-18 receptors, Fc receptors, granulocyte-macrophage colony stimulating factor receptor, granulocyte colony stimulating factor receptor, receptors for oncostatin-M and leukemia inhibitory factor, receptor activator of NF-kappa B (RANK, WO 01/36637 and U.S. Pat. No. 6,271,349), osteoprotegerin (U.S. Pat. No. 6,015,938), receptors for TRAIL (including TRAIL receptors 1, 2, 3, and 4), and receptors that comprise death domains, such as Fas or Apoptosis-Inducing Receptor (AIR).

In some aspects, a GOI comprises a polynucleotide sequence encoding a protein comprising all or part of the amino acid sequences of differentiation antigens (referred to as CD proteins) or their ligands or proteins substantially similar to either of these. Examples of such antigens include CD22, CD27, CD30, CD39, CD40, and ligands thereto (CD27 ligand, CD30 ligand, etc.). Several of the CD antigens are members of the TNF receptor family, which also includes 41BB and OX40. The ligands are often members of the TNF family, as are 41BB ligand and OX40 ligand.

In some aspects, a GOI comprises a polynucleotide sequence encoding an enzymatically active protein or its ligands can also be produced using the methods disclosed herein. Examples include proteins comprising all or part of one of the following proteins or their ligands or a protein substantially similar to one of these: a disintegrin and metalloproteinase domain family members including TNF-alpha Converting Enzyme, various kinases, glucocerebrosidase, superoxide dismutase, tissue plasminogen activator, Factor VIII, Factor IX, apolipoprotein E, apolipoprotein A-I, globins, an IL-2 antagonist, alpha-1 antitrypsin, ligands for any of the above-mentioned enzymes, and numerous other enzymes and their ligands.

In some aspects, a GOI comprises a polynucleotide sequence encoding an antibody or an antigen-binding portion thereof. Examples of antibodies include, but are not limited to, those that recognize any one or a combination of proteins including, but not limited to, the above-mentioned proteins and/or the following antigens: CD2, CD3, CD4, CD8, CD11a, CD14, CD18, CD20, CD22, CD23, CD25, CD33, CD40, CD44, CD52, CD80 (B7.1), CD86 (B7.2), CD147, IL-1α, IL-1, IL-2, IL-3, IL-7, IL-4, IL-5, IL-8, IL-10, IL-2 receptor, IL-4 receptor, IL-6 receptor, IL-13 receptor, IL-18 receptor subunits, FGL2, PDGF-β and analogs thereof (see U.S. Pat. Nos. 5,272,064 and 5,149,792), VEGF, TGF, TGF-β2, TGF-β1, EGF receptor (see U.S. Pat. No. 6,235,883) VEGF receptor, hepatocyte growth factor, osteoprotegerin ligand, interferon gamma, B lymphocyte stimulator (BlyS, also known as BAFF, THANK, TALL-1, and zTNF4; see Do and Chen-Kiang (2002), Cytokine Growth Factor Rev. 13(1): 19-25), C5 complement, IgE, tumor antigen CA125, tumor antigen MUC1, PEM antigen, LCG (which is a gene product that is expressed in association with lung cancer), HER-2, HER-3, RAS (e.g., K-RAS), a tumor-associated glycoprotein TAG-72, the SK-1 antigen, tumor-associated epitopes that are present in elevated levels in the sera of patients with colon and/or pancreatic cancer, cancer-associated epitopes or proteins expressed on breast, colon, squamous cell, prostate, pancreatic, lung, and/or kidney cancer cells and/or on melanoma, glioma, or neuroblastoma cells, the necrotic core of a tumor, integrin alpha 4 beta 7, the integrin VLA-4, B2 integrins, TRAIL receptors 1, 2, 3, and 4, RANK, RANK ligand, TNF-α, the adhesion molecule VAP-1, epithelial cell adhesion molecule (EpCAM), intercellular adhesion molecule-3 (ICAM-3), leukointegrin adhesin, the platelet glycoprotein gp IIb/IIIa, cardiac myosin heavy chain, parathyroid hormone, rNAPc2 (which is an inhibitor of factor VIIa-tissue factor), MHC I, carcinoembryonic antigen (CEA), alpha-fetoprotein (AFP), tumor necrosis factor (TNF), CTLA-4 (which is a cytotoxic T lymphocyte-associated antigen), Fc-7-1 receptor, HLA-DR 10 beta, HLA-DR antigen, sclerostin, L-selectin, Respiratory Syncitial Virus, human immunodeficiency virus (HIV), hepatitis B virus (HBV), Streptococcus mutans, and Staphlycoccus aureus. Specific examples of known antibodies which can be produced using the methods of the invention include but are not limited to adalimumab, bevacizumab, infliximab, abciximab, alemtuzumab, bapineuzumab, basiliximab, belimumab, briakinumab, canakinumab, certolizumab pegol, cetuximab, conatumumab, denosumab, eculizumab, gemtuzumab ozogamicin, golimumab, ibritumomab tiuxetan, labetuzumab, mapatumumab, matuzumab, mepolizumab, motavizumab, muromonab-CD3, natalizumab, nimotuzumab, ofatumumab, omalizumab, oregovomab, palivizumab, panitumumab, pemtumomab, pertuzumab, ranibizumab, rituximab, rovelizumab, tocilizumab, tositumomab, trastuzumab, ustekinumab, vedolizomab, zalutumumab, and zanolimumab.

In some aspects, a GOI comprises a polynucleotide sequence encoding a recombinant fusion protein comprising, for example, any of the above-mentioned proteins. For example, recombinant fusion proteins comprising one of the above-mentioned proteins plus a multimerization domain, such as a leucine zipper, a coiled coil, an Fc portion of an immunoglobulin, or a substantially similar protein, can be produced using the methods of the invention. See e.g. WO94/10308; Lovejoy et al. (1993), Science 259:1288-1293; Harbury et al. (1993), Science 262:1401-05; Harbury et al. (1994), Nature 371:80-83; Hikansson et al. (1999), Structure 7:255-64. Specifically included among such recombinant fusion proteins are proteins in which a portion of a receptor is fused to an Fc portion of an antibody such as etanercept (a p75 TNFR:Fc), abatacept, or belatacept (CTLA4:Fc). In some aspects, a GOI comprises a polynucleotide sequence encoding a marker, e.g., a screenable marker disclosed above such as GFP or luciferase.

### Methods of Identifying Candidate Parental Cell Suitable to Generate a Landing Pad Cell Line

The present disclosure also provides method of efficiently identifying candidate parental cells suitable to generate landing pad cells according to the methods disclosed herein. The methods disclosed herein greatly simplify the selection and development of the cell suitable for expression of a biologic of interest, e.g., an antibody. For example, a typical selection process may require up to 10 or more different cell line generation workflows, identifying the top producing clones (e.g., 5-10 clones) for each cell line, characterizing each clone via Southern blot and/or determination of gene copy number, and then selecting the top candidate(s) as parental cell line(s).

In some aspects, the method comprises screening a library of cell lines comprising a plasmid, wherein the plasmid contains at least one expression cassette comprising a polynucleotide encoding a GOI (parental plasmid). In some aspects, the parental plasmid can be integrated at different genomic locations in the parental cell's genome.

In some aspects, the cell line library is a historical set of cell lines, i.e., cells that have previously been modified by integrating a parental plasmid, e.g., a cell line that has been developed to express a biologic, such as an antibody. In other aspects, the cell line library is generated, e.g., via random integration of a parental plasmid at multiple locations in the genome of the parental cell.

Next, the candidate cells, i.e., the cells in the library, can be screened for the presence of specific criteria, the goal being the selection of a cell line that (i) is a “hot cell,” i.e., it has an advantageous property, e.g., it has a high yield of recombinant protein compared to other cells expressing the same GOI, and (ii) has the parental plasmid inserted at a “hot spot,” i.e., a genomic location (locus) were parental plasmid is transcribed at high levels, or some other desirable characteristic.

In some aspects, the specific criteria considered to selected a cell in the library as a suitable parental cell to develop a landing pad cell comprise:
(a) Cell titer: Amount of recombinant protein of interest expressed by a candidate cell, generally in grams/L;
(b) Parental plasmid copy number: Number of copies of the parental plasmid integrated in a candidate cell, e.g., measured using qPCR using GAPDH as an internal control;
(c) RNA expression level: Amount of the RNA expressed by the candidate cell, determined, for example, using Southern blot;
(d) Plasmid configuration: Orientation of the parental plasmid in the genome of a candidate cell measured, e.g., using spPCR (splinkeret PCR), a technique that allows for the identification of plasmid junction sequences;
(e) Specific properties of the expressed product (e.g. a recombinant protein encoded by a GOI): For example, specific glycosylation patters, immunogenicity, affinity, binding specificity, aggregation, thermal stability, etc.; or,
(f) any combination thereof.

In some aspects, a candidate cell is selected for the generation of a landing pad cell line if cell titer is above a threshold level. The cell titer is an amount that depends on the gene of interest expressed; thus, an amount that may be considered high for a certain gene of interest, may be considered low for another, and vice versa. For example, in some aspects the cell titer threshold to select a candidate cell for the generation of a landing pad cell line is at least about 1 g/L, at least about 2 g/L, at least about 3 g/L, at least about 4 g/L, at least about 5 g/L, at least about 6 g/L, at least about 7 g/L, at least about 8 g/L, at least about 9 g/L, at least about 10 g/L, at least about 11 g/L, at least about 12 g/L, at least about 13 g/L, at least about 14 g/L, at least about 15 g/L, at least about 16 g/L, at least about 17 g/L, at least about 18 g/L, at least about 19 g/L or at least about 20 g/L. In some aspects the cell titer threshold to select a candidate cell for the generation of a landing pad cell line is about 1 g/L, about 2 g/L, about 3 g/L, about 4 g/L, about 5 g/L, about 6 g/L, about 7 g/L, about 8 g/L, about 9 g/L, about 10 g/L, about 11 g/L, about 12 g/L, about 13 g/L, about 14 g/L, about 15 g/L, about 16 g/L, about 17 g/L, about 18 g/L, about 19 g/L or about 20 g/L. In some aspects the cell titer threshold to select a candidate cell for the generation of a landing pad cell line is about 1 g/L to about 2 g/L, about 2 g/L to about 3 g/L, about 3 g/L to about 4 g/L, about 4 g/L to about 5 g/L, about 5 g/L to about 6 g/L, about 6 g/L to about 7 g/L, about 7 g/L to about 8 g/L, about 8 g/L to about 9 g/L, about 9 g/L to about 10 g/L, about 10 g/L to about 11 g/L, about 11 g/L to about 12 g/L, about 12 g/L to about 13 g/L, about 13 g/L to about 14 g/L, about 14 g/L to about 15 g/L, about 15 g/L to about 16 g/L, about 16 g/L to about 17 g/L, about 17 g/L to about 18 g/L, about 18 g/L to about 19 g/L, or about 19 g/L to about 20 g/L.

In some aspects, the cell titer threshold to select a candidate cell for the generation of a landing pad cell line is at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, or at least about 200% higher than the titer observed in a reference cell line expressing the same gene of interest. In some aspects, the cell titer threshold to select a candidate cell for the generation of a landing pad cell line is about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, or about 200% higher than the titer observed in a reference cell line expressing the same gene of interest. In some aspects, the cell titer threshold to select a candidate cell for the generation of a landing pad cell line is about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 110%, about 110% to about 120%, about 120% to about 130%, about 130% to about 140%, bout 140% to about 150%, about 150% to about 160%, about 160% to about 170%, about 170% to about 180%, about 180% to about 190%, or about 190% to about 200% higher than the titer observed in a reference cell line expressing the same gene of interest.

In some aspects, the cell titer threshold to select a candidate cell for the generation of a landing pad cell line is at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold higher than the titer observed in a reference cell line expressing the same gene of interest. In some aspects, the cell titer threshold to select a candidate cell for the generation of a landing pad cell line is about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, or about 20-fold higher than the titer observed in a reference cell line expressing the same gene of interest. In some aspects, the cell titer threshold to select a candidate cell for the generation of a landing pad cell line is about 2-fold to about 3-fold, about 3-fold to about 4-fold, about 4-fold to about 5-fold, about 5-fold to about 6-fold, about 6-fold to about 7-fold, about 7-fold to about 8-fold, about 8-fold to about 9-fold, about 9-fold to about 10-fold, about 10-fold to about 11-fold, about 11-fold to about 12-fold, about 12-fold to about 13-fold, about 13-fold to about 14-fold, about 14-fold to about 15-fold, about 15-fold to about 16-fold, about 16-fold to about 17-fold, about 17-fold to about 18-fold, about 18-fold to about 19-fold, or about 19-fold to about 20-fold higher than the titer observed in a reference cell line expressing the same gene of interest.

In some aspects, the copy number threshold to select a candidate cell for the generation of a landing pad cell line is the presence of only one copy of the parental plasmid. In some aspects, the copy number threshold to select a candidate cell for the generation of a landing pad cell line is the presence of two copies of the parental plasmid.

In some aspects, in particular when there is more than one copy of the parental plasmid integrated at a location in the genome of the candidate cell, the plasmid configuration to select a candidate cell for the generation of a landing pad cell line is a head-to-tail configuration, i.e., both copies of the parental plasmid are in the same orientation.

In some aspects, a candidate cell is selected for the generation of a landing pad cell line if the RNA expression level of the parental plasmid is above a threshold level. In some aspects the RNA expression level threshold to select a candidate cell for the generation of a landing pad cell line is at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, or at least about 200% higher than the RNA expression level observed in a reference cell line expressing the same gene of interest.

In some aspects, the RNA expression level threshold to select a candidate cell for the generation of a landing pad cell line is at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold higher than the RNA expression level observed in a reference cell line expressing the same gene of interest.

The identification of suitable cell lines allowed the identification of potential homologous recombination locations within the parental plasmid, which could then be used to derive landing pad cell lines from the parental lines. Thus, this methodology allows transitioning from a random integration cell line development program to a targeted integration strategy.

### Kits

The present disclosure also provides kits and articles of manufacture for practicing any of the methods disclosed herein, e.g., kits and articles of manufacture comprising a cell (e.g., a landing pad cell or a parental cell), a landing pad plasmid, a plasmid to make a second GOI plasmid to be used to make the expression cell generated according to the methods disclosed herein, or any combination thereof, and optionally instructions for use. In some aspects, the kit comprises at least one guide RNA, a plasmid that expresses the site-specific recombinase, the recombinase protein itself, a plasmid to make a transcript that encodes the recombinase, or any combination thereof.

### EXAMPLES

### Example 1

### Identification of Parental Cell Lines to Generate Landing Pad Cell Lines

A strategy was used to identify one or more suitable parental cell lines to be used as landing pad cell lines without the need to construct new cell lines. This was accomplished by analyzing historical set of cell lines generated with conventional random integration for desired productivity and performance capabilities in which the expression cassette is integrated at but one locus in the genome. This analysis efficiently identified “hot cells” and their respective “hot spots” (genomic locations) using a biologically relevant protein of interest

Southern blot data, and expression plasmid copy number data determined by qPCR for a number cell line development projects were screened to identify parental cell lines with a single integration site containing a low copy number of 1-2 expression plasmids.

Example of identification of two suitable cell lines is given in FIG. 2, which summarizes the strategy used to find two suitable cell lines. The parental cell lines Cell Line 1 and Cell Line 2 are cell lines that express a mAb directed at a specific target, respectively. LC=light chain, and HC=heavy chain. Copy number=number expression plasmids in the cell line. Each expression plasmid contained a LC and HC expression cassette. Copy number was determined by qPCR using GAPDH as internal control. spPCR=splinkeret PCR. This technology allowed the identification of plasmid junction sequences. The level of LC RNA and HC RNA was normalized to that found for Cell Line 1. The transcript levels in Cell Line 2 were therefore 20% higher than that of Cell Line 1.

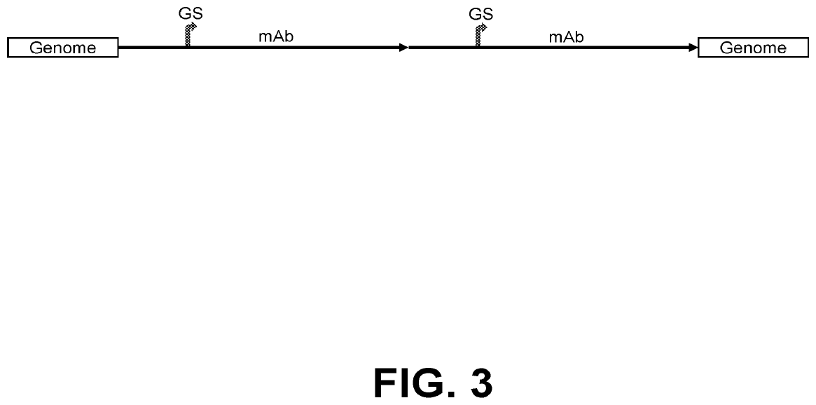

The configuration of the monoclonal antibody expression plasmids observed in each cell line is given in FIG. 2 and FIG. 3. FIG. 3 is a simplified depiction of the parental plasmids showing the configuration found in both cell line 1 and cell line 2. The parental plasmids in both cell lines were in a head to tail configuration. The configuration in cell line 1 and cell line 2 was established by Southern blot analysis and determination of plasmid sequence junctions in which the plasmid-plasmid fusion was detected. The arrow and GS in FIG. 3 represent glutamine synthetase complementation.

The identification of suitable cell lines allowed the identification of potential homologous recombination locations within the parental plasmid, which could then be used to derive landing pad cell lines from the parental lines. Thus, this methodology allows transitioning from a random integration cell line development program to a targeted integration strategy.

The 5′ and 3′ plasmid sequence junctions for parental cell line 1 were identified. The CHO genomic sequence corresponding to the 5′ junction is provided in the sequence set forth in SEQ ID NO:18, and the CHO genomic sequence corresponding to the 3′ junction is provided in the sequence set forth in SEQ ID NO:19.

### Example 2

### Generation of Landing Pad Cell Lines from Candidate Parental Cell Lines

When a cell line is identified as described above, the desire is to either replace the expression cassette in the parental cell line with an alternative plasmid such as the landing pad plasmid (FIG. 4A) or use it directly as the landing pad cell line (FIG. 4B). Using this approach, homologous recombination reactions occur by using the sequences flanking the original plasmid in the equivalent of the parental cell line thus requiring knowledge of the cellular (genomic) flanking sequences. The parental cell line in FIG. 4B has been established as supporting high expression from low copy number of expression plasmids.

### Example 3

### Universal Landing Pad Cells

Direct insertion of expression or landing pad plasmids into the cellular genome by homologous recombination as known in the art requires identification of the cellular sequences to be used for homologous recombination. This strategy runs the risk of missing potentially good parental cell lines since often times there is a sister chromosome(s) with the same sequence restricted by the site specific nuclease(s) potentially resulting in deleterious effects.

An alternative method has been developed for making landing pad cells that is independent of knowledge about cellular (genomic) flanking sequences, and for using the parental plasmid in the parental cell line as the landing pad itself. This strategy provides multiple advantages over current industrial strategies by having no need for (1) identifying sufficient flanking cellular sequence to allow design of a suitable site specific endonuclease, (2) the generation and cloning of the regions for homologous recombination onto the landing pad, (3) avoiding potential deleterious sister chromatid restriction. The method is (4)universal in nature as it is applicable to all expression cell lines, (5) and faster and cheaper than the alternative genome dependent strategies. Combining the parental cell selection method disclosed above with the method to generate universal landing pad cells provided herein results in a particularly efficient strategy for generation and validation of landing pads and their associated “hot clones” or “hot cell line” for use as landing pad cells. Using this strategy, the same plasmid vector setup is used, without requiring the identification of flanking chromosomal regions.

An exemplary schematic the method is presented in FIG. 5A. We used a parental cell line that was expressing a monoclonal antibody and used the plasmid sequences flanking the expression cassette in the parental cell line as the sites of homologous recombination. Site specific endonucleases targeted plasmid sequences in the parental cell line, thereby avoiding sites in sister chromosomes. The sequences targeted by the site directed endonuclease were absent in the second plasmid (landing pad plasmid). If the targeted sequences were in the landing pad plasmid, they are removed. As show in FIG. 5A, the second plasmid (P2) carried Lox sites, encoded for a fluorescent marker (b1mCherry), and expressed a selection marker (puromycin resistance) that was different from the original expression plasmid present in the parental cell line.

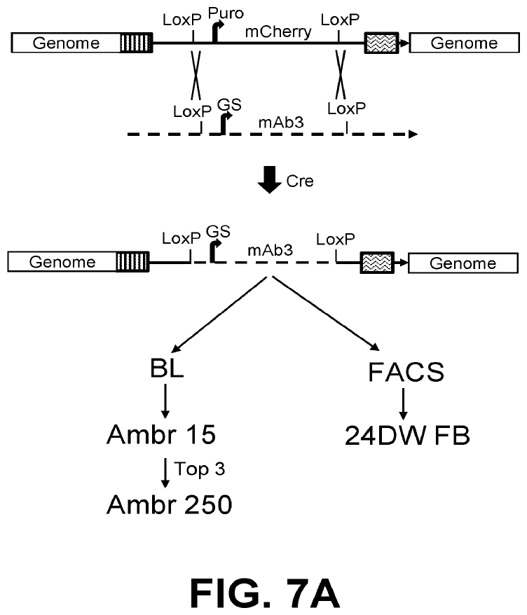

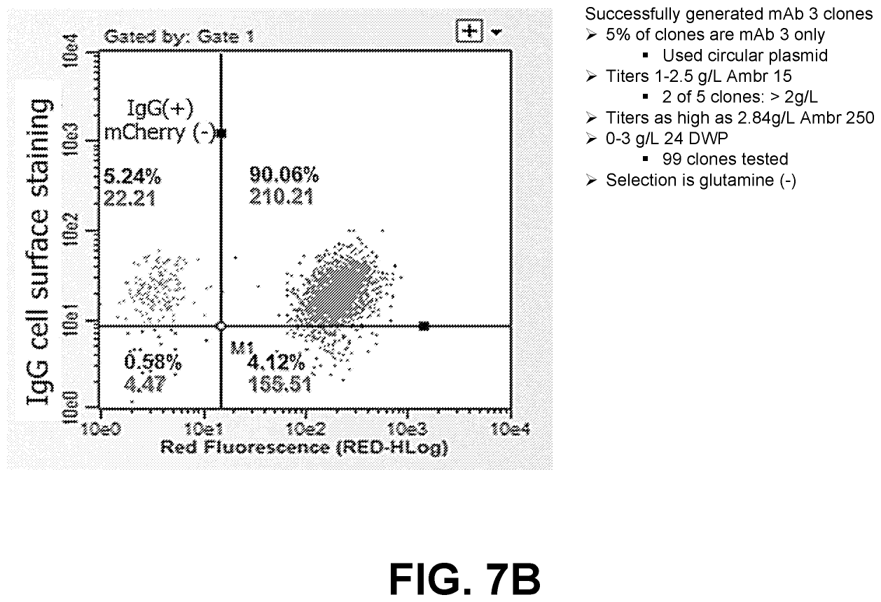

In the presence of the site specific endonuclease (CRISPR/Cas) and the second plasmid, landing pad cells lines were generated (FIG. 6A). Using the Lox sites present in the landing pad cell line a third plasmid (P3) with an expression cassette for a biologic flanked by Lox sites was used to replace the mCherry/puromycin coding sequences by Cre directed recombination (FIG. 7A).

Accordingly, all the steps that were represented FIG. 5A were conducted by starting with the parental cell line selected from one of the two parental cell lines tested in Example 1, which was expressing an antibody (a first gene of interest), and making a expressing cell line capable of expressing a gene (a second gene of interest) encoding the mAb3 antibody at >2.5 g/L (FIGS. 7A and 7B). The second parental cell line was used to create landing pad cell line.

In the experimental data presented, the entire process presented in FIG. 5A was conducted. However, the step necessary to generate the mCherry/puromycin intermediate landing pad cell line can be skipped entirely as shown in FIG. 5B. In this case the parental cell line's mAb expression plasmid functions as the landing pad. Thus, the mAb expression plasmid functioning as the landing pad is replaced in this case with a different mAb expression plasmid directly by homologous recombination stimulated by site specific endonucleases. This method takes less time than that disclosed in FIG. 5A and allows direct assessment of the parental cell line's suitability for targeted integration.

A schematic of two alternative formats that use site specific recombination in the presumptive invention are shown in FIG. 8A. In both versions, a landing pad plasmid encodes for a fluorescent marker (b1mCherry), expresses a selection marker (puromycin resistant) that is different from the parental plasmid present in the parental cell line and are flanked, e.g., by heterologous site specific recombination sites (SSRS). The site-specific recombination sites are shown as Lox P and Lox 511 in FIG. 8A which are targets of the Cre recombinase. In the presence of the site specific endonuclease (CRISPR/Cas) and the Landing Pad Plasmid, the mAb expression cassette in the Parental Cell Line is either replaced with the landing pad shown as mCherry flanked by Lox site, or is deleted and the landing pad is integrated into an alternative locus, FIGS. 8A and 8B, respectively. In the format of FIG. 8A, the landing pad is in a hot spot which supports high expression. In format of FIG. 8B alternative hot spots can be identified. Since the parental cell line is a hot cell and identification of additional hot spots will result in Landing Pad Cell Lines able to generate Expression Cell Lines with a preferred attribute such as high titer.

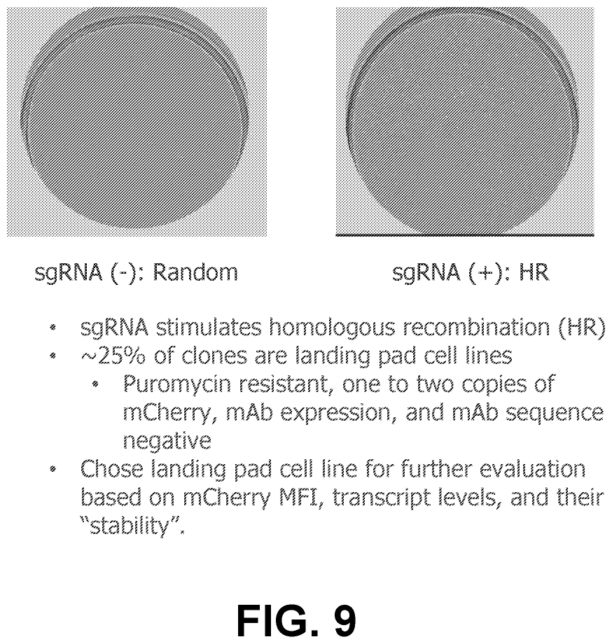

A screening strategy to identifying landing pad cell lines shown in FIGS. 8A and 8B was established (FIG. 9). Landing Pad Plasmid along with the CRISPR/Cas site-specific endonuclease were transfected into the parental cell line and Puromycin resistant cells were selected for. The use of CRISPR/Cas can stimulate generation of landing pad cell lines by promoting recombination, see FIG. 9 compare with (+) and without (−) sgRNA in the left and right pictures respectively. The presence of the sgRNA increased the numbers of mCherry positive cells indicating stimulation of recombination. Using FACS, the mCherry positive (Red+) Puromycin resistant cells were single cell cloned. Those cells that no longer express the mAb of the parental cell line were expanded and screened for the landing pad and presence of any residual light chain and heavy chain genes by a PCR based quantitative gene copy number assay. Those with no mAb sequences and only 1-2 copies of the landing pad were further evaluated. Approximately 25% of the Puromycin resistant cells are landing pad cell lines. The cells were passaged to ensure the median fluorescent intensity (MFI) and transcript levels of mCherry remained constant. Of 28 clones screened 14 had a single landing pad replacing the mAb sequence as depicted in FIG. 8A as determined by a junction specific PCR and gene copy number assessed by ddPCR. The remainder of the landing pad cell lines were in alternative loci as depicted in FIG. 8B.

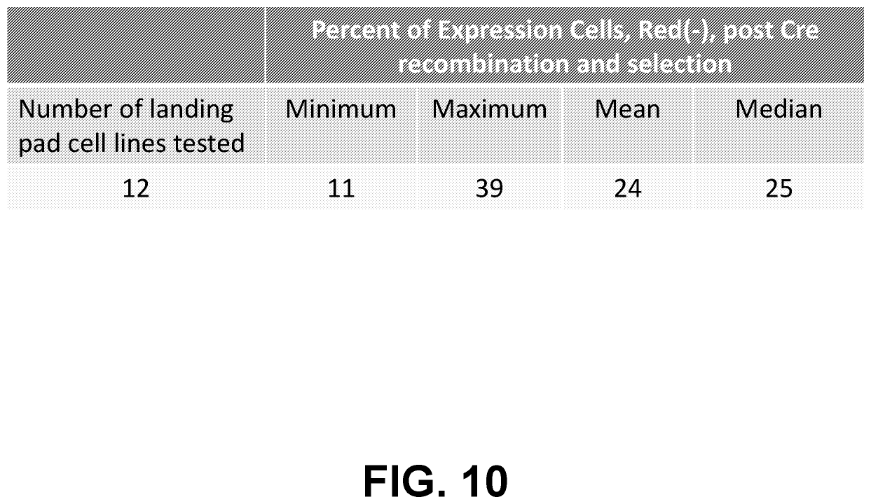

All steps represented in Strategies A, B, C, and D in FIG. 8A and FIG. 8B were successfully conducted. The performance of 12 landing pad cell lines were evaluated using a second GOI plasmid comprised of two light and two heavy chain expression cassettes to make a mAb. The first parameter evaluated was the percent of Expression Cells after Cre recombination. This was done by measuring the percent of Red(−) cells present in the bulk population after selection but before the step of single cell cloning. The percent of Red(−) Expression Cells ranged from 11 to 39 with an average of 24 for the 12 Landing Pad cell lines tested (FIG. 10). In the absence of Cre nearly all Landing Pad Cell Lines are >99% Red(+). This demonstrates the Landing Pad Cell Lines and their Lox sites are functional for Cre directed recombination.

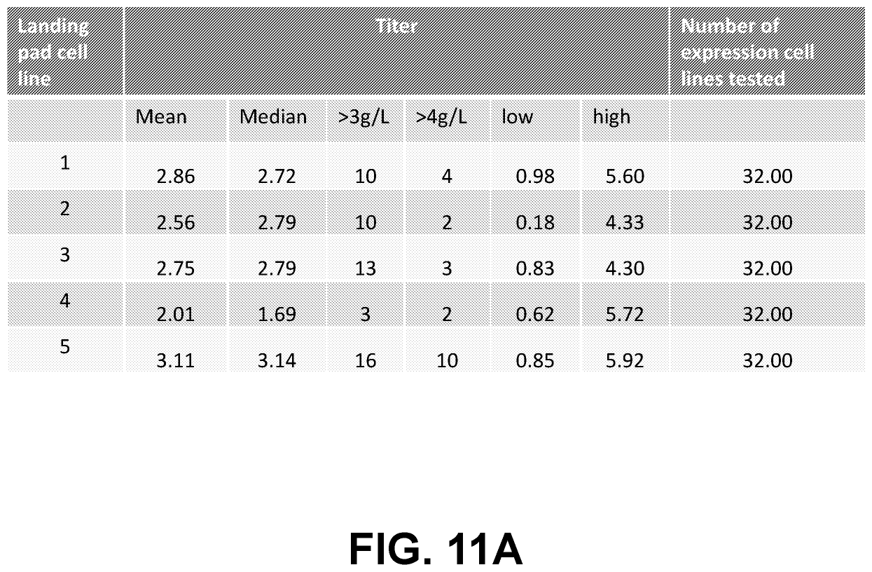

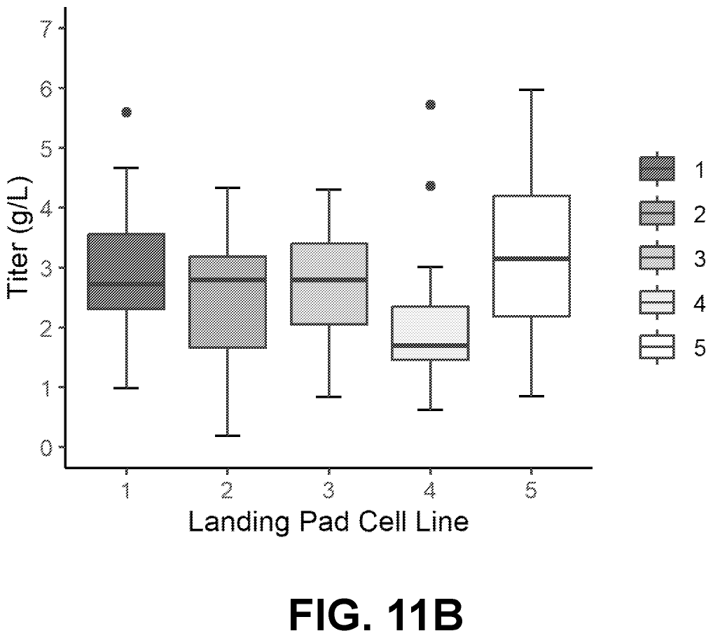

Expression Cell Lines representative of FIG. 8A were generated from 5 of the 12 Landing Pad Cell Lines by FACS sorting on Red(−) cells. Thirty two Expression Cell Lines per landing pad cell line were picked at random, expanded and their productivities determined using a 24 deep well pate (DWP) fed batch assay the results of which are shown in FIGS. 11A and 11B. All Landing Pad Cell Lines generated multiple Expression Cell Lines with median titers >1.69 g/L, with multiple clones each having titers >3 g/L, and a few with titers >4 g/L demonstrating all of the Landing Pad Cell Lines are capable of generating Expression Cell Lines suitable for manufacturing purposes.

These high expressing cell lines were identified with no intervening screen after single cell cloning from only 32 randomly chosen clones saving weeks of time and drastically reducing number of clones needed to be screened both of which are of high value. In addition, the 5 Landing Pad Cell Lines tested are statistically indistinguishable from each other. These data demonstrate the Parental Plasmid locus is a hot spot, and the Universal TI strategy outlined in FIGS. 8A and 8B is valid since multiple Landing Pad Cell Lines were generated out of this one locus with relatively minimal screening.

The technology to produce Expression Cell Lines as shown in FIGS. 8A and 8B replaces at least a portion of the landing pad. It is known in the art of landing pad technology where no replacement is required including (Thyagarajan, B., Olivares, E. C., Hollis, R. P., Ginsburg, D. S. and Calos, M. P. (2001) Site-specific genomic integration in mammalian cells mediated by phage phiC31 integrase. Mol. Cell. Biol., 21, 3926-3934; Gaidukov, L., Wroblewska, L., Teague, B., Nelson, T., Zhang, X., Liu, Y., Jagtap, K., Mamo, S., Tseng, W. A., Lowe, A. et al. (2018) A multi-landing pad DNA integration platform for mammalian cell engineering. Nucleic Acids Res., 46, 4072-4086; Sauer, B. and Henderson, N. (1990) Targeted insertion of exogenous DNA into the eukaryotic genome by the Cre recombinase. New Biol, 2, 441-449; Fukushige, S. and Sauer, B. (1992) Genomic targeting with a positive-selection lox integration vector allows highly reproducible gene expression in mammalian cells. Proc Natl Acad Sci USA, 89, 7905-7909). These alternative landing pads and associated technology can be used in place of the Cre/Lox landing pad design disclosed in FIG. 8A.

### Example 4

### Duo-Landing Pad Cells

Above, landing pad cell lines are described that contain a single landing pad. However, landing pad cell lines with more than one landing pad provide an opportunity to further refine expression of multisubunit biologics such as bispecific monoclonal antibodies. We therefor screened for landing pad cell lines with two landing pads in the same locus, a duo-landing pad. This would ensure equal expression from both landing pads as they reside in the same locus. The duo-landing pads can integrate in four different orientations head-to-head, tail-to-tail, tail-to-head and head-to-tail (FIG. 12B). When a single site directed recombinase such as Cre or Flp are used the head-to-head and tail-to-tail configurations are preferred and functionally undistinguishable from each other. Unlike in the tail to head and head to tail configurations that in the presence of Cre can result in deletion of one of the landing pads, the other two configurations will simply go through inversion resulting in the same starting configuration (FIG. 12B). We generated such a duo-landing pad cell line in the head-to-head configuration. It is in an alternate locus other than where the mAb of the parental cell line resided as described in FIG. 8B.

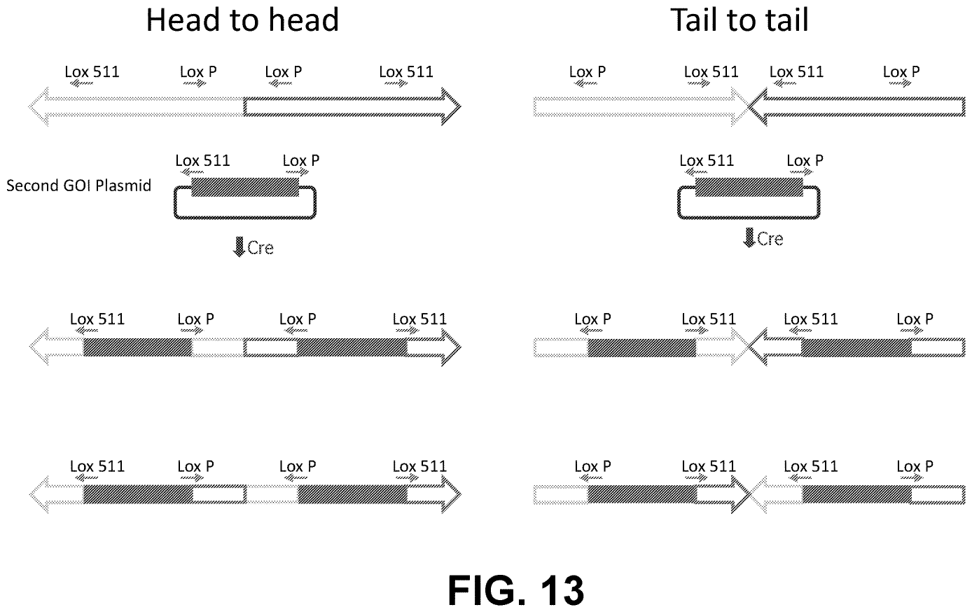

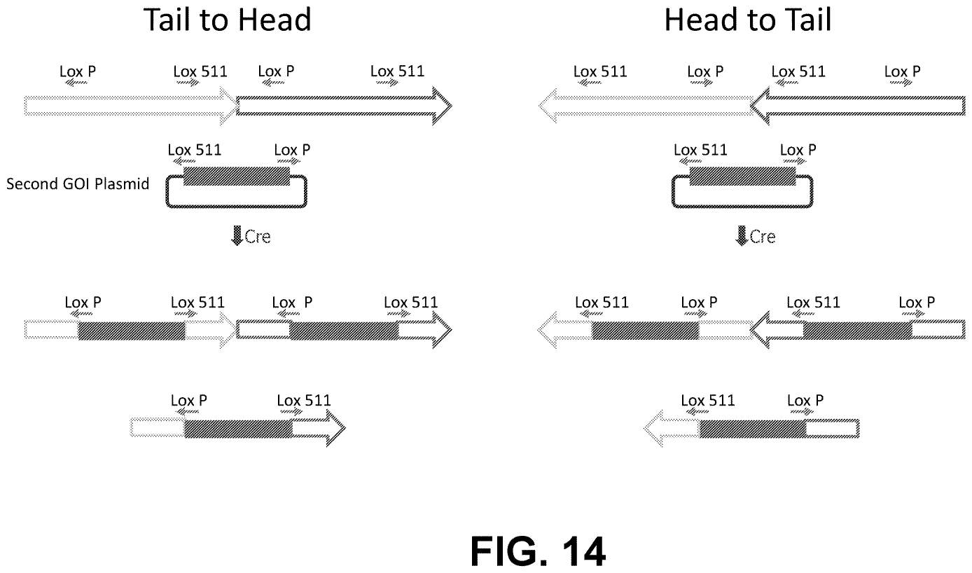

When a Second GOI Plasmid is used with each of the four duo-landing pad configurations of FIG. 12B, the head-to-head and tail-to-tail configurations can each generate two cell lines where the sequences between the two recombination sites flanking the plasmid junction can be inverted, otherwise the two cell lines are the same (FIG. 13). When the head to tail or tail to head configurations are used with the Second GOI Plasmid cell lines with two Second GOI are produced. However, if there is sufficient amounts of Cre activity present one of the Second GOI can be removed resulting in a Second GOI Plasmid cell line with a single Second GOI (FIG. 14).

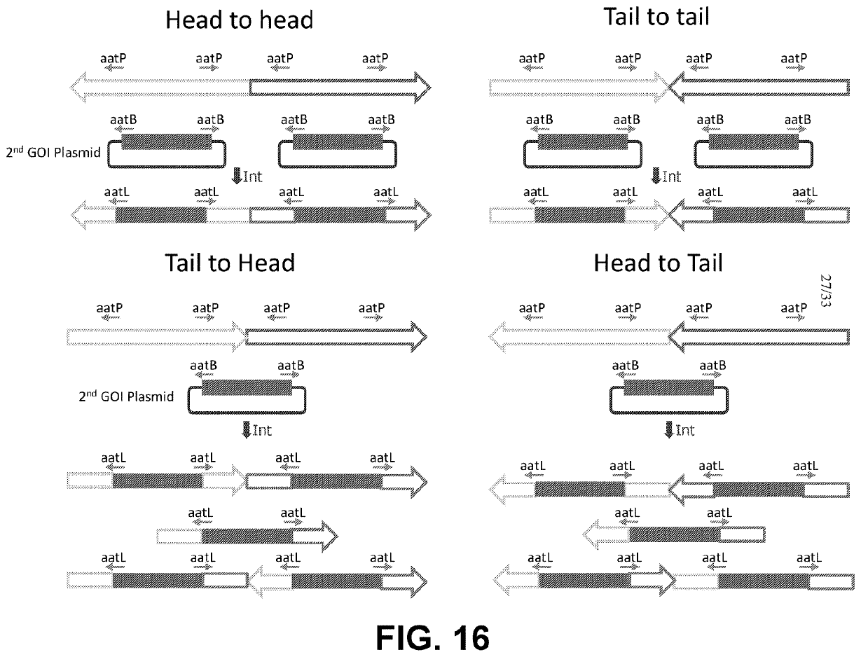

If the landing pad uses a Frt recognition site for Flp in place of say Lox 511 in FIG. 12B, and both Cre and Flp are used, the same outcome will result, compare FIG. 12A with FIG. 15, with deletion in tail to head and head to tail orientations, while the head to head and tail to tail orientations go through inversions (FIG. 15). However, recombining the Second GOI into the duo-landing pad using attP/attB with integrase in the tail to tail and head to head configurations results in no inversions, but in the tail to head and head to tail configurations the deletion of one of the landing pads can still occur (FIG. 16). If each of the landing pads has but say one attP site then a single integration of a circular Second GOI Plasmid with a single attB site would occur resulting in no deletions occurring in any of the four duo-landing pad configurations of FIG. 16.

The duo landing pad can be used simultaneously with multiple different GOI plasmid. It has been disclosed the use of a Landing Pad Cell Line with a single landing pad with multiple different expression cassettes needed to make a biologic. The use of a duo-Landing Pad Cell Line has advantages over a landing pad cell line with a single landing pad. In the case of the single landing pad cell line, all expression cassettes needed to make a multicomponent biologic must be placed in a single Second GOI Plasmid as the cell line only accommodates a single Second GOI. That is not the case with the duo-Landing Pad Cell Line. The duo-Landing Pad Cell line affords the opportunity to design in greater expression diversity levels providing the opportunity to create an Expression Cell Line with superior characteristics.

The diversity can be generated in multiple ways using different configurations of the Second GOI Plasmids. In one instance the Second GOI Plasmids contain all expression cassettes needed to make the complex biologic in unique configurations. In a second instance the Second GOI Plasmids may contain a subset of the expression cassettes that need to reside in the same cell to make an expression cell line. In a third instance a combination of the two previous instances where one or more Second GOI Plasmids having all the expression cassettes in unique configurations needed to make the complex biologic along with a set of Second GOI Plasmids that contains a subset of all the expression cassettes in unique configuration(s).

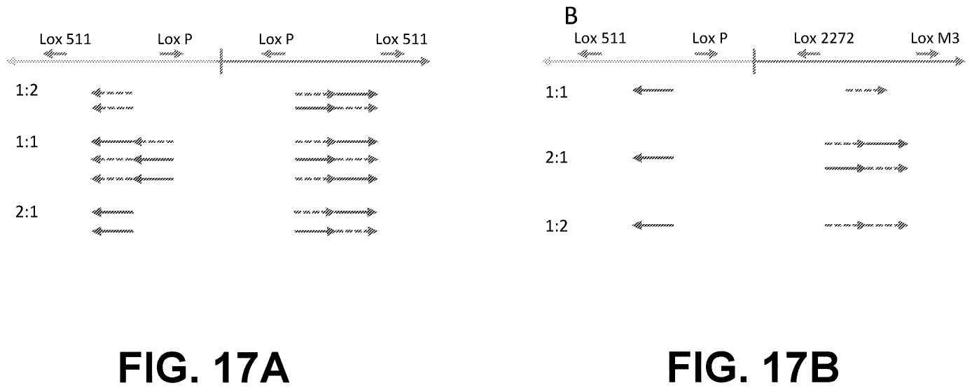

For illustration purposes only, a simplified rendition of the diversity that can be achieved is shown in FIG. 17A. Each landing pad is comprised of a Lox 511 and Lox P pairing. Here the expression cassettes needed to make the complex biologic is divided into two sets one represented by the solid arrow and the other by the dashed arrow. When both sets are found in a single Second GOI Plasmid they can be in different configurations as illustrated by the tandem arrows in a solid-dashed and dashed-solid arrangement. Also shown as single arrows are Second GOI Plasmids that contain only one of the two sets of expression plasmids. Not shown is a single solid and dashed arrows in each landing pad. Seven different Expression Cell Lines are shown with different combinations of the two sets of expression cassettes. The ratio of the two sets in an Expression Cell Line is shown at the left. As is readily evident the complexity increases greatly compared to having a single landing pad. It is possible to readily screen such a diverse set of Expression Cell Lines to find one of superior characteristics (Altamura, R., Doshi, J. and Benenson, Y. (2022) Rational design and construction of multi-copy biomanufacturing islands in mammalian cells. Nucleic Acids Res., 50, 561-578).

Although in FIGS. 12A to 17A show duo-landing pad configurations where both landing pads have the same recombinase or Int recognition sequence it is possible to make each landing pad have a unique recombination “address”. In the case of recombinases such as Cre and Flp four unique recognition sequences would be used. Each landing pad would have a unique pairing of recognition sites. An example is in shown in FIG. 18 using four incompatible Lox sites (Langer, S. J., Ghafoori, A. P., Byrd, M. and Leinwand, L. (2002) A genetic screen identifies novel non-compatible loxP sites. Nucleic Acids Res., 30, 3067-3077; Missirlis, P. I., Smailus, D. E. and Holt, R. A. (2006) A high-throughput screen identifying sequence and promiscuity characteristics of the loxP spacer region in Cre-mediated recombination. BMC Genomics, 7, 73; Siegel, R. W., Jain, R. and Bradbury, A. (2001) Using an in vivo phagemid system to identify non-compatible loxP sequences. FEBS Lett., 505, 467-473). Examples of additional strategies include replacing two Lox sites in FIG. 18 with two incompatible Frt sites and using Cre with Frt (Lauth, M., Spreafico, F., Dethleffsen, K. and Meyer, M. (2002) Stable and efficient cassette exchange under non-selectable conditions by combined use of two site-specific recombinases. Nucleic Acids Res., 30, e115), using an integrase with two to four incompatible aat sites (Jusiak, B., Jagtap, K., Gaidukov, L., Duportet, X., Bandara, K., Chu, J., Zhang, L., Weiss, R. and Lu, T. K. (2019) Comparison of Integrases Identifies Bxb1-GA Mutant as the Most Efficient Site-Specific Integrase System in Mammalian Cells. ACS Synth Biol, 8, 16-24.), using more than one integrase for example that of BxB1 (Jusiak, B., Jagtap, K., Gaidukov, L., Duportet, X., Bandara, K., Chu, J., Zhang, L., Weiss, R. and Lu, T. K. (2019) Comparison of Integrases Identifies Bxb1-GA Mutant as the Most Efficient Site-Specific Integrase System in Mammalian Cells. ACS Synth Biol, 8, 16-24.) and phiC31(Smith, M. C., Brown, W. R., McEwan, A. R. and Rowley, P. A. (2010) Site-specific recombination by phiC31 integrase and other large serine recombinases. Biochem. Soc. Trans., 38, 388-394), and combinations thereof. The use of a single att site in each landing pad is sufficient for insertion of the Second GOI Plasmids into each landing pad (FIG. 19). In this case the Second GOI Plasmid is required to be circular as a linear plasmid would effectively restrict the chromosome. It is also clear the landing pad can contain multiple att sites so that each contains a unique address. These examples are not meant to be limiting in scope.

The duo-landing pad configuration with the landing pads with unique addresses can also be used to generate a more defined diversity of Expression Cell Lines compared to when they are not addressable (see FIGS. 17A and 17B), and higher diversity to a Landing Pad cell line with a single landing pad. A simplified illustration using landing pads with unique addresses is shown in FIG. 17B. One landing pad is comprised of Lox 511 and Lox P, and the second with Lox sites 2272 and M3. The description of arrows is the same as that for FIG. 17A given above. In this example it is known a particular Second GOI is desired but the remainder of what is needed to express the complex biologic is not well defined so four different Second GO are placed in the adjacent landing pad resulting in four different Expression Cell Lines. It is possible to readily screen such a diverse set of Expression Cell Lines to find one of superior characteristics (Altamura, R., Doshi, J. and Benenson, Y. (2022) Rational design and construction of multi-copy biomanufacturing islands in mammalian cells. Nucleic Acids Res., 50, 561-578).

An additional application of the addressable landing pads is the option to have two independent biologics expressed each with its own independent function. One of the biologics could help the Expression Cell Line express the second biologic, or the first biologic could cause a particular post translational modification of the second biologic or modify some other component of the Expression Cell Line. These are simply examples and are not intended to be to be limiting in nature. It is clear these same uses apply when the two landing pads do not have a unique address as in FIG. 17A just a higher level of diversity is obtained.

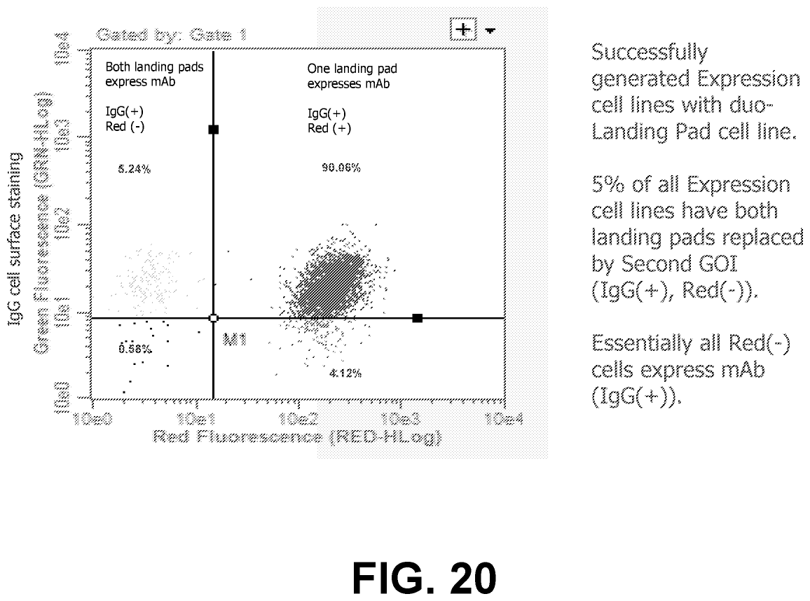

The utility of the duo-Landing pad cell line was reduced to practice using a head to head configuration in an alternative locus to that of the Parental Plasmid of the Parental Cell Line. The Second GOI Plasmid contains a single copy of light chain and heavy chain genes, and GS selection cassette as shown in FIGS. 8A and 8B. The percent of Expression Cells after Cre recombination was determined. This was done by measuring the number of Red(−) mAb(+) cells where mAb expression was detected by IgG cell surface staining and the results are shown in (FIG. 20). After recovery from selection 6.24% of both landing pads were replaced by the Second GOI. Since essentially all Red(−) cells are mAb(+), single cell cloning on Red(−) cells by FACS for example enables isolation of only Expression cell lines.

This allows for elimination of the expansion and C50 productivity screen during Selection phase, and static screen during Clone Development in cell line development (FIG. 1). It also reduces the number of Expression Cell lines needed to be screened during the Clone Development phase (FIG. 1). FIG. 1 depicts historical cell line development using random integration. This was a standard cell line development strategy in which a cell line was transfected with a linearized expression plasmid resulting in it integrating at random locations in the cell's genome. After transfection the cells were subdivided into plates and subjected to selection such as drug (puromycin) or auxotroph complementation (glutamine synthetase (GS)). Only cells with the expression plasmid survived and were expanded during master well development. Thousands of cells from the top productive master wells were single cell cloned for clone development to ensure a high expressing clone could be found. The clones were expanded and subjected to multiple rounds of screening until the top candidate clones were identified. The top 6 clones were identified (Top 6 RCB) and further evaluated (RCB Clone Selection) for suitability for manufacturing purposes and the end of which the top clone was identified.

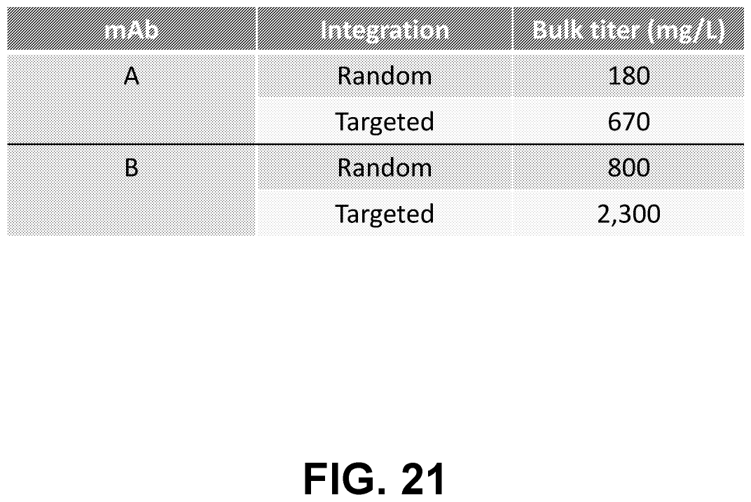

The value of targeted integration over random integration was evaluated by making Expression Cell Lines for two mAbs. The duo-Landing Pad cell line and a CHO host cell line were transfected with the respective Second GOI Plasmids with or without Cre recombinase respectively. Following transfection the cells went through selection and expanded till sufficient cells were available to seed a c50 tube and test their productivity. The results are presented in FIG. 21. Targeted integration generated titers >3 fold that of the random integrated cells. Since these are total populations it demonstrates that targeted integration on average makes significantly higher expressing cell lines compared to random integration.

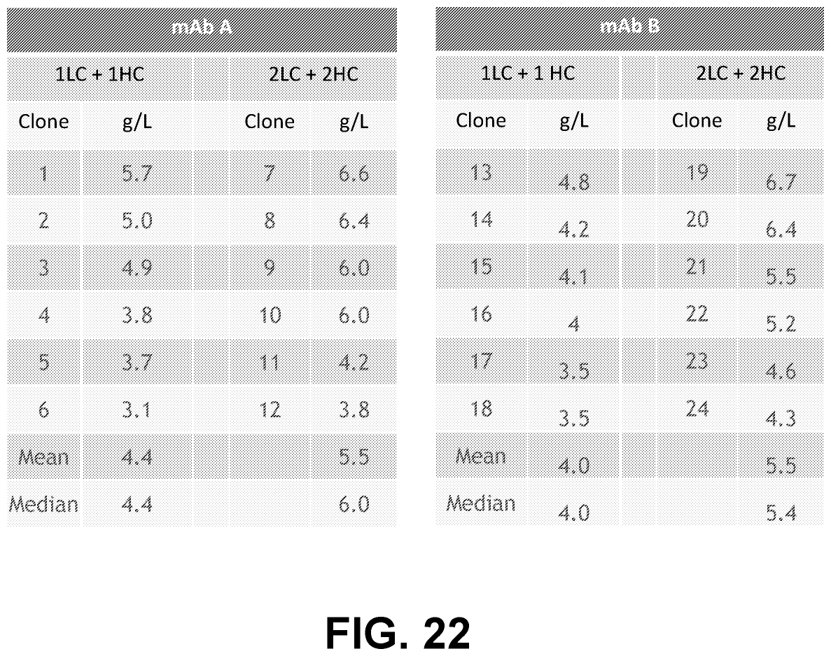

The duo-Landing Pad cell line is able to produce biologics at relevant levels. Two different Second GOI Plasmid configurations having either 1 LC and 1 HC, or 2 LC and 2 HC expression cassettes were used with the duo-Landing Pad cell line to make mAb A and mAb B. The top 6 clones from each were evaluated for each mAb in a scale down model of a manufacturing bioreactor (FIG. 22). The titers ranged from 3.1 to 5.7 g/L and 3.5 to 4.8 g/L for mAb A and mAb B respectively for the 1 LC and 1 HC configuration. Titers for both mAb A and mAb B increased when the 2 LC and 2 HC Second GOI plasmid was used generating titers that ranged from 3.8 to 6.6 g/L and 4.3 to 6.7 g/L respectively. This represents a 25% and 38% average titer increase for mAb A and mAb B respectfully demonstrating changes in GOI Plasmid configuration can increase titers. The data also demonstrates the duo-Landing Pad reproducibly generates high titer Expression Cell Lines. Genetic characterization by Southern blot and long read DNA sequencing of 12 Expression Cell Lines demonstrated they all arose from Cre directed recombination into the landing pads (data not shown).

These data validate the universal strategy to make a TI Landing Pad cell lines as outlined in FIGS. 5B and 8 with clear utility, generating populations with higher expressing Expression Cell lines and reducing time to make Expression Cell lines with relevant productivities compared to random integration technology. It also validates the functionality of the duo-landing pad design, and the locus where the duo-landing pad resides as a hot spot.

### Example 5

### Landing Pad Hot Spots

In addition to the previous disclosures we also provide the loci of the “hot spots” that have been identified. These “hot spot” loci are unique and provide locations in which one or multiple landing pads can be inserted. The loci can be used independently of each other or in combination. The present disclosure provides two landing pad hot spots (HOT SPOT 1 and HOT SPOT 2),

HOT SPOT 1 is located within gi|14971555981ref|NW_020822499.1 from Cricetulus griseus (SEQ ID NO:22). In some aspects, HOT SPOT 1 is located within SEQ NO:20. 5′ sequences suitable for homologus recombination are provided in SEQ ID NOS: 16 and 18. 3′ sequences suitable for homologus recombination are provided in SEQ ID NOS: 17 and 19. In some aspects, the integration site in HOT SPOT 1 comprises or consist of the sequence set forth in SEQ ID NO: 21.

HOT SPOT 2 is located within ref|NW_020822577.1 from Cricetulus griseus (SEQ ID NO:118). In some aspects, HOT SPOT 2 is located within SEQ ID NO: 116. 5′ sequences suitable for homologus recombination are provided in SEQ ID NOS: 112 and 114. 3′ sequences suitable for homologus recombination are provided in SEQ ID NOS: 113 and 115. In some aspects, the integration site in HOT SPOT 2 comprises or consist of the sequence set forth in SEQ ID NO: 117. HOT SPOT 2 is particularly advantageous because no open reading frames are included in its sequence.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

The contents of all cited references (including literature references, patents, patent applications, and websites) that may be cited throughout this application are hereby expressly incorporated by reference in their entirety for any purpose, as are the references cited therein, in the versions publicly available on Nov. 15, 2022. Protein and nucleic acid sequences identified by database accession number and other information contained in the subject database entries (e.g., non-sequence related content in database entries corresponding to specific Genbank accession numbers) are incorporated by reference, and correspond to the corresponding database release publicly available on Nov. 15, 2022.

What is claimed is:

1. A method to select a parental cell suitable for the development of a landing pad cell line comprising:
(i) screening and selecting a cell line with a high expression titer of a gene of interest (GOI); and,
(ii) further screening a cell of (i) and selecting a cell with a low copy number of a parental plasmid comprising the nucleic acid encoding the GOI, wherein the copy number is one or two.

2. The method of claim 1, wherein the parental plasmid comprises two site-specific recombination sites (SSRS), one SSRS, or no SSRS.

3. A method to select a landing pad cell comprising:
(i) screening for the loss of the parental plasmid or a portion thereon in a parental cell line, and selecting a cell with such a loss (deletion); and,
(ii) further screening a cell of (i) for the presence of a landing pad, and selection a cell in which a landing pad is present.

4. A method to select a landing pad cell comprising:
(i) screening for the loss of at least one parental plasmid or a portion thereon in a parental cell line, and selecting a cell with such a loss (deletion); and,
(ii) further screening a cell of (i) for the presence of at least one landing pad, and selection a cell in which a landing pad is present.

5. The method of claim 3 or claim 4 further comprising screening the landing pad sequence in the landing pad cell for characteristics selected from the group consisting of
(i) presence or absence of regions of low complexity or high complexity;
(ii) presence or absence of retrotransposon sequences;
(iii) presence or absence of Alu repeats;
(iv) presence or absence of long interspersed nuclear elements (LINE);
(v) presence or absence of CpG islands;
(vi) levels of cytosine methylation;
(vii) levels of histone acetylation;
(viii) presence or absence of active transcription; and,
(ix) any combination thereof.

6. A method of generating a landing pad cell comprising
(i) deleting at least one parental plasmid or a portion thereof comprising a first GOI in a parental cell line, and
(ii) introducing into the cell, following the at least one deletion, a landing pad plasmid or portion thereof comprising a landing pad.

7. The method of claim 6, wherein the landing pad plasmid or portion thereof comprising a landing pad is inserted at the site of a deletion of (i).

8. The methos of claim 6, wherein the landing pad plasmid or portion thereof comprising a landing pad is inserted at a site which is not the site of a deletion of (i).

9. A method of generating a landing pad cell comprising:
integrating a landing pad plasmid into the genome of a parental cell at a targeted-integration site using homologous recombination, wherein the sequences targeted for homologous recombination are located in the parental plasmid
wherein each landing pad plasmid comprises
(1) a polynucleotide sequence comprising a nucleic acid encoding at least one selection marker and/or at least one nucleic acid sequence encoding a detectable marker;
(2) two site-specific recombination sites (SSRS) flanking the polynucleotide sequence of (1); and,
(3) two homologous recombination sites located 5′ and 3′ terminally with respect to the SSRSs of (2), which are homologous to corresponding homologous recombination sites in a parental plasmid; and,
wherein the homologous recombination sites of the landing pad plasmid recombine with the corresponding homologous recombination sites of the parental plasmid, thereby integrating the landing pad plasmid at an internal location within the parental plasmid inserted in the parent cell genomic DNA.

10. The method of any one of claims 6 to 9, wherein the parental plasmid is located in more than one genomic locus.

11. A method for identifying a landing pad cell comprising
(1) removing at least a portion of the First GOI from a parental plasmid integrated in the genomic sequence of a parental cell;
(2) integrating a landing pad plasmid at alternative genomic loci;
(3) screening a library of candidate cells comprising at least one copy of the landing pad plasmid integrated at at least one alternative genomic loci, wherein a candidate cell line is evaluated for one or more of the following properties
(a) cell titer is above a predetermined threshold level;
(b) landing pad plasmid or landing pad copy number is at predetermined value;
(c) RNA expression level above a predetermined threshold level,
(d) multiple plasmid copies, if present, have a specific plasmid configuration;
(e) deletion of at least a portion of the First GOI from a parental plasmid; and,
(f) presence of at least one landing pad with functional SSRS.

12. The method of claim 11, wherein the parental cell is a historical cell line.

13. The method of claim 11, wherein the library of candidate cells is a library generated via random integration of the landing pad sequence at multiple locations in the genome of a parental cell.

14. The method of any one of claims 11 to 13, wherein the method selects a hot cell with the landing pad sequence integrated in a hot spot.

15. The method of any one of claims 11 to 14, wherein the parental cell line is a CHO cell line.

16. A method of generating an expression cell comprising integrating a second GOI plasmid into the genome of a landing pad cell according to claims 3-15 using site-specific recombinase recombination, wherein the resulting expression plasmid comprises
(1) a polynucleotide sequence comprising a nucleic acid encoding a second GOI; and,
(2) two SSRS flanking the polynucleotide of (1);
wherein the site-specific recombination sites of the landing pad plasmid recombine with the corresponding site-specific recombination sites of the second GOI plasmid, thereby integrating the GOI plasmid at an internal location within the landing pad plasmid of the landing pad cell.

17. A method of generating an expression cell comprising:
(a) integrating a landing pad plasmid or portion thereof into the genome of a parental cell at a targeted-integration site using homologous recombination, wherein the sequences targeted for homologous recombination are located in the parental plasmid,
wherein each landing pad plasmid or portion thereof comprises
(1a) a polynucleotide sequence comprising a nucleic acid encoding at least one selection marker and/or at least one nucleic acid sequence encoding a detectable marker;
(2a) two SSRS flanking the polynucleotide sequence of (1a); and,
(3a) two homologous recombination sites located 5′ and 3′ terminally with respect to the SSRSs of (2a), which are homologous to corresponding homologous recombination sites in a parental plasmid; and,
wherein the homologous recombination sites of the landing pad plasmid or portion thereof recombine with the corresponding homologous recombination sites of the parental plasmid in the landing pad at a different genomic locus, thereby integrating the landing pad plasmid or portion thereof at an internal location within the landing pad at the different genomic locus in the parent cell genomic DNA; and,
(b) integrating a second GOI plasmid into the genome of the landing pad cell using site-specific recombinase recombination, wherein the expression plasmid comprises
(1b) a polynucleotide sequence comprising a nucleic acid encoding a GOI; and,
(2b) two SSRS flanking the polynucleotide of (1b);
wherein the site-specific recombination sites of the landing pad plasmid recombine with the corresponding site-specific recombination sites of GOI plasmid, thereby integrating the GOI plasmid at an internal location within the landing pad plasmid of the landing pad cell.

18. A method of generating a landing pad cell comprising:
(a) removing at least a portion of a parental plasmid from a first hot spot location in a parental cell line; and,
(b) integrating a landing pad plasmid into a second hot spot location in the genome of the parental cell at a targeted-integration site using homologous recombination or random integration, wherein the sequences targeted for homologous recombination or random integration were present in the landing pad plasmid
wherein each landing pad plasmid comprises
(1) a polynucleotide sequence comprising a nucleic acid encoding at least one selection marker and/or at least one nucleic acid sequence encoding a detectable marker;
(2) two site-specific recombination sites (SSRS) flanking the polynucleotide sequence of (1); and,
(3) two homologous recombination sites located 5′ and 3′ terminally with respect to the SSRSs of (2), which are homologous to corresponding homologous recombination sites in parental cell line genome.

19. A method of generating an expression cell comprising:
(a) removing a parental plasmid or a portion thereof from a first hot spot location in a parental cell line,
(b) integrating a landing pad plasmid into a second hot spot location in the genome of the parental cell at a targeted-integration site using homologous recombination, wherein the sequences targeted for homologous recombination were present in the parent cell line
wherein each landing pad plasmid comprises
(1b) a polynucleotide sequence comprising a nucleic acid encoding at least one selection marker and/or at least one nucleic acid sequence encoding a detectable marker;
(2b) two site-specific recombination sites (SSRS) flanking the polynucleotide sequence of (1b); and,
(3b) two homologous recombination sites located 5′ and 3′ terminally with respect to the SSRSs of (2b), which are homologous to corresponding homologous recombination sites in a parental cell line,
wherein the homologous recombination sites of the landing pad plasmid recombine with the corresponding homologous recombination sites of the parental cell line, thereby integrating the landing pad plasmid at an internal location within the parental cell genomic DNA, and,
(c) integrating a GOI plasmid into the genome of the landing pad cell using site-specific recombinase recombination, wherein the expression plasmid comprises
(1c) a polynucleotide sequence comprising a nucleic acid encoding a first GOI; and,
(2c) two SSRS flanking the polynucleotide of (1c);
wherein the site-specific recombination sites of the landing pad plasmid recombine with the corresponding site-specific recombination sites of GOI plasmid, thereby integrating the GOI plasmid at an internal location within the landing pad plasmid of the landing pad cell.

20. The method of any one of claims 1-19, wherein the landing pad cell comprises a plasmid having a topology corresponding to the description 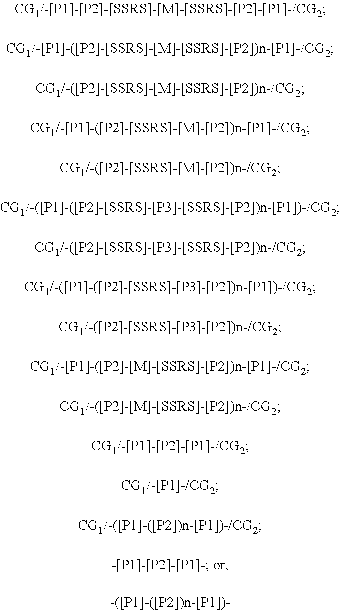
wherein
CG₁ and CG₂ are parental cell genomic sequences flanking the inserted plasmid;
[P1] is a polynucleotide sequence derived from a parental plasmid;
[P2] are polynucleotide sequences derived from a landing pad plasmid;
[M] is a polynucleotide sequence comprising at least one selection marker and/or at least one nucleic acid sequence encoding a detectable marker; and,
[SSRS] are site-specific recombination sites (SSRS).
n is an integer between 1 and 10 between 1 and 10, such as 1, 2, 3, 4, 5, 6, 7, 8, 9 or 10.

21. The method of any one of claims 16, 17 or 19, wherein the topology of the plasmid integrated in the expression cells corresponds to the description 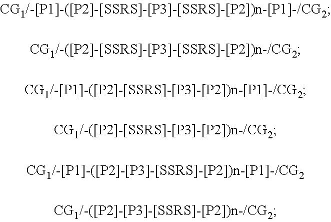
wherein
CG₁ and CG₂ are parental cell genomic sequences flanking the inserted plasmid;
[P1] is a polynucleotide sequence derived from a parental plasmid;
[P2] are polynucleotide sequences derived from a landing pad plasmid;
[P3] is a polynucleotide sequence derived from a plasmid comprising a gene of interest (GOI); and,
[SSRS] are site-specific recombination sites (SSRS).
n is an integer between 1 and 10, such as 1, 2, 3, 4, 5, 6, 7, 8, 9 or 10.

22. The method of any one of claims 9 or 17-21, wherein the homologous recombination is mediated by a CRISPR/Cas system, a TALEN system, or a ZFN system.

23. The method of claim 22, wherein the CRISPR/Cas system further comprises a single guide RNA (sgRNA).

24. The method of any one of claims 2 or 9-23, wherein the site-specific recombinase recombination site (SSRS) is a Tyr-recombinase site, a Tyr-integrase site, a Serine-resolvase/invertase site, or a Serine-integrase site.

25. The method of claim 24, wherein the Tyr-recombinase site comprises a Cre, Dre, Flp, KD, B3, or B3 Tyr-recombinase site.

26. The method of claim 24, wherein the Tyr-integrase site comprises a λ (Lambda), HK022, or HP1 Tyr-integrase site.

27. The method of claim 24, wherein the Serine-resolvase/invertase site comprises a γδ (Gammadelta), ParA, Tn3, or Gin Serine-resolvase/integrase site.

28. The method of claim 24, wherein the Serine-integrase site comprises a PhiC31, Bxb1, pr R4 Serine-integrase site.

29. The method of claim 24, wherein the Tyr-recombinase site comprises a Cre Tyr-recombinase site.

30. The method of claim 24, wherein the SSRS is a LoxP site.

31. The method of claim 30, wherein the LoxP site comprises a nucleic acid sequence set forth in SEQ ID NO:1 (wild type LoxP).

32. The method of claim 30, wherein the LoxP site comprises a mutant LoxP site.

33. The method of claim 32, wherein the mutant LoxP site comprises a nucleic acid sequence set forth in SEQ ID NO:2 (mutant LoxP).

34. The method of claim 32, wherein the mutant LoxP site comprises a nucleic acid selected from the group consisting of SEQ ID NO: 3 (Lox 511); SEQ ID NO: 4 (Lox 5171); SEQ ID NO: 5 (Lox 2272); SEQ ID NO: 6 (Lox M2); SEQ ID NO: 7 (Lox M3); SEQ ID NO: 8 (Lox M7); SEQ ID NO: 9 (Lox M11); SEQ ID NO: 10 (Lox 71); and, SEQ ID NO: 11 (Lox 66).

35. The method of claim 24, wherein the Tyr-recombinase site comprises a Flp Tyr-recombinase site.

36. The method of claim 35, wherein the SSRS is a short flippase recognition target (FRT) site.

37. The method of claim 24, wherein the Serine-integrase site comprises an attP or attB site.

38. The method of method of any one of claims 9, 10, or 17-37, wherein the at least one selection marker and/or at least one nucleic acid sequence encoding a detectable marker is glutamine synthetase (GS) and/or dihydrofolate reductase (DHFR).

39. The method of method of any one of claims 9, 10, or 17-38, wherein the at least one selection marker and/or at least one nucleic acid sequence encoding a detectable marker is a drug resistance gene.

40. The method of claim 39, wherein the drug resistance gene is an antibiotic resistance gene.

41. The method of claim 40, wherein the antibiotic resistance gene is a puromycin resistance gene.

42. The method of claim 41, wherein the puromycin resistance gene is puromycin-N-acetyltransferase.

43. The method of method of any one of claims 9, 10, or 17-42, wherein the at least one selection marker and/or at least one nucleic acid sequence encoding a detectable marker comprises a protein.

44. The method of claim 43, wherein the protein is a fluorescent protein.

45. The method of claim 44, wherein the fluorescent protein is mCherry.

46. The method of claim 44, wherein the fluorescent protein comprises GFP, ZsGreen1, AcGFP1, EGFP, GFPuv, AcGFP, EBFP, EYFP, ECFP, tdTomato, mCherry, DsRed, AmCyan, ZsGreen, ZsYellow, DsRed2, DsRed-Express, HcRed, AsRed, mOrange, mOrange2, mPlum, mStrawberry, mBanana, YFP, mRaspberry, HcRed1, E2-Crimson, or any combination thereof.

47. The method of any one of claims 1 to 46, wherein the cell is a Chinese Hamster Ovary (CHO) cell.

48. The method of any one of claims 1 to 46, wherein the cell is HEK293 or NS0.

49. The method of any one of claims 1, 2, 6-8, 11-17, or 19-48, wherein the nucleic acid encoding the GOI encodes at least one polypeptide.

50. The method of claim 49, wherein the at least one polypeptide is an antibody or a fusion protein.

51. The method of any one of claims 16, 17 or 19-50, wherein the expression plasmid comprises one, two, or more than two copies of the GOI, a detectable marker, or a combination thereof.

52. The method of claim 51, further comprising determining the expression of the GOI, detectable marker, or combination thereof.

53. The method of claim 52, wherein the expression of the GOI is determined quantitatively and/or qualitatively.

54. The method of claim 52 or claim 53, wherein the expression of the GOI is determined by cell sorting, FACS, cell surface staining, Western blot, Northern blot, column chromatography, capillary electrophoresis, microfluidics, UV absorbance, immunohistochemistry, cell size, secreted protein levels, transcript levels, or any combination thereof.

55. The method of any one of claims 3 to 54, wherein the landing pad plasmid or expression plasmid is integrated with a copy number of 1 in the genome of the cell.

56. The method of any one of claims 3 to 55, wherein the landing pad plasmid or expression plasmid is integrated with a copy number of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 in the genome of the cell.

57. The method of any one of claims 9-10 or 17-56, wherein
(i) the 5′ homologous recombination site comprises a polynucleotide sequence of SEQ ID NO: 18 or a subsequence thereof, and the 3′ homologous recombination site comprises a polynucleotide sequence of SEQ ID NO: 19 or a subsequence thereof,
(ii) the 5′ homologous recombination site comprises a polynucleotide sequence of SEQ ID NO: 114 or a subsequence thereof, and the 3′ homologous recombination site comprises a polynucleotide sequence of SEQ ID NO: 115 or a subsequence thereof; or,
(iii) the 5′ homologous recombination site and the 3′ homologous recombination site comprise polynucleotide sequences flanking the parental plasmid.

58. The method of any one of claims 1 to 57, wherein the parental plasmid comprises an open reading frame (ORF) encoding a first GOI such as an antibody.

59. A landing pad cell comprising a plasmid having a topology corresponding to the description 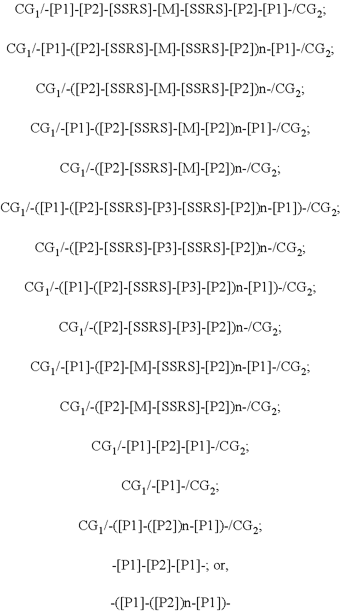
wherein
CG₁ and CG₂ are parental cell genomic sequences flanking the inserted plasmid;
[P1] is a polynucleotide sequence derived from a parental plasmid;
[P2] are polynucleotide sequences derived from a landing pad plasmid;
[M] is a polynucleotide sequence comprising at least one selection marker and/or at least one nucleic acid sequence encoding a detectable marker; and,
[SSRS] are site-specific recombination sites (SSRS).
n is an integer between 1 and 10 between 1 and 10, such as 1, 2, 3, 4, 5, 6, 7, 8, 9 or 10.

60. An expression cell comprising a plasmid with a topology corresponding to the description 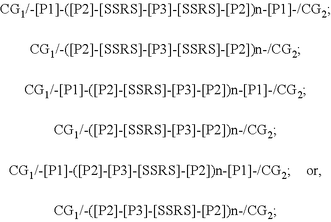
wherein
CG₁ and CG₂ are parental cell genomic sequences flanking the inserted plasmid;
[P1] is a polynucleotide sequence derived from a parental plasmid;
[P2] are polynucleotide sequences derived from a landing pad plasmid;
[P3] is a polynucleotide sequence derived from a plasmid comprising a gene of interest (GOI); and,
[SSRS] are site-specific recombination sites (SSRS).
n is an integer between 1 and 10, such as 1, 2, 3, 4, 5, 6, 7, 8, 9 or 10.

61. A cell line produced by the methods of any one of claims 3 to 60.

62. A kit comprising a cell of claim 61 or a cell generated according to the method of any one of claims 1 to 61 and instructions for their use.

63. An isolated cell comprising a polynucleotide sequence which comprises a nucleic acid encoding a gene of interest (GOI) integrated at a specific locus of the genome of the cell, wherein the locus is a nucleotide position or polynucleotide subsequence within SEQ ID NO: 20 or SEQ ID NO: 116.

64. A method comprising introducing into CHO cells a polynucleotide sequence which comprises a nucleic acid encoding a gene of interest (GOI) and obtaining a CHO cell wherein the nucleic acid is integrated at a specific locus of the genome of the CHO cell, wherein the locus is a nucleotide position or polynucleotide subsequence within SEQ ID NO: 20 or SEQ ID NO: 116.

65. A method comprising providing a cell comprising a polynucleotide sequence which comprises a nucleic acid encoding a gene of interest (GOI), wherein the polynucleotide sequence comprises the nucleic acid encoding a gene of interest (GOI) operably linked to a promoter, wherein the nucleic acid is integrated at a specific locus of the genome of the CHO cell, and wherein the locus is a nucleotide position or polynucleotide subsequence within SEQ ID NO: 20 or SEQ ID NO: 116.

66. The isolated cell of claim 63 or method of claim 64 or 65, wherein (i) the nucleotide subsequence within SEQ ID NO: 20 comprises the sequence set forth in SEQ ID NO:21; or (ii) the nucleotide subsequence within SEQ ID NO: 116 comprises the sequence set forth in SEQ ID NO: 117.

67. The isolated cell of claim 63 or method of claim 64 or 65, wherein (i) the nucleotide subsequence from within SEQ ID NO: 20 consists of the sequence set forth in SEQ ID NO:21; or (ii) the nucleotide subsequence from within SEQ ID NO: 116 consists of the sequence set forth in SEQ ID NO: 117.

68. The isolated cell of claim 63 or method of claim 64 or 65, wherein (i) the nucleotide subsequence from within SEQ ID NO: 20 is a subsequence upstream (towards the 5′ end of SEQ ID NO: 20) with respect to the sequence set forth in SEQ ID NO:21; or (ii) the nucleotide subsequence from within SEQ ID NO: 116 is a subsequence upstream (towards the 5′ end of SEQ ID NO: 116) with respect to the sequence set forth in SEQ ID NO: 117.

69. The isolated cell of claim 63 or method of claim 64 or 65, wherein (i) the nucleotide subsequence from within SEQ ID NO: 20 is a subsequence downstream (towards the 3′ end of SEQ ID NO: 20) with respect to the sequence set forth in SEQ ID NO:21, or (ii) the nucleotide subsequence from within SEQ ID NO: 116 is a subsequence downstream (towards the 3′ end of SEQ ID NO: 116) with respect to the sequence set forth in SEQ ID NO:117.

70. The method of any one of claims 1-58 or 64-69, cell of claims 59-60, 63 or 66-69, cell line of claim 61, or kit of claim 62, comprising at least two landing pad plasmids or at least two expression plasmids.

71. The method, cell, cell line, or kit of claim 70, wherein the two landing pad plasmids or two expression plasmids are in a configuration selected from the group consisting of head-to-head, tail-to-tail, tail-to-head, and head-to-tail.

72. The method, cell, cell line, or kit of any one of claim 70 or 71, wherein each expression plasmid comprises at least a nucleic acid encoding a gene of interest (GOI).

73. The method, cell, cell line, or kit of claim 72, wherein all GOI are the same.

74. The method, cell line, kit, or isolated cell of claim 72, wherein all GOI are different.

75. The method, cell, cell line, or kit of claim 72, wherein at least one GOI is different from the rest.

76. The method, cell, cell line, or kit of any one of claim 74 or 75, wherein a first GOI comprises a heavy chain (HC) of an antibody, and a second GOI comprises a light (LC) of an antibody.

77. The method, cell, cell line, or kit of any one of claims 70 to 76, wherein at least one expression plasmid is bicistronic.

78. The method, cell, cell line, or kit of claim 77, wherein the bicistronic expression plasmid encodes a first GOI comprising a HC of an antibody, and a second GOI comprising a LC of an antibody.

79. The method, cell, cell line, or kit of any one of claims 70 to 78, wherein at least one landing pad plasmid is addressable.

80. The method, cell, cell line, or kit of any one of claims 70 to 79, wherein each landing pad plasmid comprises two Lox sites.

81. The method, cell, cell line, or kit of claim 80, wherein the Lox sites are Lox P and Lox 511.

82. The method, cell, cell line, or kit of any one of claims 70 to 81, wherein each landing pad plasmid comprises a Lox site and an Frt site.

83. The method, cell, cell line, or kit of any one of claims 70 to 81, wherein each landing pad plasmid comprises one or two aat sites.

84. The method, cell, cell line, or kit of any one of claims 70 to 83, wherein each landing pad plasmid is addressable.

85. The method, cell, cell line, or kit of claim 84, wherein each addressable landing pad plasmid comprises a pair of addressable SSRS which are unique to the landing pad.

86. The method, cell, cell line, or kit of claim 85, wherein at least one pair of addressable SSRS is a pair of Lox sites.

87. The method, cell, cell line, or kit of claim 86, wherein at least one pair of Lox sites is Lox 511 and Lox P.

88. The method, cell, cell line, or kit of claim 86, wherein at least one pair of Lox sites is Lox m3 and Lox m7.

89. The method, cell, cell line, or kit of any one of claims 84 to 88, comprising a first addressable landing pad plasmid comprises an Lox 511 and Lox P pair of Lox sites, and a second addressable landing pad plasmid comprises an Lox m3 and Lox m7 pair of Lox sites.

90. The method, cell, cell line, or kit of claim 84, wherein each addressable landing pad plasmid comprises a non cross-compatible att site.

91. A cell comprising a heterologous polynucleotide sequence which comprises a nucleic acid encoding a gene of interest (GOI), a selection marker, a detectable marker, or a combination thereof integrated at a specific locus of the genome of the cell, wherein the locus is a nucleotide position or polynucleotide subsequence within SEQ ID NO: 20 or SEQ ID NO: 116 or an orthologous sequence thereof.

92. A cell comprising a heterologous polynucleotide sequence which comprises a nucleic acid encoding a gene of interest (GOI), a selection marker, a detectable marker, or a combination thereof integrated at a specific locus of the genome of the cell, wherein the locus is a nucleotide position or polynucleotide subsequence within SEQ ID NO: 21 or SEQ ID NO: 117 or an orthologous sequence thereof.

93. A cell comprising a heterologous polynucleotide sequence which comprises a nucleic acid encoding a gene of interest (GOI), a selection marker, a detectable marker, or a combination thereof integrated at a specific locus of the genome of the cell, wherein the locus is a polynucleotide subsequence that overlaps or encompasses SEQ ID NO: 20 or SEQ ID NO: 116 or an orthologous sequence thereof.

94. A cell comprising a heterologous polynucleotide sequence which comprises a nucleic acid encoding a gene of interest (GOI), a selection marker, a detectable marker, or a combination thereof integrated at a specific locus of the genome of the cell, wherein the locus is a polynucleotide subsequence that overlaps or encompasses SEQ ID NO: 21 or SEQ ID NO: 117 or an orthologous sequence thereof.

95. The cell of any one of claims 91 to 94, wherein the cell is a CHO cell.

96. The cell of any one of claims 91 to 95, wherein the orthologous sequence has about 70%, about 75%, about 80%, about 85%, about 90%, about 95%, about 96%, about 96%, about 97%, about 98% or about 99% sequence identity to SEQ ID NO: 20, 21, 116, 117 or subsequence thereof.

97. The cell of claim 98, wherein sequence identity is determined via pairwise alignment using an implementation of the Needleman-Wunsch algorithm.

98. The cell of any one of claims 91 to 95, where the cell comprises two landing pad plasmids or two expression plasmids.

99. The cell of any one of claims 91 to 98, wherein the cell comprises more than two landing pad plasmids or more than two expression plasmids.

100. The cell of claim 98 or 99, wherein the two landing pad plasmids are addressable.
